(19) United States
(12) Patent Application Publication
(10) Pub. No.: US 2006/0079696 A1
(43) Pub. Date: Apr. 13, 2006

(54) Substituted compounds derived from N-(benzyl)phenylacetamide, preparation and uses

(75) Inventors: Christophe Masson, Lambersart (FR); Karine Caumont-Bertrand, Frelinghien (FR)

Correspondence Address: NIXON & VANDERHYE, PC, 901 NORTH GLEBE ROAD, 11TH FLOOR, ARLINGTON, VA, 22203, US

(73) Assignee: GENFIT, Loos (FR)

(21) Appl. No.: 11/235,707

(22) Filed: Sep. 27, 2005

(30) Foreign Application Priority Data
Sep. 27, 2004 (FR) 04 10192

(51) Int. Cl. C07C 311/44; C07D 263/08

(52) U.S. Cl. 548/237; 564/86; 562/450; 562/104; 564/123

(57) ABSTRACT

This invention relates to poly-substituted derivatives of the N-(benzyl)phenylacetamide type, pharmaceutical compositions comprising same, therapeutic uses thereof, more particularly in the fields of human and animal health. This invention also relates to a process for the preparation of such derivatives.

This invention relates to polysubstituted derivatives of the N-(benzyl)phenylacetamide type, pharmaceutical compositions comprising same, therapeutic uses thereof, more particularly in the fields of human and animal health. This invention also relates to a process for preparing these derivatives.

The inventive compounds represent a beneficial therapeutic tool for improving pathologies linked to disorders of the lipidic and/or glucidic metabolism (hyperlipidemia, hyperglycemia, etc.). They can be used more particularly in order to prevent or treat pathologies associated with Syndrome X, insulin resistance, diabetes, dyslipidemias, atherosclerosis, cardiovascular diseases, obesity, hypertension, inflammatory diseases, etc.

By their simultaneous action upon several risk factors for cardiovascular diseases, the inventive compounds allow to reduce the global cardiovascular risk.

### BACKGROUND OF THE INVENTION

Coronary diseases, cerebral ischemia and peripheral arterial diseases are the main cardiovascular diseases according to the International Atherosclerosis Society (IAS 2003). Cardiovascular diseases are nowadays the main cause of death in adults in most developed countries and in certain developing countries. There are many factors contributing to their causes, more particularly linked to modern life-style: tobacco, eating habits (atherogenic), sedentarity (lack of physical activity), etc. The discovery of strategies for effectively treating and/or preventing these pathologies has become a matter of urgency worldwide.

Diabetes, obesity, dyslipidemias (high plasmatic rates of LDL cholesterol and triglycerides, low HDL cholesterol, etc.) and hypertension are some of the cardiovascular risk factors which have been clearly identified (IAS 2003). Epidemiological studies have shown that there is a synergic effect among these different factors. The concomitant presence of several of them leads to a dramatic aggravation of cardiovascular risk. It is appropriate therefore to speak of global risk for cardiovascular diseases. The current therapeutic strategies consist of associating several medicines so as to reduce the different risk factors individually. Nevertheless, the combination of drugs can sometimes bring about serious secondary reactions: for example, the simultaneous administration of fibrates and statins increases the risk of myopathy (Denke 2003).

The necessity of obtaining compounds which act both upon the metabolic pathologies and upon the cardiovascular risk has been demonstrated (Laight 2003). There is nowadays a real need for products capable of acting concomitantly upon the different cardiovascular risk factors. Such products would allow:
to reduce the risk of cardiovascular disease, and
to treat each disorder and its consequences independently (dyslipidemias, diabetes, pathologies associated with Syndrome X, hypertension, obesity, atherosclerosis, etc.).

### SUMMARY OF THE INVENTION

Unexpectedly, the inventors discovered a family of novel molecules having a <<multimodal>> action mechanism. More particularly, the inventive compounds showed that they had an effect upon at least two types of receptors involved in major regulation processes:
The PPAR nuclear receptors (Peroxisome Proliferator-Activated Receptor) involved in numerous biological processes: regulation of lipids and glucids, inflammation, etc.;
ATP-dependent potassic channels of β-pancreatic cells, involved in the regulation of insulin secretion.

Surprisingly, the inventors showed that the inventive compounds are activators of PPAR nuclear receptors. They therefore represent a beneficial therapeutic tool (Verges 2004).

### BRIEF DESCRIPTION OF THE FIGURES

FIGS. 1a, 1b, 1c: In vitro evaluation of the PPAR activating properties of the inventive compounds according to the dose

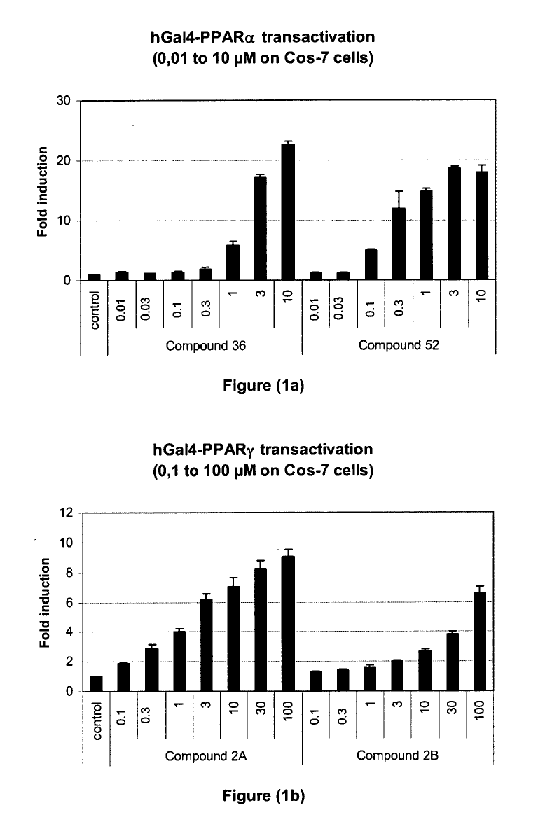

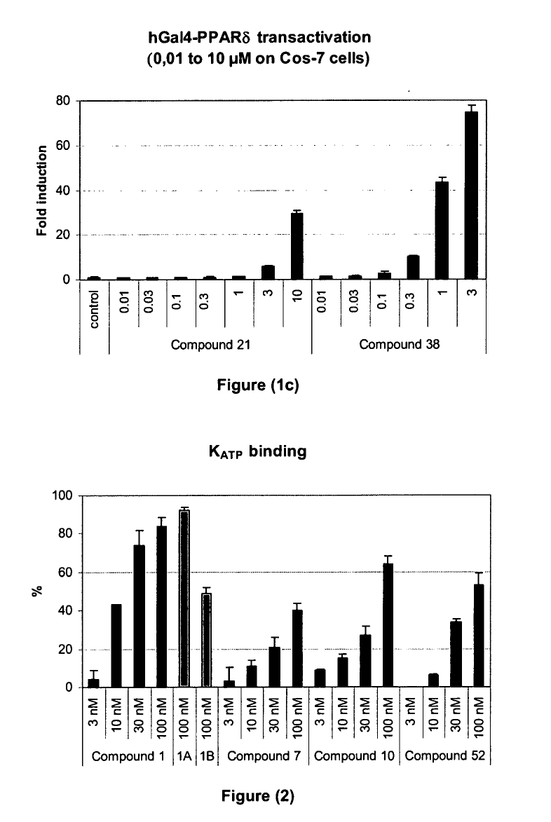

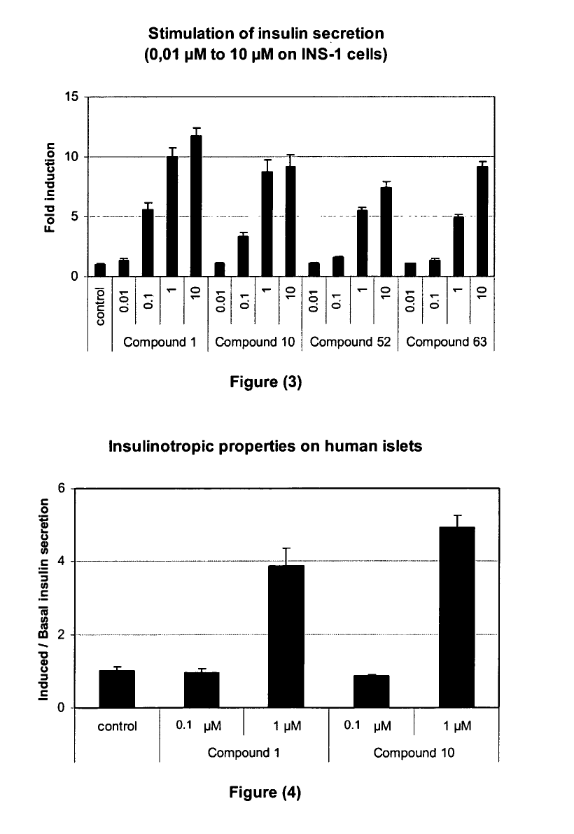

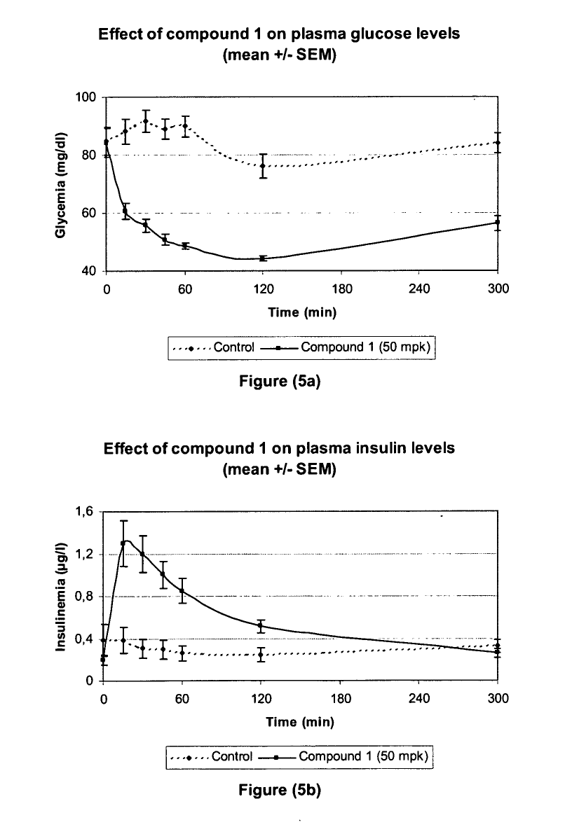

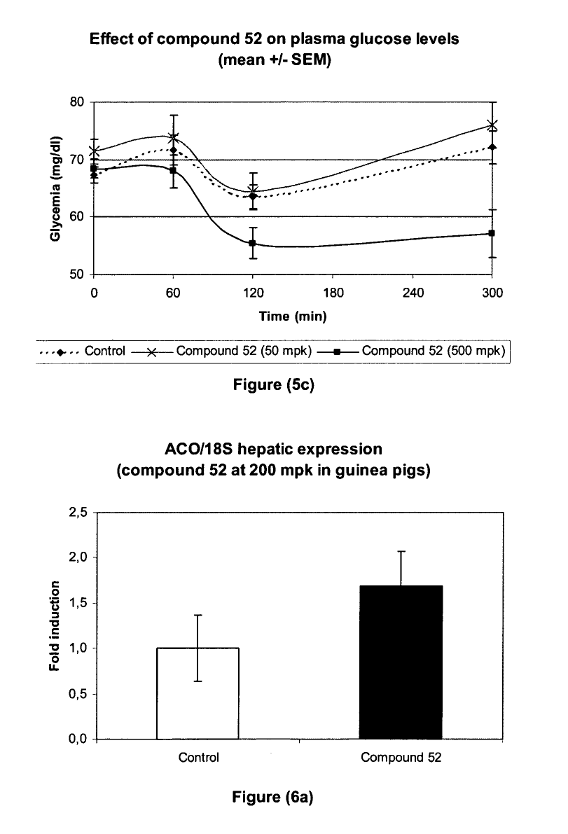

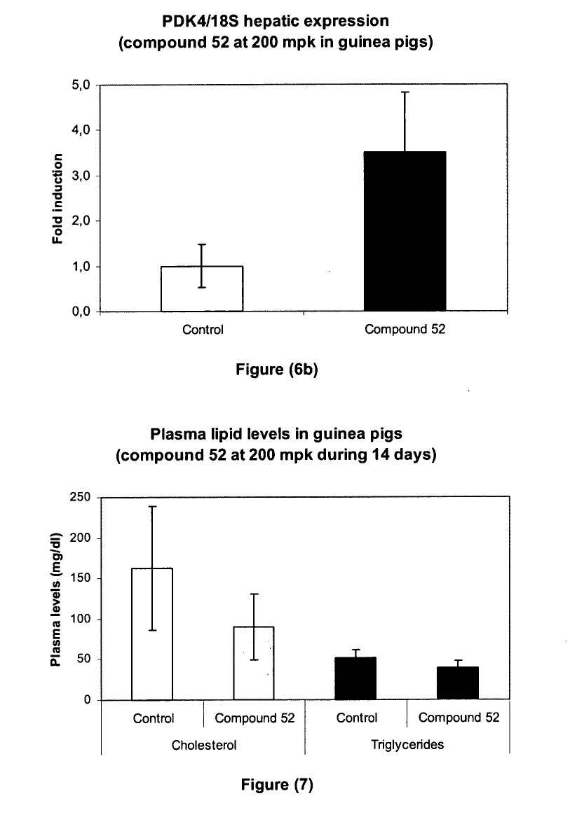

The PPAR activation was evaluated in vitro on a monkey kidney fibroblast line (COS-7), by measuring the transcriptional activity of chimeras made up of the DNA binding domain of the Gal4 transcription factor of yeast and of the binding domain to the ligand of the different PPARs.

The compounds were tested at doses between 0.01 and 100 μM on Gal4-PPARα, γ, δ chimeras. The results are shown by the induction factor for each condition, i.e. the ratio between the luminescence induced by the compound and the luminescence induced by the control: the higher this factor, the more the compound has PPAR-activating properties.

FIG. 2: In vitro evaluation of the affinity of the inventive compounds for the ATP-dependent potassium channels based upon the dose

The results presented reflect the specific affinity of the inventive compounds on the ATP-dependent potassium channels (K⁺ ATP). The specific binding measured corresponds to the difference between the total binding and the non-specific binding determined in the presence of an excess of non-marked reference ligand (glibenclamide 1 μM). The inventive compounds were tested at doses between 3 and 100 nM: the higher the percentage measured, the stronger the affinity of the compound for the ATP-dependent potassium channels.

FIG. 3: In vitro evaluation of the insulinotropic properties of the compounds according to the invention

The activation of the secretion of insulin was evaluated in vitro on a INS-1 pancreatic cell line by measuring the concentration of insulin secreted in the culture medium containing glucose at 2.8 mM concentration. The compounds were tested at doses between 0.01 and 10 μM. The results are shown by the induction factor for each condition, i.e. the ratio between the concentration of insulin induced by the compound and the concentration induced by the 2.8 mM glucose on its own: the higher this factor, the more the compound has insulinotropic properties.

FIG. 4: Evaluation of the insulinotropic properties of the inventive compounds on human islets

Isolated human islets were put together with compounds according to the invention. They were incubated for an hour, and then the supernatant was isolated. The insulin contained in the supernatant (which corresponds to the insulin secreted by the islets during 1 hr) was titrated. It was compared to the secretion of basal insulin, i.e. to the quantity of insulin secreted by the islets during 1 hour in the absence of the compound. The higher the induced insulin/basal insulin ratio, the more the compound has a strong insulinotropic potential.

FIGS. 5a, 5b, 5c: In vivo evaluation of the insulinotropic properties of the compounds in rats

The purpose of this study was to evaluate in vivo the insulinotropic properties of the compounds according to the invention. In normal animals (as in man), a consequence of the oral administration of insulinotropic compounds is a significant increase in the plasmatic level of insulin. This induced hyperinsulinemia induces a reduction in glycemia. The curves presented show the insulinotropic and hypoglycemic effects of the inventive compounds (administered orally in a fasting rat).

FIGS. 6a, 6b: In vivo evaluation of the regulation of the expression of genes by the compounds according to the invention

The capacity of the inventive compounds to induce transcriptional activity was evaluated in vivo in Hartley guinea pigs. Treatment of these animals with a PPARα agonist must induce an hepatic over-expression of the target genes directly under the control of the PPARα receptor. The genes studied in this experiment were ACO (acyl Co-enzymeA oxidase, a key enzyme in the mechanism of the β-oxidation of fatty acids) and PDK-4 (pyruvate dehydrogenase kinase isoform 4, enzyme of glucidic metabolism). The higher the induction factor measured, the more the tested compound increases the expression of the gene being studied.

FIG. 7: In vivo evaluation of the lipid-lowering properties of the compounds according to the invention

The lipid-lowering effect of the inventive compounds was evaluated in vivo in Hartley guinea pigs. In guinea pigs, as in man, food enriched in cholesterol and in fatty acids induces an increase of the plasmatic levels of lipids. The presented figure depicts plasmatic lipid levels (cholesterol and triglycerides) after 14 days treatment. These levels can be compared with those observed with non-treated animals (controls): the difference between both groups demonstrates the hypolipemic effect of the compounds according to the invention.

### DETAILED DESCRIPTION OF THE INVENTION

PPARs belong to the family of nuclear receptors which are defined as transcription factors activated by a ligand. Three isotypes of PPAR, called α, γ and δ (also called β) have been described untill now (Willson, Brown et al. 2000). Agonists of these receptors are marketed for the treatment of diseases linked to metabolic disorders such as diabetes, dyslipidemias, atherosclerosis, etc. Numerous PPAR modulators, agonists or antagonists, selective or not, are currently in advanced development for these types of pathologies (Etgen and Mantlo 2003).

It is well known nowadays that PPARs play a central role in the regulation of the metabolism of lipids and glucids (Desvergne and Wahli 1999).

Fibrates (fenofibrate, gemfibrozil, etc.) are a family of medicines which make it possible to effectively correct dyslipidemias: their PPARα activating properties allow them to regulate plasmatic cholesterol as well as the rates of triglycerides and free fatty acids. More generally, the activation of PPARα by these lipid-lowering agents regulates numerous biological processes (Chapman 2003): they bring about an increase in the oxidation of fatty acids on the hepatic level (β-oxidation); they induce an increase in the plasmatic concentrations of HDL cholesterol (notably by increasing the expression of Apo AI, Apo AII, SRB1, ABCA1); they favour the clearance of VLDLs (notably by reducing ApoCIII and LPL), etc. It has also been shown that the PPARα agonists play a role in the regulation of glycemia: the activation of PPARα increases sensitivity to insulin (Guerre-Millo, Gervois et al. 2000) and it has recently been described that the modulation of PPARα has an influence upon the secretion of insulin (Sugden and Holness 2004).

Glitazones (pioglitazone, rosiglitazone), also called thiazolidinediones (TZD), are currently marketed for the treatment of type II diabetes: they have insulin-sensitizing properties linked to their PPARγ activating potential (Spiegelman 1998). More generally, it has been shown that PPARγ activators allow to restore the sensitivity to insulin of the target tissues and to reduce the plasmatic rates of glucose, lipids and insulin, in animal models of type II diabetes as well as in man (Ram 2003).

No medicine is currently being marketed for its PPARδ activating properties. Even if this receptor is the least studied nowadays of the PPARs, its primary role in the regulation of lipidic metabolism has been demonstrated (Dressel, Allen et al. 2003).

Beyond the direct role played by the PPAR ligands in the regulation of lipids and glucids, these molecules have a pleiotropic action spectrum due to the great diversity of PPAR target genes. Recent discoveries show that PPARs are involved in the regulation of the expression of numerous genes participating in major biological processes such as inflammation (Chinetti, Fruchart et al. 2003), angiogenesis, cellular proliferation and differentiation, apoptosis, activities of <<Nitric Oxide Synthases >> (NOS) and metalloproteases (MMPs), etc.

These multiple properties make PPARs therapeutic targets which are of interest for the treatment of diseases such as vascular occulsive diseases (atherosclerosis, etc.), cerebral ischemia, hypertension, diseases linked to a neovascularisation (diabetic retinopathies, etc.), inflammatory and autoimmune diseases (Crohn's disease, psoriasis, multiple sclerosis, etc.) (Pershadsingh 2004), neoplastic diseases (carcinogenesis, etc.), asthma, etc.

In addition, the inventors have shown, surprisingly, that the inventive compounds stimulate the secretion of insulin: they are insulin secretagogues. The inventive compounds therefore represent a beneficial therapeutic tool.

Stimulation of the secretion of insulin makes it possible to regulate glycemia and is used within the scope of major pathologies such as type II diabetes. Mainly 2 families of insulin-secretagogues are currently being marketed: sulphonylureas and meglitinides (also called glinides) (McCormick and Quinn 2002). The main targets of the insulinotropic molecules are the ATP-dependent potassium channels of the pancreatic β cells, which play an important role in the control of the membrane potential of these cells (Proks, Reimann et al. 2002). Closure of this channel induced by glucose and/or an insulinotropic agent (via membrane receptors, for example <<SulphonylUrea Receptors >> or SUR) induces a depolarisation of the plasmic membrane which causes the opening of the voltage-dependent calcium channels. Impulse of calcium ions (Ca⁺⁺) produced by this phenomenon causes the secretion of insulin. Numerous sulphonylureas are being or have been marketed: for example tolbutamide, glibenclamide, glipizide, gliclazide, glimepiride, etc. Meglitinides are a new class of medicines represented by repaglinide, nateglinide and mitiglinide.

Among the insulinotropic compounds, repaglinide (Grell, Hurnaus et al. 1993) is a benzoic acid derivative. Numerous molecules containing a benzoic acid function have been described in the literature over the past 25 years, following the discovery of meglitinide (HB699) by chemical modification of glibenclamide (Rufer and Losert 1979). 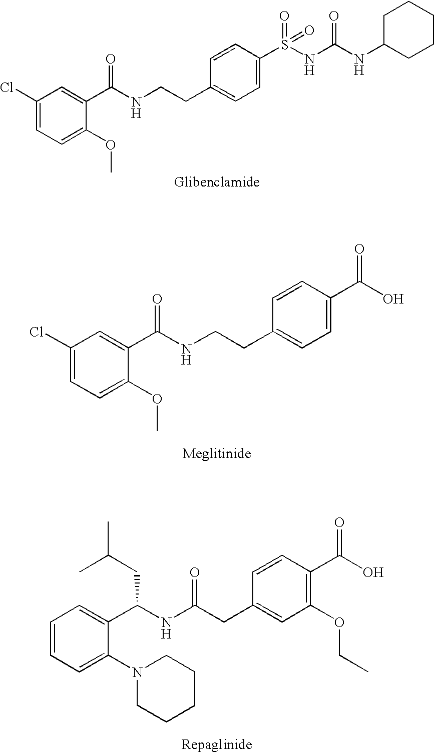
Notably, it has been shown that the meglitinide analogues in which the benzoic acid function is replaced by other functions (phenol, aniline, chlorophenyl, etc.) no longer had insulinotropic properties (Brown and Foubister 1984). Similarly, during the work which led to the discovery of repaglinide (Grell, Hurnaus et al. 1998), numerous compounds were synthesized and evaluated. It was demonstrated that replacement of the acid function by an isosteric group such as tetrazolyl inhibits activity, thus reminding the essential role of the benzoic acid function.

This invention relates to polysubstituted derivatives of the N-(benzyl)phenylacetamide type, pharmaceutical compositions comprising same, therapeutic uses thereof, in particular in the fields of human and animal health. This invention also relates to a process for preparing these derivatives.

The inventors have showed, surprisingly, that the inventive compounds intrinsically possess PPAR activating and insulinotropic properties. The inventive compounds therefore act by at least two complementary and independent ways in order to prevent or attend to pathologies linked to lipidic and glucidic disorders.

The molecules described in the invention, by their insulinotropic and PPAR agonist properties, are of particular interest for the treatment of type 11 diabetes, since this pathology currently is treated either by insulinotropic molecules (eg: meglitinides), or by PPAR agonist molecules (eg: thiazolidinediones), or by a combination of these types of molecules (Gin and Rigalleau 2002).

In addition, the molecules described in the invention, more particularly by their PPAR agonist properties, are particularly interesting for the treatment of dyslipidemias, usually treated by molecules such as fibrates.

More generally, by acting simultaneously upon several regulation processes, the inventive compounds represent a beneficial therapeutic way of preventing and/or treating, as well as diabetes and dyslipidemias, the complications associated with Syndrome X, atherosclerosis, cardiovascular diseases, obesity, hypertension, inflammatory diseases, etc.

Finally, the inventive compounds represent a beneficial therapeutic tool for preventing and/or treating several cardiovascular risk factors linked to disorders of lipidic and/or glucidic metabolism (hyperlipidemia, hyperglycemia, etc.). They allow to reduce the global risk.

These aims and others are achieved with this invention, the object of which more particularly is polysubstituted derivatives of N-(benzyl)phenylacetamide with the general formula (I): 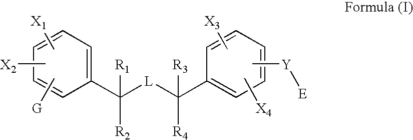
In which,
G represents
   a NRₐR_{b} radical, cyclic or not; or
   a —NR′ₐCOR′_{b}, —NR′ₐCOOR_{c}, —NR′ₐCONR′_{b}R_{c}, —NR′ₐCSR′_{b}, —NR′ₐCOSR_{c}, —NR′ₐCSOR_{c}, —NR′ₐCSSR_{c} or —NR′ₐCSNR′_{b}R_{c} radical; or
   a —OR′ₐ, —SR′ₐ, —OOCR′ₐ, —SOCR′ₐ, —OSCR′ₐ, —SSCR′ₐ, —OOCOR_{c}, —SOCOR_{c}, —OSCOR_{c}, —SSCOR_{c}, —OOCSR_{c}, —SOCSR_{c}, —OSCSR_{c}, —SSCSR_{c}, —OOCNR′_{b}R_{c}, —SOCNR′_{b}R_{c}, —OSCNR′_{b}R_{c} or —SSCNR′_{b}R_{c} radical; or
   a —SOR_{c} or —SO₂R_{c} radical;
Rₐ and R_{b}, identical or different, substituted or not, representing a hydrogen atom or an alkyl, alkenyl, aryl, aralkyl radical or a heterocycle;
Alternatively, Rₐ and R_{b} can form together a heterocycle with the nitrogen atom to which they are attached;
R′ₐ and R′_{b}, identical or different, substituted or not, represent a hydrogen atom or an alkyl, alkenyl, aryl, aralkyl radical or a heterocycle;
Alternatively, R′ₐ, R′_{b} and/or R_{c} can form together a heterocycle with the nitrogen atom,
R_{c} representing an alkyl, alkenyl, aryl, aralkyl type radical or a heterocycle;
G possibly being able to form a heterocycle with X1;
R1 represents an alkyl, alkenyl, aryl, aralkyl type radical or a heterocycle;
R2, R3, R4 independently represent a hydrogen atom, an alkyl, alkenyl, aryl, aralkyl type radical or a heterocycle;
R1 and R4 each, independently, being able to be linked to the molecular skeleton by a double linkage, R2 or R3 then being absent;
L represents an —NR—CO— or —CO—NR— amide function in which R represents a hydrogen atom or an alkyl, alkenyl, aryl, aralkyl type radical or a heterocyle;
Y represents an oxygen atom, a sulphur atom (possibly oxidised to sulphoxide or sulphone function), an amine group of the NR type (R being as defined above, in particular a hydrogen atom or an alkyl radical) or a selenium atom (possibly oxidised to selenoxide or selenone function);
E represents:
   An alkyl or alkenyl chain, substituted by one or more W groups as defined below; or
   A chain corresponding to the formula —(CH₂)ₘ-Y1-Z-Y2-(CH₂)ₙ—CR₅R₆—W; in which,
R5 and R6 independently represent a hydrogen atom, a halogen atom or an alkyl, alkenyl, aryl, aralkyl, —OR′ₐ or —SR′ₐ radical;
alternatively R5 and R6 may form together a cycle,
m and n independently represent an integer between 0 and 10;
Y1 and Y2 independently represent a covalent linkage or a heteroatom chosen from nitrogen (of the NR type, R being as defined above, in particular a hydrogen atom or an alkyl radical), oxygen or sulphur;
Z represents a covalent linkage or an alkyl, alkenyl, aryl, aralkyl radical;
W represents:
   a carboxyl radical or a derivative, preferably of the —COOH, —COOR′ₐ, —COSR′ₐ, —CONR′ₐR′_{b}, —CSNR′ₐR′_{b} type; or
   an isosteric group of the carboxyl radical, preferably an acylsulphonamide (—CONHSO₂R_{c}) or tetrazolyl radical; or
   sulphonic acid (—SO₃H) or a derivative of the —SO₃R′ₐ or —SO₂NR′ₐR′_{b} type;
R′ₐ, R′_{b} and R_{c} being as defined above;
X1, X2, X3 and X4 independently represent a hydrogen or halogen atom, an —NO₂ or —CN function, an alkyl, alkenyl, aryl, aralkyl, —OR′ₐ, —SR′ₐ, —NR′ₐR′_{b}, —NR′ₐCOR′_{b}, —NR′ₐCOOR_{c}, —NR′ₐCONR′_{b}R_{c}, —NR′ₐCSR′_{b}, —NR′ₐCOSR_{c}, —NR′ₐCSOR_{c}, —NR′ₐCSSR_{c}, —NR′ₐCSNR′_{b}R_{c}, —SOR_{c} or —SO₂R_{c}, radical or a heterocycle;
R′ₐ, R′_{b} and R_{c} being as defined above;
X1 and X3 each being able to form a cycle (aromatic or not, heterocyclic or not) with X2 and X4 respectively.

Within the scope of this invention, the term <<alkyl>> denotes a saturated hydrocarbon radical, linear, branched or cyclic, substituted or not, more particularly having between 1 and 24, preferably between 1 and 10, carbon atoms. One can cite, for example, the methyl, ethyl, n-propyl, isopropyl, n-butyl, isobutyl, tertbutyl, sec-butyl, pentyl, neopentyl, n-hexyl or cyclohexyl radical.

The term <<alkenyl>> denotes a non-saturated hydrocarbon radical, linear, branched or cyclic, substituted or not, more particularly having between 2 and 24, preferably between 2 and 10 carbon atoms. One can cite, for example, the ethenyl, 1-propenyl, 2-propenyl, 1-butenyl, 2-butenyl, 1-pentenyl, 2-pentenyl, 3-methyl-3-butenyl, ethynyl, 1-propynyl, 2-propynyl, 1-butynyl, 2-butynyl, 1 -pentynyl or 2-pentynyl radical.

The term alkyloxy refers to an alkyl chain linked to the molecule by means of an oxygen atom (ether linkage). The alkyl chain corresponds to the definition given above. As an example, one can cite the methoxy, ethoxy, n-propyloxy, isopropyloxy, n-butoxy, iso-butoxy, tert-butoxy, sec-butoxy, hexyloxy radicals.

The term alkylthio refers to an alkyl chain linked to the molecule by means of a sulphur atom (thioether linkage). The alkyl chain corresponds to the definition given above. As an example, one can cite the methylthio, ethylthio, n-propylthio, isopropylthio, n-butylthio, iso-butylthio, tert-butylthio, sec-butylthio, hexylthio radicals.

The term <<aryl>> denotes an aromatic hydrocarbon radical, substituted or not, preferably having between 6 and 14 carbon atoms. In particular, it may be substituted by at least one halogen atom, an alkyl, hydroxyl, thiol, alkyloxy, alkylthio radical, a nitro function. Preferably, the aryl radicals according to this invention are chosen from phenyl, naphtyl (for example 1-naphtyl or 2-naphtyl), biphenyl (for example, 2-, 3-, or 4- biphenyl), anthryl or fluorenyl. The phenyl groups, substituted or not, are particularly preferred.

The term <<aralkyl>> denotes a radical of the alkyl type substituted by an aryl group, substituted or not. The benzyl and phenethyl groups which are possibly substituted are particularly preferred.

The term <<heterocycle>> denotes a mono- or polycyclic radical, saturated, non-saturated or aromatic, comprising one or more heteroatoms, such as nitrogen, sulphur or oxygen. They can be substituted advantageously by at least one alkyl, alkenyl, aryl, alkyloxy, alkylthio group as defined above, or a halogen atom. Pyridyl, furyl, thienyl, isoxazolyl, oxadiazolyl, oxazolyl, benzimidazol, indolyl, benzofuranyl, morpholino, pyrrolidino, piperidino, piperazino, 2-oxo-pyperidine-1-yl, 2-oxo-pyrrolidine-1-yl, hexamethylamine and tetrazol radicals are particularly preferred.

By the term <<cycle>> is understood more particularly a hydrocarbon cycle, possibly having at least one heteroatom (such as, notably, a nitrogen, sulphur or oxygen atom), saturated, non-saturated or aromatic. More particularly, the cycles include aryl or heterocycle groups, as defined above.

The radicals defined thus can be substituted, in particular by at least one halogen atom, an alkyl, hydroxyl, thiol, alkyloxy, alkylthio, hydroxyl, thiol radical, a nitro function. Thus, the alkyl radical can be a perhalogenoalkyl radical, in particular perfluoroalkyl, such as notably —CF₃.

The halogen atom is selected from a chlorine, bromine, iodine or fluorine atom.

Preferably, G represents a cyclic —NRₐR_{b} radical, G beneficially forming a nitrogenous heterocycle of the cyclic alkyleneimino type, substituted or not (pyrrolidine, piperidine, 3,5-dimethylpiperidine, 1-cyclohexamethyleneimino, etc.) possibly containing several heteroatoms (morpholine, piperazine, etc.) or an aromatic nitrogenous heterocycle (indole, etc.), substituted or not.

Preferably, a particular object of the invention relates to compounds with the general formula (II): 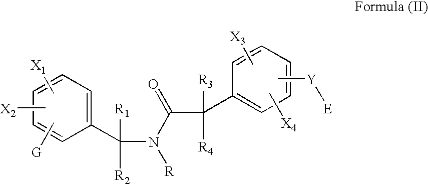
in which G, X1, X2, X3, X4, R, R1, R2, R3, R4, Y, E are as defined above.

Preferably, a particular object of the invention relates to compounds with the general formula (III): 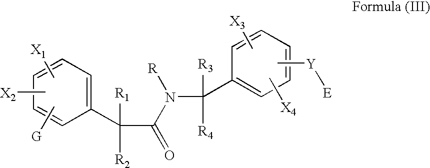
in which G, X1, X2, X3, X4, R, R1, R2, R3, R4, Y, E are as defined above.

Even more preferably, an object of the invention relates to compounds with the general formula (I), beneficially (II) or (III), in which at least one of the following conditions, preferably all of the conditions, are met.

G represents:
A —NRₐR_{b} radical, cyclic or not; or
A —NR′ₐCOR′_{b}, —NR′ₐCOOR_{c}, —NR′ₐCONR′_{b}R_{c}, —NR′ₐCSR′_{b}, —NR′ₐCOSR_{c}, —NR′ₐCSOR_{c}, —NR′ₐCSSR_{c} or —NR′ₐCSNR′_{b}R_{c} radical; or
A —OR′ₐ, —SR′ₐ radical;
Rₐ, R_{b}, R′ₐ, R′_{b} and R_{c} being as defined above, and/or
R1 represents a radical of the alkyl, alkenyl, aryl or aralkyl type; and/or
R2, R3, R4, R independently represent a hydrogen atom, an alkyl, alkenyl, aryl or aralkyl type radical; and/or
Y represents an oxygen or sulphur atom; and/or
E represents:
   An alkyl or alkenyl chain, substituted by one or more W groups as defined below; or
   A chain corresponding to the formula —(CH₂)ₘ-Y1-Z-Y2-(CH₂)ₙ—CR₅R₆—W; in which,
R5 and R6 independently represent a hydrogen atom, a halogen atom or an alkyl, alkenyl, aryl, aralkyl, —OR′ₐ or —SR′ₐ radical;
alternatively R5 and R6 may form together a cycle,
m and n independently represent an integer between 0 and 10;
Y1 and Y2 independently represent a covalent linkage or a heteroatom chosen from nitrogen (of the NR type, R being as defined above, in particular a hydrogen atom or an alkyl radical), oxygen or sulphur;
Z represents a covalent linkage or an alkyl, alkenyl, aryl, aralkyl radical; and/or
W represents:
   a carboxyl radical or a derivative, preferably of the —COOH, —COOR′ₐ, —COSR′ₐ, —CONR′ₐR′_{b}, —CSNR′ₐR′_{b} type: or
   an isosteric group of the carboxyl radical, preferably an acylsulfonamide (—CONHSO₂R_{c}) or tetrazolyl radical;
R′ₐ, R′_{b} and R_{c} being as defined above; and/or
X1, X2, X3 and X4 independently represent a hydrogen or halogen atom, a —NO₂ or —CN function, an alkyl, alkenyl, aryl, aralkyl, —OR′ₐ, —SR′ₐ, —NR′ₐR′_{b}, —NR′ₐCOR′_{b}, —NR′ₐCOOR_{c}, —NR′ₐCONR′_{b}R_{c}, —NR′ₐCSR′_{b}, —NR′ₐCOSR_{c}, —NR′ₐCSOR_{c}, —NR′ₐCSSR_{c}, —NR′ₐCSNR′_{b}R_{c}, —SOR_{c} or —SO₂R_{c} radical or a heterocycle;
R′ₐ, R′_{b} and R_{c} being as defined above.

Preferably, compounds with the formula (I) have a G group in ortho position (with respect to the —CR1R2- motif) of the phenyl radical to which it is attached.

A particular object of the invention relates to compounds with the general formula (IIa): 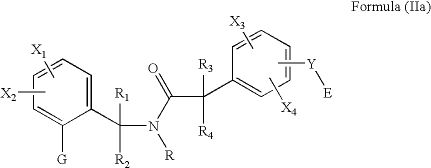
in which G, X1, X2, X3, X4, R, R1, R2, R3, R4, Y, E are as defined above.

Another particular object of the invention relates to compounds with the general formula (IIIa): 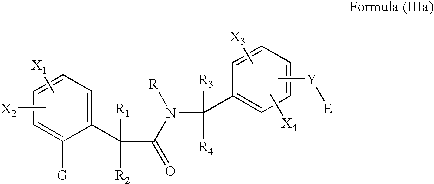
in which G, X1, X2, X3, X4, R, R1, R2, R3, R4, Y, E are as defined above.

According to another particular aspect of the invention, compounds with the formula (I) have a Y-E group in para position (with respect to the —CR3R4- motif) of the phenyl radical to which it is attached. Beneficially, according to this variation, compounds with the formula (I) have a G group in ortho position (with respect to the —CR1R2- motif) of the phenyl radical to which it is attached.

Thus, preferably, a particular object of the invention relates to compounds with the general formula (IIb): 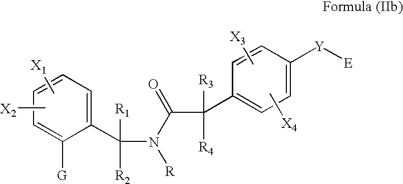
in which G, X1, X2, X3, X4, R, R1, R2, R3, R4, Y, E are as defined above.

Preferably, another particular object of the invention relates to compounds with the general formula (IIIb): 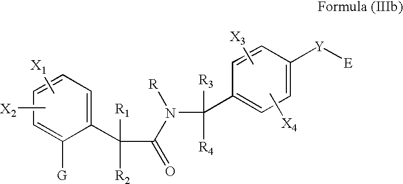
in which G, X1, X2, X3, X4, R, R1, R2, R3, R4, Y, E are as defined above.

According to one particular aspect of the invention, compounds with the formula (I) have an E group representing a chain corresponding to the formula —(CH₂)ₘ-Y1-Z-Y2-(CH₂)ₙ—CR₅R₆—W, in which at least one of the radicals R₅ and R₆ is different from the hydrogen atom.

Even more preferably, an object of the invention relates to compounds with the general formula (I), beneficially (II) or (III), in which at least one of the following conditions, preferably all of the conditions, are met:
G represents an —NRₐR_{b} radical, cyclic or not;
Rₐ and R_{b} being as defined above;
G preferably forming a nitrogenous heterocycle of the cyclic alkylneimino type, substituted or not (pyrrolidine, piperidine, 3,5-dimethylpiperidine, 1-cyclohexamethyleneimino, etc.) possibly containing several heteroatoms (morpholine, piperazine, etc.) or an aromatic nitrogenous heterocycle (indole, etc.) substituted or not; and/or
R1 represents a radical of the alkyl, alkenyl, aryl or aralkyl type; and/or
R2, R3, R4 independently represent a hydrogen atom, a radical of the alkyl, alkenyl, aryl or aralkyl type; and/or
R represents a hydrogen atom; and/or
Y represents an oxygen or sulphur atom; and/or
E represents a chain corresponding to the formula —(CH₂)ₘ-Y1-Z-Y2-(CH₂)ₙ—CR₅R₆—W in which:
R5 represents a halogen atom or an alkyl, alkenyl, aryl, aralkyl, —OR′ₐ or —SR′ₐ radical;
R6 represents a hydrogen or halogen atom or an alkyl, alkenyl, aryl, aralkyl, —OR′ₐ or —SR′ₐ radical;
alternatively R5 and R6 may form together a cycle,
m and n independently represent an integer between 0 and 10;
Y1 and Y2 independently represent a covalent linkage or a heteroatom chosen from oxygen or sulphur;
Z represents a covalent linkage or an alkyl, alkenyl, aryl, aralkyl radical; and/or
W represents:
   a carboxyl radical or a derivative, preferably of the type —COOH or —COOR′ₐ; or
   an acylsulfonamide radical (—CONHSO₂R_{c});
R′ₐ and R_{c} being as defined above; and/or
X1, X2, X3 and X4 independently represent a hydrogen or halogen atom, a function —NO₂ or —CN, an alkyl, alkenyl, aryl, aralkyl, —OR′ₐ, —SR′ₐ, —NR′ₐR′_{b}, —NR′ₐCOR′_{b}, —NR′ₐCOOR_{c}, —NR′ₐCONR′_{b}R_{c}, —NR′ₐCSR′_{b}, —NR′ₐCOSR_{c}, —NR′ₐCSOR_{c}, —NR′ₐCSSR_{c}, —NR′ₐCSNR′_{b}R_{c} radical;
R′ₐ, R′_{b} and R_{c} being as defined above.

According to another particular aspect of the invention, compounds with the formula (I) have an E group which represents a chain corresponding to the formula —(CH₂)ₙ—CR₅R₆—W, with n, R₅, R₆ and W being as defined above, W preferably being a —COOH radical or an acylsulfonamide (—CONHSO₂R_{c}) radical.

Preferably, a particular object of the invention relates to compounds with the general formula (IIc): 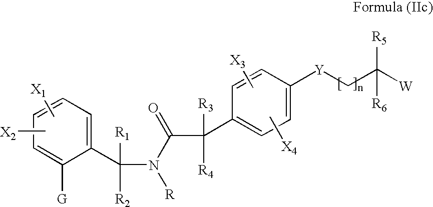
in which G, X1, X2, X3, X4, R, R1, R2, R3, R4, R5, R6, Y, W and n are as defined above.

Preferably, a particular object of the invention relates to compounds with the general formula (IIc): 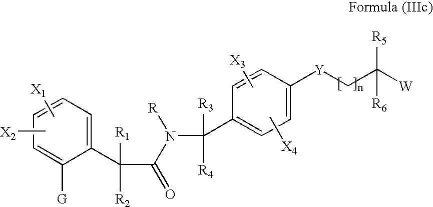
in which G, X1, X2, X3, X4, R, R1, R2, R3, R4, R5, R6, Y, W and n are as defined above.

Preferably, compounds with the formula (I) have an E group which represents a chain corresponding to the formula —(CH₂)ₙ—CR₅R₆—W with n=0 and with R₅, R₆ and W being as defined above, W preferably being a —COOH radical or an acylsulfonamide (—CONHSO₂R_{c}) radical.

Preferably, one particular object of the invention relates to compounds with the general formula (IId). 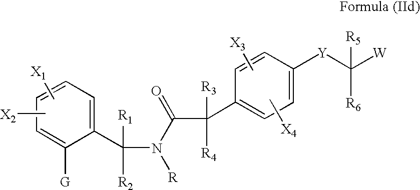
in which G, X1, X2, X3, X4, R, R1, R2, R3, R4, R5, R6, W and Y are as defined above.

Preferably, one particular object of the invention relates to compounds with the general formula (IIId). 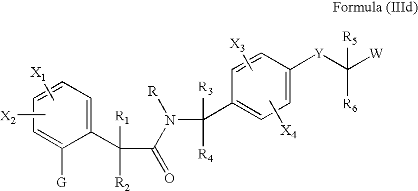
in which G, X1, X2, X3, X4, R, R1, R2, R3, R4, R5, R6, W and Y are as defined above.

Even more preferably, an object of the invention is compounds with the general formula (I), preferably with the general formulae (II) or (III), beneficially with the general formulae (IIc), (IId), (IIId) and (IIIc), in which at least one of the following conditions, preferably all of the conditions are met:
G is a derivative chosen from dimethylamine, diethylamine, pyrrolidine, 2-methylpyrrolidine, 2,5-pyrrolidine, 3-hydroxypyrrolidine, piperidine, 2-methylpiperidine, 3-methylpiperidine, 4-methylpiperidine, 3,5-dimethylpiperidine, 2,6 dimethylpiperidine, 2,2,6,6 tetramethylpiperidine, 2-ethylpiperidine, 4-phenylpiperidine, 4-benzylpiperidine, 2-(hydroxymethyl)piperidine, 2-(2-hydroxyethyl)piperidine, 4-(2-hydroxyethyl)piperidine, 3-Hydroxypiperidine, 4-hydroxypiperidine, decahydroisoquinoline, morpholine, homomorpholine, hexamethyleneimine, heptamethyleneimine, pyrrole, indole; and/or
R1, R5 and R6 independently are chosen from the methyl, ethyl, n-propyl, isopropyl, cyclopropyl, n-butyl, isobutyl, tert-butyl, sec-butyl, cyclobutyl, pentyl, neopentyl, cyclopentyl, n-hexyl, cyclohexyl, phenyl, benzyl or phenethyl groups; and/or
alternatively R5 and R6 form together a cycle, wherein said cycle is selected from the cyclopropyl, cyclobutyl, cyclopentyl, and cyclohexyl, and/or
R2, R3, R4 and R represent a hydrogen atom; and/or
Y represents an oxygen or sulphur atom; and/or
X1, X2, X3 and X4 independently represent a hydrogen or halogen atom, a methyl, trifluoromethyl, methoxy, trifluoromethoxy, thiomethoxy, nitro, methylamino, dimethylamino or cyano radical.

According to one particular embodiment of the invention, the preferred compounds are indicated below:

### Compound 1: 2-[4-[1-(3-methyl-1-(2-(1-piperidinyl)phenyl)butyl)aminocarbonylmethyl]phenoxy]-2-methylpropanoic acid

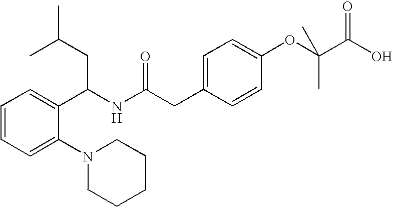

### Compound 2: 2-[4-[1-(1-(2-(1-piperidinyl)phenyl)ethyl)aminocarbonylmethyl]phenoxy]-2-methylpropanoic acid

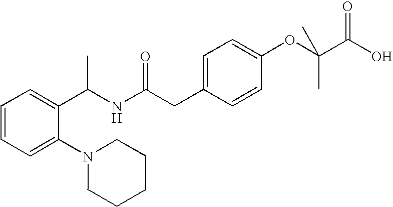

### Compound 3: 2-[4-[1-(1-phenyl-1-(2-(1-piperidinyl)phenyl)methyl)aminocarbonylmethyl]phenoxy]-2-methylpropanoic acid

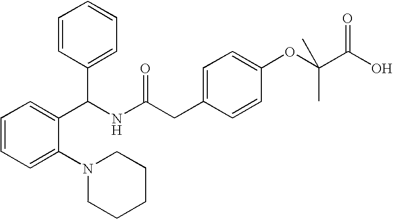

### Compound 4: 2-[2-methoxy-4-[1-(3-methyl-1-(2-(1-piperidinyl)phenyl)butyl)aminocarbonylmethyl]phenoxy]-2-methylpropanoic acid

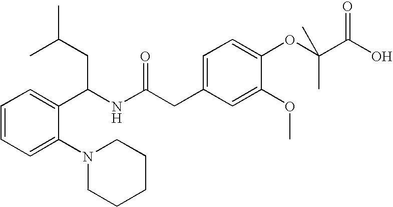

### Compound 5: 2-[4-[1-(1-(2-(1-piperidinyl)phenyl)heptyl)aminocarbonylmethyl]phenoxy]-2-methylpropanoic acid

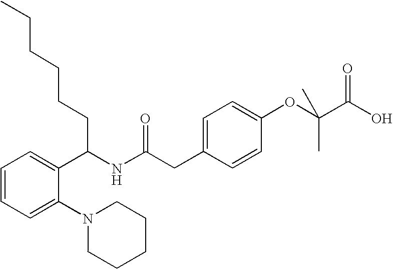

### Compound 6: 2-[4-[1-(1-cyclohexyl-1-(2-(1-piperidinyl)phenyl)methyl)aminocarbonylmethyl]phenoxy]-2-methylpropanoic acid

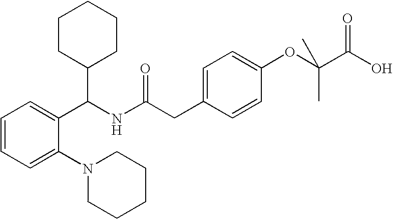

### Compound 7: 2-[4-[1-(2-phenyl-1-(2-(1-piperidinyl)phenyl)ethyl)aminocarbonylmethyl]phenoxy]-2-methylpropanoic acid

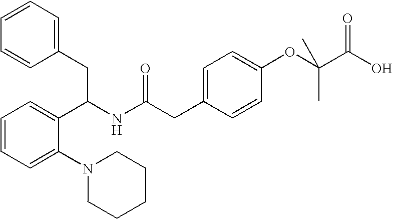

### Compound 8: 2-[4-[1-(3-methyl-1-(3-(1-piperidinyl)phenyl)butyl)aminocarbonylmethyl]phenoxy]-2-methylpropanoic acid

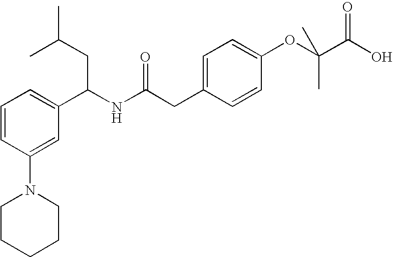

### Compound 9: 2-[4-[1-(3-methyl-1-(4-(1-piperidinyl)phenyl)butyl)aminocarbonylmethyl]phenoxy]-2-methylpropanoic acid

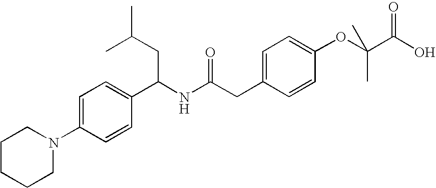

### Compound 10: 2-[4-[1-(1(2-(1-piperidinyl)phenyl)pentyl)aminocarbonylmethyl]phenoxy]-2-methylpropanoic acid

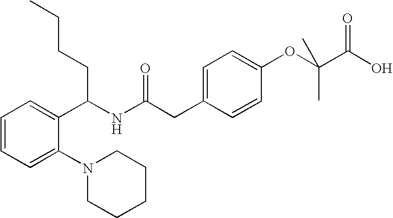

### Compound 11: 2-[2,6-dimethyl-4-[1-(3-methyl-1-(2-(1-piperidinyl)phenyl)butyl)aminocarbonylmethyl]phenoxy]-2-methylpropanoic acid

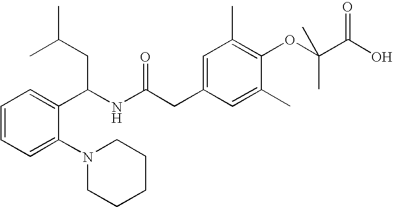

### Compound 12: 2-[4-[1-(3-methyl-1-(2-(1-cyclohexylamino)phenyl)butyl)aminocarbonylmethyl]phenoxy]-2-methylpropanoic acid

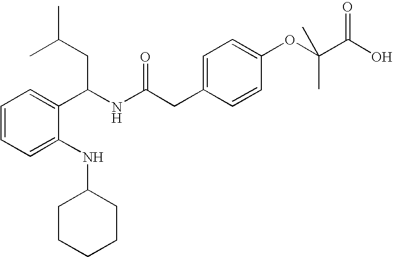

### Compound 13: 2-[4-[1-(1-(2-(1-pyrrolidinyl)phenyl)ethyl)aminocarbonylmethyl]phenoxy]-2-methylpropanoic acid

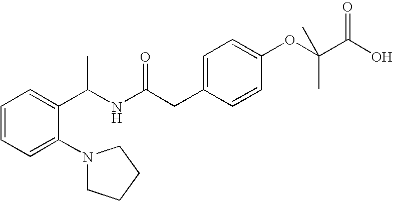

### Compound 14: 2-[4-[1-(1-(2-(4-morpholinyl)phenyl)ethyl)aminocarbonylmethyl]phenoxy]-2-methylpropanoic acid

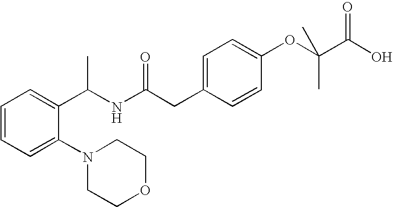

### Compound 15: 2-[4-[1-(1-(2-(1-hexamethyleneimino)phenyl)ethyl)aminocarbonylmethyl]phenoxy]-2-methylpropanoic acid

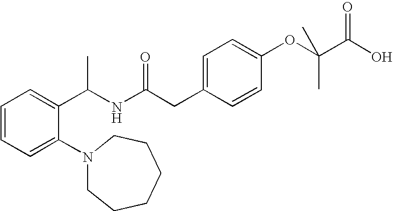

### Compound 16: 2-[4-[1-(3-methyl-1-(2-(1-pyrrolidinyl)phenyl)butyl)aminocarbonylmethyl]phenoxy]-2-methylpropanoic acid

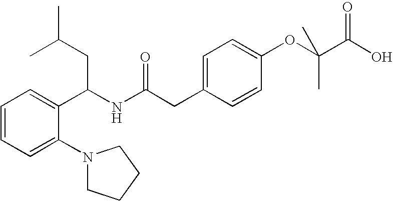

### Compound 17: 2-[4-[1-(3-methyl-1-(2-(4-morpholinyl)phenyl)butyl)aminocarbonylmethyl]phenoxy]-2-methylpropanoic acid

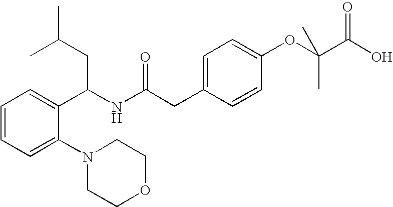

### Compound 18: 2-[4-[1-(3-methyl-1-(2-(1-hexamethyleneimino)phenyl)butyl)aminocarbonylmethyl]phenoxy]-2-methylpropanoic acid

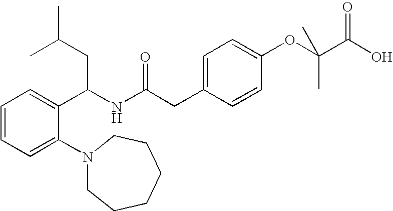

### Compound 19: 2-[4-[1-(2-cyclohexyl-1-(2-(1-piperidinyl)phenyl)ethyl)aminocarbonylmethyl]phenoxy]-2-methylpropanoic acid

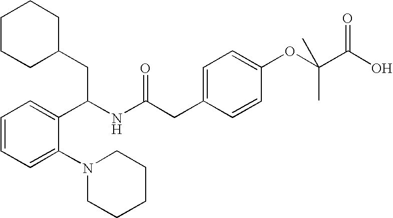

### Compound 20: 2-[4-[1-(3-methyl-1-(2-(diethylamino)phenyl)butyl)aminocarbonylmethyl]phenoxy]-2-methylpropanoic acid

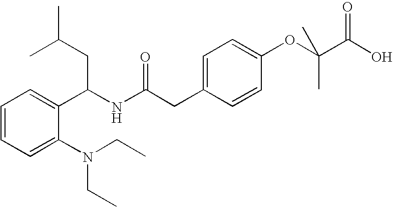

### Compound 21: 2-[4-[1-(3-methyl-1-(2-(1-piperidinyl)-4-(trifluoromethyl)phenyl)butyl)aminocarbonylmethyl]phenoxy]-2-methylpropanoic acid

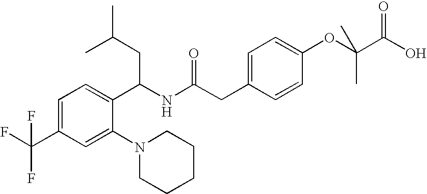

### Compound 22: 2-[4-[1-(3-methyl-1-(2-(1-piperidinyl)-5-(chloro)phenyl)butyl)aminocarbonylmethyl]phenoxy]-2-methylpropanoic acid

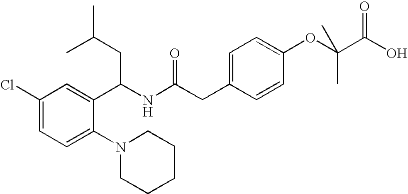

### Compound 23: 2-[4-[1-(3-methyl-1-(2-(1-piperidinyl)phenyl)butyl)aminocarbonylmethyl]phenoxy]acetic acid

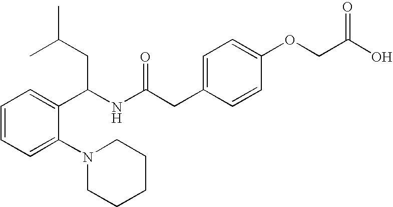

### Compound 24: 2-[4-[1-(3-methyl-1-(2-(1-piperidinyl)phenyl)butyl)aminocarbonylmethyl]phenoxy]propanoic acid

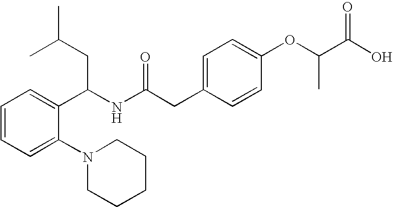

### Compound 25: 2-[4-[1-(3-methyl-1-(2-(1-piperidinyl)phenyl)butyl)aminocarbonylmethyl]phenoxy]butanoic acid

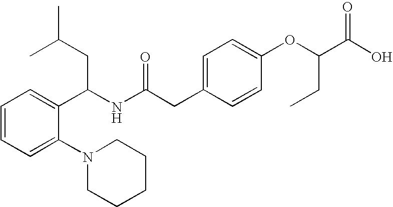

### Compound 26: 2-[4-[1-(3-methyl-1-(2-(1-piperidinyl)phenyl)butyl)aminocarbonylmethyl]phenoxy]-3-methylbutanoic acid

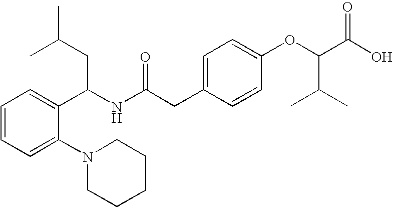

### Compound 27: 2-[4-[1-(3-methyl-1-(2-(1-piperidinyl)phenyl)butyl)aminocarbonylmethyl]phenoxy]-2-phenylacetic acid

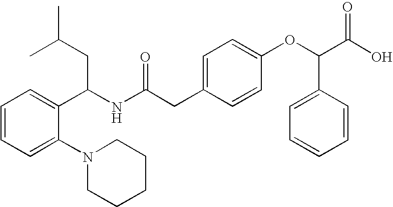

### Compound 28: 5-[4-[1-(3-methyl-1-(2-(1-piperidinyl)phenyl)butyl)aminocarbonylmethyl]phenoxy]-2,2-dimethylpentanoic acid

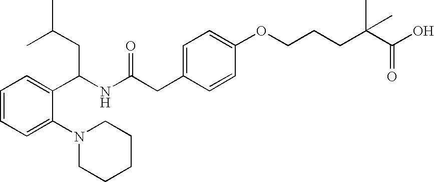

### Compound 29: 2-[3-[1-(3-methyl-1-(2-(1-piperidinyl)phenyl)butyl)aminocarbonylmethyl]phenoxy]-2-methylpropanoic acid

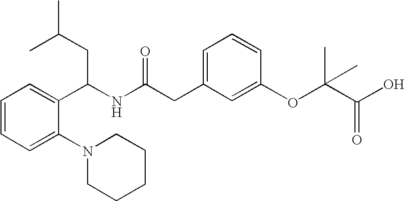

### Compound 30: 2-[3-[1-(1-(2-(1-piperidinyl)phenyl)ethyl)aminocarbonylmethyl]phenoxy]-2-methylpropanoic acid

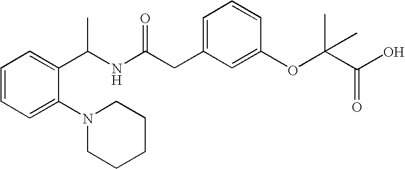

### Compound 31: 2-[3-[1-(1-phenyl-1-(2-(1-piperidinyl)phenyl)methyl)aminocarbonylmethyl]phenoxy]-2-methylpropanoic acid

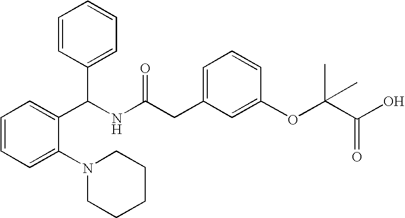

### Compound 32: 2-[4-[1-(3-methyl-1-(2-(1-piperidinyl)phenyl)butyl)carbonylaminomethyl]phenoxy]-2-methylpropanoic acid

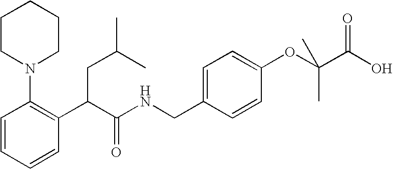

### Compound 33: 2-[4-[1-(1-(2-(1-piperidinyl)-4-(bromo)phenyl)pentyl)aminocarbonylmethyl]phenoxy]-2-methylpropanoic acid

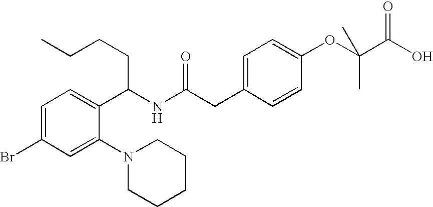

### Compound 34: 2-[4-[1-methyl-1-[1-(3-methyl-1-(2-(1-piperidinyl)phenyl)butyl)aminocarbonyl]methyl]phenoxy]-2-methylpropanoic acid

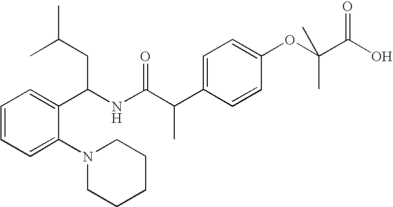

### Compound 35: 2-[4-[N-isobutyl-N-(1-(2-(1-piperidinyl)phenyl)pentyl)aminocarbonylmethyl]phenoxy]-2-methylpropanoic acid

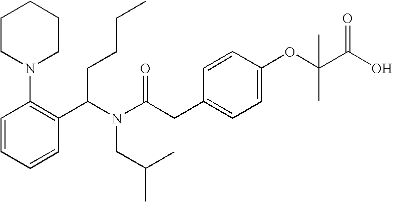

### Compound 36: 2-[4-[1-(1-(2-(phenylthio)phenyl)pentyl)aminocarbonylmethyl]phenoxy]-2-methylpropanoic acid

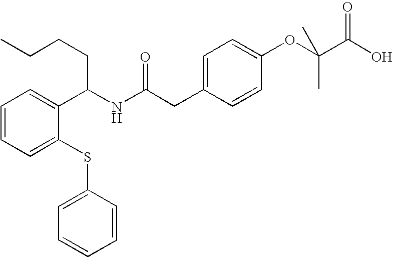

### Compound 37: 2-[4-[1-(3-methyl-1-(2-(1-piperidinyl)-5-(bromo)phenyl)butyl)aminocarbonylmethyl]phenoxy]-2-methylpropanoic acid

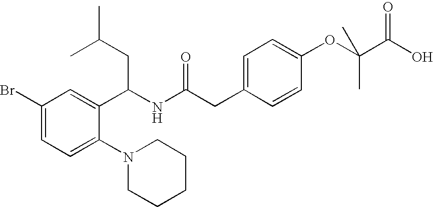

### Compound 38: 2-[4-[1-(1-(2-(1-piperidinyl)-4-(1H-indolyl)phenyl)pentyl)aminocarbonylmethyl]phenoxy]-2-methylpropanoic acid

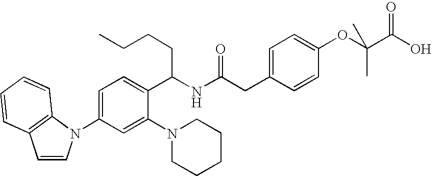

### Compound 39: 2-[4-[1-(3-methyl-1-(2-(1-piperidinyl)-4-(phenyl)phenyl)butyl)aminocarbonylmethyl]phenoxy]-2-methylpropanoic acid

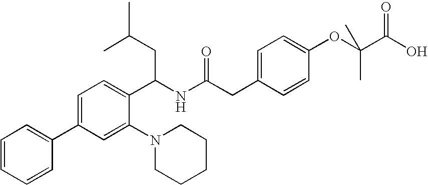

### Compound 40: 2-{4-{4-[4-[1-(3-methyl-1-(2-(1-piperidinyl)phenyl)butyl)aminocarbonylmethyl]phenoxy]butoxy}phenoxy}-2-methylpropanoic acid

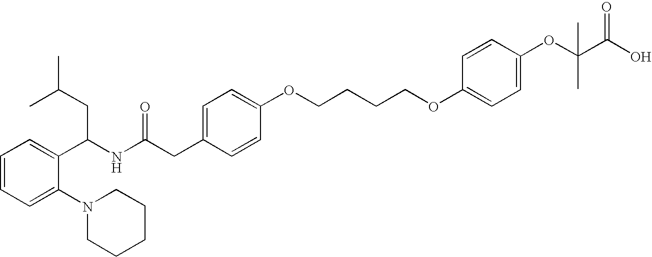

### Compound 41: 2-[4-[1-(1-(2-(phenylsulfonyl)phenyl)pentyl)aminocarbonylmethyl]phenoxy]-2-methylpropanoic acid

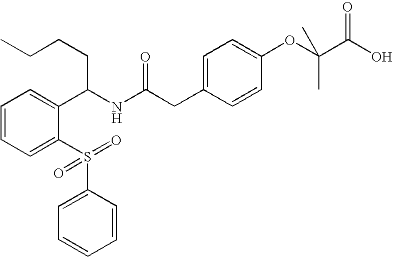

### Compound 42: 3-[4-[1-(1-(2-(1-piperidinyl)phenyl)pentyl)aminocarbonylmethyl]phenoxy]-2,2-dimethylpropanoic acid

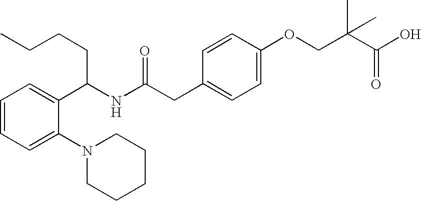

### Compound 43: 2-[4-[1-(1-(2-((3S,5R)-3,5-dimethylpiperidine-1-yl)phenyl)pentyl)aminocarbonylmethyl]phenoxy]-2-methylpropanoic acid

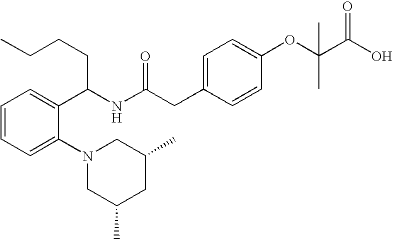

### Compound 44: 2-[4-[1-(1-(2-(hydroxy)phenyl)pentyl)aminocarbonylmethyl]phenoxy]-2-methylpropanoic acid

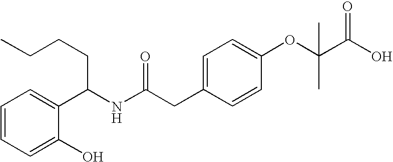

### Compound 45: 2-[4-[1-(3-fluoro-2-(1-piperidinyl)phenyl)pentyl)aminocarbonylmethyl]phenoxy]-2-methylpropanoic acid

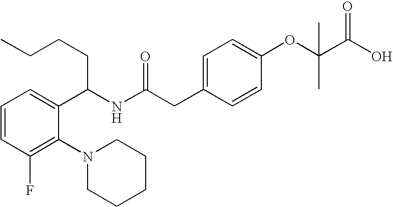

### Compound 46: 5-{N-ethyl-N-[4-[1-(3-methyl-1-(2-(1-piperidinyl)phenyl)butyl)aminocarbonylmethyl]phenyl]amino}-2,2-dimethylpentanoic acid

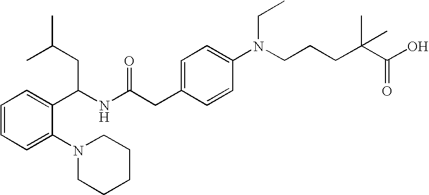

### Compound 47: 5-[4-[1-(1-(2-(1-piperidinyl)phenyl)pentyl)aminocarbonylmethyl]phenoxy]-2,2-dichloropentanoic acid

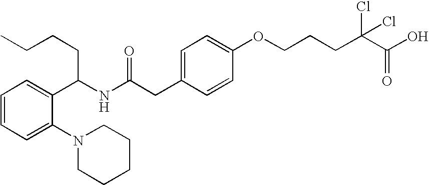

### Compound 48: 2-[4-[1-(2-(4-methoxyphenyl)-1-(2-(1-piperidinyl)phenyl)ethyl)aminocarbonylmethyl]phenoxy]-2-methylpropanoic acid

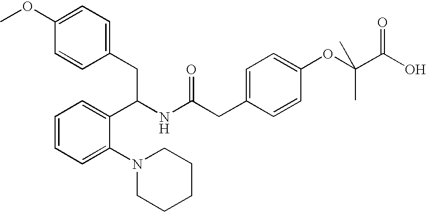

### Compound 49: 2-[4-[1-(1-(2-(1-piperidinyl)-4-(1H-pyrrolyl)phenyl)pentyl)aminocarbonylmethyl]phenoxy]-2-methylpropanoic acid

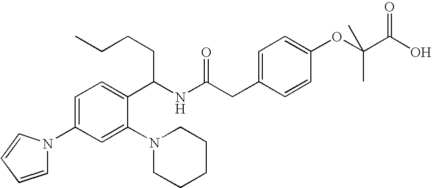

### Compound 50: 2-[4-[1-(1-(2-(1-piperidinyl)-4-(phenethyl)phenyl)pentyl)aminocarbonylmethyl]phenoxy]-2-methylpropanoic acid

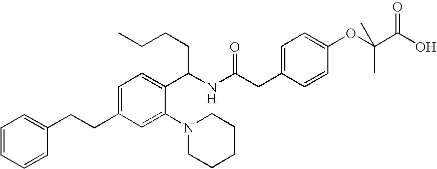

### Compound 51: 2-[4-[1-(3-phenyl-1-(2-(1-piperidinyl)phenyl)propyl)aminocarbonylmethyl]phenoxy]-2-methylpropanoic acid

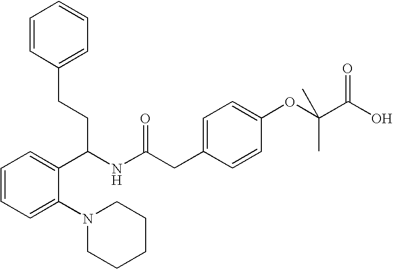

### Compound 52: N-[methylsulfonyl]-2-[4-[1-(1-(2-(1-piperidinyl)phenyl)pentyl)aminocarbonylmethyl]phenoxy]-2-methylpropanamide

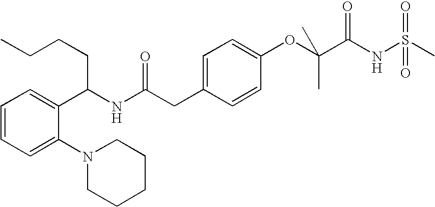

### Compound 53: 5-2-[1-(1-piperidinyl)phenyl)pentyl)aminocarbonylmethyl]phenoxy]-2,2-dimethylpentanoic acid

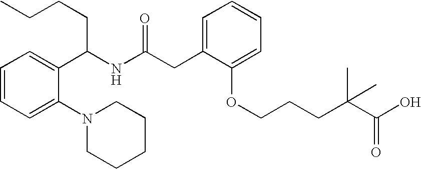

### Compound 54: 2-[4-[1-(1-(2-(methoxy)phenyl)pentyl)aminocarbonylmethyl]phenoxy]-2-methylpropanoic acid

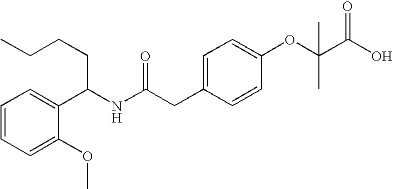

### Compound 55: 2-[4-[1-(2-(3-methylphenyl)-1-(2-(1-piperidinyl)phenyl)ethyl)aminocarbonylmethyl]phenoxy]-2-methylpropanoic acid

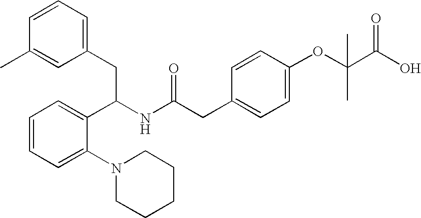

### Compound 56: 2-[2-chloro-4-[1-(3-methyl-1-(2-(1-piperidinyl)phenyl)butyl)aminocarbonylmethyl]phenoxy]-2-methylpropanoic acid

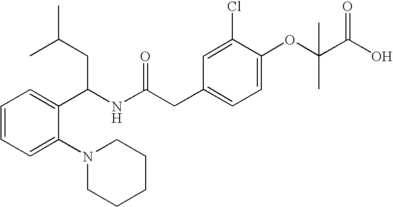

### Compound 57: 6-[4-[1-(3-methyl-1-(2-(1-piperidinyl)phenyl)butyl)aminocarbonylmethyl]phenoxy]-2,2-dimethylhexanoic acid

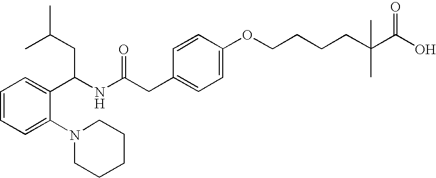

### Compound 58: N-[methylsulfonyl]-2-[4-[1-(3-methyl-1-(2-(1-piperidinyl)phenyl)butyl)aminocarbonylmethyl]phenoxy]-2-methylpropanamide

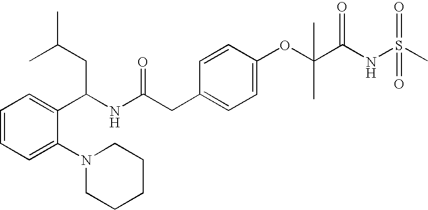

### Compound 59: N-[trifluoromethylsulfonyl]-2-[4-[1-(3-methyl-1-(2-(1-piperidinyl)phenyl)butyl)aminocarbonylmethyl]phenoxy]-2-methylpropanamide

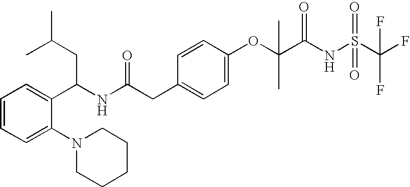

### Compound 60: N-[trifluoromethylsulfonyl]-2-[4-[1-(1-(2-(1-piperidinyl)phenyl)pentyl)aminocarbonylmethyl]phenoxy]-2-methylpropanamide

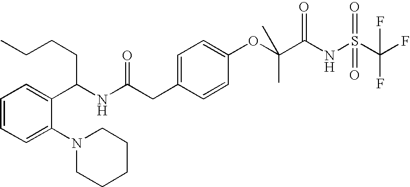

### Compound 61 N-[phenylsulfonyl]-2-[4-[1-(1-(2-(1-piperidinyl)phenyl)pentyl)aminocarbonylmethyl]phenoxy]-2-methylpropanamide

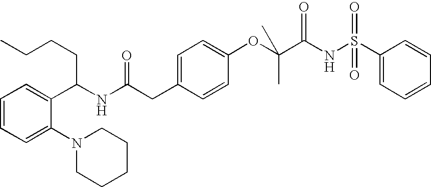

### Compound 62: N-[methylsulfonyl]-2-[4-[1-(2-phenyl-1-(2-(1-piperidinyl)phenyl)ethyl)aminocarbonylmethyl]phenoxy]-2-methylpropanamide

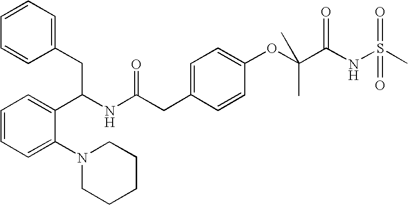

### Compound 63: N-[benzylsulfonyl]-2-[4-[1-(1-(2-(1-piperidinyl)phenyl)pentyl)aminocarbonylmethyl]phenoxy]-2-methylpropanamide

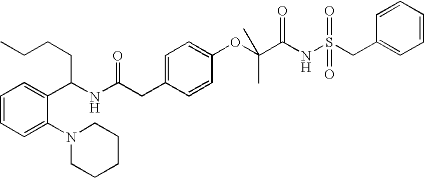

### Compound 64: N-[phenylsulfonyl]-2-[4-[-(3-methyl-1-(2-(1-piperidinyl)phenyl)butyl)aminocarbonylmethyl]phenoxy]-2-methylpropanamide

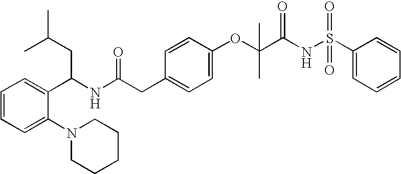

### Compound 65: 2-[4-[1-(1-(2-(1-piperidinyl)phenyl)butyl)aminocarbonylmethyl]phenoxy]-2-methylpropanoic acid

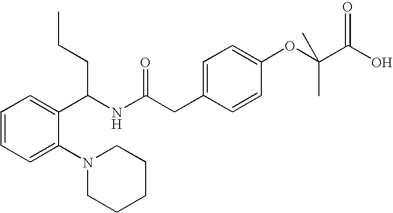

### Compound 66: N-[methylsulfonyl]-2-[4-[1-(1-(2-(1-piperidinyl)phenyl)butyl)aminocarbonylmethyl]phenoxy]-2-methylpropanamide

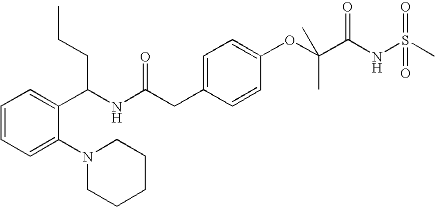

### Compound 67: N-[methylsulfonyl]-2-[4-[1-(1-(2-(methoxy)phenyl)pentyl)aminocarbonylmethyl]phenoxy]-2-methylpropanamide

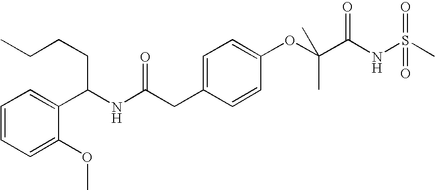

### Compound 68: 2-[4-[1-(1-(2-(isobutyloxy)phenyl)pentyl)aminocarbonylmethyl]phenoxy]-2-methylpropanoic acid

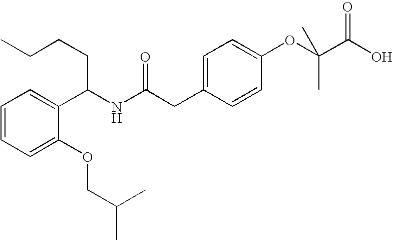

### Compound 69: 2-[4-[1-(1-(2-(1-piperidinyl)phenyl)pentyl)aminocarbonylmethyl]phenoxy]-2-(ethoxycarbonyl)acetic acid

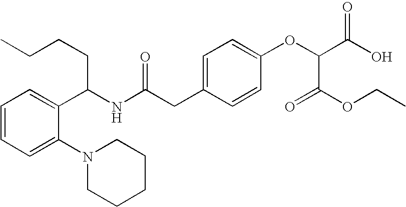

### Compound 70: 2-(4-((1H-tetrazol-5-yl)methoxy)phenyl)-N-[1-(1-(2-(1-piperidinyl)phenyl)pentyl)]acetamide

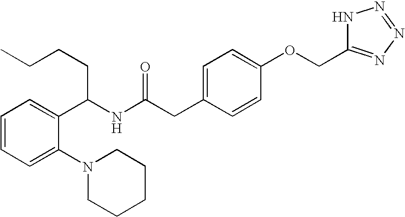

### Compound 71: 2-[4-[1-(1-(2-(1-piperidinyl)phenyl)pentyl)aminocarbonylmethyl]phenoxy]acetic acid

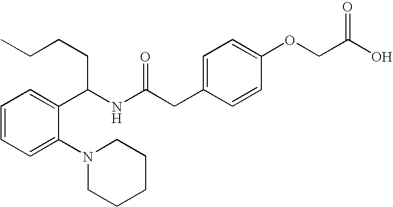

### Compound 72: N-[methylsulfonyl]-2-[4-[1-(1-(2-(1-piperidinyl)phenyl)pentyl)aminocarbonylmethyl]phenoxy]acetamide

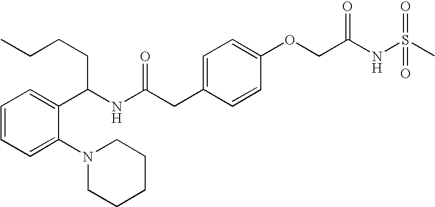

### Compound 73: 2-[2-methoxy-4-[1-(1-(2-(1-piperidinyl)phenyl)pentyl)aminocarbonylmethyl]phenoxy]-2-methylpropanoic acid

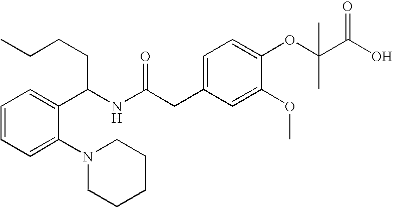

### Compound 74: 2-[4-[1-(2-phenyl-1-(2-(1-piperidinyl)phenyl)ethyl)aminocarbonylmethyl]phenylthio]-2-methylpropanoic acid

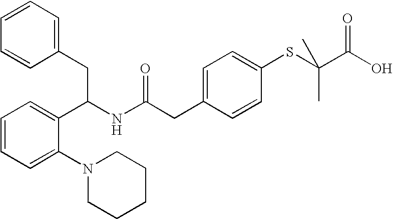

### Compound 75: 2-[4-[1-(1-(2-(diethylamino)phenyl)pentyl)aminocarbonylmethyl]phenoxy]-2-methylpropanoic acid

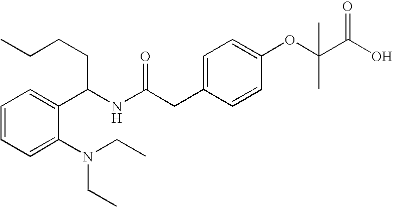

### Compound 76: N-[methylsulfonyl]-2-[4-[1-(1-(2-(diethylamino)phenyl)pentyl)aminocarbonylmethyl]phenoxy]-2-methylpropanamide

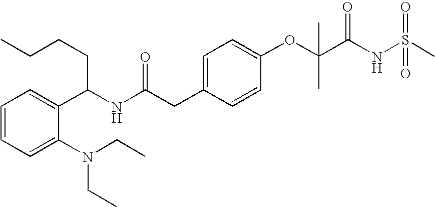

### Compound 77: N-[methylsulfonyl]-2-[2-methoxy-4-[1-(1-(2-(1-piperidinyl)phenyl)pentyl)aminocarbonylmethyl]phenoxy]-2-methylpropanamide

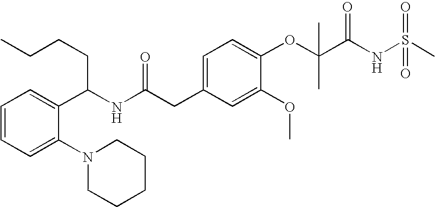

### Compound 78: 2-[4-[1-(1-(2-(acetamido)phenyl)pentyl)aminocarbonylmethyl]phenoxy]-2-methylpropanoic acid

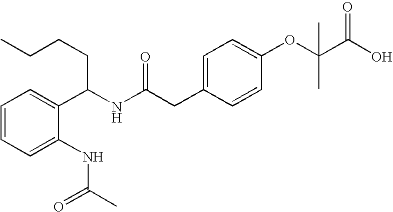

### Compound 79: 2-[4-[1-(1-(2-(1-piperidinyl)phenyl)pentyl)carbonylaminomethyl]phenoxy]-2-methylpropanoic acid

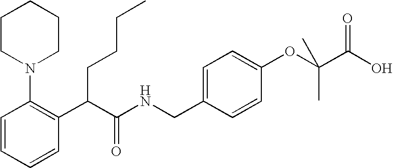

### Compound 80: N-[methylsulfonyl]-2-[4-[1-(1-(2-(1-piperidinyl)phenyl)pentyl)carbonylaminomethyl]phenoxy]-2-methylpropanamide

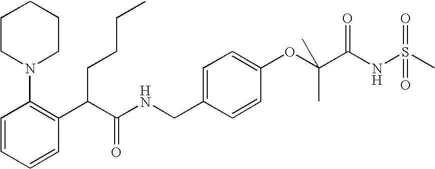

### Compound 81: 1-[4-[1-(1-(2-(1-piperidinyl)phenyl)pentyl)aminocarbonylmethyl]phenoxy]-1-cyclobutane carboxylic acid

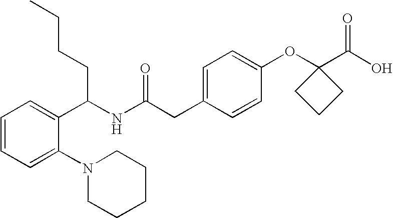

### Compound 82: N-[methylsulfonyl]-1-[4-[1-(1-(2-(1-piperidinyl)phenyl)pentyl)aminocarbonylmethyl]phenoxy]cyclobutanecarboxamide

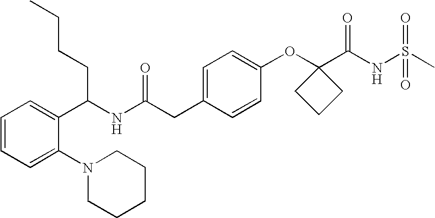

The inventive compounds also include their optical and geometric isomers, salts, solvates and prodrugs.

The inventive compounds can contain one or more asymetrical centres. This invention includes all optical and geometric isomers (enantiomers, diastereoisomers), including optically pure or enriched mixtures as well as racemic mixtures. When an enantiomerically pure (or enriched) mixture is wanted, it can be obtained either by purification of the final product or from chiral intermediate compounds, or by asymetric synthesis according to methods known to experts in the field (using for example chiral reagents and catalysts). Some inventive compounds can have different stable tautomeric forms and all of these forms as well as their mixtures are included in the invention.

This invention also relates to <<pharmaceutically acceptable>> salts of the compounds according to the invention. Generally, this term indicates salts which are slightly or non-toxic obtained from organic or inorganic bases or acids. These salts can be obtained during the final purification stage of the compound according to the invention or by incorporating the salt into the compound which has already been purified.

More specifically, the E group as described above can present an acidic character. The corresponding salts are chosen from metal salts (for example, aluminium, zinc, chrome), alkaline salts (lithium, sodium, potassium) or alkaline earth salts (calcium, magnesium). They can be, for example, organic salts such as ammonium derivatives and non-toxic amines:ammonium, quaternary ammonium (tetramethylammonium, tetraethylammonium), alkylamines (methylamine, dimethylamine, trimethylamine, triethylamine, ethylamine, etc.), hydroxyalkylamines (2-hydroxyethylamine, bis-(2-hydroxyethyl)amine, tri-(2-hydroxyethyl)amine, etc.), cycloalkylamines (bicyclohexylamine, glucamine, etc.), pyridines and analogues (collidine, quinine, quinoline, etc.) of amino acid salts with basic character (lysine, arginine, etc.).

The G group as described above can present a basic character. The corresponding salts are beneficially chosen from mineral acids (hydrochloric, hydrobromic, sulfuric, boric, nitric, phosphoric, etc.) or organic acids (for example, carboxylic or sulfonic acids such as formic, acetic, methylsulfonic, proprionic, toluenesulfonic, valeric, oleic, palmitic, stearic, lactic, lauric, oxalic, citric, maleic, succinic, glycolic, tartaric, etc.) or further salts obtained from amino acids with an acidic character such as glutamic acid.

Some inventive compounds could be isolated in the form of zwitterions and each of these forms are included in the invention, as are their mixtures.

Some inventive compounds and their salts could be stable in several solid forms. This invention includes all of the solid forms of the compounds according to the invention, including amorphous, polymorphous, mono- and poly-crystalline forms.

The inventive compounds can exist in free form or in solvated form, for example with pharmaceutically acceptable solvents such as water (hydrates) or ethanol.

This invention also includes the prodrugs of the inventive compounds which, having been administered to a patient, transform into compounds as described in the invention or into their metabolites which have therapeutic activities comparable to the compounds according to the invention.

The inventive compounds labeled by one or more isotopes are also included in the invention: these compounds are structurally identical but different by the fact that at least one atom of the structure is replaced by an isotope (radioactive or not). Examples of isotopes which can be included in the structure of the inventive compounds can be chosen from hydrogen, carbon, nitrogen, oxygen, sulphur such as ²H, ³H, ¹³C, ¹⁴C, ¹⁵N, ¹⁸O, ¹⁷O, ³⁵S respectively. The ³H and ¹⁴C radioactive isotopes are particularly preferred because they are simple to prepare and to detect within the scope of studies into the in vivo bioavailability of substances. Heavy isotopes (such as ²H) are particularly preferred because they are used as internal standards in analytical studies.

Another object of this invention is compounds such as described above as medicines.

Another object of this invention is a pharmaceutical composition comprising, in a support which is acceptable on a pharmaceutical level, at least one compound as described above, possibly in association with one or more other therapeutic and/or cosmetic active ingredients. This is beneficially a pharmaceutical composition for the treatment of diabetes, dyslipidemias, insulin resistance, pathologies associated with Syndrome X, atherosclerosis, cardiovascular diseases, obesity, hypertension, inflammatory diseases, etc. Inflammatory pathologies relate in particular to asthma. It is preferably a pharmaceutical composition for preventing and/or treating cardiovascular risk factors linked to disorders of lipidic and/or glucidic metabolism (hyperlipidemia, type II diabetes, obesity etc.), making it possible to reduce the global risk.

Another object of the invention relates to the use of at least one compound such as described above for the preparation of pharmaceutical compositions intended for treating and/or preventing various pathologies, more particularly linked to problems with metabolism among which one can cite dyslipidemias, diabetes, insulin resistance, pathologies associated with Syndrome X, atherosclerosis, cardiovascular diseases, obesity, hypertension, inflammatory diseases, etc.

More generally, an object of the invention is the use of at least one compound as described above for the preparation of pharmaceutical compositions intended for preventing and/or treating the risk factors for cardiovascular diseases linked to disorders of metabolism of the lipids and/or glucids and thus intended to reduce the global risk.

As an example (and not limitatively), compounds described above, like the insulin secreting compounds and PPAR activating compounds currently being marketed (or under advanced development) for the treatment of metabolic diseases, can advantageously be administered in combination with other therapeutic and/or cosmetic agents, marketed or in development, such as:
Antidiabetics: secretagogues (sulfonylureas (glibenclamide, glimepiride, gliclazide, etc.), glinides (repaglinide, nateglinide, etc.)), alpha-glucosidase inhibitors, PPARγ agonists (thiazolidinediones such as rosiglitazone or pioglitazone), mixed PPARα/PPARγ agonists (tesaglitazar, muraglitazar), pan-PPARs (activate PPARα, PPARγ and PPAR_{β}), biguanides (mefformin), DPP-IV (Dipeptidyl Peptidase IV) inhibitors, GLP-1 (Glucagon-Like Peptide 1) analogues (exenatide), etc.
   insulin
   lipid-lowering and/or cholesterol-lowering molecules: Fibrates (fenofibrate, gemfibrozil), HmGCoA (Hydroxymethylglutaryl coenzyme A reductase) inhibitors (statins such as atorvastatin, simvastatin, fluvastatin), cholesterol absorption inhibitors (ezetimibe, phytosterols), CETP (Cholesteryl ester Transfer Protein) inhibitors (torcetrapib), ACAT (AcylCoA-Cholesterol Acyl Transferase) inhibitors, MTP (Microsomal Triglyceride Transfer Protein) inhibitors, sequestering agents of biliary acids (cholestyramine), vitamin E, polyunsaturated fatty acids, omega 3 fatty acids, nicotinic acid derivatives (niacin), etc.
   anti-hypertension agents and hypotension agents: ACE (Angiotensin-Converting Enzyme) inhibitors (captopril, enalapril, ramipril or quinapril), the angiotensin II receptor antagonists (losartan, valsartan, telmisartan, eposartan, irbesartan, etc.), beta blockers (atenolol, metoprolol, labetalol, propranolol), thiazidic and non-thiazidic diuretics (furosemide, indapamide, hydrochlorthiazide, anti-aldosterone), vasodilatators, calcium channel blockers (nifedipine, felodipine or amlodipine, diltizem or verapamil), etc.
   anti-platelet agents: Aspirin, Ticlopidine, Dipyridamol, Clopidogrel, flurbiprofen, etc.
   anti-obesity agents: Sibutramine, lipase inhibitors (orlistat), PPAR_{δ} agonists and antagonists, cannabinoid CB1 receptor antagonists (rimonabant), etc.
   anti-inflammatories: for example, corticoids (prednisone, betamethazone, dexamethazone, prednisolone, methylprednisolone, hydrocortisone, etc.), NSAID or Non-Steroidian Anti-Inflammatories Drugs derived from indole (indomethacin, sulindac), NSAID of the arylcarboxylic group (thiaprofenic acid, diclofenac, etodolac, flurbiprofen, ibuprofen, ketoprofen, naproxen, nabumetone, alminoprofen), NSAID derived from oxicam (meloxicam, piroxicam, tenoxicam), NSAID from the fenamate group, selective COX2 inhibitors (celecoxib, rofecoxib), etc.
   anti-oxidant agents: for example probucol, etc.
agents used in the treatment of cardiac insufficiency: thiazidic or non-thiazidic diuretics (furosemide, indapamide, hydrochlorthiazide, anti-aldosterone), Angiotensin converting enzyme inhibitors (captopril, enalapril, ramipril or quinapril), digitalics (digoxin, digitoxin), beta blockers (atenolol, metoprolol, labetalol, propranolol), Phosphodiesterase inhibitors (enoximone, milrinone), etc.
   agents used for the treatment of coronary insufficiency: beta blockers (atenolol, metoprolol, labetalol, propranolol), calcium channel blockers (nifedipine, felodipine or amlodipine, bepridil, diltiazem or verapamil), NO releasing agents (trinitrin, isosorbide dinitrate, molsidomine), Amiodarone, etc.
   antineoplastics:cytotoxic agents (agents interacting with DNA, alkylating agents, cisplatin and derivatives), cytostatic agents (GnRH analogues, somatostatin analogues, progestins, anti-oestrogens, aromatase inhibitors, etc.), immune reaction modulators (interferons, IL2, etc.), etc.
   anti-asthmatics such as bronchodilatators (beta 2 receptor agonists), corticoids, cromoglycate, leucotriene receptor antagonists (montelukast), etc.
   corticoids used in the treatment of pathologies of the skin such as psoriasis and dermatitis
   vasodilatators and/or anti-ischemic agents (buflomedil, extract of Ginkgo Biloba, naftidrofuryl, pentoxifylline, piribedil, etc.).

The invention also relates to a method for treating pathologies linked to metabolism of the lipids and/or glucids including the administration to a subject, more particularly human, of an effective quantity of a compound or of a pharmaceutical composition as defined above. By treatment, one understands both preventive and curative treatments.

The pharmaceutical compositions according to the invention advantageously comprise one or more excipients or vehicles, acceptable on a pharmaceutical level. One can cite for example saline, physiological, isotonic, buffered solutions, etc., compatible with a pharmaceutical use and known to experts in the field. The compositions may contain one or more agents or vehicles chosen from dispersants, solubilisers, stabilizers, preservatives, etc. Agents or vehicles which may be used in formulations (liquid and/or injectable and/or solid) are notably methylcellulose, hydroxymethylcellulose, carboxymethylcellulose, polysorbate 80, mannitol, gelatine, lactose, vegetable oils, acacia, liposomes, etc. The compositions may be formulated in the form of injectable suspensions, gels, oils, tablets, suppositories, powders, gelatine capsules, capsules, aerosols, etc., possibly by means of galenic forms or devices which ensure that liberation is prolonged and/or delayed. For this type of formulation, one beneficially uses an agent such as cellulose, carbonates or starches.

The compounds or compositions according to the invention can be administered in different ways and in different forms. Thus, they can for example be administered systemically, orally, parenterally, by inhalation or by injection, as for example by intraveinous, intramuscular, subcutaneous, transdermic, intra-arterial routes, etc. For the injections, the compounds are generally presented in the form of liquid suspensions, which can be injected using syringes or perfusions, for example.

Of course the flow rate and/or the dose injected can be adapted by an expert in the field according to the patient, the pathology, the delivery system, etc. Typically, the compounds are administered at doses which can vary between 1 μg and 2 g per administration, preferably between 0.1 mg and 1 g per administration. Administrations can be daily, and even repeated several times a day, if so required. Moreover, the compositions according to the invention can further comprise other agents or active ingredients.

Another object of the invention are processes for the preparation of substituted compounds derived from N-(benzyl)phenylacetamide.

The compounds of the invention can be prepared from commercial products, using a combination of chemical reactions.

More specifically, several steps of synthesis are necessary in order to obtain compounds according to the invention.

The key step is the formation of the amide linkage of the substituted compounds derived from N-(benzyl)phenylacetamide which are the object of the invention, beneficially by the condensation of a derivative of the mono- or polysubstituted benzylamine type with a derivative of the mono- or polysubstituted phenylacetic acid (or derivative) type. This condensation can be carried out using methods known to experts in the field, and more particularly those which have been developed within the scope of peptide synthesis.

Others steps consist of incorporating or transforming different functional groups, which can take place before and/or after the condensation step as illustrated in the methods shown below.

The object of this invention is therefore a process for the preparation of the inventive compounds as described above including at least the following step:
(i) A condensation step, preferably of a mono- or polysubstituted benzylamine type derivative with a mono- or polysubstituted phenylacetic acid (or derivative) type derivative; and possibly, before or after step (i),
(ii) one or more insertion and/or transformation steps of functional groups.

Preferably, the process for the preparation of inventive compounds allows to obtain compounds in an optically pure (or enriched) form.

The process for the preparation of the inventive compounds makes it possible to prepare compounds called in the following intermediate compounds. Another object of this invention is certain raw materials and intermediate compounds obtained within the scope of this invention.

More specifically, these intermediate compounds are chosen from:
4-[1-(3-methyl-1-(2-(1-piperidinyl)phenyl)butyl)aminocarbonylmethyl]phenol;
4-[1-methyl-1-[1-(3-methyl-1-(2-(1-piperidinyl)phenyl)butyl)aminocarbonyl]methyl]phenol;
4-[1-(1-(2-(1-piperidinyl)phenyl)pentyl)aminocarbonylmethyl]phenol;
4-[1-(1-(2-(1-piperidinyl)phenyl)heptyl)aminocarbonylmethyl]phenol;
N-ethyl-N-[4-[1-(3-methyl-1-(2-(1-piperidinyl)phenyl)butyl)aminocarbonylmethyl]phenyl]amine;
2-[1-(1-(2-(1-piperidinyl)phenyl)pentyl)aminocarbonylmethyl]phenol;
2-chloro-4-[1-(3-methyl-1-(2-(1-piperidinyl)phenyl)butyl)aminocarbonylmethyl]phenol;
4-[1-(1-(2-(1-piperidinyl)phenyl)butyl)aminocarbonylmethyl]phenol;
4-[1-(2-phenyl-1-(2-(1-piperidinyl)phenyl)ethyl)aminocarbonylmethyl]thiophenol;
4-[1-(1-(2-(diethylamino)phenyl)pentyl)aminocarbonylmethyl]phenol.

The preparation processes given below are given as examples and are in no way limiting with regard to the way of preparing the compounds according to the invention.

Method A

The compounds with the general formula (I) according to the invention are preferably obtained by condensation between an amine (IVa or IVb) and a carboxylic acid (respectively Va or Vb).

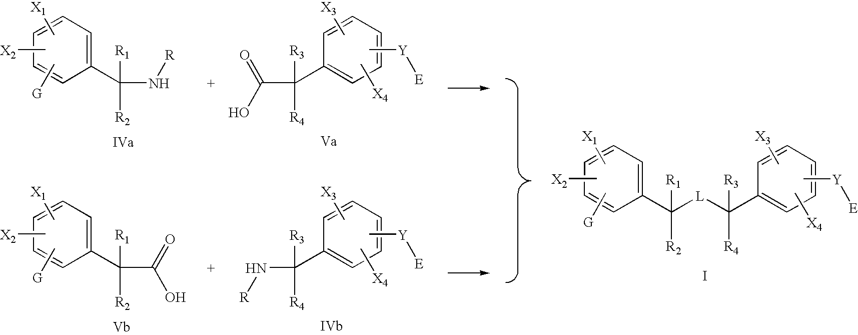

The groups G, X1, X2, X3, X4, R, R1, R2, R3, R4, Y, E, L are as defined above.

The condensation reaction can be made by numerous ways, known to experts in the field. As an example, one can cite the use of coupling agents (Py-BOP, DCC, EDC, HBTU, CDI, etc.), the use of activated acid derivatives (in this case, the acid is first transformed into an active derivative of the acyl chloride, activated ester, mixed anhydride type, etc.), mass condensation (bringing together the two entities when hot, without solvent), condensation by azeotropic distillation of water formed during reaction, etc.

A preferred route consists of working in a solvent such as dichloromethane, chloroform, diethyl ether, diisopropyl ether, methyl-tert-butyl-ether, tetrahydrofurane, dioxane, toluene, acetonitrile, or dimethylformamide. Such reactions are carried out in the presence of agents which activate the acid function (DCC/HOBt, Py-BOP, etc.) or in the presence of a preactivated form of the acid (acyl chloride, mixed anhydride, etc.). A base is often necessary: these can be inorganic bases such as (sodium, cesium) carbonates or potassium hydroxide; organic bases such as trialkylamines (triethylamine, diisopropylethylamine, etc.) or pyridine can also be used. These reactions can be made at temperatures comprised between −25 and 250° C, preferably between −10° C. and the boiling point of the solvent used.

Another route consists of working in the absence of a solvent. In this case, the reactions are carried out by eliminating water as the condensation progresses. These reactions can be carried out at temperatures comprised between 25° C. and 250° C. Water can be eliminated by evaporation (reaction under reduced pressure for example).

Another route consists also of eliminating the water formed as the condensation advances by azeotropic distillation: in this case, the reaction is carried out by reflux of a solvent such as toluene.

Method B

Compounds with the general formula (I) according to the invention are preferably obtained by hydrolysis, thermolysis, hydrogenolysis or functionalisation of the intermediate (I′). 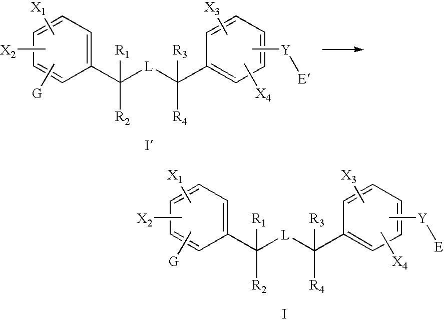
The groups G, X1, X2, X3, X4, R1, R2, R3, R4, Y, E, L are as defined above. Group E′ is by definition a group which, by hydrolysis, thermolysis, hydrogenolysis, or functionalisation makes it possible to generate group E.

According to a preferred aspect of the invention, E contains at least one carboxylic acid function. In this case, E′ is a group containing a chemical function which can be transformed into a carboxylic derivative by hydrolysis, thermolysis or hydrogenolysis.

Examples of chemical functions which are hydrolysable in carboxylic acid are acid derivatives (esters, thioesters, orthoesters, etc.) and nitrile, tetrazolyl, 1,3-oxazol-2-yl, 1,3-oxazolin-2-yl functions, etc.

Examples of chemical functions generating through thermolysis an acid function are tertiary alkyl esters, preferably tert-butylic esters.

Examples of chemical functions generating through hydrogenolysis an acid function are aralkyl esters, preferably benzylic esters.

The hydrolysis reactions can advantageously be made in the presence of an organic acid (eg: trifluoroacetic acid) or an inorganic acid (eg: hydrochloric acid) or in the presence of a base (eg: sodium hydroxide) in water or a mixture of solvents containing water (water/methanol, water/ethanol, water/THF(TetraHydroFurane), water/dioxane, etc.). They are carried out to temperatures comprised between −10° C. and 120° C., preferably between 20° C. and the reflux temperature of the solvent used. The thermolysis reactions are preferably made in the absence of a solvent (mixture in fusion) or in an inert solvent such as dichloromethane, chloroform, toluene, tetrahydrofurane or dioxane. The addition of catalytic quantities of strong acids such as paratoluenesulfonic acid is generally necessary for thermolysis. These reactions are preferably made when hot, beneficially at the boiling temperature of the solvent.

The hydrogenolysis reactions are made in the presence of a metallic catalyst (Pd/C, Pt, etc.) in an adapted solvent such as methanol, ethanol, tetrahydrofurane (THF), acetic acid, ethyl acetate, etc. They are made at temperatures comprised between 0° C. and 60° C., preferably at room temperature, under hydrogen pressure comprised between 1 and 6 bars. An alternative consists of liberating the hydrogen in situ by means of ammonium formiate.

Preferably, E′ contains the acid function or functions in protected form. It is left to the expert to choose the most appropriate protective groups based upon the nature of the different substituents (Greene and Wuts 1999). According to one preferred embodiment of the invention, the compounds (I′) correspond to the esterified form of the compounds (I). According to the nature of the ester function or functions contained in the E′ group, different methods are applicable in order to regenerate the E acid:
Basic hydrolysis: this methodology is applicable to alkyl esters such as methyl and ethyl esters;
Acid hydrolysis: this methodology is applicable to alkyl esters such as tert-butyl esters;
Hydrogenolysis: this methodology is applicable to esters of the benzylic type and analogues.

According to another preferred aspect of the invention, E contains at least one function derived from acid (COOR′ₐ, —COSR′ₐ, —CONR′ₐR′_{b}, —CSNR′ₐR′_{b}) or an isosteric function (an acylsulfonamide or tetrazolyl radical). In this case, E′ is a group containing a chemical function which can be transformed into an acid derivative or an acid isostere by functionalisation.

Preferably, E′ contains a carboxylic acid function. This function can easily be converted into an acid derivative according to the methods known to experts in the field: for example, the preparation of ester from carboxylic acid can be achieved using numerous well-documented methods. According to one preferred aspect, the carboxylic acid function is functionalised by condensation according to the methodologies described in the previous paragraph (method A). This type of methodology is also preferably applied for the preparation of isosteres of the acylsulfonamide type.

Preferably, E′ contains a nitrile function. This function can easily be converted into a tetrazolyl derivative according to the methods known to experts in the field: for example, the action of an azide derivative in an anhydrous solvent (toluene, THF, etc.) at temperatures comprised between 0° C. and the boiling temperature of the considered solvent.

Method C

The inventive compounds with the general formula (I) are preferably obtained by functionalisation of the intermediate (VI).

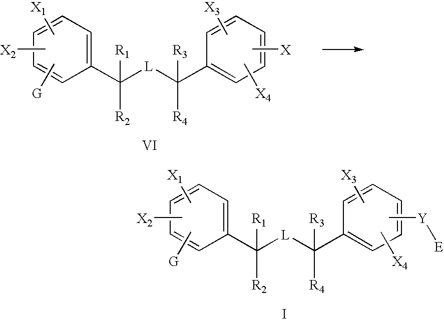

Groups G, X1, X2, X3, X4, R1, R2, R3, R4, L, Y, E are as defined above.

Group X is:
Either a leaving group: for example, a halogen atom or a triflate group;
Or a nucleophilic group: for example, a hydroxyl, thiol or amino radical.

If X is a leaving group, it can beneficially be substituted by different methods known to experts in the field.

A preferred access route is aromatic nucleophilic substitution. This type of reaction takes place under heating in the presence of a large nucleophilic excess (which can, if so required, act as the solvent). Bases are often used to activate the nucleophile (for example, cesium carbonate, sodium tert-butylate, etc.). This type of reaction is preferably made in standard solvents such as dimethyformamide, acetonitrile, tetrahydrofurane (THF), dioxane or toluene. It can be made at temperatures comprised between 20° C. and the reflux temperature of the chosen solvent.

Another preferred access route is transition metal-catalysed coupling. According to a particular embodiment of the invention, molecules with the general formula (I) can be obtained by a coupling reaction catalysed by palladium (such as the Buchwald-Hartwig reaction and its variations). These reactions are well known to experts in the field. This type of reaction requires the presence of a palladium catalyst (for example, Pd(OAc)₂, Pd₂(dba)₃), a ligand (for example, BINAP, X-Phos, tri-tert-butylphosphine) and a base (for example, Cs₂CO3, sodium tert-butylate). It can be made at temperatures comprised between 20° C. and the reflux temperature of the chosen solvent. Different solvents can be used, for example toluene, dimethylformamide, tetrahydrofurane (THF), N-methyl-2-pyrrolidone, or dioxane.

If X is a nucleophilic group, different methods known to experts in the field can be considered.

In this case, X can beneficially be alkylated so as to obtain a compound with the formula (I). For example, if X is a nitrogen atom (possibly substituted alkyl), it can be functionalised in secondary (or tertiary) amine function by nucleophilic substitution of a halogen derivative. Similarly, if X is an oxygen (or sulphur) atom, it can be functionalised in ether (or thioether) function by nucleophilic substitution of a halogen derivative. These reactions are preferably made in dichloromethane, chloroform, diethyl ether, diisopropyl ether, methyl tert-butyl ether, tetrahydrofurane (THF), dioxane, toluene, acetonitrile or dimethylformamide. A base is often necessary: they can be inorganic bases such as (sodium, cesium) carbonates or potassium hydroxide; organic bases such as trialkylamines (triethylamine, diisopropylethylamine, etc.) or pyridine can also be used. These reactions can be made at temperatures comprised between −25 and 250° C., preferably between −10° C. and the boiling point of the considered solvent.

According to a preferred embodiment, if X is a nitrogen atom (possibly alkyl-substituted), it can be functionalised by reductive alkylation: condensation of an aldehyde or of a ketone on the amine (formation of an imine) which can be reduced in situ (or after isolation) by a reducing agent (for example, NaBH₃CN, NaBHOAc₃).

According to a preferred embodiment, if X is an oxygen or sulphur atom, it can be functionalised according to Mitsunobu's conditions (triphenylphosphine, diethyl azodicarboxylate). This methodology makes it possible to generate an ether function from the intermediate (VI) and from an alcohol type derivative.

According to a particular embodiment, if X is an oxygen atom, it can be functionalised in the form of isobutyric acid (E=—C(Me)₂COOH) from 2-trichloromethyl-2-propanol in acetone in the presence of sodium hydroxide. Alternatively, a preferred strategy consists of treating the phenolic derivative with sodium hydroxide in acetone with chloroform at a temperature comprised between 20° C. et 50° C.

If one wishes to obtain sulfoxide or sulfonic derivatives, the thioether (—S-E) function can be oxidised by methods known to experts in the field. For example, oxone® can be used in a solvent such as water, methanol or dichloromethane at ambient temperature.

Method D

The inventive compounds with the general formula (I) are preferably obtained by functionalisation of the intermediate (VII).

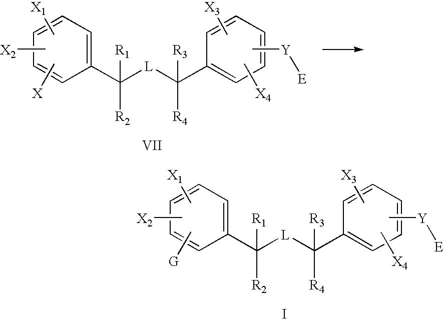

Groups X1, X2, X3, X4, R1, R2, R3, R4, Y, E, L, G are as defined above.

Group X is:
Either a leaving group: for example, a halogen atom or a triflate group;
Or a nucleophilic group: for example, a hydroxyl, thiol or amino radical.

Group G can be introduced according to the methods proposed in the previous paragraph (method C).

In the case where X is a nucleophilic group, another preferred route consists of condensing a carboxylic acid type derivative (or activated derivative), chloroformate, or isocyanate (and sulfurated analogues: isothiocyanate, etc.): for example, if X is a nitrogen atom, these strategies allow to respectively generate groups of the amide, carbamate or ureido type respectively. These reactions are preferably carried out in dichloromethane, chloroform, diethyl ether, diisopropyl ether, methyl tert-butyl ether, tetrahydrofurane (THF), dioxane, toluene, acetonitrile or dimethylformamide. A base is often necessary: these can be inorganic bases such as (sodium, cesium) carbonates or potash; organic bases such as trialkylamines (triethylamine, diisopropylethylamine, etc.) or pyridine can also be used. These reactions can be carried out at temperatures comprised between −25 and 250° C., preferably between −10° C. and the boiling point of the considered solvent.

Method E

The inventive compounds are preferably obtained by functionalisation of the intermediate (VIII).

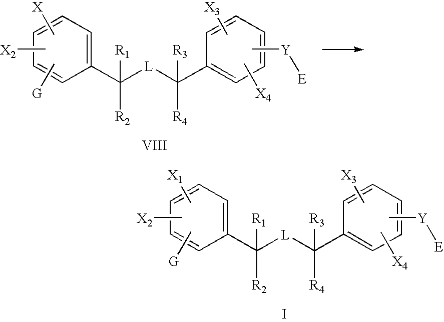

Groups G, X1, X2, X3, X4, R1, R2, R3, R4, Y, E, L are as defined above.

Group X is:
Either a leaving group: for example, a halogen atom or a triflate group;
Or a nucleophilic group: for example, a hydroxyl, thiol or amino radical.

Group X1 can be introduced according to the methods proposed in the previous paragraph (method D).

In the case where X is a leaving group, another preferred route consists of creating a carbon-carbon coupling catalysed by transition metals. According to a particular embodiment of the invention, molecules with the general formula (I) can be obtained by a coupling reaction catalysed by palladium chosen from the reactions of Suzuki-Miyaura, Heck, Stille, Sonogashira, etc. These reactions are well known to experts in the field. For example, the Suzuki reaction consists of coupling an organoboron derivative (for example, a boronic acid) in the presence of a palladium catalyst (for example, Pd(PPh₃)₄, Pd(OAc)₂), a ligand (for example BINAP) and a base (for example, Cs₂CO₃, CsF). It can be carried out at temperatures comprised between 20° C. and the reflux temperature of the chosen solvent. Differents solvents can be used, for example dimethylformamide, toluene, tetrahydrofurane, N-methyl-2-pyrrolidone or dioxane.

Method F

The inventive compounds with the general formula (I) are preferably obtained by functionalisation of the intermediate (IX).

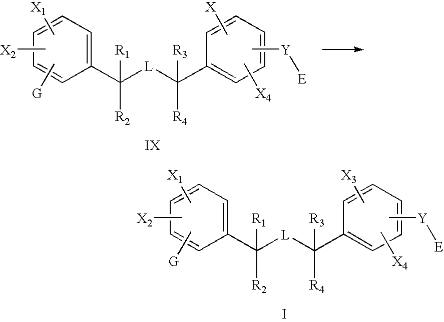

Groups G, X1, X2, X3, X4, R1, R2, R3, R4, Y, E, L are as defined above.

Groupe X is:
Either a leaving group: for example, a halogen atom or a triflate group;
Or a nucleophilic group: for example, a hydroxyl, thiol or amino radical.

Group X3 can be introduced according to the methods proposed in the previous paragraph (method E).

Method G

The inventive compounds with the general formula (I) wherein R represents a hydrogen atom are preferably obtained by reaction between an alcohol type derivative (Xa or Xb) and a nitrile type derivative (respectively XIa or XIb).

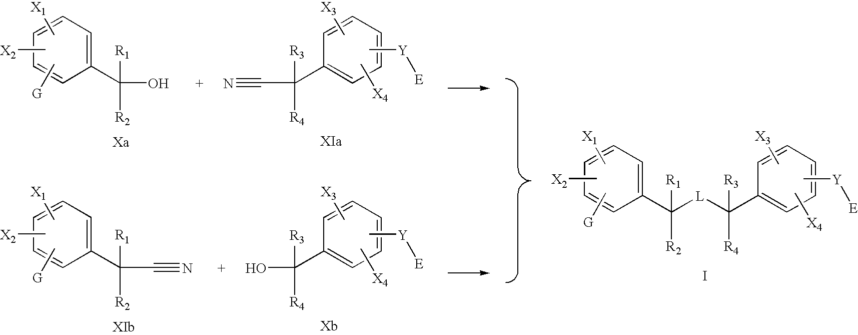

Groups G, X1, X2, X3, X4, R1, R2, R3, R4, Y, E, L are as defined above.

This type of reaction can beneficially be carried out in a highly acidic medium (for example, sulfuric acid), preferably an aqueous medium, at temperatures comprised between 20° C. and 100° C.

Method H

The inventive compounds with the general formula (I), for which R represents a hydrogen atom and R1 or R4 form a double linkage with the skeleton of the molecule are named here (I″) and (I′″) respectively. They are preferably obtained by condensation between an imine type derivative (XIIa or XIIb) and an acid type derivative respectively (Va or Vb).

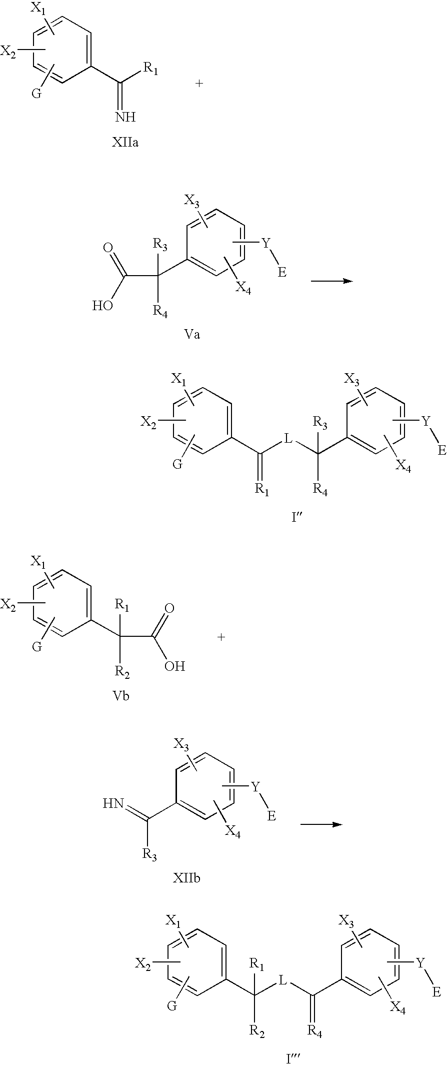

Groups G, X1, X2, X3, X4, R1, R2, R3, R4, Y, E, L are as defined above.

This type of reaction can beneficially be implemented according to the known condensation methods (see method A for more details). A favoured route consists of using a coupling agent (for example CDI, DCC) in an anhydrous solvent (for example dichloromethane or tetrahydrofurane (THF)).

Of course the unsaturated compounds (I″) and (I′″) obtained according to this route can easily be reduced during an extra step for generating compounds with the general formula (I) according to the invention (for which: R═H; R2 or R3=H).

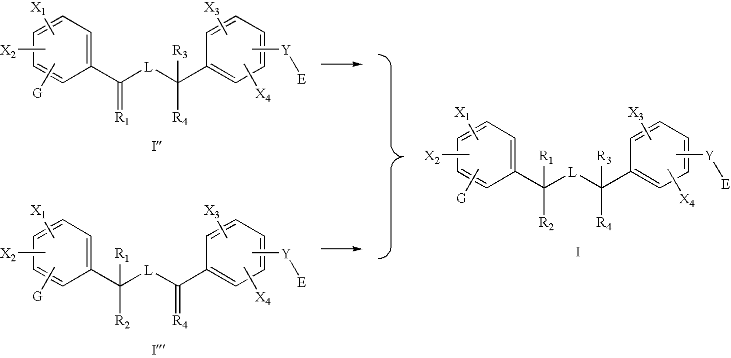

Different methodologies are applicable for reducing a double linkage. One can cite for example chemical reduction (in homogeneous catalysis for example), catalytic hydrogenation (in the presence of Pd/C for example), etc.

The use of chiral reducing agents (for example, the use of transition metals in the presence of chiral ligands) can preferably be considered for synthesizing preferably one or the other of the stereoisomers at R1 or R4. For example, one can consider a reduction catalysed by rhodium in the presence of a chiral phosphine ligand such as BINAP.

Within the scope of the invention, when the molecule comprises one or more chiral centers, the optically pure compounds (or the enriched mixtures) can be prepared or purified according to usual methods known to experts in the field: asymetrical synthesis using chiral agents (catalysts, reagents); purification of the compounds or intermediates by stereoselective methods (chromatography on chiral phases, resolution via diastereoisomer crystallization); etc.

Synthesis of substituted compounds derived from N-(benzyl)phenylacetamide according to the invention preferably includes a step of condensing a mono- or polysubstituted derivative of the benzylamine type with a mono- or polysubstituted derivative of phenylacetic acid (or derivative) type. Preferably, these two reactives will be synthesized or purified in optically pure (or enriched) form before condensation. A favoured route consists of purifying the amine or the acid by a chromatographic technique (for example, on a chiral column). Another favoured route consists of purifying the amine or acid by crystallisation of the salts formed respectively with enantiopure chiral carboxylic acids (tartaric acid, etc.) or enantiopure chiral organic bases (ephedrine, etc.). Another favoured route consists of protecting the amine or acid by a protective group containing an asymetrical centre. The mixture can then be enriched in one or other of the diastereoisomers by crystallisation, which makes it possible, after deprotection, to isolate the optically pure (or enriched) amine or acid.

More generally, all of the methods presented (A to H) can be implemented using optically pure (or enriched) reagents (IV to XII). So these methods make it possible to isolate the inventive compounds in optically pure (or enriched) form.

Another methodology consists of purifying (or enriching) a racemic mixture of compounds with the formula (I) according to the invention. A favoured route consists of purifying the compounds by chromatography. Certain of the inventive compounds can contain an acid function (notably with group E) and/or a basic function (notably with group G). They can therefore beneficially be purified by crystallisation as described above: formation of salts or protection of the molecule by chiral agents.

If the compound is wanted in the form of a salt, the latter will be obtained during a final stage known to experts in the field, not mentioned in the synthesis routes presented above. For example, a favoured method consists of using an ion-exchange resin in order to obtain the desired salts.

The preparation processes indicated above are given as an illustration, and any other equivalent process can of course also be implemented.

As well as the above specification, this invention also includes other features and benefits which can be seen from the following examples and figures, and which must be considered as illustrating the invention without limiting its scope.

### ABBREVIATIONS

BINAP 2,2′-Bis(diphenylphosphino)-1,1′-binaphthyle
CDI N,N-Carbonyidiimidazole
DCC N,N-Dicyclohexylcarbodiimide
DCU N,N-Dicyclohexylurea
EDC N-ethyl-N-(3-dimethylaminopropyl)carbodiimide chloride
DMF Dimethylformamide
HBTU 2-(1H-Benzotriazol-1-yl)-1,1,3,3-tetramethyluronium hexafluorophosphate
HOBt 1-Hydroxybenzotriazole
Pd Palladium
Pd(OAc)2 Palladium (II) acetate
Pd2(dba)3 Tris(dibenzylideneacetone)dipalladium (0)
PyBOP Hexafluorophosphate de benzotriazol-1-yl-oxytrispyrrolidinophosphonium
THF Tetrahydrofurane
X-Phos 2-dicyclohexylphosphino-2′-4′-6′-triisopropylbiphenyle

Other advantages and aspects of the invention will become evident from reading the following examples, which must be considered as illustrative and not limiting.

### EXAMPLES

The usual reagents and catalysts are commercially available (Aldrich, Alfa Aesar, Acros, Fluka or Lancaster, according to the particular case).

The organometallic (magnesium organic compound) derivatives used are available commercially or can be synthesized from corresponding halides according to methods known to experts in the field.

The washings were carried out using aqueous solutions saturated in sodium chloride (NaClₛₐₜ), molar sodium hydroxide solutions (1M NaOH) or hydrochloric acid (1M HCl).

The Proton Nuclear Magnetic Resonance spectra (¹H NMR) were recorded on a Bruker AC300P spectrometer. The chemical shifts are expressed in ppm (part per million) and the multiplicities by the usual abbreviations.

### Example 1

### Description of the Aeneral Synthesis Protocols According to the Invention

Protocol A: Aromatic Nucleophilic Substitution

The halogenobenzonitrile (1 eq) derivative was dissolved with the amine (0.5 to 5 mol/l) and the mixture was refluxed for 16 hours. The solvent was evaporated under reduced pressure and/or by washings with an aqueous 1M hydrochloric acid solution of the residue previously taken in ethyl acetate. According to the particular case, the expected product was purified by silica gel chromatography or by crystallization of the corresponding salt (for example, by preparing chlorhydrate in diethyl ether).

Protocol B: C-Alkylation/Reduction of the Nitrile Function

In an anhydrous atmosphere, nitrile (1 eq) was solubilized in anhydrous THF (0.1 to 1 mol/l) and a Grignard reagent was added (2.5 eq, in solution in THF). The mixture was brought to reflux for 16 hours. The crude was then poured onto an equivolumetric mixture of concentrated ammonia and saturated ammonium chloride at 0° C. After stirring for 15 min, the crude was filtered on celitee then extracted with ethyl acetate. The organic phase was washed with a saturated ammonium chloride solution, dried on magnesium sulfate (MgSO₄) and evaporated.

The resulting oil was taken in methanol (0.1 to 1 mol/l) and cooled to 0° C. with an ice bath. Sodium borohydride (2 eq) was slowly added, and then the crude was stirred at room temperature for 1 hour. The crude was then cooled to 0° C. with an ice bath and slowly acidified by an aqueous 10% hydrochloric acid solution. The residue was washed with dichloromethane and sodium hydroxide was added until a basic pH was re-established. The aqueous phase was then extracted with dichloromethane. The resulting organic phase was washed with NaClₛₐₜ, dried on magnesium sulfate (MgSO₄), and concentrated. According to the particular case, the expected product was purified by silica gel chromatography or by recrystallization.

Protocol C: Formation of a Benzyl Ester

Carboxylic acid (1 eq) was solubilized in DMF (0.1 to 1 mol/l), and potassium carbonate (1 eq) was added. Benzyl bromide (1 eq) was added to this suspension and the mixture was stirred for 16 hrs at room temperature. The salts were eliminated by filtration and washed with diethyl ether. Water was added and the mixture was extracted 3 times with diethyl ether. The organic phases were combined and successively washed with 1M HCl and NaClₛₐₜ, then dried on magnesium sulfate (MgSO₄). According to the particular case, the expected product was purified by silica gel chromatography or by recrystallization.

Protocol D: Substitution of a Halide Derivative with a Phenolic Derivative

The phenol type derivative (1 eq) was solubilized in acetonitrile (0.1 to 1 mol/l) and potassium carbonate (4 eq) was added. The suspension was stirred for 30 min at reflux, and then the halide derivative (2 eq) was added dropwise. Heating was maintained for 16 hrs. Solvent was evaporated and the residue was treated with ethyl acetate. The residue was filtered and the filtrate was successively washed with 1M HCl, 1M NaOH and NaClₛₐₜ. The crude was then dried on magnesium sulfate (MgSO₄), filtered and evaporated. According to the particular case, the expected product was purified by silica gel chromatography or by recrystallization.

Protocol E: Catalytic Hydrogenation

The benzyl derivative (1 eq) was solubilized in methanol (0.1 to 1 mol/l) and a catalytic amount of palladium on carbon (Pd/C 10%) was added. The reactional medium was placed under hydrogen at atmospheric pressure for 16 hours. The crude was then filtered on celite® and the filtrate evaporated. According to the particular case, the expected product was purified by silica gel chromatography or by recrystallization.

Protocol F: PyBOP Condensation

The amine (1 eq) and the acid (1 eq) were mixed in dichloromethane (0.1 to 1 mol/l). Triethylamine (3 eq) was added followed by PyBOP (1 eq). The resulting solution was stirred for 1 hour at room temperature. The crude was successively washed with 1M HCl (3 times), 1M NaOH (3 times) and NaClₛₐₜ (3 times). The organic phase was dried on magnesium sulfate (MgSO₄), filtered, and evaporated. According to the particular case, the expected product was purified by silica gel chromatography or by recrystallization.

Protocol G: Condensation by DCC/HOBt

The amine (1 eq) was solubilized in dichloromethane (0.1 to 1 mol/l), and then DCC (1 eq) and HOBt (1 eq) were added at room temperature. The acid (1 eq) was added and the reactional mixture was stirred at room temperature for 2 hours. The precipitate (DCU) was filtered and the filtrate was successively washed with 1M HCl (3 times), 1M NaOH (3 times) and NaClₛₐₜ (3 times). The organic phase was dried on magnesium sulfate(MgSO₄), filtered, and evaporated. According to the particular case, the expected product was purified by silica gel chromatography or by recrystallization.

Protocol H: Saponification

The ester (1 eq) was put in solution into an equivolumetric ethanol/2M sodium hydroxide mixture (0.1 to 1 mol/l) and the crude was stirred vigorously for 3 hrs at room temperature. The mixture was acidified by adding hydrochloric acid, concentrated, and extracted with dichloromethane (3 times). The resulting organic phase was washed with NaClₛₐₜ, dried on magnesium sulfate (MgSO₄), filtered and concentrated. According to the particular case, the expected product was purified by silica gel chromatography or by recrystallization.

Protocol I: Carbon-Nitrogen Couplings (Buchwald-Hartwig Type Reaction)

In an anhydrous atmosphere, the halide derivative (1 eq), the nitrogen derivative (1 eq), Pd₂(dba)₃ (0.04 eq), tri-tert-butylphophine (0.04 eq) and cesium carbonate (1.5 eq) were mixed in anhydrous toluene (0.1 to 1 mol/l). The suspension was brought to reflux while stirring vigorously for 16 hours. The medium was cooled to room temperature, acidified by adding 1M hydrochloric acid, and extracted with ethyl acetate (3 times). The resulting organic phase was washed with NaClₛₐₜ, dried on magnesium sulfate (MgSO₄), filtered and concentrated. According to the particular case, the expected product was purified by silica gel chromatography or by recrystallization.

Protocol J: Carbon-Carbon Couplings (Suzuki Type Reaction)

In an anhydrous atmosphere, the halide derivative (1 eq), boronic acid (1.1 eq) and Pd(Ph₃)₄ (0.01 eq) were solubilized in anhydrous DMF (0.1 to 1 mol/l). Cesium carbonate (1.2 eq) was added and the suspension was brought to reflux for 16 hours while stirring vigorously. The medium was cooled to room temperature, acidified by adding 1M hydrochloric acid, and extracted with ethyl acetate (3 times). The resulting organic phase was washed with NaClₛₐₜ, dried on magnesium sulfate (MgSO₄), filtered and concentrated. According to the particular case, the expected product was purified by silica gel chromatography or by recrystallization.

Protocol K: Condensation of a Sulfonamide Derivative with DCC/DMAP

At room temperature, sulfonamide (1 eq) was solubilized in dichloromethane (0.1 to 1 mol/l). DMAP (1.1 eq) and acid to be condensed (1 eq) were successively added. The crude was cooled to 0° C. and DCC (1.1 eq) was added. Stirring was maintained for one night at room temperature. The resulting precipitate (DCU) was filtered and the filtrate was successively washed with 1M HCl (3 times) and NaClₛₐₜ (3 times). The organic phase was dried on magnesium sulfate (MgSO₄), filtered, and evaporated. According to the particular case, the expected product was purified by silica gel chromatography or by recrystallization.

Protocol L: Condensation of a Sulfonamide Derivative with EDC/DMAP

At room temperature, the sulfonamide (1 eq) was solubilized in dichloromethane (0.1 to 1 mol/l). DMAP (1.1 eq) and acid to be condensed (1 eq) were successively added. The crude was cooled to 0° C. and EDC (1.1 eq) was added. Stirring was maintained for one night at room temperature. The resulting mixture was successively washed with 1M HCl (3 times) and NaClₛₐₜ (3 times). The organic phase was dried on magnesium sulfate (MgSO₄), filtered, and evaporated. According to the particular case, the expected product was purified by silica gel chromatography or by recrystallization.

Protocol M: Conversion of a Phenolic Derivative in a 2-methyl-phenoxy-propanoic Type Acid Derivative

Phenol (1 eq) was solubilized in acetone (36 eq). Sodium hydroxide (9 eq) was added and the resulting suspension was heated at 35° C. for 30 min. Chloroform (4.5 eq) was added dropwise to the medium while stirring at 35° C. (strongly exothermic reaction). The reaction was continued for 1 hour, cooled to room temperature, acidified by adding 1M HCl, and extracted with dichloromethane (3 times). The organic phases were combined and the resulting solution was successively washed with NaClₛₐₜ, dried on magnesium sulfate (MgSO₄), filtered, and evaporated. According to the particular case, the expected product was purified by silica gel chromatography or by recrystallization.

### Example 2

### Synthesis of Inventive Intermediate Compounds of Formula (IV)

### Example 2-1

### 3-methyl-1-(2-(1-piperidinyl)phenyl)butylamine

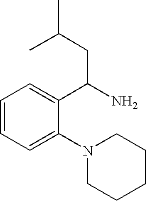

This amine was synthesized in 2 steps:

### Step 1: 2-(1-piperidinyl)benzonitrile

This intermediate compound was synthesized from 2-chlorobenzonitrile according to protocol A as previously described. It was purified by recrystallization in a hydrochloric acid saturated diethyl ether solution, the free base being regenerated by extraction in a sodium hydroxide / ethyl acetate system.

Yield: 68%

Aspect: colorless oil

¹H NMR (300 MHz, CDCl₃, δ in ppm): 1.61 (m, 2H); 1.79 (m, 4H); 3.17 (m, 4H); 6.95 (m, 2H), 7.46 (m, 1H); 7.54 (d, 7.8 Hz, 1H).

### Step 2: 3-methyl-1-(2-(1-piperidinyl)phenyl)butylamine

This amine was obtained from the nitrile intermediate according to protocol B.

It was isolated by silica gel chromatography (dichloromethane/methanol 95/5).

Yield: 79%

Aspect: yellow oil

¹H NMR (300 MHz, DMSO d6/D₂O, δ in ppm): 0.85 (d, 6.6 Hz, 3H); 0.92 (d, 6.6 Hz, 3H); from 1.30 to 1.80 (m, 9H); 2.63 (m, 2H); 2.91 (m, 2H); 4.65 (t, 7.1 Hz, 1H); from 7.14 to 7.29 (m, 3H); 7.53 (d, 6.6 Hz, 1H).

### Example 2-2:

### 1-(2-(1-piperidinyl)phenyl)ethylamine

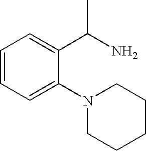

This amine was obtained from the intermediate 2-(1-piperidinyl)benzonitrile (see example 2-1) according to protocol B. It was isolated by silica gel chromatography (dichloromethane/methanol 95/5).

Yield: 48%

Aspect: pale yellow solid

¹H NMR (300 MHz, DMSO d6, δ in ppm): 1.42 (d, 6.6 Hz, 3H); 1.53 (m, 2H); 1.65 (m, 4H); from 2.71 to 2.84 (m, 4H); 4.67 (q, 6.6 Hz, 1H); 6.81 (s(broad), 2H); from 7.13 to 7.31 (m, 3H); 7.61 (d, 7.6 Hz, 1H).

### Example 2-3:

### 1-phenyl-1-(2-(1-piperidinyl)phenyl)methylamine

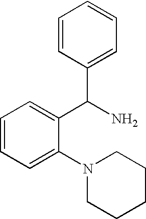

This amine was obtained from the intermediate 2-(1-piperidinyl)benzonitrile (see example 2-1) according to protocol B. It was isolated by silica gel chromatography (dichloromethane/methanol 95/5).

Yield: 80%

Aspect: colorless oil

¹H NMR (300 MHz, CDCl₃, δ in ppm): 1.56 (m, 2H); 1.66 (m, 4H); 2.15 (s(broad), 2H); 2.75 (m, 4H); 5.70 (s, 1H); from 7.10 to 7.40 (m, 9H).

### Example 2-4

### 1-(2-(1-piperidinyl)phenyl)heptylamine

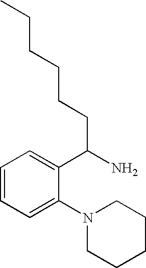

This amine was obtained from the intermediate 2-(1-piperidinyl)benzonitrile (see example 2-1) according to protocol B. It was isolated by silica gel chromatography (dichloromethane/methanol 9/1).

Yield:64%

Aspect: pale yellow oil

¹H NMR (300 MHz, CDCl₃, δ in ppm): 0.88 (t, 6.7 Hz, 3H); from 1.20 to 1.40 (m, 8H); from 1.50 to 1.80 (m, 8H); 1.96 (m, 2H); 2.81 (m, 4H); 4.34 (t, 6.7 Hz, 1H); from 7.10 to 7.30 (m, 4H).

### Example 2-5:

### 1-cyclohexyl-1-(2-(1-piperidinyl)phenyl)methylamine

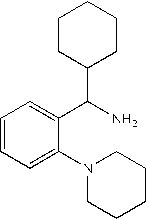

This amine was obtained from the intermediate 2-(1-piperidinyl)benzonitrile (see example 2-1) according to protocol B. It was isolated by silica gel chromatography (dichloromethane/methanol 9/1).

Yield: 53%

Aspect: pale red oil

¹H NMR (300 MHz, CDCl₃, δ in ppm): from 0.90 to 2.11 (m, 19H); 2.75 (m, 2H); 2.87 (m, 2H); 4.06 (d, 8.6 Hz, 1H); from 7.00 to 7.30 (m, 4H).

### Example 2-6

### 2-phenyl-1-(2-(1-piperidinyl)phenyl)ethylamine

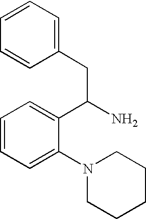

This amine was obtained from the intermediate 2-(1-piperidinyl)benzonitrile (see example 2-1) according to protocol B. It was isolated by silica gel chromatography (dichloromethane/methanol 95/5).

Yield:21%

Aspect: pale yellow oil

¹H NMR (300 MHz, CDCl₃, δ in ppm): from 1.50 to 2.00 (m, 8H); from 2.70 to 2.90 (m, 5H); 3.08 (dd, 4.6 Hz, 13.3 Hz, 1H); 4.70 (dd, 4.6 Hz, 9.2 Hz, 1H); from 7.13 to 7.35 (m, 8H); 7.54 (d, 7.3 Hz, 1 H).

### Example 2-7

### 3-methyl-1-(3-(1-piperidinyl)phenyl)butylamine

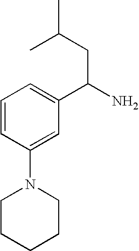

This amine was obtained in 2 steps;

### Step 1: 3-(1-piperidinyl)benzonitrile

This intermediate compound was synthesized from 3-fluorobenzonitrile according to protocol A as previously described. It was isolated by silica gel chromatography (cyclohexane/ethyl acetate 9/1).

Yield: 25%

Aspect: pale yellow oil

¹H NMR (300 MHz, CDCl₃, δ in ppm): from 1.60 to 1.70 (m, 6H); 3.18 (m, 4H); 7.01 (d, 7.7 Hz, 1H); 7.10 (m, 2H), from 7.25 to 7.36 (m, 1H).

### Step 2: 3-methyl-1-(3-(1-piperidinyl)phenyl)butylamine

This amine was obtained from the nitrile intermediate according to protocol B. It was isolated by silica gel chromatography (dichloromethane/methanol 98/2).

Yield: 48%

Aspect: pale yellow oil

¹H NMR (300 MHz, CDCl₃, δ in ppm): 0.92 (m, 6H); from 1.45 to 1.60 (m, 5H); from 1.65 to 1.80 (m, 4H); 1.92 (s(broad), 2H); 3.16 (m, 4H); 3.90 (t, 6.8 Hz, 1H); 6.77 (d, 7.6 Hz, 1H); 6.82 (dd, 8.2 Hz, 2.2 Hz, 1H); 6.92 (s, 1H); 7.21 (m, 1H).

### Example 2-8

### 3-methyl-1-(4-(1-piperidinyl)phenyl)butylamine

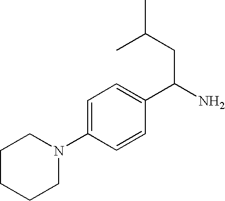

This amine was obtained in 2 steps:

### Step 1: 4-(1-piperidinyl)benzonitrile

This intermediate compound was synthesized from 4-fluorobenzonitrile according to protocol A as previously described. It was isolated by silica gel chromatography (cyclohexane/ethyl acetate 95/5).

Yield : quantitative

Aspect: pale yellow crystals

¹H NMR (300 MHz, CDCl₃, δ in ppm): 1.66 (m, 6H); 3.33 (m, 4H); 6.84 (d, 8.9 Hz, 2H); 7.46 (d, 8.9 Hz, 2H).

### Step 2: 3-methyl-1-(4-(1-piperidinyl)phenyl)butylamine

This amine was obtained from the nitrile intermediate according to protocol B. It was isolated by silica gel chromatography (dichloromethane/methanol 95/5).

Yield: 31%

Aspect: white solid

¹H NMR (300 MHz, CDCl₃, δ in ppm): 0.90 (m, 6H); from 1.40 to 1.80 (m, 9H); 2.12 (s(broad), 2H); 3.13 (m, 4H); 3.88 (t, 6.8 Hz, 1H); 6.90 (d, 8.6 Hz, 2H); 7.19 (d, 8.6 Hz, 2H).

### Example 2-9

### 1-(2-(1-piperidinyl)phenyl)pentylamine

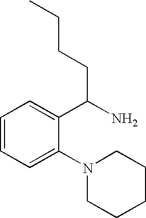

This amine was obtained from the intermediate 2-(1-piperidinyl)benzonitrile (see example 2-1) according to protocol B. It was isolated by silica gel chromatography (dichloromethane/methanol 95/5).

Yield: 71%

Aspect: colorless oil

¹H NMR (300 MHz, CDCl₃, δ in ppm): 0.90 (t, 7.4 Hz, 3H); from 1.20 to 1.80 (m, 14H); 2.82 (m, 4H); 4.34 (t, 7.1 Hz, 1H); from 7.05 to 7.25 (m, 3H); 7.32 (dd, 7.4 Hz and 1.6 Hz, 1H).

### Example 2-10

### 3-methyl-1-(2-(1-cyclohexylamino)phenyl)butylamine

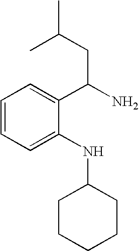

This amine was obtained in 2 steps

### Step 1: 2-(1-cyclohexylamino)benzonitrile

This intermediate compound was synthesized from 2-chlorobenzonitrile according to protocol A as previously described. It was isolated by silica gel chromatography (cyclohexane/ethyl acetate 98/2).

Yield: 8%

Aspect: pale orange oil

¹H NMR (300 MHz, CDCl₃, δ in ppm): from 1.20 to 2.07 (m, 10H); 3.35 (m, 1H); 4.45 (m, 1H); from 6.60 to 6.69 (m, 2H), from 7.33 to 7.39 (m, 2H).

### Step 2: 3-methyl-1-(2-(1-cyclohexylamino)phenyl)butylamine

This amine was obtained from the nitrile intermediate according to protocol B. No further purification was necessary after washings.

Yield: 78%

Aspect: white powder

¹H NMR (300 MHz, CDCl₃, δ in ppm): 0.91 (d, 6.1 Hz, 3H); 0.95 (d, 6.1 Hz, 3H); from 1.25 to 2.05 (m, 16H); 3.31 (m, 1H); 4.07 (t, 6.7 Hz, 1H); 6.57 (m, 1H); 6.64 (d, 7.8 Hz, 1H); 7.02 (dd, 7.8 Hz and 1.7 Hz, 1H); 7.11 (m, 1H).

### Example 2-11

### 1-(2-(1-pyrrolidinyl)phenyl)ethylamine

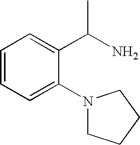

This amine was obtained in 2 steps

### Step 1: 2-(1-pyrrolidinyl)benzonitrile

This intermediate compound was synthesized from 2-chlorobenzonitrile according to protocol A as previously described. It was isolated by silica gel chromatography (cyclohexane/ethyl acetate 98/2).

Yield : 42%

Aspect: pale orange oil

¹H NMR (300 MHz, CDCl₃, δ in ppm): 2.01 (m, 4H); 3.61 (m, 4H); from 6.63 to 6.67 (m, 2H); from 7.30 to 7.37 (m, 1H); 7.44 (dd, 8.2 Hz and 1.6 Hz, 1H).

### Step 2: 1-(2-(1-pyrrolidinyl)phenyl)ethylamine

This amine was obtained from the nitrile intermediate according to protocol B. It was isolated by silica gel chromatography (dichloromethane/methanol/ammonium hydroxide 9/1/0.1).

Yield: 40%

Aspect: pale yellow oil

¹H NMR (300 MHz, CDCl₃, δ in ppm): 1.44 (d, 6.6 Hz, 3H); from 1.88 to 2.01 (m, 6H); 3.05 (m, 2H); 3.21 (m, 2H); 4.53 (q, 6.6 Hz, 1H); 7.05 (m, 2H); 7.20 (m, 1H); 7.40 (dd, 7.8 Hz and 1.7 Hz, 1H).

### Example 2-12

### 1-(2-(4-morpholinyl)phenyl)ethylamine

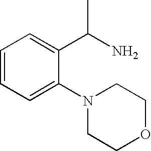

This amine was obtained in 2 steps

### Step 1: 2-(4-morpholinyl)benzonitrile

This intermediate compound was synthesized from 2-chlorobenzonitrile according to protocol A as previously described. It was isolated by silica gel chromatography (cyclohexane/ethyl acetate 8/2).

Yield: 38%

Aspect: pale yellow oil

¹H NMR (300 MHz, CDCl₃, δ in ppm): 3.22 (m, 4H); 3.92 (m, 4H); from 7.01 to 7.08 (m, 2H); from 7.49 to 7.55 (m, 1H); 7.59 (dd, 7.6 Hz and 1.7 Hz, 1H).

### Step 2: 1-(2-(4-morpholinyl)phenyl)ethylamine

This amine was obtained from the nitrile intermediate according to protocol B. It was isolated by silica gel chromatography (dichloromethane/methanol/ammonium hydroxide 9/1/0.2).

Yield: 45%

Aspect: pale yellow oil

¹H NMR (300 MHz, CDCl₃, δ in ppm): 1.43 (d, 6.5 Hz, 3H); 2.11 (s(broad), 2H); 2.92 (m, 4H); 3.87 (m, 4H); 4.62 (q, 6.6 Hz, 1H); 7.17 (m, 2H); 7.26 (m, 1H); 7.45 (dd, 6.8 Hz and 2.0 Hz, 1H).

### Example 2-13

### 1-(2-(1-hexamethyleneimino)phenyl)ethylamine

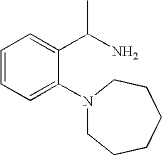

This amine was obtained in 2 steps

### Step 1: 2-(1-hexamethyleneimino)benzonitrile

This intermediate compound was synthesized from 2-fluorobenzonitrile according to protocol A as previously described. It was isolated by silica gel chromatography (cyclohexane/ethyl acetate 95/5).

Yield: 45%

Aspect: pale yellow oil

¹H NMR (300 MHz, CDCl₃, δ in ppm): 1.63 (m, 4H); 1.89 (m, 4H); 3.61 (m, 4H); 6.71 (m, 1H), 6.84 (d, 8.7 Hz, 1H); 7.35 (m, 1H); 7.59 (dd, 7.8 Hz and 1.7 Hz, 1H).

### Step 2: 1-(2-(1-hexamethyleneimino)phenyl)ethylamine

This amine was obtained from the nitrile intermediate according to protocol B. It was isolated by silica gel chromatography (dichloromethane/methanol/ammonium hydroxide 9/1/0.2).

Yield : 57%

Aspect: pale yellow oil

¹H NMR (300 MHz, CDCl₃, δ in ppm): 1.43 (d, 6.9 Hz, 3H); 1.75 (m, 8H); 2.27 (s(broad), 2H); 3.05 (m, 4H); 4.64 (q, 6.5 Hz, 1H); from 7.08 to 7.21 (m, 3H); 7.38 (dd, 7.4 Hz and 1.5 Hz, 1H).

### Example 2-14

### 3-methyl-1-(2-(1-pyrrolidinyl)phenyl)butylamine

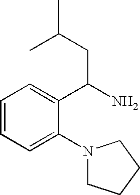

This amine was obtained from the intermediate 2-(1-pyrrolidinyl)benzonitrile (see example 2-11) according to protocol B. It was isolated by silica gel chromatography (dichloromethane/methanol/ammonium hydroxide 9/1/0.2).

Yield: 12%

Aspect: pale yellow oil

¹H NMR (300 MHz, CDCl₃, δ in ppm): 0.93 (m, 6H); from 1.52 to 1.65 (m, 3H); 1.93 (m, 6H); 3.09 (m, 4H); 4.47 (t, 6.6 Hz, 1H); 7.08 (m, 2H); 7.19 (m, 1H); 7.36 (dd, 7.6 Hz and 1.6 Hz, 1H).

### Example 2-15

### 3-methyl-1-(2-(4-morpholinyl)phenyl)butylamine

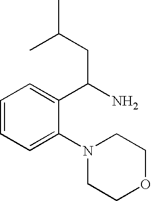

This amine was obtained from the intermediate 2-(4-morpholinyl)benzonitrile (see example 2-12) according to protocol B. It was isolated by silica gel chromatography (dichloromethane/methanol 9/1).

Yield: 45%

Aspect: pale yellow oil

¹H NMR (300 MHz, CDCl₃, δ in ppm): 0.94 (m, 6H); from 1.48 to 1.63 (m, 3H); 1.95 (s(broad), 2H); 2.84 (m, 2H); 2.98 (m, 2H); 3.85 (m, 4H); 4.57 (t, 6.6 Hz, 1H); 7.16 (m, 2H); 7.24 (m, 1H); 7.40 (d, 7.2 Hz, 1H).

### Example 2-16

### 3-methyl-1-(2-(1-hexamethyleneimino)phenyl)butylamine

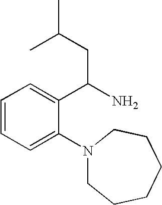

This amine was obtained from the intermediate 2-(1-hexamethyleneimino)benzonitrile (see example 2-13) according to protocol B. It was isolated by silica gel chromatography (dichloromethane/methanol 9/1).

Yield: 24%

Aspect: pale yellow oil

¹H NMR (300 MHz, CDCl₃/D₂O, δ in ppm): 0.94 (m, 6H); from 1.52 to 1.67 (m, 3H); 1.74 (m, 8H); 3.03 (m, 4H); 4.56 (t, 6.7 Hz, 1H); from 7.07 to 7.23 (m, 3H); 7.33 (dd, 7.6 Hz and 1.5 Hz, 1H).

### Example 2-17

### 2-cyclohexyl-1-(2-(1-piperidinyl)phenyl)ethylamine

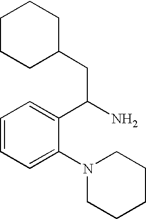

This amine was obtained from the intermediate 2-(1-piperidinyl)benzonitrile (see example 2-1) according to protocol B. It was isolated by silica gel chromatography (dichloromethane/methanol 9/1).

Yield: 23%

Aspect: pale yellow oil

¹H NMR (300 MHz, CDCl₃, δ in ppm): 0.94 (m, 2H); from 1.10 to 1.25 (m, 4H); from 1.45 to 1.90 (m, 15H); 2.81 (m, 4H); 4.53 (t, 6.7 Hz, 1H); from 7.09 to 7.24 (m, 3H); 7.35 (d, 7.7 Hz, 1H).

### Example 2-18

### 3-methyl-1-(2-(diethylamino)phenyl)butylamine

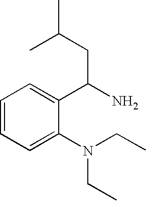

This amine was obtained in 2 steps

### Step 1: 2-(diethylamino)benzonitrile

The 2-aminobenzonitrile (10 g, 84.6 mmol) was solubilized in DMF (100 ml) and the mixture was cooled to 0° C. Sodium hydride (60% in oil, 11.3 g, 282 mmol) was added portionwise and the mixture was stirred at room temperature for 1 hour. The mixture was cooled down again, iodoethane (20.3 ml, 254 mmol) was added dropwise and the mixture was stirred at room temperature overnight. The crude was hydrolyzed (400 ml water) and extracted with ethyl acetate (2×200 ml). The organic layer was washed with 1M HCl (1×150 ml), 1M NaOH (2×150ml) and NaClₛₐₜ (2×150 ml). It was dried with magnesium sulfate (MgSO4), filtered and concentrated. The resulting residue was then purified by silica gel chromatography (heptane/ethyl acetate 95/5) to afford the expected product.

Yield: 49%

Aspect: pale yellow oil

¹H NMR (300 MHz, CDCl₃, δ in ppm): 1.19 (t, 7.1 Hz, 6H); 3.41 (q, 7.1 Hz, 4H); 6.84 (m, 1H); 6.93 (d, 8.4 Hz, 1H); 7.40 (m, 1H); 7.52 (dd, 7.7 Hz and 1.9 Hz, 1H).

### Step 2: 3-methyl-1-(2-(diethylamino)phenyl)butylamine

This amine was obtained from the nitrile intermediate according to protocol B. It was isolated by silica gel chromatography (dichloromethane/methanol 9/1).

Yield : 67%

Aspect: pale yellow oil

¹H NMR (300 MHz, CDCl₃, δ in ppm): 0.95 (m, 6H); 1.01 (t, 7.3 Hz, 6H); from 1.41 to 1.71 (m, 5H); 2.96 (q, 7.3 Hz, 4H); 4.64 (m, 1H); from 7.10 to 7.23 (m, 3H); 7.37 (d, 7.8 Hz, 1H).

### Example 2-19

### 3-methyl-1-(2-(1-piperidinyl)-4-(trifluoromethyl)phenyl)butylamine

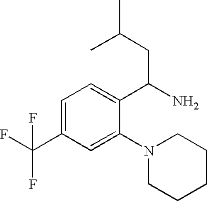

This amine was obtained in 2 steps

### Step 1: 2-(1-piperidinyl)-4-(trifluoromethyl)benzonitrile

This intermediate compound was synthesized from 2-fluoro-4-(trifluoromethyl)benzonitrile according to protocol A as previously described. The crude product was evaporated, solubilized in ethyl acetate, washed with 1M HCl and dried. No further purification was necessary.

Yield : 97%

Aspect: beige powder

¹H NMR (300 MHz, CDCl₃, δ in ppm): 1.65 (m, 2H); 1.81 (m, 4H); 3.25 (m, 4H); 7.20 (m, 2H), 7.65 (d, 8.2 Hz, 1H).

### Step 2: 3-methyl-1-(2-(1-piperidinyl)-4-(trifluoromethyl)phenyl)butylamine

This amine was obtained from the nitrile intermediate according to protocol B. It was isolated by silica gel chromatography (dichloromethane/methanol 9/1).

Yield : 5%

Aspect: pale orange oil

¹H NMR (300 MHz, CDCl₃, δ in ppm): 0.96 (m, 6H); from 1.47 to 1.73 (m, 11H); 2.82 (m, 4H); 4.53 (m, 1H); 7.35 (m, 2H); 7.47 (d, 8.7 Hz, 1H).

### Example 2-20

### 3-methyl-1-(2-(1-piperidinyl)-5-(chloro)phenyl)butamine

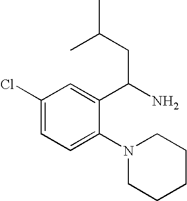

This amine was obtained in 4 steps

### Step 1: 2-(1-piperidinyl)-5-(nitro)benzonitrile

This intermediate compound was synthesized from 2-chloro-5-nitrobenzonitrile according to protocol A as previously described. It was isolated by silica gel chromatography (cyclohexane/ethyl acetate 8/2).

Yield : quantitative

Aspect: beige powder

¹H NMR (300 MHz, CDCl₃, δ in ppm): 1.78 (m, 6H); 3.51 (m, 4H); 6.96 (d, 9.4 Hz, 1H); 8.21 (dd, 9.4 Hz and 2.0 Hz, 1H); 8.41 (d, 2.0 Hz, 1H).

### Step 2: 2-(1-piperidinyl)-5-(amino)benzonitrile

The 2-(1-piperidinyl)-5-(nitro)benzonitrile (25 g, 108 mmol) was solubilized in methanol (400 ml). A spatula tip of Pd/C was added to the mixture and the crude was stirred for 16 hours under hydrogen atmosphere. The mixture was filtered on Celite®, concentrated and the expected product was isolated by recrystallization in an equimolar volume of dichloromethane and cyclohexane.

Yield: 87%

Aspect: pale yellow powder

¹H NMR (300 MHz, CDCl₃/D₂O, δ in ppm): 1.75 (m, 2H); 1.76 (m, 4H); 2.99 (m, 4H); from 6.81 to 6.92 (m, 3H).

### Step 3: 2-(1-piperidinyl)-5-(chloro)benzonitrile

The 2-(1-piperidinyl)-5-(amino)benzonitrile (10 g, 49.7 mmol) was solubilized in an aqueous hydrochloric acid solution (50 ml, 6N). NaNO₂ (3.4 g, 49.7 mmol) in water (30 ml) was added and the mixture was cooled to 0° C. This solution was added dropwise to Cu₂Cl₂ (14.8 g, 74.5 mmol) [freshly prepared by addition of an aqueous solution (50 ml) of NaHSO₃ (10.3 g, 99.4 mmol) on a solution of CuSO₄.5H₂O (49.6 g, 199 mmol) and NaCl (17.4 g, 298 mmol) in water (200 ml) at 35° C., filtration and water rinsing] solubilized in an aqueous concentrated hydrochloric acid solution (20 ml) at 0° C. Once the addition was finished, the crude mixture was heated to 50° C. until the end of the gaseous nitrogen evolution. The mixture was then cooled to 20° C., hydrolyzed by water addition (500 ml) and extracted with chloroform (3×200 ml). The organic layer was dried with magnesium sulfate (MgSO4), filtered and concentrated. The expected product was purified by silica gel chromatography (cyclohexane/ethyl acetate 95/5).

Yield: 76%

Aspect: pale yellow oil

¹H NMR (300 MHz, CDCl₃, δ in ppm): 1.61 (m, 2H); 1.77 (m, 4H); 3.14 (m, 4H); 6.92 (d, 8.8 Hz, 1H); 7.39 (dd, 8.8 Hz and 2.7 Hz, 1H); 7.48 (d, 2.7 Hz, 1H).

### Step 4 : 3-methyl-1-(2-(1-piperidinyl)-5-(chloro)phenyl)butamine

This amine was obtained from the nitrile intermediate according to protocol B. It was isolated by silica gel chromatography (dichloromethane/methanol 9/1).

Yield: 28%

Aspect: pale yellow oil

¹H NMR (300 MHz, CDCl₃, δ in ppm): from 0.93 to 1.00 (m, 6H); from 1.41 to 1.70 (m, 11H); 2.79 (m, 4H); 4.44 (t, 6.6 Hz, 1H); 7.05 (d, 8.4 Hz, 1H); 7.14 (dd, 8.4 Hz and 2.3 Hz,1H); 7.31 (d, 2.3 Hz, 1H).

### Example 2-21

### Ethyl 2-((4-aminomethyl)phenoxy)-2-methylpropanoate

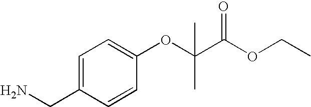

This amine was obtained in 3 steps from 4-hydroxybenzylamine

### Step 1: (4-(tert-butoxycarbonyl)aminomethyl)phenol

Di-tert-butyldicarbonate (11.1 g, 50.8 mmol) was added portionwise to a solution of 4-hydroxybenzylamine (6.25 g, 50.8 mmol) in tert-butanol (200 ml) and 1M sodium hydroxide (100 ml). The solution was stirred for 1 hour at room temperature. Water was added, pH was ajusted to 7 by addition of 1M hydrochloric acid, and the product was extracted by ethyl acetate. The organic layer was dried with magnesium sulfate (MgSO₄) and evaporated. The expected product was purified by silica gel chromatography (cyclohexane/ethyl acetate 7/3).

Yield: 83%

Aspect: white solid

¹H NMR (300 MHz, DMSO d6/D₂O, δ in ppm): 1.41 (s, 9H); 4.22 (s, 2H); 6.71 (d, 8.2 Hz, 2H), 7.01 (d, 8.2 Hz, 2H).

### Step 2: Ethyl 2-((4-(tert-butoxycarbonyl)aminomethyl)phenoxy)-2-methylpropanoate

Ethyl bromoisobutyrate was substituted by the (4-(tert-butoxycarbonyl)aminomethyl)phenol according to protocol D. The crude mixture was used for the next step without further purification.

Yield: 82%

Aspect: yellow oil

### Step 3: Ethyl 2-((4-aminomethyl)phenoxy)-2-methylpropanoate

Ethyl 2-((4-(tert-butoxycarbonyl)aminomethyl)phenoxy)-2-methylpropanoate (7.4 g, 21.9 mmol) was solubilized in dichloromethane (100 ml). Trifluoroacetic acid (10 ml) was added and the solution was stirred at room temperature for 2 hours. The crude mixture was successively washed with 1M NaOH (3×40 ml) and NaClsat (3×40 ml), dried with magnesium sulfate (MgSO₄), and evaporated. The expected product was purified by silica gel chromatography (dichloromethane/methanol 85/15).

Yield: 98%

Aspect: colorless oil

¹H NMR (300 MHz, CDCl₃, δ in ppm): 1.25 (t, 7.0 Hz, 3H); 1.57 (s, 6H); 2.76 (m, 2H); 3.80 (s, 2H); 4.22 (q, 7.0 Hz, 2H); 6.80 (d, 8.2 Hz, 2H); 7.19 (d, 8.2 Hz, 2H).

### Example 2-22

### 1-(2-(1-piperidinyl)-4-(bromo)phenyl)pentylamine

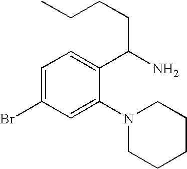

This amine was obtained in 2 steps from 4-bromo-2-fluorobenzonitrile

### Step 1: 2-(1-piperidinyl)-4-(bromo)benzonitrile

This intermediate compound was synthesized from 4-bromo-2-fluorobenzonitrile according to protocol A as previously described. The expected product was purified by silica gel chromatography (cyclohexane/ethyl acetate 95/5).

Yield: 53%

Aspect: white powder

¹H NMR (300 MHz, CDCl₃, δ in ppm): 1.62 (m, 2H); 1.78 (m, 4H); 3.19 (m, 4H); from 7.06 to 7.13 (m, 2H); 7.38 (d, 8.0 Hz, 1H).

### Step 2 : 1-(2-(1-piperidinyl)-4-(bromo)phenyl)pentylamine

This amine was obtained from the nitrile intermediate according to protocol B. It was isolated by silica gel chromatography (dichloromethane/methanol 9/1).

Yield: 12%

Aspect: yellow oil

¹H NMR (300 MHz, CDCl₃, δ in ppm): 0.89 (t, 7.2 Hz, 3H); from 1.18 to 1.36 (m, 4H); from 1.57 to 1.74 (m, 10H); from 2.74 to 2.86 (m, 4H); 4.29 (t, 7.2 Hz, 1H); 7.22 (m, 3H).

### Example 2-23

### N-isobutyl-N-(1-(2-(1-piperidinyl)phenyl)pentylamine

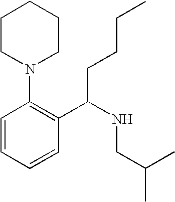

This amine was obtained in 1 step from the intermediate 1-(2-(1-piperidinyl)phenyl)pentylamine (see example 2-9).

Under anhydrous atmosphere, amine 2-9 (0.55 g, 2.24 mmol) and isobutyraldehyde (0.16 g, 2.24 mmol) were solubilized in a mixture of 1,2-dichloroethane (10 ml) and acetic acid (130 μl, 2.24 mmol). The mixture was cooled with an ice bath and sodium triacetoxyborohydure (0.66 g, 3.14 mmol) was added by portions. The mixture was warmed to room temperature and stirred for 24 hours. A molar solution of sodium hydroxide was added (20 ml), the mixture was stirred for 20 minutes and the whole was extracted with dichloromethane (2×50 ml). The resulting organic layer was dried with magnesium sulfate (MgSO4), filtered and concentrated. The expected product was purified by silica gel chromatography (dichloromethane/methanol 9/1).

Yield: 39%

Aspect: yellow oil

¹H NMR (300 MHz, CDCl₃, δ in ppm): from 0.84 to 0.90 (m, 9H); from 1.12 to 1.38 (m, 5H); from 1.58 to 1.90 (m, 9H); 2.19 (m, 1H); 2.32 (m, 1H); 2.80 (m, 4H); 4.19 (t, 7.5 Hz, 1H); from 7.11 to 7.25 (m, 3H); 7.33 (dd, 7.8 Hz, 1.8 Hz, 1H).

### Example 2-24

### 1-(2-(phenylthio)phenyl)pentylamine

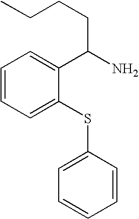

This amine was obtained in 2 steps from 2-fluorobenzonitrile

### Step 1: 2-(phenylthio)benzonitrile

Thiophenol (4.5 g, 41.3 mmol) was solubilized in acetonitrile (50 ml) and potassium carbonate (17.1 g, 124 mmol) was added. After 30 minutes stirring at room temperature, 2-fluorobenzonitrile (5.0 g, 41.3 mmol) was added dropwise and the mixture was refluxed for 16 hours. Salts were filtrated, rinsed, and the filtrate was concentrated. The residue was taken in ethyl acetate (100 ml) and successively washed with 1M NaOH washed with (3×50 ml), 1M HCl (3×50 ml), and NaClsat (3×50 ml). The organic layer was dried with magnesium sulfate (MgSO4), filtered and concentrated. The expected product was purified by silica gel chromatography (cyclohexane/ethyl acetate 9/1).

Yield: 86%

Aspect: yellow oil

¹H NMR (300 MHz, CDCl₃, δ in ppm): 7.14 (d, 7.6 Hz, 1H); 7.27 (m, 1H); from 7.38 to 7.51 (m, 6H); 7.65 (dd, 7.6 Hz, 1.6 Hz, 1H).

### Step 2: 1-(2-(phenylthio)phenyl)pentylamine

This amine was obtained from the nitrile intermediate according to protocol B. It was isolated by silica gel chromatography (dichloromethane/methanol 9/1).

Yield: 40%

Aspect: beige oil

¹H NMR (300 MHz, CDCl₃, δ in ppm): 0.85 (t, 6.7 Hz, 3H); from 1.17 to 1.38 (m, 4H); from 1.58 to 1.76 (m, 4H); 4.53 (t, 6.7 Hz, 1H); from 7.15 to 7.37 (m, 8H); 7.54 (dd, 8.0 Hz, 1.8 Hz, 1H).

### Example 2-25

### 3-methyl-1-(2-(1-piperidinyl)-5-(bromo)phenyl)butamine

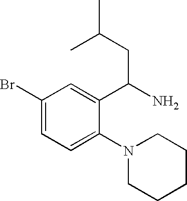

This product was synthesized in 2 steps from 2-(1-piperidinyl)-5-(amino)benzonitrile (synthesis of this intermediate has been described previously in example 2-20).

### Step 1: 2-(1-piperidinyl)-5-(bromo)benzonitrile

2-(1-piperidinyl)-5-(amino)benzonitrile (10 g, 49.7 mmol) was solubilized in an aqueous hydrochloric acid solution (30 ml, 6N). NaNO₂ (5.1 g, 74.5 mmol) in solution in water (10 ml) was added and the mixture was cooled to 0° C. with an ice bath. This solution was added dropwise to Cu₂Cl₂ (14.8 g, 74.5 mmol) [freshly prepared by addition of an aqueous solution (50 ml) of NaHSO₃ (10.3 g, 99.4 mmol) in a solution of CuSO₄.5H₂O (49.6 g, 199 mmol) and NaCl (17.4 g, 298 mmol) in water (200 ml) at 35° C., filtration then water rinsing] solubilized in an aqueous concentrated hydrochloric acid solution (20 ml) at 0° C. Once the addition was finished, the crude mixture was heated to 50° C. until the end of the gaseous nitrogen evolution. The mixture was then cooled to 20° C., hydrolyzed by water addition (500 ml), and extracted with chloroform (3×200 ml). The organic layer was dried with magnesium sulfate (MgSO4), filtered and concentrated. The expected product was purified by silica gel chromatography (cyclohexane/ethyl acetate 9/1).

Yield: 36%

Aspect: pale yellow oil

¹H NMR (300 MHz, CDCl₃, δ in ppm): 1.59 (m, 2H); 1.77 (m, 4H); 3.14 (m, 4H); 6.93 (d, 8.8 Hz, 1H); 7.39 (dd, 8.8 Hz and 2.3 Hz, 1H); 7.49 (d, 2.3 Hz, 1H).

### Step 2 : 3-methyl-1-(2-(1-piperidinyl)-5-(bromo)phenyl)butamine

This amine was obtained from the nitrile intermediate according to protocol B. It was isolated by silica gel chromatography (dichloromethane/methanol 95/5).

Yield: 29%

Aspect: pale yellow oil

¹H NMR (300 MHz, CDCl₃, δ in ppm): 0.94 (m, 6H); from 1.41 to 1.72 (m, 11H); 2.76 (m, 4H); 4.44 (t, 7.0 Hz, 1H); 7.05 (d, 8.4 Hz, 1H); 7.14 (dd, 8.4 Hz and 2.5 Hz, 1H); 7.31 (d, 2.5 Hz, 1H).

### Example 2-26

### 3-methyl-1-(2-(1-piperidinyl)-4-(bromo)phenyl)butamine

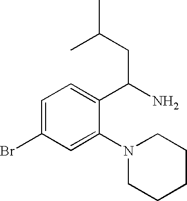

This amine was obtained from 2-(1-piperidinyl)-4-(bromo)benzonitrile (synthesis of this intermediate has been described previously in example 2-22) according to protocol B. It was isolated by silica gel chromatography (dichloromethane/methanol 9/1).

Yield: 8%

Aspect: pale yellow oil

¹H NMR (300 MHz, CDCl₃, δ in ppm): 0.91 (m, 6H); from 1.47 to 1.68 (m, 11H); from 2.73 to 2.83 (m, 4H); 4.48 (t, 7.0 Hz, 1H); from 7.16 to 7.26 (m, 3H).

### Example 2-27

### 1-(2-((3S,5R)-3,5-dimethylpiperidin-1-yl)phenyl)pentylamine

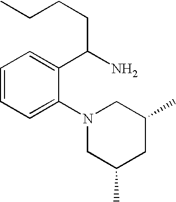

This amine was synthesized in 2 steps:

### Step 1: 2-((3S,5R)-3,5-dimethylpiperidin-1-yl)benzonitrile

2-fluorobenzonitrile (7.88 g, 65.1 mmol) and (3S,5R)-3,5-dimethylpiperidine (8.84 g, 78.1 mmol, prepared from commercial cis/trans mixture by recrystallization of the chlorhydrate form in toluene) were solubilized in acetonitrile (30 ml) and the mixture was refluxed for 5 days. The mixture was cooled to room temperature and treated by 1M aqueous citric acid (50 ml). The crude mixture was partly evaporated and then extracted with dichloromethane (2×50 ml). The organic layer was dried with magnesium sulfate, filtered and concentrated. The resulting residue was purified by silica gel chromatography (cyclohexane/ethyl acetate 98/2).

Yield: 34%

Aspect: pale yellow oil

¹H NMR (300 MHz, CDCl₃, δ in ppm): 0.69 (m, 1H); 0.94 (d, 6.6 Hz, 6H); from 1.84 to 2.01 (m, 3H); 2.31 (dd, 11.0 Hz and 11.0 Hz, 2H); 3.52 (m, 2H); from 6.92 to 7.01 (m, 2H); 7.45 (m, 1H); 7.54 (dd, 7.4 Hz and 1.5 Hz, 1H).

### Step 2: 1-(2-((3S,5R)-3,5-dimethylpiperidin-1-yl)phenyl)pentylamine

This amine was obtained from the previous nitrile intermediate according to protocol B. No further purification was necessary to get the pure product.

Yield: 70%

Aspect: pale yellow oil

¹H NMR (300 MHz, CDCl₃, δ in ppm): 0.67 (m, 1H); from 0.86 to 0.91 (m, 9H); from 1.18 to 1.43 (m, 3H); from 1.64 to 1.71 (m, 4H); from 1.79 to 1.88 (m, 3H); from 2.17 to 2.27 (m, 2H); from 2.86 to 2.98 (m, 2H); 4.31 (t, 7.1 Hz, 1H); from 7.08 to 7.23 (m, 3H); 7.32 (dd, 7.3 Hz and 1.6 Hz, 1H).

### Example 2-28

### 1-(2-(hydroxy)phenyl)pentylamine

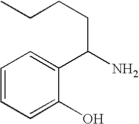

This amine was synthesized in 2 steps:

### Step 1: 2-(benzyloxy)benzonitrile

2-hydroxybenzonitrile (10.0 g, 83.9 mmol) and benzyl bromide (15.0 ml, 126 mmol) were solubilized in acetonitrile (100 ml) and potassium carbonate was added (23.2 g, 168 mmol). The mixture was refluxed overnight and cooled to room temperature. Salts were filtered and the filtrate was evaporated. The residue was treated by 1M HCl (100 ml) and extracted by ethyl acetate (3×50 ml). The organic layer was dried with magnesium sulfate, filtered and concentrated. The resulting residue was purified by silica gel chromatography (cyclohexane/ethyl acetate 8/2).

Yield: 80%

Aspect: white powder

¹H NMR (300 MHz, CDCl₃, δ in ppm): 5.23 (s, 2H); from 7.00 to 7.05 (m, 2H); from 7.32 to 7.61 (m, 7H).

### Step 2: 1-(2-(hydroxy)phenyl)pentylamine

This amine was obtained from the previous nitrile intermediate according to protocol B. It was isolated by silica gel chromatography (dichloromethane/methanol 9/1). The hydroxy group (initially protected by a benzyl group) was regenerated by this reaction.

Yield: 35%

Aspect: pale yellow oil

¹H NMR (300 MHz, DMSO d6/D2O, δ in ppm): 0.79 (m, 3H); from 1.07 to 1.59 (m, 6H); 3.96 (m,l H); 6.67 (m, 2H); 6.99 (m, 2H).

### Example 2-29

### 1-(3-fluoro-2-(1-piperidinyl)phenyl)pentylamine

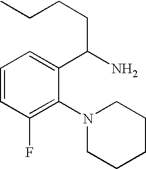

This amine was synthesized in 2 steps:

### Step 1: 3-fluoro-2-(1-piperidinyl)benzonitrile

2,3-difluorobenzonitrile (4.76 g, 34.2 mmol) was solubilized in acetonitrile (20 ml). Piperidine (3.4 ml, 34.2 mmol) was added and the mixture was stirred for 4 hours at room temperature. The crude mixture was added by 1M citric acid (50 ml), partly evaporated, and extracted with DCM. The organic layer was dried with magnesium sulfate, filtered and concentrated. The resulting residue was purified by silica gel chromatography (cyclohexane/ethyl acetate 98/2).

Yield: 21%

Aspect: white powder

¹H NMR (300 MHz, CDCl₃, δ in ppm): from 1.60 to 1.66 (m, 2H); from 1.71 to 1.79 (m, 4H); 3.27 (m, 4H); from 6.93 to 7.00 (m, 1H); from 7.16 to 7.23 (ddd, 12.1 Hz, 8.2 Hz, 1.3 Hz, 1H); 7.33 (d, 7.4 Hz, 1H).

### Step 2: 1-(3-fluoro-2-(1-piperidinyl)phenyl)pentylamine

This amine was obtained from the previous nitrile intermediate according to protocol B. No further purification was performed after the washing steps described in protocol B.

Yield: 6%

Aspect: pale yellow oil

¹H NMR (300 MHz, CDCl₃, δ in ppm): 0.89 (t, 7.0 Hz, 3H); from 1.16 to 1.40 (m, 4H); from 1.66 to 1.84 (m, 8H); 2.37 (s(broad), 2H); 2.86 (m, 2H); 3.17 (m, 2H); 4.30 (t, 7.0 Hz, 1H); from 6.87 to 6.95 (m, 1H); from 7.03 to 7.14 (m, 2H).

### Example 2-30

### 2-(4-methoxyphenyl)-1-(2-(1-piperidinyl)phenyl)ethylamine

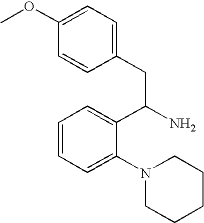

This amine was obtained from the intermediate 2-(1-piperidinyl)benzonitrile (synthesis of this intermediate has been previously described in example 2-1) according to protocol B. It was isolated by silica gel chromatography (dichloromethane/methanol 9/1).

Yield: 72%

Aspect: pale yellow oil

¹H NMR (300 MHz, CDCl₃, δ in ppm): from 1.65 to 1.75 (m, 8H); from 2.67 to 3.03 (m, 6H); 3.80 (s, 3H); 4.64 (m, 1H); 6.85 (d, 8.8 Hz, 2H); from 7.12 to 7.25 (m, 5H); 7.47 (d, 7.6 Hz, 1H).

### Example 2-31

### 3-phenyl-1-(2-(1-piperidinyl)phenyl)propylamine

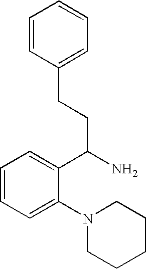

This amine was obtained from the intermediate 2-(1-piperidinyl)benzonitrile (synthesis of this intermediate has been previously described in example 2-1) according to protocol B. It was isolated by silica gel chromatography (dichloromethane/methanol 9/1).

Yield: 44%

Aspect: pale yellow oil

¹H NMR (300 MHz, CDCl₃, δ in ppm): from 1.56 to 1.66 (m, 6H); from 2.00 to 2.14 (m, 4H); from 2.56 to 2.90 (m, 6H); 4.36 (t, 7.0 Hz, 1H); from 7.11 to 7.37 (m, 9H).

### Example 2-32

### 2-(3-methylphenyl)-1-(2-(1-piperidinyl)phenyl)ethylamine

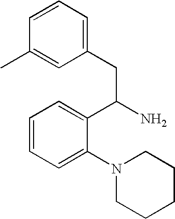

This amine was obtained from the intermediate 2-(1-piperidinyl)benzonitrile (synthesis of this intermediate has been previously described in example 2-1) according to protocol B. No further treatment was necessary to get the pure amine.

Yield: 72%

Aspect: pale yellow oil

¹H NMR (300 MHz, CDCl₃, δ in ppm): from 1.65 to 1.78 (m, 8H); 2.36 (s; 3H); from 2.72 to 2.95 (m, 5H); 3.04 (dd, 13.1 Hz, 4.4 Hz, 1H); 4.70 (m, 1H); from 7.04 to 7.25 (m, 7H); 7.51 (d, 7.3 Hz,1H).

### Example 2-33

### 1-(2-(1-piperidinyl)phenyl)butylamine

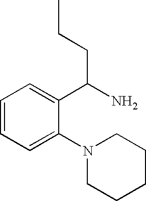

This amine was obtained from the intermediate 2-(1-piperidinyl)benzonitrile (see example 2-1) according to protocol B. The salt was obtained by treatment with glutaric acid in acetone at 50° C. and recrystallized upon cooling. The free amine was then regenerated by solubilization of the salt in 1M sodium hydroxide and extraction by ethyl acetate.

Yield: 77%

Aspect: pale yellow oil

¹H NMR (300 MHz, CDCl₃, δ in ppm): 0.95 (t, 7.3 Hz, 3H); from 1.22 to 1.91 (m, 12H); 2.83 (m, 4H); 4.38 (t, 7.0 Hz, 1H); from 7.05 to 7.25 (m, 3H); 7.32 (d, 7.6 Hz, 1H).

### Example 2-34

### 1-(2-(diethylamino)phenyl)pentylamine

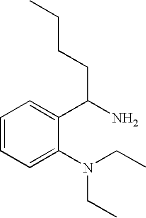

This amine was obtained from the 2-(diethylamino)benzonitrile (as described in example 2-18) according to protocol B. It was isolated by silica gel chromatography (dichloromethane/methanol 9/1).

Yield: 71%

Aspect: pale yellow oil

¹H NMR (300 MHz, CDCl₃, δ in ppm): 0.90 (t, 7.3 Hz, 3H); 1.00 (t, 7.0 Hz, 6H); from 1.21 to 1.45 (m, 4H); from 1.63 to 1.71 (m, 4H); 2.96 (q, 7.0 Hz, 4H); 4.55 (t, 7.0 Hz, 1H); from 7.11 to 7.24 (m, 3H); 7.37 (d, 7.6 Hz, 1H).

### Example 2-35

### 1-(2-(benzylamino)phenyl)pentylamine

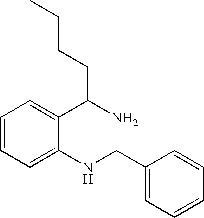

This amine was obtained in 2 steps:

### Step 1: 2-(benzylamino)benzonitrile

Benzaldehyde (9.0 g, 85 mmol) and 2-aminobenzonitrile (10 g, 85 mmol) were solubilized in absolute ethanol (150 ml). The mixture was refluxed for 3 days and cooled with an ice bath. Sodium borohydride (3.2 g, 85 mmol) was added and the crude was stirred overnight at room temperature. The precipitate was filtered, washed with ethanol, and dried under reduced pressure.

Yield: 39%

Aspect: white powder

¹H NMR (300 MHz, CDCl₃, δ in ppm): 4.45 (d, 5.6 Hz, 2H); 5.06 (s(broad), 1H); from 6.64 to 6.74 (m, 2H); from 7.30 to 7.38 (m, 7H).

### Step 2: 1-(2-(benzylamino)phenyl)pentylamine

This amine was obtained from the previous nitrile intermediate according to protocol B. It was isolated by silica gel chromatography (dichloromethane/methanol 95/5).

Yield: 60%

Aspect: pale yellow oil

¹H NMR (300 MHz, CDCl₃, δ in ppm): 0.94 (t, 7.0 Hz, 3H); from 1.15 to 1.65 (m, 6H); 1.77 (m, 1H); 2.02 (m, 1H); 4.05 (t, 7.0 Hz, 1H); 4.40 (m, 2H); 6.64 (m, 2H); 7.05 (d, 7.3 Hz, 1H); 7.13 (m, 1H); from 7.26 to 7.45 (m, 5H).

### Example 3

### Synthesis of Inventive Intermediate Compounds of Formula (V)

### Example 3-1

### Ethyl 2-((4-hydroxycarbonylmethyl)phenoxy)-2-methylpropanoate

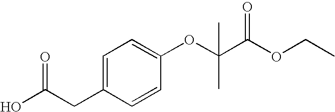

This acid was synthesized in 3 steps:

### Step 1: Benzyl 2-(4-hydroxyphenyl)acetate

The 2-(4-hydroxyphenyl)acetic acid was benzylated according to protocol C. The expected product was precipitated in petroleum ether, filtered and dried. No further purification was performed.

Yield: 94%

Aspect: white powder

¹H NMR (300 MHz, CDCl₃, δ in ppm): 3.62 (s, 2H); 5.16 (s, 2H); 6.76 (d, 8.1 Hz, 2H); 7.14 (d, 8.1 Hz, 2H); 7.36 (m, 5H).

### Step 2: Ethyl 2-((4-benzyloxycarbonylmethyl )phenoxy)-2-methylpropanoate

Ethyl bromoisobutyrate was substituted by benzyl 2-(4-hydroxyphenyl)acetate according to protocol D. The expected product was purified by silica gel chromatography (cyclohexane/ethyl acetate 9/1).

Yield: 95%

Aspect: colorless oil

¹H NMR (300 MHz, CDCl₃, δ in ppm): 1.26 (t, 6.8 Hz, 3H); 1.61 (s, 6H); 3.61 (s, 2H); 4.24 (q, 6.8 Hz, 2H); 5.14 (s, 2H); 6.81 (d, 8.4 Hz, 2H); 7.16 (d, 8.4 Hz, 2H); 7.33 (m, 5H).

### Step 3: Ethyl 2-((4-hydroxycarbonylmethyl)phenoxy)-2-methylpropanoate

The ethyl 2-((4-benzyloxycarbonylmethyl)phenoxy)-2-methylpropanoate was debenzylated according to protocol E. The expected product was purified by silica gel chromatography (dichloromethane/methanol 97/3).

Yield: 65%

Aspect: colorless oil

¹H NMR (300 MHz, CDCl₃, δ in ppm): 1.25 (t, 7.1 Hz, 3H); 1.59 (s, 6H); 3.57 (s, 2H); 4.23 (q, 7.1 Hz, 2H); 6.79 (d, 8.7 Hz, 2H); 7.15 (d, 8.7 Hz, 2H).

### Example 3-2

### Ethyl 2-((4-hydroxycarbonylmethyl-2-methoxy)phenoxy)-2-methylpropanoate

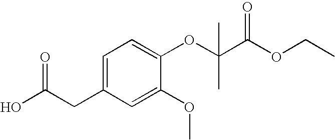

This acid was synthesized in 3 steps:

### Step 1: Benzyl 2-(4-hydroxy-3-methoxyphenyl)acetate

The 2-(4-hydroxy-3-methoxyphenyl)acetic acid was benzylated according to protocol C. The expected product was purified by silica gel chromatography (cyclohexane/ethyl acetate 8/2).

Yield: 91%

Aspect: colorless oil

¹H NMR (300 MHz, CDCl₃, δ in ppm): 3.61 (s, 2H); 3.86 (s, 3H); 5.15 (s, 2H); 5.60 (s, 1H); 6.80 (m, 2H); 6.87 (d, 8.0 Hz, 1H); 7.35 (m, 5H).

### Step 2: Ethyl 2-((4-benzyloxycarbonylmethyl-2-methoxy)phenoxy)-2-methylpropanoate

The phenol derivative was treated according to protocol D. The expected product was purified by silica gel chromatography (cyclohexane/ethyl acetate 9/1).

Yield: 60%

Aspect: colorless oil

¹H NMR (300 MHz, CDCl₃, δ in ppm): 1.28 (t, 7.0 Hz, 3H); 1.57 (s, 6H); 3.61 (s, 2H); 3.77 (s, 3H); 4.24 (q, 7.0 Hz, 2H); 5.14 (s, 2H); 6.74 (d, 8.4 Hz, 1H); 6.81 (m, 2H); 7.33 (m, 5H).

### Step 3: Ethyl 2-((4-hydroxycarbonylmethyl-2-methoxy)phenoxy)-2-methylpropanoate

The benzylic ester was deprotected according to protocol E. The expected product was purified by silica gel chromatography (dichloromethane/methanol 95/5).

Yield: 97%

Aspect: colorless oil

¹H NMR (300 MHz, CDCl₃, δ in ppm): 1.27 (t, 7.1 Hz, 3H); 1.57 (s, 6H); 3.58 (s, 2H); 3.81 (s, 3H); 4.24 (q, 7.1 Hz, 2H); 6.74 (d, 8.2 Hz, 1H); 6.81 (m, 2H).

### Example 3-3

### Ethyl 2-((2,6-dimethyl-4-hydroxycarbonylmethyl)phenoxy)-2-methylpropanoate

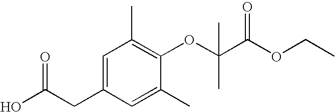

This acid was synthesized in 9 steps:

### Step 1: 3,5-dimethyl-4-benzyloxybenzaldehyde

3,5-dimethyl-4-hydroxybenzaldehyde (50.0 g; 0.333 mol) was solubilized in acetonitrile (600 ml) and potassium carbonate (92.0 g; 0.666 mol) was added. Benzyl bromide (39.6 ml; 0.333 mol) was slowly poured into the mixture under vigourous stirring at room temperature. The crude was refluxed for 16 h. Salts were filtered, washed with acetonitrile, and the filtrate was evaporated. The residue was taken in ethyl acetate, washed with 1M NaOH and NaClsat. The resulting organic layer was dried with magnesium sulfate (MgSO4) and evaporated. The resulting oil was used without further purification.

Yield: 99%

Aspect: colorless oil

¹H NMR (300 MHz, CDCl₃, δ in ppm): 2.37 (s, 6H); 4.89 (s, 2H); from 7.30 to 7.50 (m, 5H); 7.60 (s, 2H); 9.91 (s, 1H).

### Step 2: 3,5-dimethyl-4-benzyloxyphenylmethanol

Sodium borohydride (14.2 g; 0.374 mol) was slowly added to a solution of this aldehyde (60.0 g; 0.375 mol) in methanol (200 ml) previously cooled to 0° C. with an ice bath. The ice bath was then removed and stirring was maintained at room temperature until the end of gas evolution. Solvant was evaporated and 10% hydrochloric acid was slowly added to neutralize the mixture. The product was extracted with dichloromethane and washed with NaClsat. The organic layer was dried with magnesium sulfate (MgSO4) and evaporated. The obtained oil was used without further purification.

Yield: 96%

Aspect: colorless oil

¹H NMR (300 MHz, CDCl₃, δ in ppm): 2.34 (s, 6H); 4.59 (s, 2H); 4.84 (s, 2H); 7.06 (s, 2H); from 7.30 to 7.50 (m, 5H).

### Step 3: 1-((4-chloromethyl-2,6-dimethylphenoxy)methyl)benzene

Thionyl chloride (19.3 ml; 264 mmol) was added to a solution of this alcohol (53.3 g; 220 mmol) in dichloromethane (300 ml) previously cooled to 0° C. with an ice bath. The ice bath was then removed and stirring was maintained at room temperature for 2 h. The crude was then washed with a saturated aqueous solution of sodium bicarbonate and then with NaClsat. The organic layer was dried with magnesium sulfate (MgSO₄) and evaporated. The resulting solid was used without further purification.

Yield: 97%

Aspect: white solid

¹H NMR (300 MHz, CDCl₃, δ in ppm): 2.38 (s, 6H); 4.58 (s, 2H); 4.87 (s, 2H); 7.14 (s, 2H); from 7.30 to 7.50 (m, 5H).

### Step 4: 2-(4-benzyloxy-3,5-dimethylphenyl)acetonitrile

The previous chloride derivative (55.7 g; 214 mmol) was solubilized in DMF (300 ml) and potassium cyanide (15.7 g; 320 mmol; in 20 ml of water) was added. The mixture was heated to 80° C. for 3 h. The crude mixture was then poured in water (1.5 l) and extracted twice with ethyl acetate (300 ml). The organic layer was successively washed with water and NaClsat, dried with magnesium sulfate (MgSO4) and evaporated. The resulting crude solid was used without further purification.

Yield: 91%

Aspect: pale yellow solid

¹H NMR (300 MHz, CDCl₃, δ in ppm): 2.32 (s, 6H); 3.66 (s, 2H); 4.82 (s, 2H); 7.01 (s, 2H); from 7.30 to 7.50 (m, 5H).

### Step 5: 2-(4-benzyloxy-3,5-dimethylphenyl)acetic acid

The previously described nitrile (20.0 g; 72.5 mmol) was heated overnight at 80° C. in a mixture of 2M NaOH (200 ml), methanol (200 ml), and tetrahydrofurane (50 ml). Solvents were concentrated. The residue was acidified and extracted by ethyl acetate. The resulting organic layer was washed with NaClₛₐₜ, dried with magnesium sulfate (MgSO₄) and evaporated. The resulting crude solid was triturated with petroleum ether, filtered and dried.

Yield: 91%

Aspect: white flakes

¹H NMR (300 MHz, CDCl₃, δ in ppm): 2.32 (s, 6H); 3.58 (s, 2H); 4.82 (s, 2H); 6.99 (s, 2H); from 7.30 to 7.50 (m, 5H).

### Step 6: 2-(4-hydroxy-3,5-dimethylphenyl)acetic acid

The previously described benzyl ester was deprotected according to protocol E. The resulting crude solid was used without purification.

Yield: 98%

Aspect: off-white solid

¹H NMR (300 MHz, CDCl₃, δ in ppm): 2.23 (s, 6H); 3.52 (s, 2H); 6.90 (s, 2H).

### Step 7: Benzyl 2-(4-hydroxy-3,5-dimethylphenyl)acetate

The acid previously described was benzylated according to protocol C. The expected product was purified by silica gel chromatography (dichloromethane/cyclohexane 1/1).

Yield: 74%

Aspect: pale yellow solid

¹H NMR (300 MHz, CDCl₃, δ in ppm): 2.23 (s, 6H); 3.56 (s, 2H); 4.67 (s, 1H), 5.15 (s, 2H), 6.90 (s, 2H), from 7.30 to 7.40 (m, 5H).

### Step 8: Ethyl 2-((4-benzyloxycarbonylmethyl-2,6-dimethyl)phenoxy)-2-methylpropanoate

The phenol derivative was treated according to protocol D. The expected product was purified by silica gel chromatography (cyclohexane/ethyl acetate 9/1).

Yield: 30%

Aspect: colorless oil

¹H NMR (300 MHz, CDCl₃, δ in ppm): 1.37 (t, 6.7 Hz, 3H); 1.47 (s, 6H); 2.18 (s, 6H); 3.55 (s, 2H); 4.30 (q, 6.7 Hz, 2H); 5.14 (s, 2H), 6.89 (s, 2H), from 7.30 to 7.40 (m, 5H).

### Step 9: Ethyl 2-((2,6-dimethyl-4-hydroxycarbonylmethyl)phenoxy)-2-methylpropanoate

The benzyl ester previously described was deprotected according to protocol E. The expected product was purified by silica gel chromatography (dichloromethane/methanol 97/3).

Yield: 47%

Aspect: colorless oil

¹H NMR (300 MHz, CDCl₃, δ in ppm): 1.35 (t, 7.1 Hz, 3H); 1.47 (s, 6H); 2.19 (s, 6H); 3.52 (s, 2H); 4.29 (q, 7.1 Hz, 2H); 6.90 (s, 2H).

### Example 3-4

### Ethyl 2-((3-hydroxycarbonylmethyl)phenoxy)-2-methylpropanoate

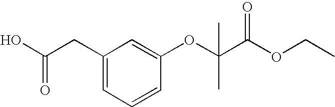

This acid was synthesized in 3 steps:

### Step 1: Benzyl 2-(3-hydroxyphenyl)acetate

2-(3-hydroxyphenyl)acetic acid was benzylated according to protocol C. The expected product was precipitated in petroleum ether, filtered and dried. No further purification was performed.

Yield: 95%

Aspect: white powder

¹H NMR (300 MHz, CDCl₃, δ in ppm): 3.63 (s, 2H); 5.07 (s, 1H); 5.15 (s, 2H); 6.75 (m, 2H); 7.85 (d, 8.0 Hz, 1H); 7.20 (m, 1H), 7.36 (m, 5H).

### Step 2: Ethyl 2-((3-benzyloxycarbonylmethyl)phenoxy)-2-methylpropanoate

Ethyl bromoisobutyrate was substituted by benzyl 2-(3-hydroxyphenyl)acetate according to protocol D. The expected product was purified by silica gel chromatography (cyclohexane/ethyl acetate 95/5).

Yield: 85%

Aspect: colorless oil

¹H NMR (300 MHz, CDCl₃, δ in ppm): 1.24 (t, 7.3 Hz, 3H); 1.59 (s, 6H); 3.62 (s, 2H); 4.23 (q, 7.3 Hz, 2H); 5.14 (s, 2H); 6.75 (d, 8.1 Hz, 1H); 6.82 (s, 1H); 6.92 (d, 7.3 Hz, 1H); 7.19 (m, 1H); 7.34 (m, 5H).

### Step 3: Ethyl 2-((3-hydroxycarbonylmethyl)phenoxy)-2-methylpropanoate

Ethyl 2-((3-benzyloxycarbonylmethyl)phenoxy)-2-methylpropanoate was debenzylated according to protocol E. The expected product was purified by silica gel chromatography (dichloromethane/methanol 97/3).

Yield: quantitative

Aspect: colorless oil

¹H NMR (300 MHz, CDCl₃, δ in ppm): 1.23 (t, 7.2 Hz, 3H); 1.60 (s, 6H); 3.59 (s, 2H); 4.23 (q, 7.2 Hz, 2H); 6.75 (dd, 8.0 Hz and 2.4 Hz, 1H); 6.81 (s, 1H); 6.91 (d, 7.6 Hz, 1H); 7.20 (m, 2H).

### Example 3-5

### 4-methyl-2-(2-(1-piperidinyl)phenyl)pentanoic acid

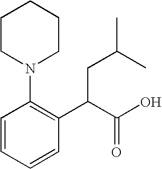

This acid was synthesized in 3 steps from the intermediate 2-(1-piperidinyl)benzonitrile (see example 2-1).

### Step 1: 3-methyl-1-(2-(1-piperidinyl)phenyl)butan-1-ol

Under anhydrous atmosphere, 2-(1-piperidinyl)benzonitrile (10 g, 53.7 mmol) was solubilized in anhydrous THF (50 ml). Freshly prepared isobutylmagnesium bromide (83 ml at 3.9 mol/l in THF, 324 mmol) was added and the mixture was refluxed overnight. The mixture was hydrolyzed with water and acidified by a 1M HCl solution until pH 7. The resulting ketone was extracted by ethyl acetate, dried with magnesium sulfate (MgSO₄), and evaporated. The residue was solubilized in methanol (100 ml) and the solution was cooled with an ice bath. Sodium borohydride (7.1 g, 188 mmol) was added portionwise at 0° C. and the reaction was slowly warmed to room temperature. Once the ketone was reduced, water was added and the alcohol was extracted by ethyl acetate, dried with magnesium sulfate (MgSO₄) and evaporated. The expected product was purified by silica gel chromatography (cyclohexane/ethyl acetate 95/5).

Yield: 60%

Aspect: pale yellow oil

¹H NMR (300 MHz, CDCl₃, δ in ppm): 0.99 (m, 6H); from 1.44 to 1.94 (m, 9H); 2.90 (m, 4H); 4.88 (m,1H); 6.67 (s(broad),1H); from 7.11 to 7.39 (m, 4H).

### Step 2: 4-methyl-2-(2-(1-piperidinyl)phenyl)pentanenitrile

3-methyl-1-(2-(1-piperidinyl)phenyl)butan-1-ol (7.0 g, 28.3 mmol) was solubilized in dichloromethane (200 ml) and the solution was cooled with an ice bath. Thionyl chloride (2.1 ml, 28.3 mmol) was added and the reaction was slowly warmed up to room temperature. Once the alcohol has disappeared (as followed by TLC), dichloromethane was evaporated under reduced pressure and the residue was taken in DMF (100 ml). Potassium cyanide (2.8 g, 43.0 mmol, solubilized in 5 ml of water) was added and the mixture was stirred at room temperature for 16 hours. Water was added and the expected product was extracted with ethyl acetate. The resulting organic layer was washed with NaClsat, dried with magnesium sulfate (MgSO₄), and evaporated. The expected product was purified by silica gel chromatography (cyclohexane/ethyl acetate 95/5).

Yield: 49%

Aspect: pale yellow oil

¹H NMR (300 MHz, CDCl₃, δ in ppm): 0.98 (d, 6.6 Hz, 3H); 1.01 (d, 6.6 Hz, 3H); from 1.44 to 1.92 (m, 9H); 2.73 (m, 2H); 2.89 (m, 2H); 4.57 (m, 1H); from 7.14 to 7.20 (m, 2H); 7.30 (m, 1H); 7.46 (d, 7.7 Hz, 1H).

### Step 3: 4-methyl-2-(2-(1-piperidinyl)phenyl)pentanoic acid

4-methyl-2-(2-(1-piperidinyl)phenyl)pentanenitrile (3 g, 11.7 mmol) was solubilized in hydrochloric acid 6N (20 ml) and heated to 80° C. for 2 days. The product was then extracted with dichloromethane (3×20 ml). Organic layers were combined and the resulting solution was washed with water (3×20 ml), dried with magnesium sulfate (MgSO₄) and evaporated. The residue was purified by silica gel chromatography (dichloromethane/methanol 95/5).

Yield: 80%

Aspect: pale yellow solid

¹H NMR (300 MHz, DMSO d6, δ in ppm): 0.85 (d, 6.6 Hz, 3H); 0.89 (d, 6.6 Hz, 3H); 1.27 (m, 1H); from 1.43 to 1.83 (m, 8H); 2.62 (m, 2H); 2.96 (m, 2H); 4.25 (t, 7.5 Hz, 1H); from 7.04 to 7.28 (m, 4H); 8.65 (s(broad), 1H).

### Example 3-6

### 2-(2-(1-piperidinyl)phenyl)hexanoic acid

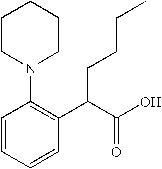

This acid was prepared in 3 steps from 2-(1-piperidinyl)benzonitrile intermediate (see example 2-1).

### Step 1: 1-(2-(1-piperidinyl)phenyl)pentan-1-ol

Under anhydrous atmosphere, 2-(1-piperidinyl)benzonitrile (15 g, 80.5 mmol) was solubilized in anhydrous THF (75 ml) and freshly prepared butylmagnesium bromide (242 mmol in 100 ml THF) was added. The mixture was refluxed overnight. The crude was then hydrolyzed with water and acidified with 1M HCl until a pH close to 7 was obtained. The resulting ketone was extracted with ethyl acetate, dried with magnesium sulfate (MgSO₄), and concentrated. The residue was taken in methanol (150 ml) and the solution was cooled with an ice bath. Sodium borohydride (6.1 g, 161 mmol) was added portionwise at 0° C. and the crude was warmed to room temperature. Upon total ketone consumption (according to TLC), water was added and the expected compound was extracted with ethyl acetate, dried with magnesium sulfate (MgSO₄), and concentrated. The expected product was isolated by silica gel chromatography (cyclohexane/ethyl acetate 95/5).

Yield: 55%

Aspect: pale yellow oil

¹H NMR (300 MHz, CDCl₃, δ in ppm): 0.93 (t, 7.0 Hz, 3H); from 1.37 to 1.78 (m, 13H); 2.91 (m, 4H); 4.79 (m, 1H); from 7.14 to 7.26 (m, 4H).

### Step 2: 2-(2-(1-piperidinyl)phenyl)hexanenitrile

1-(2-(1-piperidinyl)phenyl)pentan-1-ol (2.3 g, 9.26 mmol) was solubilized in dichloromethane (10 ml) and the solution was cooled with an ice bath. Thionyl chloride (1.0 ml, 13.9 mmol) was added and the crude was warmed to room temperature. Upon complete consumption of the reagent (according to TLC), dichloromethane was concentrated and the residue was solubilized with DMF (10 ml). Potassium cyanide (1.2 g, 18.5 mmol) was added as a solution in water (4 ml). The mixture was stirred at room temperature for 16 hours. Water was then added (50 ml) and the mixture was extrated with ethyl acetate, washed with NaClsat, dried with magnesium sulfate (MgSO₄), and concentrated. The expected compound was isolated by silica gel chromatography (cyclohexane/ethyl acetate 98/2).

Yield: 67%

Aspect: pale yellow oil

¹H NMR (300 MHz, CDCl₃, δ in ppm): 0.93 (t, 7.3 Hz, 3H); from 1.35 to 2.00 (m, 12H); 2.74 (m, 2H); 2.89 (m, 2H); 4.45 (m, 1H); from 7.14 to 7.21 (m, 2H); 7.31 (m, 1H); 7.45 (dd, 7.6 Hz, 1.5 Hz, 1H).

### Step 3: 2-(2-(1-piperidinyl)phenyl)hexanoic acid

2-(2-(1-piperidinyl)phenyl)hexanenitrile (1.5 g, 6.0 mmol) was solubilized in HCl 6M (10 ml) and heated to 80° C. overnight. pH was increased to 3 by addition of 2M NaOH and the mixture was extracted with dichloromethane (2×30 ml). Organic layers were combined and the resulting phase was washed with NaClₛₐₜ (2×30 ml), dried with magnesium sulfate (MgSO₄) and concentrated. The expected compound was isolated by silica gel chromatography (dichloromethane/methanol 98/2).

Yield: 89%

Aspect: white powder

¹H NMR (300 MHz, CDCl₃, δ in ppm): 0.92 (t, 7.3 Hz, 3H); from 1.26 to 1.47 (m, 5H); 1.86 (m, 6H); from 2.18 to 2.29 (m, 1H); from 2.65 to 3.30 (m, 4H); 3.83 (t, 7.3 Hz, 1H); from 7.19 to 7.23 (m, 4H).

### Example 4

### Synthesis of Inventive Intermediate Compounds of Formula (VI)

### Example 4-1

### 4-[1-(3-methyl-1-(2-(1-piperidinyl)phenyl)butyl)aminocarbonylmethyl]phenol

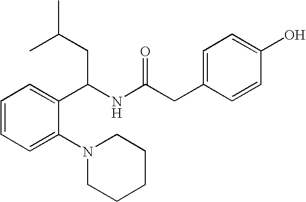

This intermediate compound was obtained by condensation of amine 2-1 and 4-hydroxyphenylacetic acid according to protocol G. It was purified by silica gel chromatography (cyclohexane/ethyl acetate 7/3).

Yield: 91%

Aspect: beige powder

¹H NMR (300 MHz, CDCl₃, δ in ppm): 0.92 (d, 6.5 Hz, 6H); from 1.38 to 1.80 (m, 9H); 2.65 (m, 2H); 2.97 (m, 2H); 3.47 (s, 2H); 5.40 (m, 1H); 6.57 (s, 1H); 6.64 (d, 8.7 Hz, 1H); 6.72 (d, 8.2 Hz, 2H); 7.04 (m, 4H); 7.20 (m, 2H).

### Example 4-2

### 4-[1-methyl-1-[1-(3-methyl-1-(2-(1-piperidinyl)phenyl)butyl)aminocarbonyl]methyl]phenol

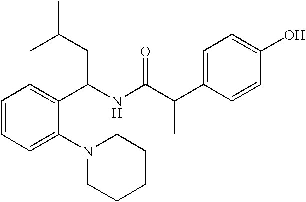

This intermediate compound was obtained by condensation of amine 2-1 and 2-(4-hydroxyphenyl)propanoic acid according to protocol G. It was purified by silica gel chromatography (cyclohexane/ethyl acetate 8/2).

Yield: 31%

Aspect: white powder

¹H NMR (300 MHz, CDCl₃, δ in ppm, diastereoisomers mixture 1/1): from 0.85 to 0.94 (m, 6H); from 1.25 to 1.71 (m, 12H); 2.55 and 2.67 (m+m, 2H); 2.96 (m, 2H); from 3.42 to 3.53 (m, 1H); from 5.30 to 5.39 (m, 1H); 5.74 and 5.80 (s+s, 1H); 6.48 (m, 1H); 6.70 and 6.78 (d+d, 8.5 Hz, 2H); from 6.96 to 7.23 (m, 6H).

### Example 4-3

### 4-[1-(1-(2-(1-piperidinyl)phenyl)pentyl)aminocarbonylmethyl]phenol

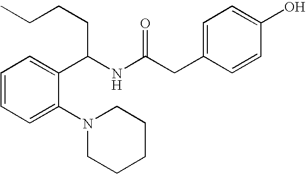

This intermediate compound was obtained by condensation of amine 2-9 and 4-hydroxyphenylacetic acid according to protocol G. The expected product was obtained pure without further purification.

Yield: 99%

Aspect: white solid

¹H NMR (300 MHz, CDCl₃, δ in ppm): 0.84 (t, 6.9 Hz, 3H); from 1.05 to 1.39 (m, 4H); from 1.46 to 1.80 (m, 8H); 2.66 (m, 2H); 2.93 (m, 2H); 3.48 (s, 2H); 5.25 (m, 1H); 6.74 (d, 8.1 Hz, 2H); from 6.99 to 7.34 (m, 8H).

### Example 4-4

### 4-[1-(1-(2-(1-piperidinyl)phenyl)heptyl)aminocarbonylmethyl]phenol

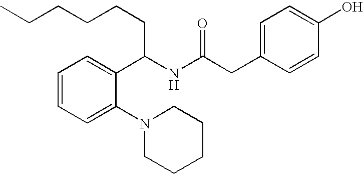

This intermediate compound was obtained by condensation of amine 2-4 and 4-hydroxyphenylacetic acid according to protocol G. It was purified by silica gel chromatography (dichloromethane/methanol 95/5).

Yield: 60%

Aspect: beige solid

¹H NMR (300 MHz, CDCl₃, δ in ppm): 0.84 (t, 6.9 Hz, 3H); from 1.05 to 2.09 (m, 16H); 2.94 (m, 4H); 3.50 (s, 2H); 5.05 (m, 1H); 6.72 (d, 8.2 Hz, 2H); from 6.90 to 7.80 (m, 8H).

### Example 4-5

### N-ethyl-N-[4-[1-(3-methyl-1-(2-(1-piperidinyl)phenyl)butyl)-aminocarbonylmethyl]phenyl]amine

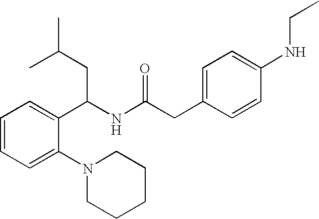

This amine was synthesized in 2 steps:

### Step 1: 4-[1-(3-methyl-1-(2-(1-piperidinyl)phenyl)butyl)aminocarbonylmethyl]-phenylamine

This intermediate compound was obtained by condensation of amine 2-1 and 4-aminophenylacetic acid according to protocol G. One molar equivalent of diisopropylethylamine per acid mole was added to allow the solubilization of the later. The resulting residue was purified by silica gel chromatography (cyclohexane/ethyl acetate 6/4).

Yield: 51%

Aspect: white powder

¹H NMR (300 MHz, CDCl₃, δ in ppm): 0.92 (d, 6.3 Hz, 6H); from 1.35 to 1.71 (m, 9H); 2.63 (m, 2H); 2.99 (m, 2H); 3.45 (m, 2H); 3.68 (m, 2H); 5.40 (m, 1H); 6.31 (d, 7.9 Hz, 1H); 6.67 (d, 8.4 Hz, 2H); 7.03 (m, 4H); from 7.15 to 7.23 (m, 2H).

### Step 2: N-ethyl-N-[4-[1-(3-methyl-1-(2-(l -piperidinyl)phenyl)butyl)-aminocarbonylmethyl]phenyl]amine

The amine previously described (1.5 g, 3.95 mmol) and acetaldehyde (333 μl, 5.93 mmol) were solubilized at 0° C. and under nitrogen atmosphere in a mixture of methanol (20 ml) and acetic acid (226 μL, 3.95 mmol). Sodium borohydride (224 mg, 5.93 mmol) was added portionwise and the mixture was stirred at room temperature overnight. The crude was hydrolyzed with water (10 ml) and extracted with dichloromethane (2×30 ml). The organic layer was dried with magnesium sulfate, filtered and concentrated. The resulting residue was purified by silica gel chromatography (cyclohexane/ethyl acetate 95/5).

Yield: 68%

Aspect: white powder

¹H NMR (300 MHz, CDCl₃, δ in ppm): 0.90 (d, 6.3 Hz, 3H); 0.91 (d, 6.3 Hz, 3H); 1.27 (t, 7.1 Hz, 3H); from 1.35 to 1.70 (m, 9H); 2.62 (m, 2H); 2.99 (m, 2H); 3.16 (q, 7.1 Hz, 2H); 3.44 (m, 2H); 3.58 (s(broad), 1H); 5.41 (m, 1H); 6.28 (d, 8.3 Hz, 1H); 6.59 (d, 8.3 Hz, 2H); from 7.00 to 7.07 (m, 4H); from 7.15 to 7.23 (m, 2H).

### Example 4-6

### 2-[1-(1-(2-(1-piperidinyl)phenyl)pentyl)aminocarbonylmethyl]phenol

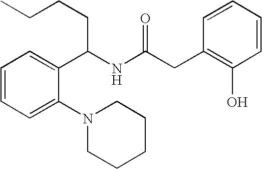

This product was synthesized in 3 steps:

### Step 1: 2-(2-(benzyloxy)phenyl)acetic acid

2-hydroxyphenylacetic acid (5 g, 32.9 mmol) was solubilized in ethanol (20 ml). 6.5M sodium hydroxide (10 ml) was added and the mixture was heated at 95° C. for 30 minutes. Benzyl bromide (11.7 ml, 98.6 mmol) was added and the crude mixture was refluxed for 16 hours. Solvant was partly evaporated and the mixture was acidified by addition of 1M citric acid (200 ml). The crude was extracted with ethyl acetate (3×100 ml) and dried with magnesium sulfate. The expected product was purified by silica gel chromatography (cyclohexane/ethyl acetate 6/4).

Yield: 100%

Aspect: pale yellow oil

¹H NMR (300 MHz, CDCl₃, δ in ppm): 3.75 (s, 2H); 5.10 (s, 2H); from 6.94 to 7.01 (m, 2H); from 7.20 to 7.43 (m, 7H).

### Step 2: [2-[1-(1-(2-(1-piperidinyl)phenyl)pentyl)aminocarbonylmethyl]phenyl]benzyl ether

This intermediate compound was obtained by condensation of amine 2-9 and 2-(2-(benzyloxy)phenyl)acetic acid according to protocol F. It was purified by silica gel chromatography (dichloromethane/ethyl acetate 9/1).

Yield: 65%

Aspect: pale yellow oil

¹H NMR (300 MHz, CDCl₃, δ in ppm): 0.79 (t, 6.7 Hz, 3H); from 1.03 to 1.30 (m, 4H); from 1.52 to 1.70 (m, 8H); 2.57 (m, 2H); 2.93 (m, 2H); 3.61 (m, 2H); 5.06 (s, 2H); 5.27 (m, 1H); 6.61 (d, 8.5 Hz, 1H); from 6.85 to 6.99 (m, 4H); from 7.08 to 7.37 (m, 9H).

### Step 3: 2-[1-(1-(2-(1-piperidinyl)phenyl)pentyl)aminocarbonylmethyl]phenol

The previously described intermediate (2.5 g, 5.31 mmol) was solubilized in methanol (25 ml) and Pd/C (10%, 565 mg, 0.53 mmol) and ammonium formiate (1.7 g, 26.6 mmol) were successively added. The mixture was stirred at room temperature overnight. The crude was filtered on Celite®. Filtrate was concentrated and purified by silica gel chromatography (cyclohexane/ethyl acetate 9/1).

Yield: 42%

Aspect: pale yellow oil

¹H NMR (300 MHz, CDCl₃, δ in ppm): 0.84 (t, 6.9 Hz, 3H); from 1.08 to 1.86 (m, 12H); 2.80 (m, 2H); 2.93 (m, 2H); 3.47 (d, 13.9 Hz, 1H); 3.66 (d, 13.9 Hz, 1H); 5.06 (m, 1H); 6.79 (m, 1H); 6.96 (m, 2H); from 7.09 to 7.31 (m, 5H); 8.39 (d, 8.7 Hz, 1H); 10.45 (s, 1H).

### Example 4-7

### 2-chloro-4-[1-(3-methyl-1-(2-(1-piperidinyl)phenyl)butyl)-aminocarbonylmethyl]phenol

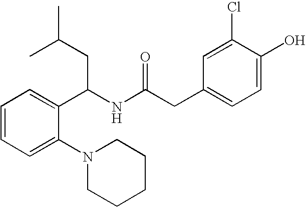

This intermediate compound was obtained by condensation of amine 2-1 and 3-chloro-4-hydroxyphenylacetic acid according to protocol F. The resulting residue was purified by silica gel chromatography (dichloromethane/ethyl acetate 9/1).

Yield: 46%

Aspect: beige powder

¹H NMR (300 MHz, CDCl₃, δ in ppm): 0.93 (m, 6H); from 1.37 to 1.80 (m, 9H); 2.65 (m, 2H); 2.94 (m, 2H); 3.45 (s, 2H); 5.37 (m, 1H); 5.98 (s, 1H); 6.74 (d, 8.8 Hz, 1H); 6.93 (d, 8.2 Hz, 1H); from 7.02 to 7.11 (m, 3H); from 7.20 to 7.28 (m, 3H).

### Example 4-8

### 4-[1-(1-(2-(1-piperidinyl)phenyl)butyl)aminocarbonylmethyl]phenol

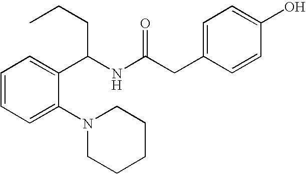

This intermediate compound was obtained by condensation of amine 2-33 and 4-hydroxyphenylacetic acid according to protocol G (slighty modified with replacing HOBt by DMAP in catalytic amounts). The resulting residue was purified by silica gel chromatography (dichloromethane/ethyl acetate 8/2).

Yield: 21%

Aspect: beige solid

¹H NMR (300 MHz, CDCl₃, δ in ppm): 0.88 (t, 7.3 Hz, 3H); from 1.02 to 1.80 (m, 10H); 2.65 (m, 2H); 2.92 (m, 2H); 3.54 (s, 2H); 5.25 (m, 1H); 6.83 (d, 8.0 Hz, 2H); from 7.00 to 7.40 (m, 8H).

### Example 4-9

### 4-[1-(2-phenyl-1-(2-(1-piperidinyl)phenyl)ethyl)-aminocarbonylmethyl]thiophenol

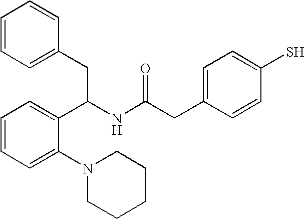

This intermediate compound was obtained by condensation of amine 2-6 and 4-mercaptophenylacetic acid according to protocol G (slighty modified with replacing DCC/HOBt by EDC/N-methylmorpholine in stoechiometric amounts). The resulting residue was purified by silica gel chromatography (dichloromethane/methanol 99/1).

Yield: 56%

Aspect: beige solid

¹H NMR (300 MHz, CDCl₃/D₂O, δ in ppm): from 1.62 to 1.72 (m, 6H); 2.62 (m, 2H); from 2.84 to 3.07 (m, 4H); 3.44 (m, 2H); 5.56 (m, 1H); from 6.92 to 7.48 (m, 13H).

### Example 4-10

### 4-[1-(1-(2-(diethylamino)phenyl)pentyl)-aminocarbonylmethyl]phenol

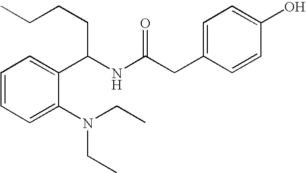

This intermediate compound was obtained by condensation of the amine 2-34 and 4-hydroxyphenylacetic acid according to protocol G. The resulting residue was purified by silica gel chromatography (dichloromethane/ethyl acetate 9/1).

Yield: 63%

Aspect: beige solid

¹H NMR (300 MHz, CDCl₃, δ in ppm): 0.83 (t, 6.9 Hz, 3H); 0.96 (t, 7.0 Hz, 6H); from 1.10 to 1.37 (m, 4H); 1.64 (m, 2H); 2.91 (m, 4H); 3.51 (m, 2H); 5.41 (m, 1H); 6.60 (m, 1H); 6.79 (d, 8.5 Hz, 2H); from 7.02 to 7.24 (m, 7H).

### Example 5

### Synthesis of Inventive Compounds

Compound 1:

### 2-[4-[1-(3-methyl-1-(2-(1-piperidinyl)phenyl)butyl)aminocarbonylmethyl]phenoxy]-2-methylpropanoic acid

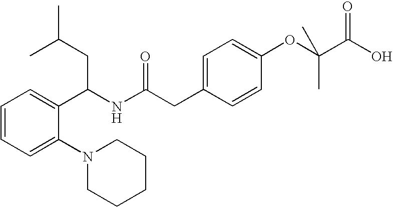

This product was synthesized in 2 steps:

### Step 1: Ethyl 2-[4-[1-(3-methyl-1-(2-(1-piperidinyl)phenyl)butyl)aminocarbonylmethyl]phenoxy]-2-methylpropanoate

This intermediate compound was obtained by condensation of amine 2-1 and acid 3-1 according to protocol G. It was purified by silica gel chromatography (dichloromethane/methanol 99/1).

Yield: 69%

Aspect: white powder

¹H NMR (300 MHz, CDCl₃, δ in ppm): 0.90 (d, 6.5 Hz, 6H); 1.26 (t, 7.1 Hz, 3H); from 1.40 to 1.80 (m, 15H); 2.63 (m, 2H); 2.96 (m, 2H); 3.48 (s, 2H); 4.24 (q, 7.1 Hz, 2H); 5.38 (m, 1H); 6.39 (d, 8.2 Hz, 1H); 6.82 (d, 8.7 Hz, 2H); 7.04 (m, 2H); from 7.12 to 7.20 (m, 4H).

### Step 2: 2-[4-[1-(3-methyl-1-(2-(1-piperidinyl)phenyl)butyl)aminocarbonylmethyl]phenoxy]-2-methylpropanoic acid

This compound was obtained by saponification of the previously described intermediate according to protocol H. The expected product was purified by recrystallization in ethyl acetate.

Yield: 74%

Aspect: white crystals

¹H NMR (300 MHz, DMSO d6, δ in ppm): 0.89 (d, 5.6 Hz, 6H); from 1.25 to 1.75 (m, 15H); 2.51 (m, 2H); 3.08 (m, 2H); 3.37 (m, 2H); 5.33 (m, 1H); 6.74 (d, 8.2 Hz, 2H); from 7.01 to 7.18 (m, 5H); 7.26 (d, 7.7 Hz, 1H); 8.35 (d, 8.7 Hz, 1H); 12.98 (s(broad), 1H).
Compounds 1A and 1B:

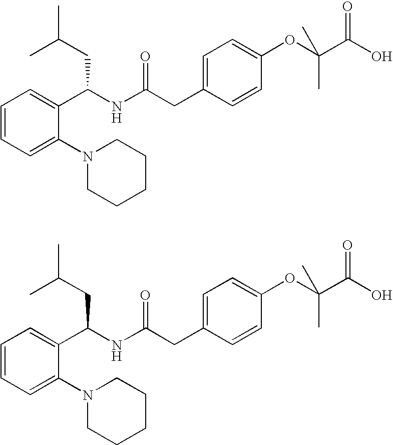

The two enantiomers of compound 1 were separated by semi-preparative HPLC on chiral column Chiralpak®AD (250*2.5 mm, 20 μm, Chiral Technologies Europe) at room temperature. The separation was performed upon gradual elution of a n-heptane-isopropanol mobile phase containing 0.1% trifluoroacetic acid. The enantiomeric purity of both enantiomers was checked by analytical HPLC: Chiralpak®AD-H column (250*4.6 mm, 5 μm, Chiral Technologies Europe) at 30° C.; n-heptane-isopropanol gradual mobile phase (95/5→90/10 in 20 min then 90/10) containing 0.1% trifluoroacetic acid; flow rate 1 ml/min; UV detection at 203 nm.

Compound 1A: R_{T}=29.5 min, 100%.

Compound 1B: R_{T}=21.9 min, 100%.

Compound 2:

### 2-[4-[1-(1-(2-(1-piperidinyl)phenyl)ethyl)aminocarbonylmethyl]phenoxy]-2-methylpropanoic acid

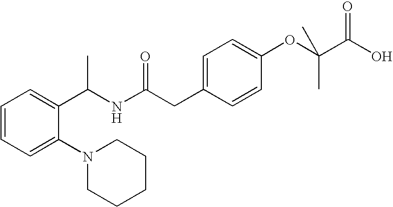

This product was synthesized in 2 steps:

### Step 1: Ethyl 2-[4-[1-(1-(2-(1-piperidinyl)phenyl)ethyl)aminocarbonylmethyl]phenoxy]-2-methylpropanoate

This intermediate compound was obtained by condensation of amine 2-2 and acid 3-1 according to protocol G. It was purified by silica gel chromatography (cyclohexane/ethyl acetate 9/1).

Yield: 74%

Aspect: white powder

¹H NMR (300 MHz, CDCl₃, δ in ppm): 1.25 (t, 7.0 Hz, 3H); 1.38 (d, 7.0 Hz, 3H); 1.50 to 1.80 (m, 12H); 2.67 (m, 2H); 2.91 (m, 2H); 3.48 (s, 2H); 4.23 (q, 7.0 Hz, 2H); 5.37 (m, 1H); 6.80 (d, 8.5 Hz, 2H); 6.95 (d, 7.2 Hz, 1H); from 7.07 to 7.26 (m, 6H).

### Step 2: 2-[4-[1-(1-(2-(1-piperidinyl)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 62%

Aspect: white powder

¹H NMR (300 MHz, CDCl₃, δ in ppm): 1.40 (d, 7.1 Hz, 3H); 1.50 to 1.72 (m, 12H); 2.66 (m, 2H); 2.88 (m, 2H); 3.47 (s, 2H); 5.32 (m, 1H); 6.87 (d, 8.2 Hz, 2H); 7.10 (m, 4H); 7.21 (m, 2H); 7.48 (s(broad), 1H).
Compounds 2A and 2B:

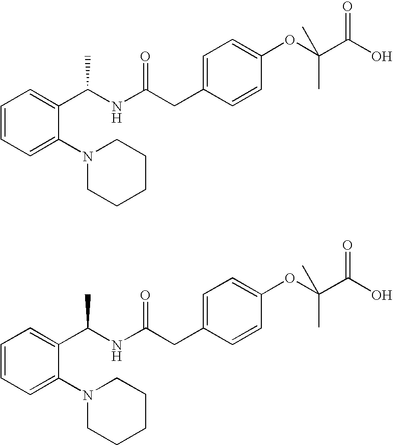

The two enantiomers of compound 2 were separated by semi-preparative HPLC on chiral column Chiralpak®AD (250*2.5 mm, 20 μm, Chiral Technologies Europe) at room temperature. The separation was performed upon gradual elution of a mobile phase n-heptane-isopropanol containing 0.1% trifluoroacetic acid.

The enantiomeric purity of both enantiomers was checked by analytical HPLC: Chiralpak®AD-H column (250*4.6 mm, 5 μm, Chiral Technologies Europe) at 30° C.; n-heptane-isopropanol gradual mobile phase (95/5→90/10 in 20 min then 90/10) containing 0.1% trifluoroacetic acid; flow rate 1 ml/min ; UV detection at 203 nm.

Compound 2A: R_{T}=31.8 min, 100%.

Compound 2B: R_{T}=25.0 min, 100%.

Compound 3:

### 2-[4-[1-(1-phenyl-1-(2-(1-piperidinyl)phenyl)methyl)aminocarbonylmethyl]phenoxy]-2-methylpropanoic acid

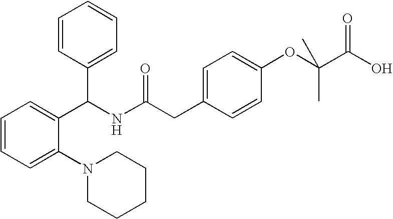

This product was synthesized in 2 steps:

### Step 1: Ethyl 2-[4-[1-(1-phenyl-1-(2-(1-piperidinyl)phenyl)methyl)aminocarbonylmethyl]phenoxy]-2-methylpropanoate

This intermediate compound was obtained by condensation of amine 2-3 and acid 3-1 according to protocol F. It was purified by silica gel chromatography (cyclohexane/ethyl acetate 9/1).

Yield: 86%

Aspect: white powder

¹H NMR (300 MHz, CDCl₃, δ in ppm): 1.25 (t, 7.2 Hz, 3H); 1.44 (m, 6H); 1.59 (s, 6H); 2.47 (m, 2H); 2.53 (m, 2H); 3.61 (m, 2H); 4.23 (q, 7.2 Hz, 2H); 6.50 (d, 8.7 Hz, 1H); 6.82 (d, 8.2 Hz, 2H); from 7.10 to 7.30 (m, 11H); 7.49 (d, 8.7Hz, 1H).

### Step 2: 2-[4-[1-(1-phenyl-1-(2-( 1-piperidinyl)phenyl)methyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 76%

Aspect: white powder

¹H NMR (300 MHz, CDCl₃, δ in ppm): from 1.30 to 1.50 (m, 6H); 1.60 (s, 6H); 2.45 (m, 4H); 3.58 (m, 2H); 6.47 (d, 8.7 Hz, 1H); 6.88 (d, 8.2 Hz, 2H); from 7.00 to 7.40 (m, 11H); 7.89 (d, 8.7 Hz, 1H).

Compound 4:

### 2-[2-methoxy-4-[1-(3-methyl-1-(2-(1-piperidinyl)phenyl)butyl)aminocarbonylmethyl]phenoxy]-2-methylpropanoic acid

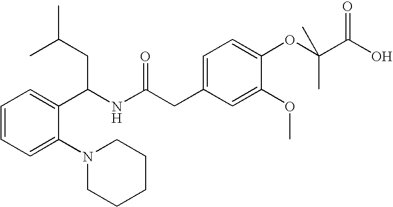

This product was synthesized in 2 steps:

### Step 1: Ethyl 2-[2-methoxy-4-[1-(3-methyl-1-(2-(1-piperidinyl)phenyl)butyl)aminocarbonylmethyl]phenoxy]-2-methylpropanoate

This intermediate compound was obtained by condensation of amine 2-1 and acid 3-2 according to protocol F. It was purified by silica gel chromatography (cyclohexane/ethyl acetate 9/1).

Yield: 84%

Aspect: white powder

¹H NMR (300 MHz, CDCl₃, δ in ppm): 0.91 (m, 6H); 1.29 (t, 7.1 Hz, 3H); from 1.30 to 1.80 (m, 15H); 2.62 (m, 2H); 2.96 (m, 2H); 3.49 (s, 2H); 3.75 (s, 3H); 4.25 (q, 7.1 Hz, 2H); 5.38 (m, 1H); 6.45 (d, 8.3 Hz, 1H); 6.70 (d, 8.2 Hz, 1H); 6.76 (s, 1H); 6.84 (d, 8.2 Hz, 1H); 7.04 (m, 2H); 7.18 (m, 2H).

### Step 2: 2-[2-methoxy-4-[1-(3-methyl-1-(2-( 1-piperidinyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 58%

Aspect: white powder

¹H NMR (300 MHz, CDCl₃, δ in ppm): 0.91 (m, 6H); from 1.30 to 1.80 (m, 15H); 2.62 (m, 2H); 2.93 (m, 2H); 3.51 (s, 2H); 3.76 (s, 3H); 5.36 (m, 1H); 6.73 (dd, 8.3 Hz, 1.6 Hz, 1H); 6.82 (m, 1H); 6.94 (d, 8.0 Hz, 1H); 7.07 (m, 2H); 7.18 (m, 2H).

Compound 5:

### 2-[4-[1-(1-(2-(1-piperidinyl)phenyl)heptyl)aminocarbonylmethyl]phenoxy]-2-methylpropanoic acid

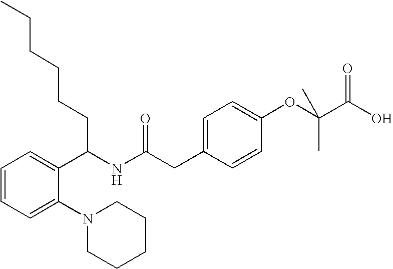

This product was synthesized in 2 steps:

### Step 1: Ethyl 2-[4-[1-(1-(2-(1-piperidinyl)phenyl)heptyl)aminocarbonylmethyl]phenoxy]-2-methylpropanoate

This intermediate compound was obtained by condensation of amine 2-4 and acid 3-1 according to protocol F. It was purified by silica gel chromatography (cyclohexane/ethyl acetate 8/2).

Yield: 92%

Aspect: white powder

¹H NMR (300 MHz, CDCl₃, δ in ppm): 0.86 (t, 6.5 Hz, 3H); from 1.00 to 1.30 (m, 11H); from 1.50 to 1.80 (m, 14H); 2.63 (m, 2H); 2.92 (m, 2H); 3.48 (s, 2H); 4.23 (q, 7.1 Hz, 2H); 5.23 (m, 1H); 6.75 (d, 8.7 Hz, 1H); 6.81 (d, 8.2 Hz, 2H); 7.04 (m, 2H); 7.13 (d, 8.2 Hz, 2H); 7.19 (m, 2H).

### Step 2: 2-[4-[1-(1-(2-(1-piperidinyl)phenyl)heptyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 99%

Aspect: white powder

¹H NMR (300 MHz, DMSO d6, δ in ppm): 0.84 (t, 6.2 Hz, 3H); from 1.10 to 1.30 (m, 8H); from 1.30 to 1.70 (m, 14H); 2.55 (m, 2H); 3.07 (m, 2H); 3.36 (m, 2H); 5.21 (m, 1H); 6.74 (d, 8.6 Hz, 2H); from 7.00 to 7.20 (m, 5H); 7.26 (d, 7.6 Hz, 1H); 8.35 (d, 8.6 Hz, 1H).

Compound 6:

### 2-[4-[1-(1-cyclohexyl-1-(2-(1-piperidinyl)phenyl)methyl)aminocarbonylmethyl]phenoxy]-2-methylpropanoic acid

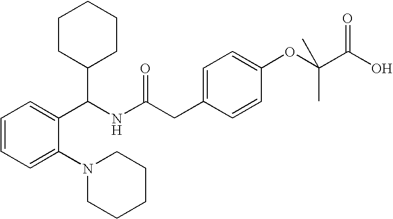

This product was synthesized in 2 steps:

### Step 1: Ethyl 2-[4-[1-(1-cyclohexyl-1-(2-(1-piperidinyl)phenyl)methyl)aminocarbonylmethyl]phenoxy]-2-methylpropanoate

This intermediate compound was obtained by condensation of amine 2-5 and acid 3-1 according to protocol F. It was purified by silica gel chromatography (cyclohexane/ethyl acetate 8/2).

Yield: 43%

Aspect: white powder

¹H NMR (300 MHz, CDCl₃, δ in ppm): from 0.80 to 1.10 (m, 6H); 1.26 (t, 7.1 Hz, 3H); from 1.50 to 1.80 (m, 17H); 2.59 (m, 2H); 2.88 (m, 2H); 3.49 (s, 2H); 4.24 (q, 7.1 Hz, 2H); 4.97 (m, 1H); 6.81 (m, 3H); from 6.95 to 7.05 (m, 2H); 7.13 (d, 8.2 Hz, 2H); 7.20 (m, 2H).

### Step 2: 2-[4-[1-(1-cyclohexyl-1-(2-(1-piperidinyl)phenyl)methyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 13%

Aspect: white powder

¹H NMR (300 MHz, CDCl₃, δ in ppm): from 0.80 to 1.10 (m, 6H); from 1.40 to 1.80 (m, 17H); 2.56 (m, 2H); 2.84 (m, 2H); 3.43 (m, 2H); 4.93 (m, 1H); 6.83 (d, 8.0 Hz, 2H); from 6.90 to 7.20 (m, 7H).

Compound 7:

### 2-[4-[1-(2-phenyl-1-(2-(1-piperidinyl)phenyl)ethyl)aminocarbonylmethyl]phenoxy]-2-methylpropanoic acid

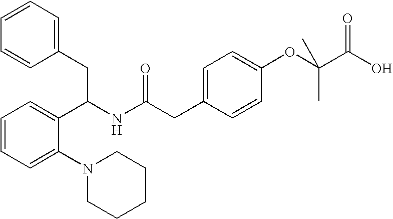

This product was synthesized in 2 steps:

### Step 1: Ethyl 2-[4-[1-(2-phenyl-1-(2-(1-piperidinyl)phenyl)ethyl)aminocarbonylmethyl]phenoxy]-2-methylpropanoate

This intermediate compound was obtained by condensation of amine 2-6 and acid 3-1 according to protocol F. It was purified by silica gel chromatography (cyclohexanelethyl acetate 8/2).

Yield: 88%

Aspect: white powder

¹H NMR (300 MHz, CDCl₃, δ in ppm): 1.27 (t, 7.1 Hz, 3H); from 1.50 to 1.80 (m, 12H); 2.62 (m, 2H); 2.84 (dd, 8.1 Hz, 13.4 Hz, 1H); 2.96 (m, 2H); 3.03 (dd, 6.4 Hz, 13.4 Hz, 1H); 3.43 (s, 2H); 4.26 (q, 7.1 Hz, 2H); 5.57 (m, 1H); 6.65 (d, 7.7 Hz, 1H); 6.81 (d, 8.8 Hz, 2H); from 6.80 to 7.10 (m, 6H); from 7.10 to 7.30 (m, 5H).

### Step 2: 2-[4-[1-(2-phenyl-1-(2-(1-piperidinyl)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 84%

Aspect: white powder

¹H NMR (300 MHz, DMSO d6, δ in ppm): from 1.50 to 1.80 (m, 12H); 2.45 (m, 2H); 2.73 (m, 1H); 2.90 (m, 1H); 3.07 (m, 2H); 3.29 (m, 2H); 5.47 (m, 1H); 6.67 (d, 8.2 Hz, 2H); 6.91 (d, 8.2 Hz, 2H); from 7.00 to 7.30 (m, 8H); 7.39 (d, 7.2 Hz, 1H); 8.53 (d, 8.6 Hz, 1H).

Compound 8:

### 2-[4-[1-(3-methyl-1-(3-(1-piperidinyl)phenyl)butyl)aminocarbonylmethyl]phenoxy]-2-methylpropanoic acid

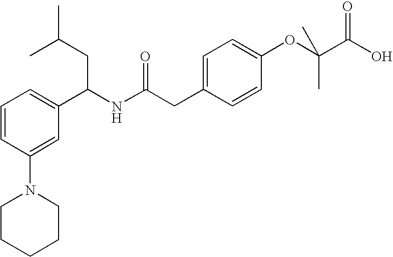

This product was synthesized in 2 steps:

### Step 1: Ethyl 2-[4-[1-(3-methyl-1-(3-(1-piperidinyl)phenyl)butyl)aminocarbonylmethyl]phenoxy]-2-methylpropanoate

This intermediate compound was obtained by condensation of amine 2-7 and acid 3-1 according to protocol F. It was purified by silica gel chromatography (dichloromethane/methanol 95/5).

Yield: 88%

Aspect: oil

¹H NMR (300 MHz, CDCl₃, δ in ppm): 0.87 (d, 6.5 Hz, 6H); 1.26 (t, 7.1 Hz, 3H); from 1.30 to 1.60 (m, 11H); 1.72 (m, 4H); 3.13 (m, 4H); 3.49 (m, 2H); 4.24 (q, 7.1 Hz, 2H); 4.97 (m, 1H); 5.54 (m, 1H); 6.60 (d, 7.7 Hz, 1H); 6.82 (m, 4H); 7.13 (m, 3H).

### Step 2: 2-[4-[1-(3-methyl-1-(3-( 1-piperidinyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 50%

Aspect: pale rose powder

¹H NMR (300 MHz, CDCl₃, δ in ppm): 0.87 (m, 6H); from 1.30 to 1.60 (m, 11H); from 1.70 to 1.80 (m, 4H); 3.12 (m, 4H); 3.45 (s, 2H); 4.97 (m, 1H); 5.52 (m, 1H); from 6.70 to 7.00 (m, 7H); 7.20 (m, 1H).

Compound 9:

### 2-[4-[1-(3-methyl-1-(4-(1-piperidinyl)phenyl)butyl)aminocarbonylmethyl]phenoxy]-2-methylpropanoic acid

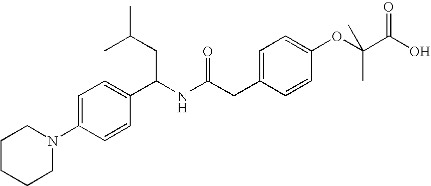

This product was synthesized in 2 steps:

### Step 1: Ethyl 2-[4-[1-(3-methyl-1-(4-(1-piperidinyl)phenyl)butyl)aminocarbonylmethyl]phenoxy]-2-methylpropanoate

This intermediate compound was obtained by condensation of amine 2-8 and acid 3-1 according to protocol F. It was purified by silica gel chromatography (dichloromethane/ethyl acetate 9/1).

Yield: 60%

Aspect: red solid

¹H NMR (300 MHz, CDCl₃, δ in ppm): 0.85 (m, 6H); 1.25 (t, 6.5 Hz, 3H); from 1.30 to 1.65 (m, 11H); 1.72 (m, 4H); 3.10 (m, 4H); 3.47 (m, 2H); 4.24 (q, 6.5 Hz, 2H); 4.90 (m, 1H); 5.84 (m, 1H); from 6.77 to 6.86 (m, 4H); 7.07 (m, 4H).

### Step 2: 2-[4-[1-(3-methyl-1-(4-(1-piperidinyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 96/4).

Yield: 50%

Aspect: red powder

¹H NMR (300 MHz, DMSO d6, δ in ppm): 0.81 (d, 6.0 Hz, 3H); 0.83 (d, 6.0 Hz, 3H); from 1.25 to 1.65 (m, 15H); 3.06 (m, 4H); 3.32 (s, 2H); 4.70 (m, 1H); 6.74 (d, 8.1 Hz, 2H); 6.84 (d, 8.6 Hz, 2H); 7.07 (m, 4H); 8.33 (d, 8.6 Hz, 1H).

Compound 10:

### 2-[4-[1-(1-(2-(1-piperidinyl)phenyl)pentyl)aminocarbonylmethyl]phenoxy]-2-methylpropanoic acid

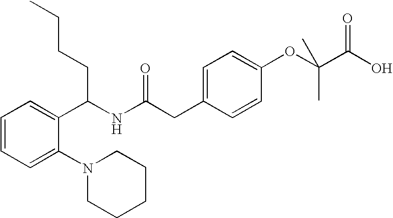

This product was synthesized in 2 steps:

### Step 1: Ethyl 2-[4-[1-(2-(2-(1-piperidinyl)phenyl)pentyl)aminocarbonylmethyl]phenoxy]-2-methylpropanoate

This intermediate compound was obtained by condensation of amine 2-9 and acid 3-1 according to protocol F. It was purified by silica gel chromatography (cyclohexane/ethyl acetate 8/2).

Yield: 92%

Aspect: off-white solid

¹H NMR (300 MHz, CDCl₃, δ in ppm): 0.83 (t, 7.0 Hz, 3H); from 1.15 to 1.75 (m, 21H); 2.63 (m, 2H); 2.92 (m, 2H); 3.48 (s, 2H); 4.23 (q, 7.0 Hz, 2H); 5.23 (m, 1H); 6.75 (d, 8.5 Hz, 1H); 6.80 (d, 8.5 Hz, 2H); from 7.00 to 7.20 (m, 6H).

### Step 2: 2-[4-[1-(1-(2-(1-piperidinyl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 73%

Aspect: beige powder

¹H NMR (300 MHz, DMSO d6, δ in ppm): 0.83 (t, 6.6 Hz, 3H); from 1.15 to 1.75 (m, 18H); 2.51 (m, 2H); 3.07 (m, 2H); 3.36 (s, 2H); 5.21 (m, 1H); 6.74 (d, 8.8 Hz, 2H); from 7.00 to 7.20 (m, 5H), 7.26 (d, 7.7 Hz, 1H); 8.35 (d, 8.3 Hz, 1H).

Compound 11:

### 2-[2,6-dimethyl-4-[1-(3-methyl-1-(2-(1-piperidinyl)phenyl)butyl)aminocarbonylmethyl]phenoxy]-2-methylpropanoic acid

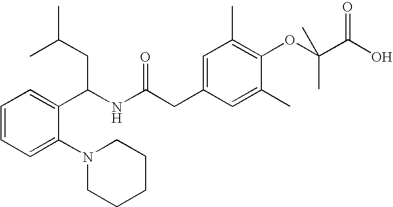

This product was synthesized in 2 steps:

### Step 1: Ethyl 2-[2,6-dimethyl-4-[1-(3-methyl-1-(2-(1-piperidinyl)phenyl)butyl)aminocarbonylmethyl]phenoxy]-2-methylpropanoate

This intermediate compound was obtained by condensation of amine 2-1 and acid 3-3 according to protocol F. It was purified by silica gel chromatography (cyclohexane/ethyl acetate 8/2).

Yield: 72%

Aspect: white solid

¹H NMR (300 MHz, CDCl₃, δ in ppm): 0.90 (m, 6H); from 1.30 to 1.80 (m, 18H); 2.18 (s, 6H); 2.63 (m, 2H); 2.97 (m, 2 H); 3.43 (s, 2H); 4.30 (q, 6.6 Hz, 2H); 5.37 (m, 1H); 6.34 (s(broad), 1H), 6.86 (s, 2H); 7.03 (m, 2H); 7.19 (m, 2H).

### Step 2: 2-[2,6-dimethyl-4-[1-(3-methyl-1-(2-(1-piperidinyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 32%

Aspect: white solid

¹H NMR (300 MHz, CDCl₃, δ in ppm): 0.91 (m, 6H); from 1.30 to 1.80 (m, 15H); 2.21 (s, 6H); 2.63 (m, 2H); 2.95 (m, 2 H); 3.46 (s, 2H); 5.38 (m, 1H); 6.55 (s(broad)m, 1H), 6.88 (s, 2H); 7.04 (m, 2H); 7.18 (m, 2H).

Compound 12:

### 2-[4-[1-(3-methyl-1-(2-(1-cyclohexylamino)phenyl)butyl)aminocarbonylmethyl]phenoxy]-2-methylpropanoic acid

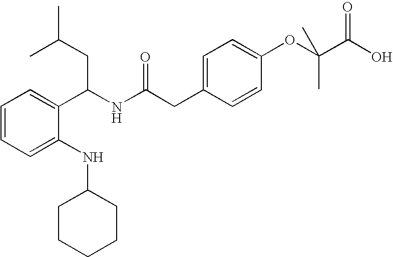

This product was synthesized in 2 steps:

### Step 1: Ethyl 2-[4-[1-(3-methyl-1-(2-(1-cyclohexylamino)phenyl)butyl)aminocarbonylmethyl]phenoxy]-2-methylpropanoate

This intermediate compound was obtained by condensation of amine 2-10 and acid 3-1 according to protocol F. It was purified by silica gel chromatography (cyclohexane/ethyl acetate 9/1).

Yield: 38%

Aspect: white powder

¹H NMR (300 MHz, CDCl₃, δ in ppm): 0.89 (d, 6.5 Hz, 3H); 0.94 (d, 6.5 Hz, 3H); from 1.17 to 2.06 (m, 22H); 3.26 (m, 1H); 3.46 (m, 2H); 4.24 (q, 7.1 Hz, 2H); 4.93 (m, 1H); 5.13 (m, 1H); 5.27 (m, 1H); from 6.55 to 6.65 (m, 2H); 6.79 (d, 8.7 Hz, 2H); from 7.01 to 7.17 (m, 4H).

### Step 2: 2-[4-[1-(3-methyl-1-(2-(1-cyclohexylamino)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 80%

Aspect: white powder

¹H NMR (300 MHz, DMSO d6/D₂O, δ in ppm): 0.85 (d, 6.6 Hz, 6H); from 1.05 to 1.87 (m, 19H); 3.14 (m, 1H); 3.31 (m, 2H); 4.87 (m, 1H); 6.54 (m, 2H); 6.72 (d, 8.3 Hz, 2H); 7.03 (m, 1H); 7.11 (m, 3H).

Compound 13:

### 2-[4-[1-(1-(2-(1-pyrrolidinyl)phenyl)ethyl)aminocarbonylmethyl]phenoxy]-2-methylpropanoic acid

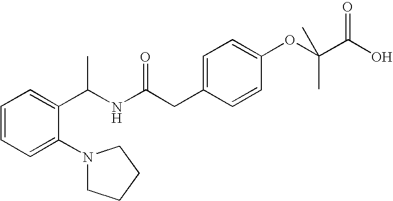

This product was synthesized in 2 steps:

### Step 1: Ethyl 2-[4-[1-(1-(2-(1-pyrrolidinyl)phenyl)ethyl)aminocarbonylmethyl]phenoxy]-2-methylpropanoate

This intermediate compound was obtained by condensation of amine 2-11 and acid 3-1 according to protocol F. It was purified by silica gel chromatography (cyclohexane/ethyl acetate 8/2).

Yield: 82%

Aspect: beige powder

¹H NMR (300 MHz, CDCl₃, δ in ppm): 1.25 (t, 7.3 Hz, 3H); 1.40 (d, 6.8 Hz, 3H); 1.59 (s, 6H); 1.87 (m, 4H); 2.91 (m, 2H); 3.15 (m, 2H); 3.49 (s, 2H); 4.24 (q, 7.3 Hz, 2H); 5.39 (m, 1H); 6.57 (d, 7.8 Hz, 1H); 6.80 (d, 8.3 Hz, 2H); 6.98 (m, 1H); from 7.08 to 7.22 (m, 5H).

### Step 2: 2-[4-[1-(1-(2-(1-pyrrolidinyl)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 31%

Aspect: beige powder

¹H NMR (300 MHz, DMSO d6/D₂O, δ in ppm): 1.25 (d, 7.0 Hz, 3H); 1.39 (s, 6H); 1.78 (m, 4H); 2.87 (m, 2H); 3.13 (m, 2H); 3.30 (s, 2H); 5.23 (m, 1H); 6.69 (d, 8.4 Hz, 2H); from 6.89 to 7.14 (m, 5H); 7.27 (d, 7.5 Hz, 1H).

Compound 14:

### 2-[4-[1-(1-(2-(4-morpholinyl)phenyl)ethyl)aminocarbonylmethyl]phenoxy]-2-methylpropanoic acid

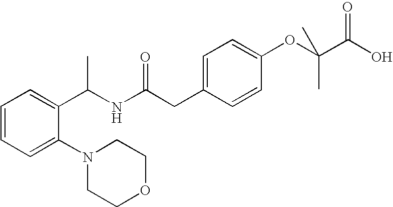

This product was synthesized in 2 steps:

### Step 1: Ethyl 2-[4-[1-(1-(2-(4-morpholinyl)phenyl)ethyl)aminocarbonylmethyl]phenoxy]-2-methylpropanoate

This intermediate compound was obtained by condensation of amine 2-12 and acid 3-1 according to protocol F. It was purified by silica gel chromatography (cyclohexane/ethyl acetate 8/2).

Yield: 69%

Aspect: white powder

¹H NMR (300 MHz, CDCl₃, δ in ppm): 1.26 (t, 7.2 Hz, 3H); 1.35 (d, 7.2 Hz, 3H); 1.60 (s, 6H); 2.70 (m, 2H); 3.05 (m, 2H); 3.49 (s, 2H); 3.74 (m, 2H); 3.82 (m, 2H); 4.24 (q, 7.2 Hz, 2H); 5.50 (m, 1H); 6.15 (m(broad), 1H), 6.82 (d, 8.3 Hz, 2H); from 7.10 to 7.28 (m, 6H).

### Step 2: 2-[4-[1-(1-(2-(4-morpholinyl)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 26%

Aspect: white powder

¹H NMR (300 MHz, DMSO d6/D₂O, δ in ppm): 1.29 (t, 6.6 Hz, 3H); 1.45 (s, 6H); 2.69 (m, 2H); 3.01 (m, 2H); 3.32 (s, 2H); 3.68 (m, 4H); 5.29 (m, 1H); 6.71 (d, 8.1 Hz, 2H); from 7.09 to 7.27 (m, 5H); 7.33 (d, 7.4 Hz, 1H).

Compound 15:

### 2-[4-[1-(1-(2-(1-hexamethyleneimino)phenyl)ethyl)aminocarbonylmethyl]phenoxy]-2-methylpropanoic acid

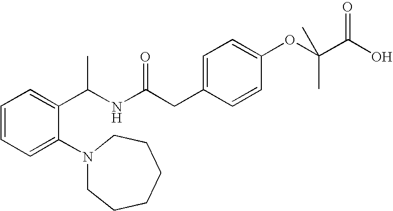

This product was synthesized in 2 steps:

### Step 1: Ethyl 2-[4-[1-(1-(2-(1-hexamethyleneimino)phenyl)ethyl)aminocarbonylmethyl]phenoxy]-2-methylpropanoate

This intermediate compound was obtained by condensation of amine 2-13 and acid 3-1 according to protocol F. It was purified by silica gel chromatography (cyclohexane/ethyl acetate 8/2).

Yield: 81%

Aspect: white powder

¹H NMR (300 MHz, CDCl₃, δ in ppm): 1.25 (t, 7.3 Hz, 3H); 1.38 (d, 6.8 Hz, 3H); 1.59 (s, 6H); 1.72 (m, 8H); 2.96 (m, 2H); 3.09 (m, 2H); 3.49 (s, 2H); 4.24 (q, 7.3 Hz, 2H); 5.48 (m, 1H); 6.75 (m(broad), 1H), 6.81 (d, 8.3 Hz, 2H); 7.05 (m, 2H); from 7.13 to 7.24 (m, 4H).

### Step 2: 2-[4-[1-(1-(2-(1-hexamethyleneimino)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 62%

Aspect: white powder

¹H NMR (300 MHz, DMSO d6/D₂O, δ in ppm): 1.26 (d, 7.0 Hz, 3H); 1.42 (s, 6H); 1.61 (m, 8H); 2.84 (m, 2H); 3.05 (m, 2H); 3.31 (s, 2H); 5.36 (m, 1H); 6.70 (d, 8.5 Hz, 2H); 6.99 (m, 1H); 7.09 (m, 4H); 7.23 (d, 7.6 Hz, 1H).

Compound 16:

### 2-[4-[1-(3-methyl-1-(2-(1-pyrrolidinyl)phenyl)butyl)aminocarbonylmethyl]phenoxy]-2-methylpropanoic acid

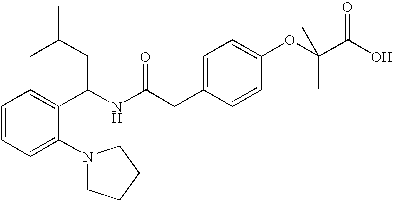

This product was synthesized in 2 steps:

### Step 1: Ethyl 2-[4-[1-(3-methyl-1-(2-(1-pyrrolidinyl)phenyl)butyl)aminocarbonylmethyl]phenoxy]-2-methylpropanoate

This intermediate compound was obtained by condensation of amine 2-14 and acid 3-1 according to protocol F. It was purified by silica gel chromatography (cyclohexane/ethyl acetate 8/2).

Yield: 95%

Aspect: beige powder

¹H NMR (300 MHz, CDCl₃/D₂O, δ in ppm): 0.87 (d, 6.8 Hz, 3H); 0.88 (d, 6.8 Hz, 3H); 1.26 (t, 7.1 Hz, 3H); 1.36 (m, 1H); 1.51 (m, 2H); 1.60 (s, 6H); 1.85 (m, 4H); 2.80 (m, 2H); 3.20 (m, 2H); 3.49 (m, 2H); 4.24 (q, 7.1 Hz, 2H); 5.37 (m, 1H); 6.82 (d, 8.6 Hz, 2H); from 6.96 to 7.05 (m, 2H); from 7.09 to 7.23 (m, 4H).

### Step 2: 2-[4-[1-(3-methyl-1-(2-(1-pyrrolidinyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 67%

Aspect: white powder

¹H NMR (300 MHz, DMSO d6/D₂O, δ in ppm): 0.82 (m, 6H); from 1.33 to 1.51 (m, 9H); 1.79 (m, 4H); 2.77 (m, 2H); 3.22 (m, 2H); 3.34 (s, 2H); 5.29 (m, 1H); 6.71 (d, 8.1 Hz, 2H); 6.95 (m, 1H); from 7.03 to 7.14 (m, 4H); 7.25 (d, 7.4 Hz, 1H).

Compound 17:

### 2-[4-[1-(3-methyl-1-(2-(4-morpholinyl)phenyl)butyl)aminocarbonylmethyl]phenoxy]-2-methylpropanoic acid

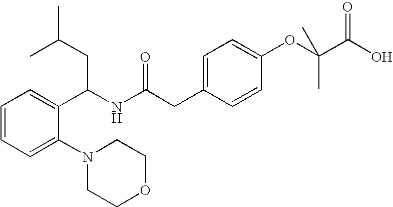

This product was synthesized in 2 steps:

### Step 1: Ethyl 2-[4-[1-(3-methyl-1-(2-(4-morpholinyl)phenyl)butyl)aminocarbonylmethyl]phenoxy]-2-methylpropanoate

This intermediate compound was obtained by condensation of amine 2-15 and acid 3-1 according to protocol F. It was purified by silica gel chromatography (cyclohexane/ethyl acetate 7/3).

Yield: 84%

Aspect: white powder

¹H NMR (300 MHz, CDCl₃, δ in ppm): 0.91 (m, 6H); 1.27 (t, 7.1 Hz, 3H); from 1.33 to 1.47 (m, 3H); 1.61 (s, 6H); 2.65 (m, 2H); 3.15 (m, 2H); 3.48 (m, 2H); 3.75 (m, 2H); 3.86 (m, 2H); 4.25 (q, 7.1 Hz, 2H); 5.52 (m, 1H); 5.86 (d, 8.2 Hz, 1H), 6.83 (d, 8.7 Hz, 2H); 7.00 (dd, 7.6 Hz and 1.7 Hz, 1H); from 7.08 to 7.26 (m, 5H).

### Step 2: 2-[4-[1-(3-methyl-1-(2-(4-morpholinyl)phenyl)butyl)aminocarbonylmethyl]phenoxy]-2-methylpropanoic acid

This compound was obtained by saponification of the previously described intermediate according to protocol H. The expected product was precipitated and washed by diethyl ether.

Yield: 73%

Aspect: white powder

¹H NMR (300 MHz, DMSO d6/D₂O, δ in ppm): 0.87 (d, 6.1 Hz, 6H); 1.30 (m, 1H); from 1.45 to 1.51 (m, 8H); 2.56 (m, 2H); 3.14 (m, 2H); 3.34 (m, 2H); 3.64 (m, 2H); 3.72 (m, 2H); 5.36 (m, 1H); 6.72 (d, 8.7 Hz, 2H); from 7.05 to 7.21 (m, 5H); 7.28 (d, 7.7 Hz, 1H).

Compound 18:

### 2-[4-[1-(3-methyl-1-(2-(1-hexamethyleneimino)phenyl)butyl)aminocarbonylmethyl]phenoxy]-2-methylpropanoic acid

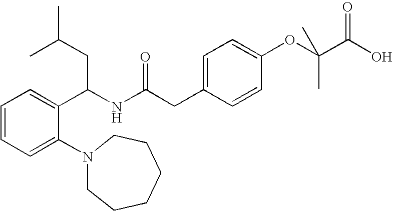

This product was synthesized in 2 steps:

### Step 1: Ethyl 2-[4-[1-(3-methyl-1-(2-(1-hexamethyleneimino)phenyl)butyl)aminocarbonylmethyl]phenoxy]-2-methylpropanoate

This intermediate compound was obtained by condensation of amine 2-16 and acid 3-1 according to protocol F. It was purified by silica gel chromatography (cyclohexane/ethyl acetate 8/2).

Yield: 61%

Aspect: white powder

¹H NMR (300 MHz, CDCl₃, δ in ppm): 0.90 (m, 6H); 1.26 (t, 7.0 Hz, 3H); from 1.39 to 1.60 (m, 9H); 1.78 (m, 8H); 2.95 (m, 2H); 3.11 (m, 2H); 3.48 (s, 2H); 4.24 (q, 7.0 Hz, 2H); 5.47 (m,1H); 6.35 (d, 8.3 Hz,1H), 6.82 (d, 8.7 Hz, 2H); 7.01 (m, 2H); from 7.12 to 7.20 (m, 4H).

### Step 2: 2-[4-[1-(3-methyl-1-(2-(1-hexamethyleneimino)phenyl)butyl)aminocarbonylmethyl]phenoxy]-2-methylpropanoic acid

This compound was obtained by saponification of the previously described intermediate according to protocol H. It was recrystallized in ethanol.

Yield: 57%

Aspect: white powder

¹H NMR (300 MHz, DMSO d6/D₂O, δ in ppm): 0.83 (m, 6H); from 1.32 to 1.72 (m, 17H); 2.80 (m, 2H); 3.07 (m, 2H); 3.33 (s, 2H); 5.40 (m, 1H); 6.71 (d, 8.1 Hz, 2H); 7.04 (m, 1H); 7.12 (m, 4H); 7.20 (d, 7.4 Hz, 1H).

Compound 19:

### 2-[4-[1-(2-cyclohexyl-1-(2-(1-piperidinyl)phenyl)ethyl)aminocarbonylmethyl]phenoxy]-2-methylpropanoic acid

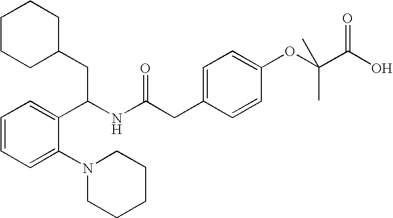

This product was synthesized in 2 steps:

### Step 1: Ethyl 2-[4-[1-(2-cyclohexyl-1-(2-(1-piperidinyl)phenyl)ethyl)aminocarbonylmethyl]phenoxy]-2-methylpropanoate

This intermediate compound was obtained by condensation of amine 2-17 and acid 3-1 according to protocol F. It was purified by silica gel chromatography (cyclohexane/ethyl acetate 85/15).

Yield: 67%

Aspect: white powder

¹H NMR (300 MHz, CDCl₃, δ in ppm): 0.90 (m, 2H); 1.04 (m, 4H); 1.26 (t, 7.1 Hz, 3H); 1.49 (m, 2H); from 1.51 to 1.76 (m, 17H); 2.62 (m, 2H); 2.97 (m, 2H); 3.48 (s, 2H); 4.24 (q, 7.1 Hz, 2H); 5.40 (m, 1H); 6.38 (d, 8.7 Hz, 1H), 6.82 (d, 8.7 Hz, 2H); 7.04 (m, 2H); from 7.12 to 7.23 (m, 4H).

### Step 2: 2-[4-[1-(2-cyclohexyl-1-(2-(1-piperidinyl)phenyl)ethyl)aminocarbonylmethyl]phenoxy]-2-methylpropanoic acid

This compound was obtained by saponification of the previously described intermediate according to protocol H. It was recrystallized in ethanol.

Yield: 82%

Aspect: white powder

¹H NMR (300 MHz, DMSO d6/D₂O, δ in ppm): 0.83 (m, 2H); from 0.93 to 1.14 (m, 4H); from 1.34 to 1.64 (m, 19H); 2.51 (m, 2H); 3.02 (m, 2H); 3.32 (m, 2H); 5.31 (m, 1H); 6.72 (d, 8.3 Hz, 2H); from 6.98 to 7.24 (m, 5H); 7.22 (d, 7.3 Hz, 1H).

Compound 20:

### 2-[4-[1-(3-methyl-1-(2-(diethylamino)phenyl)butyl)aminocarbonylmethyl]phenoxy]-2-methylpropanoic acid

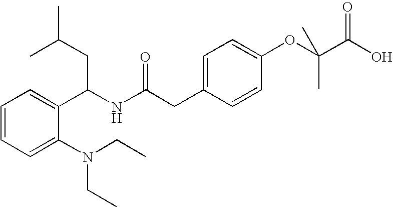

This product was synthesized in 2 steps:

### Step 1: Ethyl 2-[4-[1-(3-methyl-1-(2-(diethylamino)phenyl)butyl)aminocarbonylmethyl]phenoxy]-2-methylpropanoate

This intermediate compound was obtained by condensation of amine 2-18 and acid 3-1 according to protocol F. It was purified by silica gel chromatography (dichloromethane/ethyl acetate 95/5).

Yield: 51%

Aspect: white powder

¹H NMR (300 MHz, DMSO d6, δ in ppm): 0.85 (d, 6.6 Hz, 6H); 0.91 (t, 7.2 Hz, 6H); 1.16 (t, 7.2 Hz, 3H); 1.30 (m, 1H); 1.49 (m, 8H); from 2.86 to 3.02 (m, 4H); 3.33 (m, 2H); 4.15 (q, 7.2 Hz, 2H); 5.38 (m, 1H); 6.70 (d, 8.8 Hz, 2H); from 7.02 to 7.19 (m, 5H); 7.31 (d, 7.7 Hz, 1H); 8.27 (d, 8.8 Hz, 1H).

### Step 2: 2-[4-[1-(3-methyl-1-(2-(diethylamino)phenyl)butyl)aminocarbonylmethyl]phenoxy]-2-methylpropanoic acid

This compound was obtained by saponification of the previously described intermediate according to protocol H. It was purified by silica gel chromatography (dichloromethane/methanol 95/5).

Yield: 85%

Aspect: white powder

¹H NMR (300 MHz, DMSO d6, δ in ppm): 0.86 (d, 6.8 Hz, 6H); 0.92 (t, 7.1 Hz, 6H); from 1.24 to 1.57 (m, 9H); from 2.86 to 3.04 (m, 4H); 3.35 (m, 2H); 5.40 (m, 1H); 6.75 (d, 8.3 Hz, 2H); from 7.03 to 7.18 (m, 5H); 7.32 (d, 7.9 Hz, 1H); 8.31 (d, 7.9 Hz, 1H).

Compound 21:

### 2-[4-[1-(3-methyl-1-(2-(1-piperidinyl)-4-(trifluoromethyl)phenyl)butyl)aminocarbonylmethyl]phenoxy]-2-methylpropanoic acid

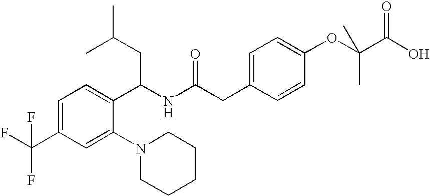

This product was synthesized in 2 steps:

### Step 1: Ethyl 2-[4-[1-(3-methyl-1-(2-(1-piperidinyl)-4-(trifluoromethyl)phenyl)butyl)aminocarbonylmethyl]phenoxy]-2-methylpropanoate

This intermediate compound was obtained by condensation of amine 2-19 and acid 3-1 according to protocol F. It was purified by silica gel chromatography (cyclohexane/ethyl acetate 85/15).

Yield: 74%

Aspect: viscous oil

¹H NMR (300 MHz, CDCl₃, δ in ppm): 0.90 (m, 6H); 1.26 (t, 7.3 Hz, 3H); from 1.57 to 1.74 (m, 15H); 2.64 (m, 2H); 3.07 (m, 2H); 3.50 (s, 2H); 4.25 (q, 7.3 Hz, 2H); 5.40 (m, 1H); 6.01 (d, 7.3 Hz, 1H); 6.84 (d, 8.7 Hz, 2H); from 7.07 to 7.15 (m, 3H); 7.27 (m, 1H); 7.37 (s, 1H).

### Step 2: 2-[4-[1-(3-methyl-1-(2-(1-piperidinyl)-4-(trifluoromethyl)phenyl)butyl)aminocarbonylmethyl]phenoxy]-2-methylpropanoic acid

This compound was obtained by saponification of the previously described intermediate according to protocol H. It was purified by silica gel chromatography (dichloromethane/methanol 95/5).

Yield: 44%

Aspect: white powder

¹H NMR (300 MHz, DMSO d6, δ in ppm): 0.89 (d, 6.1 Hz, 6H); 1.27 (m, 1H); 1.45 (m, 6H); from 1.45 to 1.66 (m, 8H); 2.55 (m, 2H); 3.14 (m, 2H); 3.36 (m, 2H); 5.28 (m, 1H); 6.72 (d, 8.2 Hz, 2H); 7.10 (d, 8.2 Hz, 2H); from 7.33 to 7.68 (m, 3H); 8.53 (d, 8.0 Hz, 1H).

Compound 22:

### 2-[4-[1-(3-methyl-1-(2-(1-piperidinyl)-5-(chloro)phenyl)butyl)aminocarbonylmethyl]phenoxy]-2-methylpropanoic acid

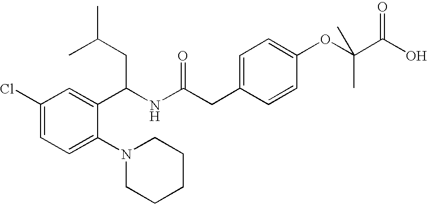

This product was synthesized in 2 steps:

### Step 1: Ethyl 2-[4-[1-(3-methyl-1-(2-(1-piperidinyl)-5-(chloro)phenyl)butyl)aminocarbonylmethyl]phenoxy]-2-methylpropanoate

This intermediate compound was obtained by condensation of amine 2-20 and acid 3-1 according to protocol F. It was purified by silica gel chromatography (cyclohexane/ethyl acetate 8/2).

Yield: 75%

Aspect: white solid

¹H NMR (300 MHz, CDCl₃, δ in ppm): 0.90 (m, 6H); 1.26 (t, 7.2 Hz, 3H); from 1.38 to 1.70 (m, 15H); 2.59 (m, 2H); 2.99 (m, 2H); 3.49 (s, 2H); 4.25 (q, 7.2 Hz, 2H); 5.33 (m, 1H); 6.10 (d, 7.7 Hz, 1H); 6.84 (d, 8.7 Hz, 2H); 6.97 (d, 2.1 Hz, 1H); from 7.06 to 7.15 (m, 4H).

### Step 2: 2-[4-[1-(3-methyl-1-(2-(1-piperidinyl)-5-(chloro)phenyl)butyl)aminocarbonylmethyl]phenoxy]-2-methylpropanoic acid

This compound was obtained by saponification of the previously described intermediate according to protocol H. It was purified by recrystallization in a dichloromethane/heptane mixture (1/1).

Yield: quantitative

Aspect: white powder

¹H NMR (300 MHz, DMSO d6, δ in ppm): 0.89 (d, 6.2 Hz, 6H); 1.28 (m, 1H); 1.47 (s, 6H); from 1.47 to 1.66 (m, 8H); 2.51 (m, 2H); 3.07 (m, 2H); 3.38 (s, 2H); 5.26 (m, 1H); 6.74 (d, 8.4 Hz, 2H); from 7.11 to 7.28 (m, 5H); 8.43 (d, 8.0 Hz, 1H).

Compound 23:

### 2-[4-[1-(3-methyl-1-(2-(1-piperidinyl)phenyl)butyl)aminocarbonylmethyl]phenoxy]acetic acid

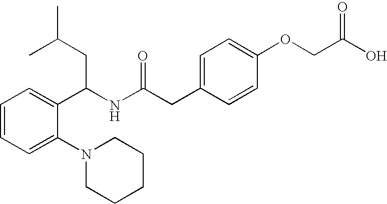

This product was prepared according to a <<one-pot>> procedure.

Bromoethyl acetate was substituted by the phenol derivative 4-1 according to protocol D. The solvant was then evaporated and the crude was saponified according to protocol H. The expected product was purified by silica gel chromatography (dichloromethane/methanol 9/1) and recrystallized in methanol.

Yield: 48%

Aspect: white powder

¹H NMR (300 MHz, DMSO d6/D₂O, δ in ppm): 0.84 (d, 6.4 Hz, 6H); from 1.19 to 1.62 (m, 9H); 2.50 (m, 2H); 2.99 (m, 2H); 3.32 (s, 2H); 4.20 (s, 2H); 5.29 (m, 1H); 6.73 (d, 8.5 Hz, 2H); from 7.98 to 7.22 (m, 6H).

Compound 24:

### 2-[4-[1-(3-methyl-1-(2-(1-piperidinyl)phenyl)butyl)aminocarbonylmethyl]phenoxy]propanoic acid

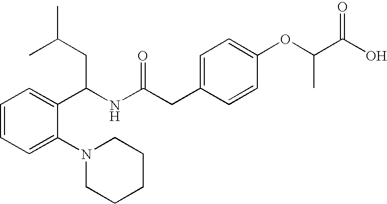

This product was prepared according to a <<one-pot>> procedure.

Ethyl 2-bromopropanoate was substituted by the phenol derivative 4-1 according to protocol D. The solvant was then evaporated and the crude was saponified according to protocol H. The expected product was purified by silica gel chromatography (dichloromethane/methanol 9/1).

Yield: 57%

Aspect: white powder

¹H NMR (300 MHz, DMSO d6/D₂O, δ in ppm): 0.86 (d, 6.3 Hz, 6H); from 1.25 to 1.65 (m, 12H); 2.50 (m, 2H); 3.05 (m, 2H); 3.32 (m, 2H); 4.34 (q, 6.7 Hz, 1H); 5.31 (m, 1H); 6.79 (d, 8.8 Hz, 2H); from 7.02 to 7.17 (m, 5H); 7.25 (d, 7.8 Hz, 1H).

Compound 25:

### 2-[4-[1-(3-methyl-1-(2-(1-piperidinyl)phenyl)butyl)aminocarbonylmethyl]phenoxy]butanoic acid

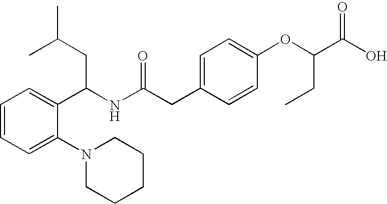

This product was prepared according to a <<one-pot>> procedure.

Ethyl 2-bromobutanoate was substituted by the phenol derivative 4-1 according to protocol D. The solvant was then evaporated and the crude was saponified according to protocol H. The expected product was purified by silica gel chromatography (dichloromethane/methanol 95/5).

Yield: 47%

Aspect: white powder

¹H NMR (300 MHz, DMSO d6/D₂O, δ in ppm): 0.87 (d, 6.5 Hz, 6H); 0.95 (t, 7.5 Hz, 3H); from 1.24 to 1.89 (m, 11H); 2.50 (m, 2H); 3.06 (m, 2H); 3.33 (m, 2H); 4.35 (m, 1H); 5.30 (m, 1H); 6.73 (d, 8.0 Hz, 2H); from 6.99 to 7.14 (m, 5H); 7.25 (d, 7.5 Hz, 1H).

Compound 26:

### 2-[4-[1-(3-methyl-1-(2-(1-piperidinyl)phenyl)butyl)aminocarbonylmethyl]phenoxy]-3-methylbutanoic acid

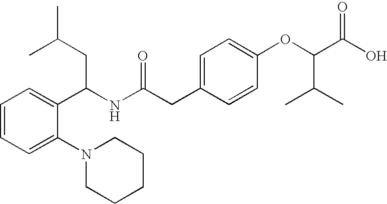

This product was prepared according to a <<one-pot>> procedure.

Ethyl 2-bromo-3-methylbutanoate was substituted by the phenol derivative 4-1 according to protocol D. The solvant was then evaporated and the crude was saponified according to protocol H. The expected product was purified by silica gel chromatography (dichloromethane/methanol 95/5).

Yield: 58%

Aspect: white powder

¹H NMR (300 MHz, DMSO d6/D₂O, δ in ppm): 0.87 (d, 6.2 Hz, 6H); 0.97 (d, 6.7 Hz, 6H); from 1.21 to 1.64 (m, 9H); 2.15 (m, 1H); 2.50 (m, 2H); 3.04 (m, 2H); 3.34 (m, 2H); 4.30 (d, 5.1 Hz, 1H); 5.30 (m, 1H); 6.75 (d, 8.7 Hz, 2H); from 6.99 to 7.17 (m, 5H); 7.23 (d, 7.7 Hz,1H).

Compound 27:

### 2-[4-[1-(3-methyl-1-(2-(1-piperidinyl)phenyl)butyl)aminocarbonylmethyl]phenoxy]-2-phenylacetic acid

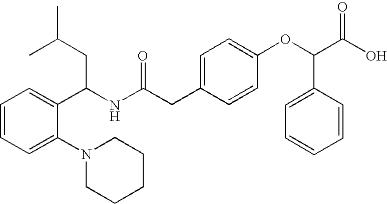

This product was prepared according to a <<one-pot>> procedure.

2-bromo-2-phenylethyl acetate was substituted by the phenol derivative 4-1 according to protocol D. The solvant was then evaporated and the crude was saponified according to protocol H. The expected product was purified by silica gel chromatography (dichloromethane/methanol 9/1).

Yield: 24%

Aspect: white powder

¹H NMR (300 MHz, DMSO d6, δ in ppm): 0.89 (d, 6.1 Hz, 6H); from 1.25 to 1.66 (m, 9H); 2.50 (m, 2H); 3.08 (m, 2H); 3.29 (m, 2H); 5.22 (s, 1H); 5.32 (m, 1H); 6.79 (d, 8.3 Hz, 2H); from 7.06 to 7.32 (m, 9H); 7.53 (d, 7.2 Hz, 2H); 8.35 (d, 8.3 Hz, 1H).

Compound 28:

### 5-[4-[1-(3-methyl-1-(2-(1-piperidinyl)phenyl)butyl)aminocarbonylmethyl]phenoxy]-2,2-dimethylpentanoic acid

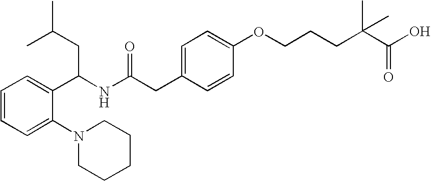

This product was synthesized in 3 steps:

### Step 1: tert-butyl 5-bromo-2,2-dimethylpentanoate

Under anhydrous atmosphere, diisopropylamine (11.4 ml, 83.2 mmol) in THF (150 ml) was cooled to 0° C. nbutyllithium (2.5M in hexane, 32.9 ml; 82.3 mmol) was added dropwise. The mixture was stirred 30 min at 0° C. and cooled to −78° C. with a dry ice bath. tert-butyl 2-methylpropanoate (12 g; 83.2 mmol) was added and the mixture was stirred for 45 min at −78° C. 1,3-dibromopropane (15.2 ml; 149.7 mmol) was added and stirring was maintained for 1 h. The mixture was slowly warmed to room temperature. It was then poured on a saturated ammonium chloride aqueous solution (250 ml). The aqueous layer was extracted with ethyl acetate (3×200 ml). Organic layers were combined, dried with magnesium sulfate (MgSO₄), and concentrated. The expected product was purified by distillation under reduced pressure.

Yield: 30%

Aspect: colorless oil

¹H NMR (300 MHz, CDCl₃, δ in ppm): 1.10 (s, 6H); 1.40 (s, 9H); 1.57 (m, 2H); 1.75 (m, 2H); 3.34 (t, 6.5 Hz, 2H).

### Step 2: tert-butyl 5-[4-[1-(3-methyl-1-(2-(1-piperidinyl)phenyl)butyl)aminocarbonylmethyl]phenoxy]-2,2-dimethylpentanoate

tert-butyl 5-bromo-2,2-dimethylpentanoate was substituted by the phenol derivative 4-1 according to protocol D. The expected product was purified by silica gel chromatography (cyclohexane/ethyl acetate 85/15).

Yield: 34%

Aspect: white powder

¹H NMR (300 MHz, CDCl₃, δ in ppm): 0.91 (d, 6.7 Hz, 6H); 1.17 (s, 6H); from 1.35 to 1.79 (m, 13H); 1.45 (s, 9H); 2.63 (m, 2H); 2.97 (m, 2H); 3.49 (m, 2H); 3.93 (t, 7.1 Hz, 2H); 5.39 (m, 1H); 6.38 (d, 8.7 Hz, 1H); 6.85 (d, 8.3 Hz, 2H); 7.04 (m, 2H); from 7.14 to 7.19 (m, 4H).

### Step 3: 5-[4-[1-(3-methyl-1-(2-(1-piperidinyl)phenyl)butyl)aminocarbonylmethyl]phenoxy]-2,2-dimethylpentanoic acid

tert-butylic ester (245 mg, 0.43 mmol) was dissolved in dichloromethane (10 ml). Trifluoroacetic acid (5 ml) was added dropwise and the mixture was stirred for 4 h at room temperature. The crude was evaporated to dryness and 1M NaOH (10 ml) was added. pH was brought to 3-4 upon addition of, hydrochloric acid and the expected product was extracted with dichloromethane (2×20 ml). The organic layer was dried with magnesium sulfate (MgSO4), filtered and concentrated. The expected product was purified by silica gel chromatography (dichloromethane/methanol 95/5).

Yield: 68%

Aspect: white powder

¹H NMR (300 MHz, DMSO d6, δ in ppm): 0.84 (d, 6.5 Hz, 6H); 1.07 (s, 6H); 1.28 (m, 1H); from 1.45 to 1.62 (m, 12H); 2.50 (m, 2H); 3.00 (m, 2H); 3.32 (m, 2H); 3.85 (t, 6.0 Hz, 2H); 5.28 (m, 1H); 6.78 (d, 8.4 Hz, 2H); from 6.97 to 7.14 (m, 5H); 7.21 (d, 8.0 Hz, 1H); 8.39 (d, 8.4 Hz, 1H).

Compound 29:

### 2-[3-[1-(3-methyl-1-(2-(1-piperidinyl)phenyl)butyl)aminocarbonylmethyl]phenoxy]-2-methylpropanoic acid

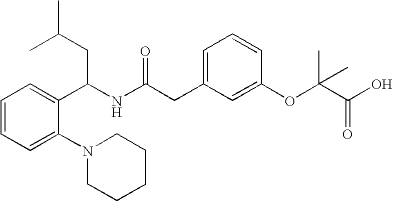

This product was synthesized in 2 steps:

### Step 1: Ethyl 2-[3-[1-(3-methyl-1-(2-(1-piperidinyl)phenyl)butyl)aminocarbonylmethyl]phenoxy]-2-methylpropanoate

This intermediate compound was obtained by condensation of amine 2-1 and acid 3-4 according to protocol F. It was purified by silica gel chromatography (cyclohexane/ethyl acetate 9/1).

Yield: 38%

Aspect: white powder

¹H NMR (300 MHz, CDCl₃, δ in ppm): 0.90 (d, 6.0 Hz, 6H); 1.24 (t, 7.1 Hz, 3H); from 1.30 to 1.80 (m, 15H); 2.63 (m, 2H); 2.96 (m, 2H); 3.49 (m, 2H); 4.22 (q, 7.1 Hz, 2H); 5.39 (m, 1H); 6.39 (d, 8.2 Hz, 1H); 6.75 (m, 2H); 6.90 (d, 7.1 Hz, 1H); 7.05 (m, 2H); 7.19 (m, 3H).

### Step 2: 2-[3-[1-(3-methyl-1-(2-(1-piperidinyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 29%

Aspect: white powder

¹H NMR (300 MHz, CDCl₃, δ in ppm): 0.89 (d, 6.6 Hz, 6H); from 1.27 to 1.80 (m, 15H); 2.66 (m, 2H); 2.93 (m, 2H); 3.45 (m, 2H); 5.32 (m, 1H); 6.84 (m, 3H); from 7.06 to 7.19 (m, 5H); 9.39 (s(broad), 1H).

Compound 30:

### 2-[3-[1-(1-(2-(1-piperidinyl)phenyl)ethyl)aminocarbonylmethyl]phenoxy]-2-methylpropanoic acid

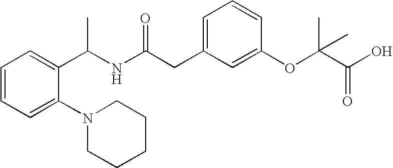

This product was synthesized in 2 steps:

### Step 1: Ethyl 2-[3-[1-(1-(2-(1-piperidinyl)phenyl)ethyl)aminocarbonylmethyl]phenoxy]-2-methylpropanoate

This intermediate compound was obtained by condensation of amine 2-2 and acid 3-4 according to protocol F. It was purified by silica gel chromatography (cyclohexane/ethyl acetate 9/1).

Yield: 12%

Aspect: beige powder

¹H NMR (300 MHz, CDCl₃, δ in ppm): 1.23 (t, 7.3 Hz, 3H); 1.38 (d, 6.8 Hz, 3H); from 1.45 to 1.80 (m, 12H); 2.67 (m, 2H); 2.91 (m, 2H); 3.49 (s, 2H); 4.20 (q, 7.3 Hz, 2H); 5.38 (m, 1H); 6.72 (dd, 8.1 Hz and 2.1 Hz, 1H); 6.79 (m, 1H); 6.89 (d, 7.7 Hz, 1H); 6.97 (d, 7.7 Hz, 1H); from 7.01 to 7.25 (m, 5H).

### Step 2: 2-[3-[1-(1-(2-(1-piperidinyl)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 68%

Aspect: white powder

¹H NMR (300 MHz, CDCl₃, δ in ppm): 1.34 (d, 6.5 Hz, 3H); 1.42 (s, 6H); from 1.42 to 1.68 (m, 6H); 2.66 (m, 2H); 2.91 (m, 2H); 3.38 (m, 2H); 5.31 (m, 1H); 6.81 (m, 3H); from 6.95 to 7.22 (m, 5H); 7.59 (s(broad), 1H); 10.12 (s(broad), 1H).

Compound 31:

### 2-[3-[1-(1-phenyl-1-(2-(1-piperidinyl)phenyl)methyl)aminocarbonylmethyl]phenoxy]-2-methylpropanoic acid

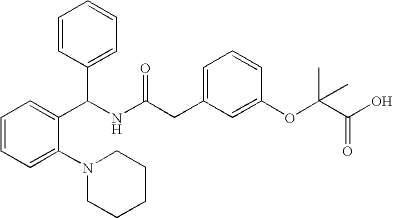

This product was synthesized in 2 steps:

### Step 1: Ethyl 2-[3-[1-(1-phenyl-1-(2-(1-piperidinyl)phenyl)methyl)aminocarbonylmethyl]phenoxy]-2-methylpropanoate

This intermediate compound was obtained by condensation of amine 2-3 and acid 3-4 according to protocol F. It was purified by trituration in diethyl ether and filtration.

Yield: 66%

Aspect: beige powder

¹H NMR (300 MHz, CDCl₃, δ in ppm): 1.22 (t, 7.1 Hz, 3H); from 1.30 to 1.70 (m, 12H); 2.46 (m, 2H); 2.53 (m, 2H); 3.61 (m, 2H); 4.20 (q, 7.1 Hz, 2H); 6.50 (d, 8.4 Hz, 1H); 6.74 (d, 8.4 Hz, 1H); 6.84 (s, 1H); 6.96 (d, 7.5 Hz, 1H); from 7.05 to 7.35 (m, 10H); 7.46 (d, 8.4 Hz, 1H).

### Step 2: 2-[3-[1-(1-phenyl-1-(2-(1-piperidinyl)phenyl)methyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 35%

Aspect: white powder

¹H NMR (300 MHz, CDCl₃, δ in ppm): from 1.20 to 1.65 (m, 12H); 2.43 (m, 2H); 2.50 (m, 2H); 3.54 (m, 2H); 6.48 (d, 8.4 Hz, 1H); from 6.80 to 7.40 (m, 13H); 7.86 (d, 8.4 Hz, 1H).

Compound 32:

### 2-[4-[1-(3-methyl-1-(2-(1-piperidinyl)phenyl)butyl)carbonylaminomethyl]phenoxy]-2-methylpropanoic acid

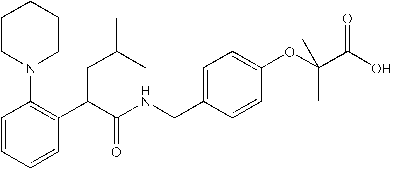

This product was synthesized in 2 steps:

### Step 1: Ethyl 2-[4-[1-(3-methyl-1-(2-(1-piperidinyl)phenyl)butyl)carbonylaminomethyl]phenoxy]-2-methylpropanoate

This intermediate compound was obtained by condensation of amine 2-21 and acid 3-5 according to protocol G. It was purified by silica gel chromatography (cyclohexane/ethyl acetate 8/2).

Yield: 10%

Aspect: colorless oil

¹H NMR (300 MHz, CDCl₃, δ in ppm): 0.94 (m, 6H); 1.25 (t, 7.0 Hz, 3H); from 1.40 to 1.70 (m, 15H); 2.45 (m, 2H); 2.85 (m, 2H) from 4.07 to 4.26 (m, 4H); 4.37 (dd, 7.1 Hz and 14.1 Hz, 1H); 6.67 (d, 8.5 Hz, 2H); 6.82 (d, 8.5 Hz, 2H); from 7.04 to 7.22 (m, 4H); 7.36 (d, 7.6 Hz,1H).

### Step 2: 2-[4-[1-(3-methyl-1-(2-(1-piperidinyl)phenyl)butyl)carbonylaminomethyl]phenoxy]-2-methylpropanoic acid

This compound was obtained by saponification of the previously described intermediate according to protocol H. The expected product was purified by silica gel chromatography (dichloromethane/methanol 9/1).

Yield: 62%

Aspect: white solid

¹H NMR (300 MHz, DMSO d6, δ in ppm): 0.85 (d, 6.6 Hz, 3H);.0.90 (d, 6.6 Hz, 3H); from 1.32 to 1.75 (m, 14H); 1.85 (m, 1H); 2.64 (m, 2H); 2.77 (m, 2H); 4.17 (m, 3H); 6.72 (d, 8.5 Hz, 2H); from 7.02 to 7.20 (m, 5H); 7.38 (d, 7.1 Hz, 1H); 8.14 (m, 1H).

Compound 33:

### 2-[4-[1-(1-(2-(1-piperidinyl)-4-(bromo)phenyl)pentyl)aminocarbonylmethyl]phenoxy]-2-methylpropanoic acid

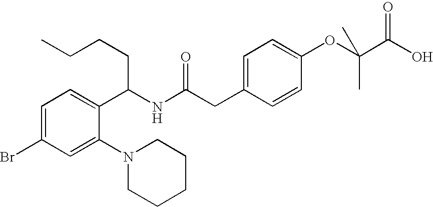

This product was synthesized in 2 steps:

### Step 1: Ethyl 2-[4-[1-(1-(2-(1-piperidinyl)-4-(bromo)phenyl)pentyl)aminocarbonylmethyl]phenoxy]-2-methylpropanoate

This intermediate compound was obtained by condensation of amine 2-22 and acid 3-1 according to protocol F. It was purified by silica gel chromatography (cyclohexane/ethyl acetate 8/2).

Yield: 79%

Aspect: white solid

¹H NMR (300 MHz, CDCl₃, δ in ppm): 0.84 (t, 7.6 Hz, 3H); from 1.02 to 1.38 (m, 7H); from 1.57 to 1.68 (m, 14H); 2.61 (m, 2H); 2.94 (m, 2H); 3.49 (m, 2H); 4.24 (q, 7.1 Hz, 2H); 5.18 (m, 1H); 6.28 (d, 8.2 Hz, 1H); from 6.81 to 6.89 (m, 3H); from 7.11 to 7.18 (m, 3H); 7.25 (d, 2.2 Hz, 1H).

### Step 2: 2-[4-[1-(1-(2-(1-piperidinyl)-4-(bromo)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 63%

Aspect: white powder

¹H NMR (300 MHz, DMSO d6/D₂O, δ in ppm): 0.82 (t, 7.1 Hz, 3H); from 1.15 to 1.28 (m, 4H); 1.43 (s, 6H); from 1.43 to 1.62 (m, 8H); 2.51 (m, 2H); 3.06 (m, 2H); 3.33 (m, 2H); 5.09 (m, 1H); 6.72 (d, 8.6 Hz, 2H); 7.07 (d, 8.6 Hz, 2H); from 7.17 to 7.23 (m, 3H).

Compound 34:

### 2-[4-[1-methyl-1-[1-(3-methyl-1-(2-(1-piperidinyl)phenyl)butyl)aminocarbonyl]methyl]phenoxy]-2-methylpropanoic acid

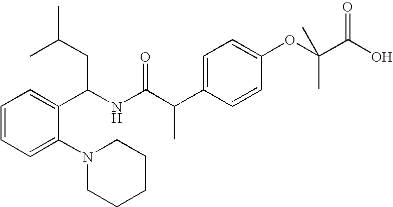

This product was synthesized in 2 steps:

### Step 1: Ethyl 2-[4-[1-methyl-1-[1-(3-methyl-1-(2-(1-piperidinyl)phenyl)butyl)aminocarbonyl]methyl]phenoxy]-2-methylpropanoate

This intermediate was obtained by substitution of ethyl bromoisobutyrate by the phenol derivative 4-2 according to protocol D. It was purified by silica gel chromatography (cyclohexane/ethyl acetate 9/1).

Yield: 61%

Aspect: white powder

¹H NMR (300 MHz, CDCl₃, δ in ppm, diastereoisomers mixture 1/1): from 0.82 to 0.94 (m, 6H); from 1.21 to 1.29 (m, 3H); from 1.39 to 1.69 (m, 18H); 2.53 and 2.66 (m+m, 2H); 2.96 (m, 2H); from 3.41 to 3.53 (m, 1H); from 4.18 to 4.29 (m, 2H); from 5.28 to 5.38 (m, 1H); 6.33 (m, 1H); 6.76 and 6.83 (d +d, 8.8 Hz, 2H); from 6.92 to 7.24 (m, 6H).

### Step 2: 2-[4-[1-methyl-1-[1-(3-methyl-1-(2-(1-piperidinyl)phenyl)butyl)aminocarbonyl]methyl]phenoxy]-2-methylpropanoic acid

This compound was obtained by saponification of the previously described intermediate according to protocol H. The expected product was purified by silica gel chromatography (dichloromethane/methanol 9/1).

Yield: 46%

Aspect: white powder

¹H NMR (300 MHz, DMSO d6, δ in ppm, diastereoisomers mixture 8/2): 0.79 and 0.91 (m+m, 6H); from 1.22 to 1.67 (m, 18H); 2.51 (m, 2H); 3.11 (m, 2H); 3.62 (m, 1H); 5.28 (m, 1H); 6.70 and 6.77 (d +d, 8,8 Hz, 2H); from 7.04 to 7.22 (m, 5H); 7.30 (dd, 7.5 Hz, 1.9 Hz, 1H); 8.21 (d, 8.4 Hz, 1H).

Compound 35:

### 2-[4-[N-isobutyl-N-(1-(2-(1-piperidinyl)phenyl)pentyl)aminocarbonylmethyl]phenoxy]-2-methylpropanoic acid

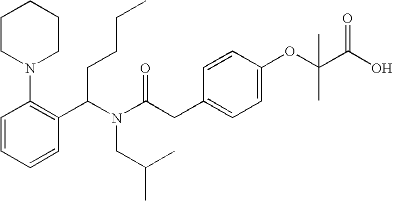

This product was synthesized in 2 steps:

### Step 1: Ethyl 2-[4-[N-isobutyl-N-(1-(2-(1-piperidinyl)phenyl)pentyl)aminocarbonylmethyl]phenoxy]-2-methylpropanoate

This intermediate compound was obtained by condensation of amine 2-23 and acid 3-1 according to protocol F. It was purified by silica gel chromatography (cyclohexane/ethyl acetate 85/15).

Yield: 80%

Aspect: white solid

¹H NMR (300 MHz, DMSO d6, 100° C., δ in ppm): 0.64 (m, 3H); 0.74 (d, 6.8 Hz, 3H); 0.83 (t, 7.2 Hz, 3H); from 1.10 to 1.29 (m, 7H); from 1.50 to 1.75 (m, 14H); 2.02 (m, 1H); from 2.66 to 2.83 (m, 4H); 2.99 (m, 2H); 3.79 (m, 2H); 4.19 (q, 7.2 Hz, 2H); 5.60 (t, 7.8 Hz, 1H); 6.78 (d, 8.3 Hz, 2H); from 7.12 to 7.33 (m, 5H); 7.55 (d, 7.3 Hz, 1H).

### Step 2: 2-[4-[N-isobutyl-N-(1-(2-(1-piperidinyl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 86%

Aspect: beige powder

¹H NMR (300 MHz, DMSO d6, 100° C., δ in ppm): 0.62 (m, 3H); 0.74 (d, 6.7 Hz, 3H); 0.83 (t, 7.2 Hz, 3H); from 1.10 to 1.17 (m, 2H); from 1.24 to 1.32 (m, 2H); 1.46 (s, 6H); from 1.46 to 1.75 (m, 8H); 2.02 (m, 1H); from 2.66 to 2.83 (m, 4H); 2.99 (m, 2H); 3.78 (m, 2H); 5.61 (t, 7.8 Hz, 1H); 6.86 (d, 8.3 Hz, 2H); 7.06 (d, 8.3 Hz, 2H); 7.14 (m, 1H); 7.28 (m, 2H); 7.55 (d, 7.2 Hz, 1H).

Compound 36:

### 2-[4-[1-(1-(2-(phenylthio)phenyl)pentyl)aminocarbonylmethyl]phenoxy]-2-methylpropanoic acid

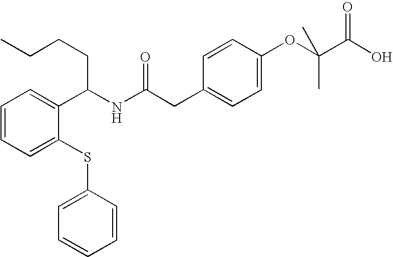

This product was synthesized in 2 steps:

### Step 1: 2-[4-[1-(1-(2-(phenylthio)phenyl )pentyl)aminocarbonylmethyl]phenoxy]-2-methylpropanoate d'ethyle

This intermediate compound was obtained by condensation of amine 2-24 and acid 3-1 according to protocol F. It was purified by silica gel chromatography (cyclohexane/ethyl acetate 8/2).

Yield: 77%

Aspect: pale yellow oil

¹H NMR (300 MHz, CDCl₃, δ in ppm): 0.80 (t, 6.9 Hz, 3H); from 1.08 to 1.21 (m, 4H); 1.26 (t, 7.4 Hz, 3H); from 1.56 to 1.71 (m, 8H); 3.33 (d, 16.1 Hz, 1H); 3.45 (d, 16.1 Hz, 1H); 4.24 (q, 7.4 Hz, 2H); 5.29 (m, 1H); 6.01 (d, 7.8 Hz, 1H); 6.80 (d, 8.6 Hz, 2H); 7.02 (d, 8.6 Hz, 2H); from 7.16 to 7.33 (m, 9H).

### Step 2: 2-[4-[1-(1-(2-(phenylthio)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 70%

Aspect: beige powder

¹H NMR (300 MHz, DMSO d6/D₂O, δ in ppm): 0.82 (t, 6.7 Hz, 3H); 1.15 (m, 4H); from 1.34 to 1.48 (m, 8H); 3.32 (m, 2H); 5.27 (m, 1H); 6.70 (d, 8.2 Hz, 2H); 7.08 (d, 8.2 Hz, 2H); from 7.20 to 7.39 (m, 9H).

Compound 37:

### 2-[4-[1-(3-methyl-1-(2-(1-piperidinyl)-5-(bromo)phenyl)butyl)aminocarbonylmethyl]phenoxy]-2-methylpropanoic acid

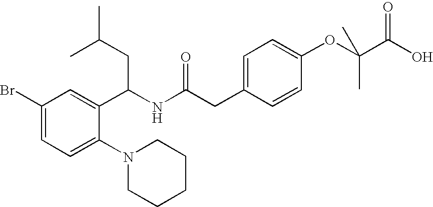

This product was synthesized in 2 steps:

### Step 1: Ethyl 2-[4-[1-(3-methyl-1-(2-(l -piperidinyl)-5-(bromo)phenyl)butyl)aminocarbonylmethyl]phenoxy]-2-methylpropanoate

This intermediate compound was obtained by condensation of amine 2-25 and acid 3-1 according to protocol F. It was purified by silica gel chromatography (cyclohexane/ethyl acetate 85/15).

Yield: 85%

Aspect: white solid

¹H NMR (300 MHz, CDCl₃, δ in ppm): 0.90 (m, 6H); 1.26 (t, 7.2 Hz, 3H); from 1.38 to 1.70 (m, 15H); 2.59 (m, 2H); 2.98 (m, 2H); 3.50 (s, 2H); 4.24 (q, 7.2 Hz, 2H); 5.32 (m, 1H); 6.07 (m, 1H); 6.84 (d, 8.7 Hz, 2H); 6.97 (d, 2.1 Hz, 1H); from 7.06 to 7.16 (m, 4H).

### Step 2: 2-[4-[1-(3-methyl-1-(2-(1-piperidinyl)-5-(bromo)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 40%

Aspect: white powder

¹H NMR (300 MHz, DMSO d6, δ in ppm): 0.88 (d, 6.5 Hz, 6H); from 1.23 to 1.71 (m, 15H); 2.51 (m, 2H); 3.07 (m, 2H); 3.35 (s, 2H); 5.25 (m, 1H); 6.75 (d, 8.3 Hz, 2H); from 7.03 to 7.11 (m, 3H); 7.18 (dd, 8.3 Hz and 2.3 Hz, 1H); 7.30 (s, 1H); 8.52 (m, 1H).

Compound 38:

### 2-[4-[1-(1-(2-(1-piperidinyl)-4-(1H-indolyl)phenyl)pentyl)aminocarbonylmethyl]phenoxy]-2-methylpropanoic acid

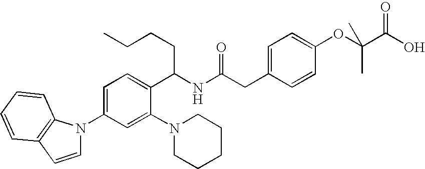

This product was synthesized in 2 steps from ethyl 2-[4-[1-(1-(2-(1-piperidinyl)-4-(bromo)phenyl)pentyl)aminocarbonylmethyl]phenoxy]-2-methylpropanoate (intermediate for the synthesis of compound 33).

### Step 1: Ethyl 2-[4-[1-(1-(2-(1-piperidinyl)-4-(1H-indolyl)phenyl)pentyl)aminocarbonylmethyl]phenoxy]-2-methylpropanoate

This intermediate compound was synthesized from ethyl 2-[4-[1-(1-(2-(1-piperidinyl)-4-(bromo)phenyl)pentyl)aminocarbonylmethyl]phenoxy]-2-methylpropanoate and indole according to protocol I as previously described. It was purified by silica gel chromatography (cyclohexane/ethyl acetate 8/2).

Yield: 49%

Aspect: beige solid

¹H NMR (300 MHz, CDCl₃, δ in ppm): 0.89 (m, 3H); from 1.12 to 1.73 (m, 21H); 2.70 (m, 2H); 3.04 (m, 2H); 3.54 (m, 2H); 4.25 (m, 2H); 5.32 (m, 1H); 6.59 (d, 8.3 Hz, 1H); 6.67 (d, 3.1 Hz, 1H); 6.85 (m, 3H); from 7.12 to 7.33 (m, 7H); 7.55 (d, 7.8 Hz, 1H); 7.70 (d, 7.8 Hz, 1H).

### Step 2: 2-[4-[1-(1-(2-(1-piperidinyl)-4-(1H-indolyl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 85/15).

Yield: 30%

Aspect: white powder

¹H NMR (300 MHz, DMSO d6, δ in ppm): 0.85 (m, 3H); from 1.20 to 1.75 (m, 18H); 2.65 (m, 2H); 3.17 (m, 2H); 3.38 (m, 2H); 5.23 (m, 1H); from 6.67 to 6.77 (m, 3H); from 7.08 to 7.27 (m, 6H); 7.44 (d, 8.0 Hz, 1H); 7.54 (d, 8.0 Hz, 1H); 7.65 (m, 2H); 8.46 (m, 1H).

Compound 39:

### 2-[4-[1-(3-methyl-1-(2-(1-piperidinyl)-4-(phenyl)phenyl)butyl)aminocarbonylmethyl]phenoxy]-2-methylpropanoic acid

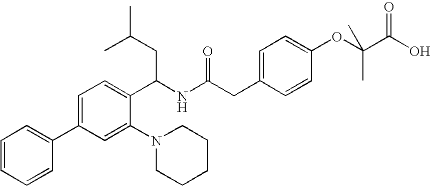

This product was synthesized in 3 steps:

### Step 1: Ethyl 2-[4-[1-(3-methyl-1-(2-( I-piperidinyl)-4-(bromo)phenyl)butyl)aminocarbonylmethyl]phenoxy]-2-methylpropanoate

This intermediate compound was obtained by condensation of amine 2-26 and acid 3-1 according to protocol F. It was purified by silica gel chromatography (cyclohexane/ethyl acetate 85/15).

Yield: 43%

Aspect: white solid

¹H NMR (300 MHz, CDCl₃, δ in ppm): 0.90 (m, 6H); 1.26 (t, 7.0 Hz, 3H); from 1.34 to 1.74 (m, 15H); 2.62 (m, 2H); 2.98 (m, 2H); 3.48 (s, 2H); 4.22 (q, 7.0 Hz, 2H); 5.31 (m, 1H); 6.03 (d, 8.2 Hz, 1H); from 6.78 to 6.87 (m, 3H); from 7.10 to 7.17 (m, 3H); 7.24 (d, 1.8 Hz, 1H).

### Step 2: Ethyl 2-[4-[1-(3-methyl-1-(2-(1-piperidinyl)-4-(phenyl)phenyl)butyl)aminocarbonylmethyl]phenoxy]-2-methylpropanoate

This compound was synthesized from the previously described intermediate and phenylboronic acid according to protocol J. It was purified by silica gel chromatography (cyclohexane/ethyl acetate 8/2).

Yield: 36%

Aspect: white powder

¹H NMR (300 MHz, CDCl₃, δ in ppm): 0.93 (m, 6H); 1.25 (t, 7.1 Hz, 3H); from 1.39 to 1.75 (m, 15H); 2.71 (m, 2H); 3.05 (m, 2H); 3.51 (s, 2H); 4.23 (q, 7.1 Hz, 2H); 5.41 (m, 1H); 6.41 (d, 8.8 Hz, 1H); 6.83 (d, 8.8 Hz, 2H); from 7.09 to 7.17 (m, 3H); from 7.26 to 7.46 (m, 5H); 7.54 (d, 8.0 Hz, 2H).

### Step 3: 2-[4-[1-(3-methyl-1-(2-(1-piperidinyl)-4-(phenyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 40%

Aspect: white powder

¹H NMR (300 MHz, DMSO d6, δ in ppm): 0.90 (m, 6H); from 1.20 to 1.75 (m, 15H); 2.62 (m, 2H); 3.17 (m, 2H); 3.38 (m, 2H); 5.35 (m, 1H); 6.74 (d, 8.5 Hz, 2H); 7.08 (d, 8.5 Hz, 2H); from 7.30 to 7.47 (m, 6H); 7.61 (d, 7.6 Hz, 2H); 8.55 (d, 8.5 Hz, 1H).

Compound 40:

### 2-{4-{4-[4-[1-(3-methyl-1-(2-(1-piperidinyl)phenyl)butyl)aminocarbonylmethyl]phenoxy]butoxy}phenoxy}-2-methylpropanoic acid

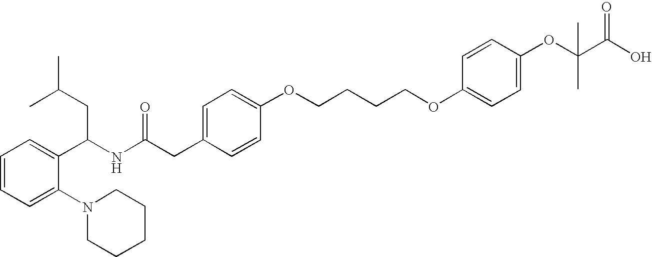

This product was synthesized in 5 steps:

### Step 1: tert-butyl 2-(4-benzyloxyphenoxy)-2-methylpropanoate

To a solution of 4-benzyloxyphenol (150 g, 0.75 mol) in acetonitrile (1.5 l) were added potassium carbonate (414 g, 3.0 mol) and tert-butyl bromoisobutyrate (216 ml, 1.5 mol). The mixture was refluxed overnight under vigourous stirring. The carbonate was filtered and acetonitrile evaporated. The expected product was purified by silica gel chromatography (cyclohexane/ethyl acetate 95/5).

Yield: 53%

Aspect: colorless oil

¹H NMR (300 MHz, CDCl₃, δ in ppm): 1.48 (s, 9H); 1.53 (s, 6H); 5.02 (s, 2H); 6.87 (m, 4H); from 7.32 to 7.46 (m, 5H).

### Step 2: tert-butyl 2-(4-hydroxyphenoxy)-2-methylpropanoate

This compound was obtained by hydrogenolysis of the previously described intermediate according to protocol E. No further purification was performed.

Yield: 99%

Aspect: pale yellow oil

¹H NMR (300 MHz, CDCl₃, δ in ppm): 1.47 (s, 9H); 1.50 (s, 6H); 6.68 (d, 8.5 Hz, 2H); 6.78 (d, 8.5 Hz, 2H).

### Step 3: tert-butyl 2-(4-(4-bromobutoxy)phenoxy)-2-methylpropanoate

The phenol intermediate previously described (30 g, 119 mmol) was solubilized in acetonitrile (1 l) and potassium carbonate (49.3 g, 357 mmol) was added. The suspension was refluxed and the bromide derivative was added (42.6 ml; 357 mmol). Heating was maintained for 16 hours and the crude mixture was cooled to room temperature. Salts were filtered, filtrate evaporated, and the expected product was purified by silica gel chromatography (cyclohexane/ethyl acetate 95/5).

Yield: 56%

Aspect: colorless oil

¹H NMR (300 MHz, CDCl₃, δ in ppm): 1.47 (s, 9H); 1.51 (s, 6H); from 1.90 to 1.96 (m, 2H); from 2.02 to 2.11 (m, 2H); 3.49 (t, 6.5 Hz, 2H); 3.94 (t, 6.1 Hz, 2H); 6.75 (m, 2H); 6.83 (m, 2H).

### Step 4: tert-butyl 2-{4-{4-[4-[1-(3-methyl-1-(2-(1-piperidinyl)phenyl)butyl)aminocarbonylmethyl]phenoxy]butoxy}phenoxy}-2-methylpropanoate

This compound was obtained by substitution of the previously described bromide with 4-[1-(3-methyl-1-(2-(1-piperidinyl)phenyl)butyl)aminocarbonylmethyl]phenol (see example 4-1 for synthesis description). This substitution was performed according to protocol D. The expected product was purified by silica gel chromatography (cyclohexane/ethyl acetate 8/2).

Yield: 55%

Aspect: beige powder

¹H NMR (300 MHz, CDCl₃, δ in ppm): 0.91 (d, 6.2 Hz, 6H); from 1.35 to 1.75 (m, 24H); 1.97 (m, 4H); 2.62 (m, 2H); 2.96 (m, 2H); 3.49 (m, 2H); from 3.97 to 4.03 (m, 4H); 5.38 (m, 1H); 6.40 (d, 8.3 Hz, 1H); 6.78 (m, 2H); from 6.83 to 6.89 (m, 4H); 7.05 (m, 2H); from 7.15 to 7.21 (m, 4H).

### Step 5: 2-{4-{4-[4-[1-(3-methyl-1-(2-(1-piperidinyl)phenyl)butyl)aminocarbonylmethyl]phenoxy]butoxy}phenoxy}-2-methylpropanoic acid

The previous tert-butyl ester (286 mg, 0.42 mmol) was dissolved in dichloromethane (10 ml) and trifluoroacetic acid (5 ml) was added. The mixture was stirred at room temperature for 16 hours. The crude was concentrated to dryness and 1M NaOH (10 ml) was added. The resulting mixture was acidified to pH 3 with hydrochloric acid and extracted with dichloromethane (2×15 ml). The organic layer was dried with magnesium sulfate, filtered and concentrated. The expected product was purified by silica gel chromatography (dichloromethane/methanol 98/2).

Yield: 65%

Aspect: beige powder

¹H NMR (300 MHz, DMSO d6/D₂O, δ in ppm): 0.89 (d, 6.0 Hz, 6H); from 1.22 to 1.70 (m, 15H); 1.80 (m, 4H); 2.51 (m, 2H); 3.05 (m, 2H), 3.34 (m, 2H); from 3.91 to 3.96 (m, 4H); 5.32 (m, 1H); from 6.71 to 6.83 (m, 6H); from 6.99 to 7.14 (m, 5H); 7.24 (d, 6.8 Hz, 1H).

Compound 41:

### 2-[4-[1-(1-(2-(phenylsulfonyl)phenyl)pentyl)aminocarbonylmethyl]phenoxy]-2-methylpropanoic acid

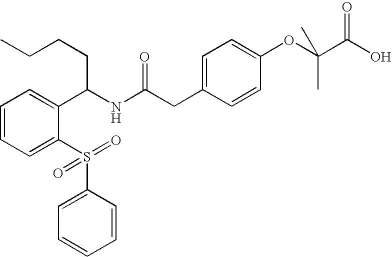

This compound was obtained by oxidation of compound 36.

2-[4-[1-(1-(2-(phenylthio)phenyl)pentyl)aminocarbonylmethyl]phenoxy]-2-methylpropanoic acid (150 mg, 0.31 mmol) was dissolved in a dichloromethane/methanol mixture (15 ml/1 ml). Oxone® (375 mg, 0.61 mmol) in solution in water (15 ml) was then added dropwise. The biphasic mixture was stirred at room temperature overnight. Layers were decanted and the aqueous layer was extracted with dichloromethane (2×50 ml). Organic phases were combined and the resulting solution was dried with magnesium sulfate, filtered and concentrated. The expected product was purified by silica gel chromatography (dichloromethane/methanol 85/15).

Yield: 92%

Aspect: white powder

¹H NMR (300 MHz, DMSO d6, δ in ppm): 0.77 (t, 6.9 Hz, 3H); from 1.14 to 1.50 (m, 12H); 3.27 (m, 2H); 5.67 (m, 1H); 6.70 (d, 8.3 Hz, 2H); 7.01 (d, 8.3 Hz, 2H); from 7.46 to 7.67 (m, 6H); from 7.93 to 8.01 (m, 3H); 8.51 (d, 8.3 Hz, 1H).

Compound 42:

### 3-[4-[1-(1-(2-(1-piperidinyl)phenyl)pentyl)aminocarbonylmethyl]phenoxy]-2,2-dimethylpropanoic acid

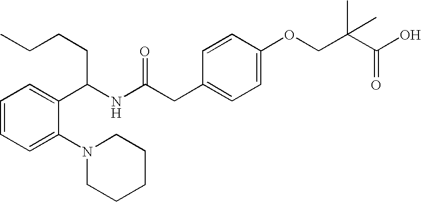

This product was synthesized in 3 steps.

### Step 1: 2-(methoxycarbonyl)-2-methylpropyl 4-methylbenzenesulfonate

Methyl 3-hydroxy-2,2-dimethylpropanoate (5.0 g, 37.83 mmol) was solubilized in dichloromethane (50 ml) and triethylamine (16.0 ml, 113.5 mmol) was added. The mixture was cooled to 0° C. and p-toluenesulfonyl chloride (7.2 g, 37.83 mmol) was added portionwise. The mixture was stirred at room temperature for 4 days and was thenhydrolyzed by water (50 ml). Layers were decanted and the organic phase was washed with 1M HCl (2×50 ml) and NaClsat (2×50 ml). It was dried with magnesium sulfate, filtered and concentrated and the resulting residue was purified by silica gel chromatography (cyclohexane/ethyl acetate 8/2).

Yield: 29%

Aspect: white powder

¹H NMR (300 MHz, CDCl₃, δ in ppm): 1.19 (s, 6H) ; 2.46 (s, 3H); 3.61 (s, 3H); 4.01 (s, 2H); 7.36 (d, 8.0 Hz, 2H); 7.78 (d, 8.0 Hz, 2H).

### Step 2: Methyl 3-[4-[1-(1-(2-(1-piperidinyl)phenyl)pentyl)aminocarbonylmethyl]phenoxy]-2,2-dimethylpropanoate

The phenol derivative 4-3 (200 mg, 0.53 mmol) was solubilized in acetonitrile (10 ml) and cesium carbonate (455 mg, 1.40 mmol) was added. The mixture was stirred at room temperature for 30 minutes and 2-(methoxycarbonyl)-2-methylpropyl 4-methylbenzenesulfonate (151 mg, 0.53 mmol) was added. The mixture was refluxed for 16 hours. The crude was cooled to room temperature, salts were filtered and rinsed with ethyl acetate and the filtrate was concentrated. The residue was taken in ethyl acetate (15 ml), washed successively with 1M NaOH (2×10 ml), 1M aqueous citric acid (2×10 ml) and NaClsat (2×10 ml). The resulting organic phase was dried with magnesium sulfate, filtered and concentrated and the resulting residue was purified by silica gel chromatography (cyclohexane/ethyl acetate 8/2).

Yield: 55%

Aspect: white solid

¹H NMR (300 MHz, CDCl₃, δ in ppm): 0.84 (t, 7.4 Hz, 3H); from 1.09 to 1.33 (m, 10H); from 1.56 to 1.69 (m, 8H); 2.64 (m, 2H); 2.93 (m, 2H); 3.49 (m, 2H); 3.70 (s, 3H); 3.95 (s, 2H); 5.23 (m, 1H); 6.73 (d, 9.0 Hz, 1H); 6.86 (d, 8.6 Hz, 2H); 7.06 (m, 2H); from 7.15 to 7.22 (m, 4H).

### Step 3: 3-[4-[1-(1-(2-(1--piperidinyl)phenyl)pentyl)aminocarbonylmethyl]phenoxy]-2,2-dimethylpropanoic acid

This compound was obtained by saponification of the previously described intermediate according to protocol H. The expected product was purified by silica gel chromatography (dichloromethane/methanol 9/1).

Yield: 82%

Aspect: white powder

¹H NMR (300 MHz, DMSO d6/D₂O, δ in ppm): 0.82 (t, 7.1 Hz, 3H); from 1.19 to 1.26 (m, 10H); from 1.51 to 1.63 (m, 8H); 2.51 (m, 2H); 3.05 (m, 2H); 3.36 (m, 2H); 3.89 (s, 2H); 5.21 (m, 1H); 6.81 (d, 8.8 Hz, 2H); from 7.01 to 7.16 (m, 5H); 7.26 (d, 7.6 Hz, 1H).

Compound 43:

### 2-[4-[1-(1-(2-((3S,5R)-3,5-dimethylpiperidin-1-yl)phenyl)pentyl)aminocarbonylmethyl]phenoxy]-2-methylpropanoic acid

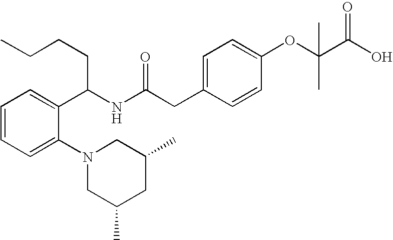

This product was synthesized in 2 steps:

### Step 1: Ethyl 2-[4-[1-(1-(2-((3S,5R)-3,5-dimethylpiperidin-1-yl)phenyl)pentyl)aminocarbonylmethyl]phenoxy]-2-methylpropanoate

This intermediate compound was obtained by condensation of amine 2-27 and acid 3-1 according to protocol F. It was purified by silica gel chromatography (cyclohexane/ethyl acetate 8/2).

Yield: 72%

Aspect: white solid

¹H NMR (300 MHz, CDCl₃, δ in ppm): 0.67 (m, 1 H); from 0.80 to 0.92 (m, 9H); 1.09 (m, 1H); 1.25 (m, 6H); from 1.52 to 1.83 (m, 11H); 2.07 (t, 10.9 Hz, 1H); 2.34 (t, 10.9 Hz, 1H); 2.77 (m, 1H); 3.03 (m, 1H); 3.46 (m, 2H); 4.23 (q, 7.7 Hz, 2H); 5.22 (m, 1H); 6.72 (d, 8.6 Hz, 1H); 6.81 (d, 8.2 Hz, 2H); from 7.02 to 7.23 (m, 6H).

### Step 2: 2-[4-[1-(1-(2-((3S,5R)-3,5-dimethylpiperidin-1-yl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 87%

Aspect: white powder

¹H NMR (300 MHz, DMSO d6/D₂O, δ in ppm): 0.59 (m, 1H); 0.75 (d, 6.0 Hz, 3H); 0.80 (t, 7.0 Hz, 3H); 0.87 (d, 6.0 Hz, 3H); from 1.15 to 1.28 (m, 4H); 1.43 (m, 6H); 1.52 (m, 2H); from 1.72 to 1.86 (m, 4H); 2.35 (t, 10.6 Hz, 1H); 2.75 (m, 1H); 3.23 (m, 1H); 3.34 (m, 2H); 5.19 (m, 1H); 6.72 (d, 8.2 Hz, 2H); from 7.00 to 7.17 (m, 5H); 7.26 (d, 7.6 Hz, 1H).

Compound 44:

### 2-[4-[1-(1-(2-(hydroxy)phenyl)pentyl)aminocarbonylmethyl]phenoxy]-2-methylpropanoic acid

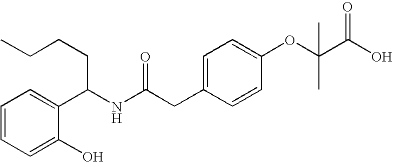

This product was synthesized in 2 steps:

### Step 1: Ethyl 2-[4-[1-(1-(2-(hydroxy)phenyl)pentyl)aminocarbonylmethyl]phenoxy]-2-methylpropanoate

This intermediate compound was obtained by condensation of amine 2-28 and acid 3-1 according to protocol F. It was purified by silica gel chromatography (cyclohexane/ethyl acetate 9/1).

Yield: 98%

Aspect: pale yellow oil

¹H NMR (300 MHz, CDCl₃, δ in ppm): 0.85 (t, 6.8 Hz, 3H); from 1.15 to 1.31 (m, 7H); 1.61 (s, 6H); from 1.74 to 1.80 (m, 2H); 3.50 (m, 2H); 4.26 (q, 7.2 Hz, 2H); 4.98 (m, 1H); 6.08 (d, 8.7 Hz, 1H); from 6.80 to 6.91 (m, 4H); from 7.04 to 7.18 (m, 4H); 8.63 (s, 1H).

### Step 2: 2-[4-[1-(1-(2-(hydroxy)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 19%

Aspect: white powder

¹H NMR (300 MHz, DMSO d6, δ in ppm): 0.82 (t, 6.5 Hz, 3H); from 1.23 to 1.65 (m, 12H); 3.38 (m, 2H); 5.04 (m, 1H); from 6.70 to 6.76 (m, 4H) from 6.98 to 7.15 (m, 4H); 8.28 (d, 8.8 Hz, 1H); 9.41 (s, 1H); 13.04 (s(broad), 1H).

Compound 45:

### 2-[4-[1-(1-(3-fluoro-2-(1-piperidinyl)phenyl)pentyl)aminocarbonylmethyl]phenoxy]-2-methylpropanoic acid

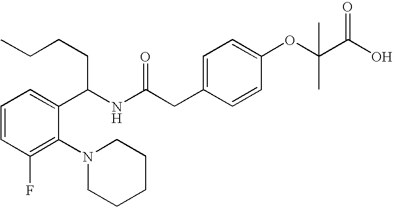

This product was synthesized in 2 steps:

### Step 1: Ethyl 2-[4-[1-(1-(3-fluoro-2-(1-piperidinyl)phenyl)pentyl)aminocarbonylmethyl]phenoxy]-2-methylpropanoate

This intermediate compound was obtained by condensation of amine 2-29 and acid 3-1 according to protocol F. It was purified by silica gel chromatography (cyclohexane/ethyl acetate 8/2).

Yield: 56%

Aspect: white solid

¹H NMR (300 MHz, CDCl₃, δ in ppm): 0.84 (t, 7.1 Hz, 3H); from 1.09 to 1.43 (m, 7H); from 1.58 to 1.82 (m, 14H); 2.69 (m, 1H); 2.95 (m, 1H); 3.05 (m, 1H); 3.18 (m, 1H); 3.48 (m, 2H); 4.23 (q, 7.1 Hz, 2H); 5.21 (m, 1H); from 6.70 to 6.95 (m, 5H); from 7.02 to 7.14 (m, 6H).

### Step 2: 2-[4-[1-(1-(3-fluoro-2-(1-piperidinyl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 62%

Aspect: white powder

¹H NMR (300 MHz, DMSO d6, δ in ppm): 0.84 (t, 6.5 Hz, 3H); from 1.24 to 1.31 (m, 4H); 1.45 (s, 6H); from 1.45 to 1.78 (m, 8H); 2.77 (m, 2H); 3.09 (m, 2H); 3.34 (m, 2H); 5.28 (m, 1H); 6.73 (d, 8.3 Hz, 2H); from 6.95 to 7.16 (m, 5H); 8.37 (d, 8.7 Hz, 1H).

Compound 46:

### 5-{N-ethyl-N-[4-[1-(3-methyl-1-(2-(1-piperidinyl)phenyl)butyl)aminocarbonylmethyl]phenyl]amino}-2,2-dimethylpentanoic acid

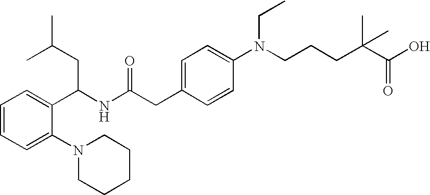

This product was synthesized in 3 steps:

### Step 1: Methyl 5-iodo-2,2-dimethylpentanoate

Under anhydrous atmosphere, diisopropylamine (2.75 ml; 19.6 mmol) in THF (5 ml) was cooled to 0° C. nButyllithium (2M in pentane, 9.8 ml; 19.6 mmol) was added dropwise. The mixture was stirred 30 min at 0° C. and cooled to −78° C. with dry ice. Methyl 2-methylpropanoate (1.0 g; 9.8 mmol) was added and the mixture was stirred for 45 min at −78° C. 1,3-diiodopropane (5.9 ml; 39.2 mmol) was added and stirring was maintained for 1 h. The mixture was slowly warmed to room temperature. The crude was neutralized upon addition of 2M HCl (25 ml) and the expected product was extracted by ethyl acetate (3×25 ml). Organic layers were combined, dried with magnesium sulfate (MgSO₄), and concentrated under reduced pressure. The expected product was purified by silica gel chromatography (cyclohexane/ethyl acetate 95/5).

Yield: 47%

Aspect: pale yellow oil

¹H NMR (300 MHz, CDCl₃, δ in ppm): 1.18 (s, 6H); from 1.58 to 1.79 (m, 4H); 3.14 (t, 6.9 Hz, 2H); 3.66 (s, 3H).

### Step 2: Methyl 5-{N-ethyl-N-[4-[1-(3-methyl-1-(2-(1-piperidinyl)phenyl)butyl)aminocarbonylmethyl]phenyl]amino}-2,2-dimethylpentanoate

Amine 4-5 (250 mg, 0.61 mmol) was solubilized in anhydrous DMF (7 ml). The mixture was cooled to 0° C. under anhydrous atmosphere and sodium methylate (39.8 mg, 0.74 mmol) was added. The mixture was stirred at room temperature for 15 minutes and was cooled again to 0° C. Methyl 5-iodo-2,2-dimethylpentanoate (166 mg, 0.61 mmol) was then added. The mixture was stirred at room temperature overnight. It was then hydrolyzed with water (50 ml) and extracted with ethyl acetate (2×20 ml). The organic layer was washed with NaClsat (2×20 ml), dried with magnesium sulfate, filtered and concentrated. The expected product was purified by silica gel chromatography (cyclohexane/ethyl acetate 8/2).

Yield: 33%

Aspect: pale yellow oil

¹H NMR (300 MHz, CDCl₃, δ in ppm): 0.90 (d, 6.3 Hz, 3H); 0.91 (d, 6.3 Hz, 3H); 1.14 (t, 7.1 Hz, 3H); 1.19 (s, 6H); from 1.35 to 1.68 (m, 13H); 2.61 (m, 2H); 2.98 (m, 2H); 3.23 (m, 2H); 3.35 (q, 7.1 Hz, 2H); 3.44 (m, 2H); 3.66 (s, 3H); 5.40 (m, 1H); 6.34 (d, 9.0 Hz, 1H); 6.61 (d, 8.6 Hz, 2H); from 7.04 to 7.08 (m, 4H); from 7.15 to 7.22 (m, 2H).

### Step 3: 5-{N-ethyl-N-[4-[1-(3-methyl-1-(2-(1-piperidinyl)phenyl)butyl)aminocarbonylmethyl]phenyl]amino}-2,2-dimethylpentanoic acid

This compound was obtained by saponification of the previously described intermediate according to protocol H. The expected product was purified by silica gel chromatography (dichloromethane/methanol 98/2).

Yield: 79%

Aspect: ochre powder

¹H NMR (300 MHz, DMSO d6, δ in ppm): 0.89 (d, 5.1 Hz, 6H); 1.03 (t, 6.5 Hz, 3H); 1.07 (s, 6H); from 1.24 to 1.67 (m, 13H); 2.50 (m, 2H); from 3.09 to 3.34 (m, 8H); 5.31 (m, 1H); 6.53 (d, 8.8 Hz, 2H); from 6.99 to 7.15 (m, 5H); 7.28 (d, 7.9 Hz, 1H); 8.25 (d, 8.8 Hz, 1H); 12.11 (s(broad), 1H).

Compound 47:

### 5-[4-[1-(1-(2-(1-piperidinyl)phenyl)pentyl)aminocarbonylmethyl]phenoxy]-2,2-dichloropentanoic acid

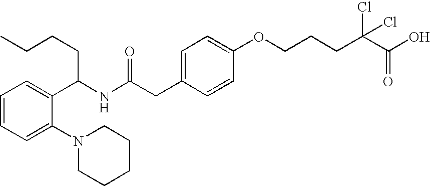

This product was synthesized in 3 steps:

### Step 1: Methyl 5-iodo-2,2-dichloropentanoate

Under anhydrous atmosphere, diisopropylamine (1.1 ml; 7.69 mmol) in THF (5 ml) was cooled to 0° C. nButyllithium (2M in pentane, 3.8 ml; 7.69 mmol) was added dropwise. The mixture was stirred 30 min at 0° C. and cooled to −78° C. with dry ice. Methyl 2,2-dichloroacetate (1.0 g; 7.00 mmol) was added and the mixture was stirred for 15 min at −78° C. 1,3-diiodopropane (3.1 ml; 21.0 mmol) was added and stirring was maintained for 1 h. The mixture was slowly warmed to room temperature. It was then neutralized by addition of HCl 2M (25 ml) and the expected product was extracted by ethyl acetate (3×25 ml). Organic layers were combined, dried with magnesium sulfate (MgSO₄), and concentrated under reduced pressure. The expected product was purified by silica gel chromatography (cyclohexane/ethyl acetate 95/5).

Yield: 56%

Aspect: pale red oil

¹H NMR (300 MHz, CDCl₃, δ in ppm): from 2.12 to 2.22 (m, 2H); from 2.54 to 2.59 (m, 2H); 3.25 (t, 6.8 Hz, 2H); 3.92 (s, 3H).

### Step 2: Methyl 5-[4-[1-(1-(2-(1-piperidinyl)phenyl)pentyl)aminocarbonylmethyl]phenoxy]-2,2-dichloropentanoate

This intermediate was obtained by substitution - of methyl 5-iodo-2,2-dichloropentanoate by phenol derivative 4-3 according to protocol D. It was purified by silica gel chromatography (dichloromethane/ethyl acetate 95/5).

Yield: 24%

Aspect: white solid

¹H NMR (300 MHz, CDCl₃, δ in ppm): 0.84 (t, 7.1 Hz, 3H); from 1.05 to 1.35 (m, 4H); 1.61 (m, 8H); 2.13 (m, 2H); 2.65 (m, 4H); 2.94 (m, 2H); 3.50 (s, 2H); 3.90 (s, 3H); 4.03 (t, 5.9 Hz, 2H); 5.25 (m, 1H); 6.75 (d, 8.8 Hz, 1H) 6.86 (d, 8.8 Hz, 2H); 7.06 (m, 2H); 7.20 (m, 4H).

### Step 3: 5-[4-[1-(1-(2-(1-piperidinyl)phenyl)pentyl)aminocarbonylmethyl]phenoxy]-2,2-dichloropentanoic acid

This compound was obtained by saponification of the previously described intermediate according to protocol H. The expected product was purified by silica gel chromatography (dichloromethane/methanol 9/1).

Yield: 40%

Aspect: white powder

¹H NMR (300 MHz, DMSO d6, δ in ppm): 0.83 (t, 6.4 Hz, 3H); 1.27 (m, 4H); 1.53 (m, 8H); 1.90 (m, 2H); from 2.41 to 2.51 (m, 4H); 3.07 (m, 2H); 3.37 (m, 2H); 3.97 (t, 6.4 Hz, 2H); 5.21 (m, 1H); 6.83 (d, 8.5 Hz, 2H); from 7.03 to 7.16 (m, 5H); 7.27 (d, 7.3 Hz, 1H); 8.33 (d, 8.5 Hz, 1H).

Compound 48:

### 2-[4-[1-(2-(4-methoxyphenyl)-1-(2-(1-piperidinyl)phenyl)ethyl)aminocarbonylmethyl]phenoxy]-2-methylpropanoic acid

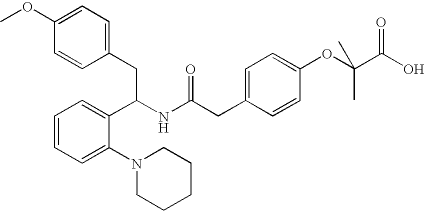

This product was synthesized in 2 steps:

### Step 1: Ethyl 2-[4-[1-(2-(4-methoxyphenyl)-1-(2-(1-piperidinyl)phenyl)ethyl)aminocarbonylmethyl]phenoxy]-2-methylpropanoate

This intermediate compound was obtained by condensation of amine 2-30 and acid 3-1 according to protocol G. It was purified by silica gel chromatography (dichloromethane/ethyl acetate 9/1).

Yield: 39%

Aspect: colorless oil

¹H NMR (300 MHz, CDCl₃, δ in ppm): 1.27 (t, 7.2 Hz, 3H); from 1.50 to 1.80 (m, 12H); 2.61 (m, 2H); 2.79 (m, 1H); 2.95 (m, 3H); 3.43 (s, 2H); 3.78 (s, 3H); 4.25 (q, 7.2 Hz, 2H); 5.53 (m, 1H); from 6.68 to 6.90 (m, 8H); 7.02 (m, 3H); 7.18 (m, 2H).

### Step 2: 2-[4-[1-(2-(4-methoxyphenyl)-1-(2-(1-piperidinyl)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 41%

Aspect: white powder

¹H NMR (300 MHz, DMSO d6/D₂O, δ in ppm): 1.45 (s, 6H); 1.60 (m, 2H); 1.81 (m, 4H); 2.51 (m, 2H); from 3.00 to 3.35 (m, 6H); 3.69 (s, 3H); 5.11 (m, 1H); 6.62 (d, 8.5 Hz, 2H); 6.79 (d, 8.5 Hz, 2H); 6.87 (d, 8.5 Hz, 2H); 7.21 (d, 8.5 Hz, 2H); from 7.45 to 7.65 (m, 4H).

Compound 49:

### 2-[4-[1-(1-(2-(1-piperidinyl)-4-(1H-pyrrolyl)phenyl)pentyl)aminocarbonylmethyl]phenoxy]-2-methylpropanoic acid

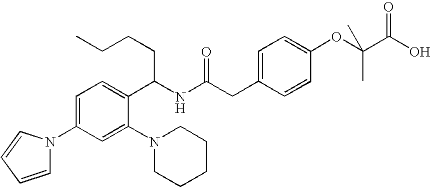

This product was synthesized in 2 steps from ethyl 2-[4-[1-(1-(2-(1-piperidinyl)-4-(bromo)phenyl)pentyl)aminocarbonylmethyl]phenoxy]-2-methylpropanoate (intermediate for the synthesis of compound 33).

### Step 1: Ethyl 2-[4-[1-(1-(2-(1-piperidinyl)-4-(1H-pyrrolyl)phenyl)pentyl)aminocarbonylmethyl]phenoxy]-2-methylpropanoate

This intermediate compound was synthesized from ethyl 2-[4-[1-(1-(2-(1-piperidinyl)-4-(bromo)phenyl)pentyl)aminocarbonylmethyl]phenoxy]-2-methylpropanoate and pyrrole according to protocol I. It was purified by silica gel chromatography (cyclohexane/ethyl acetate 75/25).

Yield: 51%

Aspect: pale yellow oil

¹H NMR (300 MHz, CDCl₃, δ in ppm): 0.84 (t, 7.0 Hz, 3H); from 1.13 to 1.33 (m, 7H); from 1.54 to 1.80 (m, 14H); 2.67 (m, 2H); 2.98 (m, 2H); 3.50 (m, 2H); 4.23 (q, 7.0 Hz, 2H); 5.22 (m, 1H); 6.33 (m, 2H); 6.41 (d, 9.1 Hz, 1H); 6.83 (d, 8.4 Hz, 2H); from 7.03 to 7.16 (m, 7H).

### Step 2: 2-[4-[1-(1-(2-(1-piperidinyl)-4-(1H-pyrrolyl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 85/15).

Yield: 21%

Aspect: pale yellow powder

¹H NMR (300 MHz, DMSO d6, δ in ppm): 0.84 (t, 7.0 Hz, 3H); from 1.28 to 1.66 (m, 18H); 2.57 (m, 2H); 3.15 (m, 2H); 3.37 (m, 2H); 5.17 (m, 1H); 6.23 (m, 2H); 6.75 (d, 8.3 Hz, 2H); from 7.12 to 7.23 (m, 4H); 7.31 (m, 3H); 8.38 (d, 8.3 Hz, 1H).

Compound 50:

### 2-[4-[1-(1-(2-(1-piperidinyl)-4-(phenethyl)phenyl)pentyl)aminocarbonylmethyl]phenoxy]-2-methylpropanoic acid

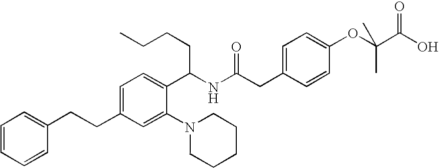

This product was synthesized in 3 steps from ethyl 2-[4-[1-(1-(2-(1-piperidinyl)-4-(bromo)phenyl)pentyl)aminocarbonylmethyl]phenoxy]-2-methylpropanoate which synthesis has been described previously (intermediate for the synthesis of compound 33).

### Step 1: Ethyl 2-[4-[1-(1-(2-(1-piperidinyl)-4-(phenylethenyl)phenyl)pentyl)aminocarbonylmethyl]phenoxy]-2-methylpropanoate

This intermediate compound was synthesized upon reaction of ethyl 2-[4-[1-(1-(2-(1-piperidinyl)-4-(bromo)phenyl)pentyl)aminocarbonylmethyl]phenoxy]-2-methylpropanoate on trans-phenylethenylboronic acid according to protocol J as previously described. It was purified by silica gel chromatography (cyclohexane/ethyl acetate 8/2).

Yield: 42%

Aspect: pale yellow oil

¹H NMR (300 MHz, CDCl₃, δ in ppm): 0.85 (m, 3H); from 1.05 to 1.80 (m, 21H); 2.61 (m, 2H); 2.97 (m, 2H); 3.50 (m, 2H); 4.24 (q, 7.0 Hz, 2H); 5.21 (m, 1H); from 6.65 to 7.39 (m, 14H); 7.51 (d, 7.3 Hz, 1H).

### Step 2: 2-[4-[1-(1-(2-(1-piperidinyl)-4-(phenylethenyl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 85/15).

Yield: 85%

Aspect: pale yellow powder

¹H NMR (300 MHz, DMSO d6, δ in ppm): 0.84 (m, 3H); from 1.10 to 1.75 (m, 18H); 2.51 (m, 2H); 3.13 (m, 2H); 3.35 (m, 2H); 5.15 (m, 1H); 6.75 (d, 8.3 Hz, 2H); from 7.06 to 7.40 (m, 11H); 7.59 (d, 7.4 Hz, 1H); 8.38 (m, 1H).

### Step 3: 2-[4-[1-(1-(2-(1-piperidinyl)-4-(phenethyl)phenyl)pentyl)aminocarbonylmethyl]phenoxy]-2-methylpropanoic acid

The previously described acid was hydrogenated according to protocol E. The expected product was purified by silica gel chromatography (dichloromethane/methanol 8/2).

Yield: 68%

Aspect: white powder

¹H NMR (300 MHz, DMSO d6, δ in ppm): 0.83 (m, 3H); from 1.18 to 1.63 (m, 18H); 2.50 (m, 2H); 2.81 (m, 4H); 3.03 (m, 2H); 3.31 (m, 2H); 5.15 (m, 1H); 6.75 (d, 8.3 Hz, 2H); from 6.90 to 7.30 (m, 10H); 8.30 (d, 8.3 Hz, 1H).

Compound 51:

### 2-[4-[1-(3-phenyl-1-(2-(1-piperidinyl)phenyl)propyl)aminocarbonylmethyl]phenoxy]-2-methylpropanoic acid

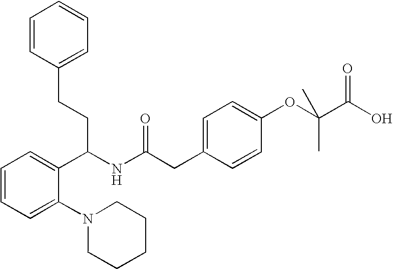

This product was synthesized in 2 steps:

### Step 1: Ethyl 2-[4-[1-(3-phenyl-1-(2-(1-piperidinyl)phenyl)propyl)aminocarbonylmethyl]phenoxy]-2-methylpropanoate

This intermediate compound was obtained by condensation of amine 2-31 and acid 3-1 according to protocol F. It was purified by silica gel chromatography (dichloromethane/cyclohexane 4/6).

Yield: 63%

Aspect: white powder

¹H NMR (300 MHz, CDCl₃, δ in ppm): 1.24 (t, 7.0 Hz, 3H); from 1.45 to 1.67 (m, 12H); from 1.93 to 2.03 (m, 2H); from 2.40 to 2.50 (m, 1H); from 2.57 to 2.65 (m, 3H); 2.91 (m, 2H); 3.49 (s, 2H); 4.22 (q, 7.0 Hz, 2H); 5.30 (m, 1H); 6.80 (m, 3H); from 7.05 to 7.29 (m, 11H).

### Step 2: 2-[4-[1-(3-phenyl-1-(2-(1-piperidinyl)phenyl)propyl)aminocarbonylmethyl]phenoxy]-2-methylpropanoic acid

This compound was obtained by saponification of the previously described intermediate according to protocol H. The expected product was purified by silica gel chromatography (dichloromethane/methanol 8/2).

Yield: 70%

Aspect: white powder

¹H NMR (300 MHz, DMSO d6, δ in ppm): from 1.38 to 1.42 (m, 12H); 1.82 (m, 2H); from 2.35 to 2.72 (m, 4H); 2.97 (m, 2H); 3.40 (m, 2H); 5.09 (m, 1H); 6.77 (d, 8.8 Hz, 2H); from 7.00 to 7.29 (m, 11H); 8.53 (d, 8.2 Hz, 1H).

Compound 52:

### N-[methylsulfonyl]-2-[4-[1-(1-(2-(1-piperidinyl)phenyl)pentyl)aminocarbonylmethyl]phenoxy]-2-methylpropanamide

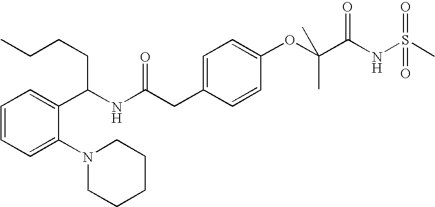

This compound was obtained by condensation between compound 10 and methanesulfonamide according to protocol K. It was purified by silica gel chromatography (dichloromethane/ethyl acetate 7/3).

Yield: 73%

Aspect: white powder

¹H NMR (300 MHz, CDCl₃, δ in ppm): 0.84 (t, 7.1Hz, 3H); from 1.13 to 1.69 (m, 18H); 2.67 (m, 2H); 2.92 (m, 2H); 3.33 (s, 3H); 3.50 (s, 2H); 5.21 (m, 1H); 6.88 (d, 8.3 Hz, 2H); from 7.09 to 7.23 (m, 7H); 9.09 (s(broad), 1H).
Compounds 52A and 52B:

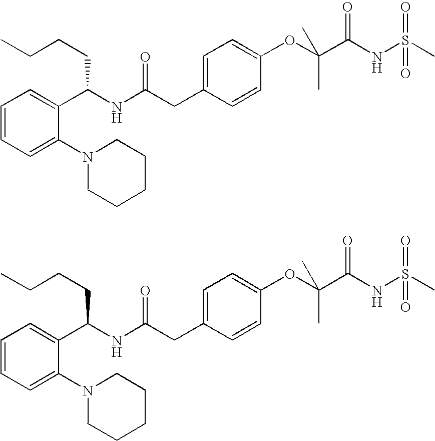

The two enantiomers of compound 52 were separated by semi-preparative HPLC on chiral column Chiralpak®AD (250*2.5mm, 20 μm, Chiral Technologies Europe) at room temperature. The separation was performed in isocratic mode using a mobile phase n-heptane-isopropanol (7/3) containing 0.1% trifluoroacetic acid.

The enantiomeric purity of both enantiomers was checked by analytical HPLC: Chiralpak®AD-H column (250*4.6mm, 5 μm, Chiral Technologies Europe) at 30° C.; isocratic mobile phase n-heptane/isopropanol (1/1) containing 0.1% trifluoroacetic acid; flow rate 0.5 ml/min; UV detection at 210 nm.

Compound 52A: R_{T}=15.5 min, 100%.

Compound 52B: R_{T}=8.7 min, 100%.

Compound 53:

### 5-[2-[1-(1-(2-(1-piperidinyl)phenyl)pentyl)aminocarbonylmethyl]phenoxy]-2,2-dimethylpentanoic acid

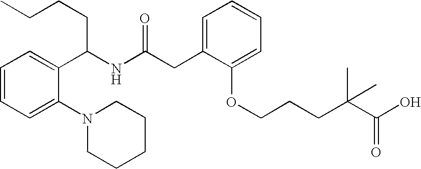

This product was synthesized in 2 steps from the phenol intermediate 4-6.

### Step 1: Methyl 5-[2-[1-(1-(2-(1-piperidinyl)phenyl)pentyl)aminocarbonylmethyl]phenoxy]-2,2-dimethylpentanoate

Phenol 4-6 (300 mg, 0.79 mmol) and methyl 5-iodo-2,2-dimethylpentanoate (intermediate for the synthesis of compound 46, 213 mg, 0.79 mmol) were solubilized in acetonitrile (10 ml). Cesium carbonate (514 mg, 1.58 mmol) was added and the crude was refluxed for 16 hours. The mixture was neutralized by addition of 1M HCl (100 ml) and extracted with ethyl acetate (3×50 ml). The organic layer was dried with magnesium sulfate, filtered and evaporated. The expected product was purified by silica gel chromatography (cyclohexane/ethyl acetate 8/2).

Yield: 85%

Aspect: pale yellow oil

¹H NMR (300 MHz, CDCl₃, δ in ppm): 0.82 (t, 7.1Hz, 3H); from 1.04 to 1.79 (m, 22H); 2.57 (m, 2H); 2.94 (m, 2H); 3.57 (s, 2H); 3.65 (s, 3H); 3.91 (m, 2H); 5.29 (m, 1H); 6.54 (d, 8.7 Hz, 1H); 6.84 (d, 8.3 Hz, 1H); 6.92 (m, 1H); from 6.99 to 7.05 (m, 2H); from 7.11 to 7.26 (m, 4H).

### Step 2: 5-[2-[1-(1-(2-(1-piperidinyl)phenyl)pentyl)aminocarbonylmethyl]phenoxy]-2,2-dimethylpentanoic acid

The methyl ester previously described (350 mg, 0.67 mmol) was dissolved in methanol (4 ml) and 2 M sodium hydroxide was added dropwise (4 ml). The mixture was heated at 50° C. overnight. The mixture was cooled, neutralized upon addition of 1M HCl (100 ml), and extracted with ethyl acetate (3×50 ml). The organic layer was dried with magnesium sulfate, filtered, and evaporated. The expected product was purified by silica gel chromatography (dichloromethane/methanol 95/5).

Yield: 14%

Aspect: white solid

¹H NMR (300 MHz, DMSO d6/D2O, δ in ppm): 0.80 (t, 6.8 Hz, 3H); from 1.01 to 1.58 (m, 22H); 2.51 (m, 2H); 3.01 (m, 2H); 3.41 (m, 2H); 3.86 (m, 2H); 5.19 (m, 1H); from 6.79 to 6.89 (m, 2H); from 6.99 to 7.25 (m, 6H).

Compound 54:

### 2-[4-[1-(1-(2-(methoxy)phenyl)pentyl)aminocarbonylmethyl]phenoxy]-2-methylpropanoic acid

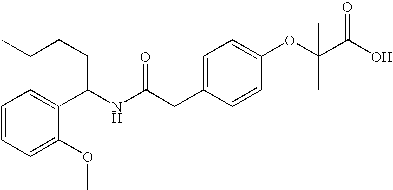

This product was synthesized in 2 steps from the phenol intermediate ethyl 2-[4-[1-(1-(2-(hydroxy)phenyl)pentyl)aminocarbonylmethyl]phenoxy]-2-methylpropanoate (intermediate for the synthesis of compound 44)

### Step 1: Ethyl 2-[4-[1-(1-(2-(methoxy)phenyl)pentyl)aminocarbonylmethyl]phenoxy]-2-methylpropanoate

Ethyl 2-[4-[1-(1-(2-(hydroxy)phenyl)pentyl)aminocarbonylmethyl]phenoxy]-2-methylpropanoate (500 mg, 1.17 mmol) was solubilized in acetonitrile (10 ml). Potassium carbonate (323 mg, 2.34 mmol) and iodomethane (249 mg, 1.75 mmol) were successively added. The mixture was refluxed for 16 hours. The crude mixture was cooled, acidified upon addition of 1M HCl (100 ml), and extracted by ethyl acetate (3×50 ml). The organic layer was dried with magnesium sulfate, filtered and concentrated. The expected product was purified by silica gel chromatography (cyclohexane/ethyl acetate 7/3).

Yield: 64%

Aspect: colorless oil

¹H NMR (300 MHz, CDCl₃, δ in ppm): 0.80 (m, 3H); from 1.06 to 1.29 (m, 7H); from 1.58 to 1.68 (m, 8H); 3.49 (m, 2H); 3.58 (m, 3H); 4.24 (m, 2H); 5.00 (m, 1H); 6.53 (m, 1H); from 6.76 to 6.89 (m, 4H); from 7.01 to 7.26 (m, 4H).

### Step 2: 2-[4-[1-(1-(2-(methoxy)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 85/15).

Yield: 43%

Aspect: white powder

¹H NMR (300 MHz, DMSO d6, δ in ppm): 0.83 (m, 3H); 1.24 (m, 4H); from 1.45 to 1.66 (m, 8H); 3.37 (s, 2H); 3.75 (s, 3H); 5.08 (m, 1H); 6.74 (d, 8.5 Hz, 2H); from 6.85 to 6.94 (m, 2H); from 7.09 to 7.22 (m, 4H); 8.28 (d, 8.8 Hz, 1H).

Compound 55:

### 2-[4-[1-(2-(3-methylphenyl)-1-(2-(1-piperidinyl)phenyl)ethyl)aminocarbonylmethyl]phenoxy]-2-methylpropanoic acid

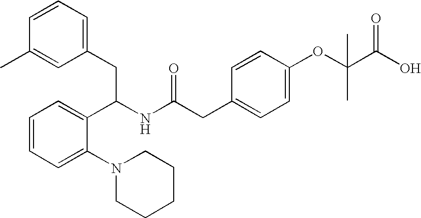

This product was synthesized in 2 steps:

### Step 1: Ethyl 2-[4-[1-(2-(3-methylphenyl)-1-(2-(1-piperidinyl)phenyl)ethyl)aminocarbonylmethyl]phenoxy]-2-methylpropanoate

This intermediate compound was obtained by condensation of amine 2-32 and acid 3-1 according to protocol F. It was purified by silica gel chromatography (cyclohexane/ethyl acetate 8/2).

Yield: 92%

Aspect: white powder

¹H NMR (300 MHz, CDCl₃, δ in ppm): 1.28 (t, 7.0 Hz, 3H); from 1.55 to 1.73 (m, 12H); 2.29 (s, 3H); 2.62 (m, 2H); from 2.77 to 2.84 (m, 1H); 2.99 (m, 3H); 3.44 (s, 2H); 4.26 (q, 7.0 Hz, 2H); 5.56 (m, 1H); 6.65 (d, 7.9 Hz, 1H); 6.73 (d, 7.3 Hz, 1H); 6.82 (m, 3H); 6.91 (d, 7.6 Hz, 1H); from 6.98 to 7.11 (m, 5H); 7.19 (m, 2H).

### Step 2: 2-[4-[1-(2-(3-methylphenyl)-1-(2-(1-piperidinyl)phenyl)ethyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 74%

Aspect: white powder

¹H NMR (300 MHz, DMSO d6, δ in ppm): 1.41 (s, 6H); 1.60 (m, 6H); 2.25 (s, 3H); 2.49 (m, 2H); 2.70 (m, 1H); 2.83 (m, 1H); 3.06 (m, 2H); 3.27 (m, 2H); 5.44 (m, 1H); 6.66 (d, 8.2 Hz, 2H); 6.89 (d, 8.2 Hz, 2H); from 7.00 to 7.18 (m, 7H); 7.37 (d, 7.3 Hz, 1H); 8.52 (d, 8.5 Hz, 1H).

Compound 56:

### 2-[2-chloro-4-[1-(3-methyl-1-(2-(1-piperidinyl)phenyl)butyl)aminocarbonylmethyl]phenoxy]-2-methylpropanoic acid

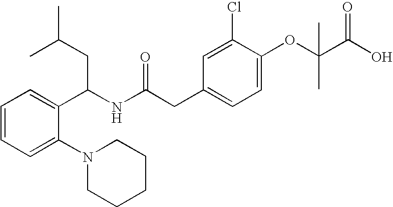

This product was synthesized in 2 steps:

### Step 1: Ethyl 2-[2-chloro-4-[1-(3-methyl-1-(2-(1-piperidinyl)phenyl)butyl)aminocarbonylmethyl]phenoxy]-2-methylpropanoate

This intermediate was obtained upon substitution of ethyl bromoisobutyrate by phenol 4-7 according to protocol D. It was purified by silica gel chromatography (dichloromethane/ethyl acetate 95/5).

Yield: 65%

Aspect : white solid

¹H NMR (300 MHz, CDCl₃, δ in ppm): 0.92 (d, 6.4 Hz, 6H); 1.29 (t, 7.1Hz, 3H); from 1.35 to 1.80 (m, 15H); 2.66 (m, 2H); 2.95 (m, 2H); 3.45 (s, 2H); 4.26 (q, 7.1 Hz, 2H); 5.35 (m, 1H); 6.66 (d, 8.2 Hz, 1H); 6.86 (d, 8.5 Hz, 1H); from 7.02 to 7.10 (m, 3H); 7.21 (m, 2H); 7.28 (m, 1H).

### Step 2: 2-[2-chloro-4-[1-(3-methyl-1-(2-(1-piperidinyl)phenyl)butyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5) and recrystallized in toluene.

Yield: 36%

Aspect: white crystals

¹H NMR (300 MHz, DMSO d6, δ in ppm): 0.89 (d, 5.3 Hz, 6H); 1.33 (m, 1H); from 1.43 to 1.75 (m, 14H); 2.51 (m, 2H); 3.07 (m, 2H); 3.38 (m, 2H); 5.32 (m, 1H); 6.84 (d, 8.5 Hz, 1H); from 7.01 to 7.18 (m, 4H); 7.25 (dd, 8.5 Hz, 2.1Hz, 1H); 7.32 (d, 2.1 Hz, 1H); 8.39 (d, 8.2 Hz, 1H); 13.19 (s(broad), 1H).

Compound 57:

### 6-[4-[1-(3-methyl-1-(2-(1-piperidinyl)phenyl)butyl)aminocarbonylmethyl]phenoxy]-2,2-dimethylhexanoic acid

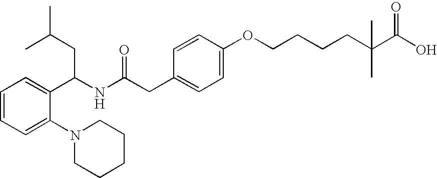

This product was synthesized in 3 steps:

### Step 1: Methyl 6-iodo-2,2-dimethylhexanoate

Under anhydrous atmosphere, diisopropylamine (4.16 ml; 29.6 mmol) in THF (50 ml) was cooled to 0° C. nButyllithium (2M in pentane, 14.8 ml; 29.6 mmol) was added dropwise. The mixture was stirred 30 min at 0° C. and cooled to −78° C. with dry ice. Methyl 2-methylpropanoate (2.75 g; 26.9 mmol) was added and the mixture was stirred for 15 min at −78° C. 1,4-diiodobutane (10.6 ml; 80.7 mmol) was added and stirring was maintained for 1 h. The crude was slowly warmed to room temperature. The mixture was neutralized upon addition of 2M HCl (100 ml) and the expected product was extracted by ethyl acetate (3×100 ml). Organic layers were combined, dried with magnesium sulfate (MgSO₄), then concentrated under reduced pressure. The expected product was purified by silica gel chromatography (cyclohexane/ethyl acetate 95/5).

Yield: 63%

Aspect: pale yellow oil

¹H NMR (300 MHz, CDCl₃, δ in ppm): 1.18 (s, 6H); from 1.25 to 1.40 (m, 2H); from 1.50 to 1.56 (m, 2H); 1.81 (m, 2H); 3.19 (t, 7.0 Hz, 2H); 3.67 (s, 3H).

### Step 2: Methyl 6-[4-[1-(3-methyl-1-(2-(1-piperidinyl)phenyl)butyl)aminocarbonylmethyl]phenoxy]-2,2-dimethylhexanoate

This intermediate was obtained by substitution of methyl 6-iodo-2,2-dimethylhexanoate by phenol 4-1 according to protocol D. It was purified by silica gel chromatography (cyclohexane/ethyl acetate 8/2).

Yield: 75%

Aspect: white solid

¹H NMR (300 MHz, CDCl₃, δ in ppm): 0.92 (d, 6.4 Hz, 6H); 1.20 (s, 6H); from 1.30 to 1.85 (m, 15H); 2.63 (m, 2H); 2.97 (m, 2H); 3.50 (s, 2H); 3.68 (s, 3H); 3.95 (t, 6.5 Hz, 2H); 5.40 (m, 1H); 6.41 (d, 8.5 Hz, 1H); 6.86 (d, 8.8 Hz, 2H); 7.06 (m, 2H); from 7.15 to 7.20 (m, 4H).

### Step 3: 6-[4-[1-(3-methyl-1-(2-(1-piperidinyl)phenyl)butyl)aminocarbonylmethyl]phenoxy]-2,2-dimethylhexanoic acid

This compound was obtained by saponification of the previously described intermediate according to protocol H. The expected product was purified by silica gel chromatography (dichloromethane/methanol 95/5).

Yield: 53%

Aspect: white solid

¹H NMR (300 MHz, CDCl₃, δ in ppm): 0.89 (d, 6.4 Hz, 6H); 1.24 (s, 6H); from 1.35 to 1.85 (m, 15H); 2.63 (m, 2H); 2.97 (m, 2H); 3.50 (s, 2H); 3.96 (t, 6.3 Hz, 2H); 5.40 (m, 1H); 6.46 (s(broad), 1H); 6.86 (d, 8.2 Hz, 2H); from 7.05 to 7.19 (m, 6H).

Compound 58:

### N-[methylsulfonyl]-2-[4-[1-(3-methyl-1-(2-(1-piperidinyl)phenyl)butyl)aminocarbonylmethyl]phenoxy]-2-methylpropanamide

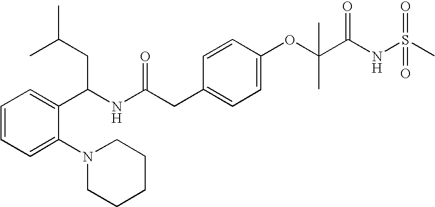

This compound was obtained by condensation between compound 1 and methanesulfonamide according to protocol K. It was purified by silica gel chromatography (dichloromethane/methanol 95/5) and recrystallized in toluene.

Yield: 56%

Aspect: white powder

¹H NMR (300 MHz, DMSO d6, δ in ppm): 0.87 (d, 5.3 Hz, 6H); 1.30 (m, 1H); from 1.44 to 1.66 (m, 14H); 2.50 (m, 2H); 3.07 (m, 2H); 3.25 (s, 3H); 3.38 (m, 2H); 5.32 (m, 1H); 6.79 (d, 8.5 Hz, 2H); from 7.00 to 7.17 (m, 5H); 7.25 (d, 7.6 Hz, 1H); 8.35 (d, 8.2 Hz, 1H); 11.97 (s(broad), 1H).

Compound 59:

### N-[trifluoromethylsulfonyl]-2-[4-[1-(3-methyl-1-(2-(1-piperidinyl)phenyl)butyl)aminocarbonylmethyl]phenoxy]-2-methylpropanamide

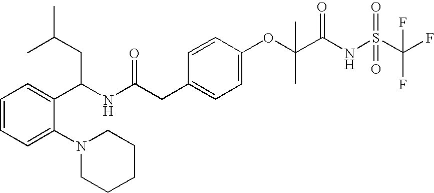

This compound was obtained by condensation between compound 1 and trifluoromethanesulfonamide according to protocol K. It was purified by silica gel chromatography (dichloromethane/methanol 95/5).

Yield: 59%

Aspect: white powder

¹H NMR (300 MHz, DMSO d6, δ min ppm): 0.88 (d, 6.2 Hz, 6H); from 1.25 to 1.67 (m, 15H); 2.50 (m, 2H); 3.08 (m, 2H); 3.33 (m, 2H); 5.31 (m, 1H); 6.68 (d, 8.2 Hz, 2H); from 7.01 to 7.12 (m, 5H); 7.25 (m, 1H); 8.32 (m, 1H).

Compound 60:

### N-[trifluoromethylsulfonyl]-2-[4-[1-(1-(2-(1-piperidinyl)phenyl)pentyl)aminocarbonylmethyl]phenoxy]-2-methylpropanamide

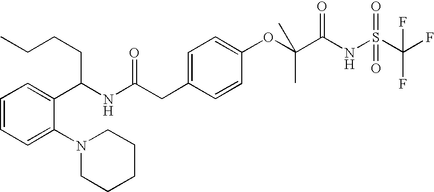

This compound was obtained by condensation between compound 10 and trifluoromethanesulfonamide according to protocol K. It was purified by silica gel chromatography (d ichloromethanelmethanol 95/5).

Yield: 60%

Aspect: white powder

¹H NMR (300 MHz, DMSO d6, δ in ppm): 0.83 (t, 7.0 Hz, 3H); from 1.19 to 1.64 (m, 18H); 2.50 (m, 2H); 3.07 (m, 2H); 3.32 (m, 2H); 5.21 (m, 1H); 6.68 (d, 8.5 Hz, 2H); from 7.02 to 7.15 (m, 5H); 7.24 (m, 1H); 8.31 (m, 1H).

Compound 61:

### N-[phenylsulfonyl]-2-[4-[1-(1-(2-(1-piperidinyl)phenyl)pentyl)aminocarbonylmethyl]phenoxy]-2-methylpropanamide

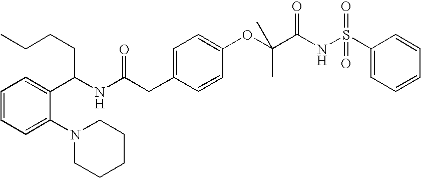

This compound was obtained by condensation between compound 10 and benzenesulfonamide according to protocol K. It was purified by silica gel chromatography (dichloromethane/methanol 98/2).

Yield: 21%

Aspect: white powder

¹H NMR (300 MHz, DMSO d6, δ in ppm): 0.80 (t, 6.4 Hz, 3H); from 1.16 to 1.61 (m, 18H); 2.50 (m, 2H); 3.03 (m, 2H); 3.32 (m, 2H); 5.19 (m, 1H); 6.51 (d, 8.5 Hz, 2H); from 6.98 to 7.13 (m, 5H); 7.23 (d, 7.6 Hz, 1H); from 7.55 to 7.67 (m, 3H); 7.82 (d, 7.6 Hz, 2H); 8.32 (d, 8.2 Hz, 1H); 12.39 (s(broad), 1H).

Compound 62:

### N-[methylsulfonyl]-2-[4-[1-(2-phenyl-1-(2-(1-piperidinyl)phenyl)ethyl)aminocarbonylmethyl]phenoxy]-2-methylpropanamide

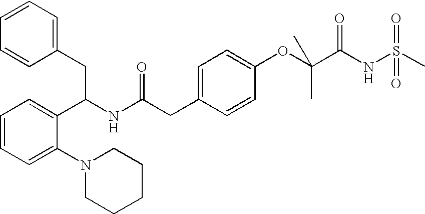

This compound was obtained by condensation between compound 7 and methanesulfonamide according to protocol K. It was purified by silica gel chromatography (dichloromethane/methanol 98/2).

Yield: 23%

Aspect: white powder

¹H NMR (300 MHz, DMSO d6, δ in ppm): 1.44 (s, 6H); 1.60 (m, 6H); 2.50 (m, 2H); 2.73 (m, 1H); 2.90 (m, 1H); 3.06 (m, 2H); 3.26 (m, 5H); 5.47 (m, 1H); 6.71 (d, 8.2 Hz, 2H); 6.95 (d, 8.2 Hz, 2H); from 7.08 to 7.26 (m, 8H); 7.38 (d, 7.3 Hz, 1H); 8.54 (d, 8.2 Hz, 1H); 11.98 (s, 1H).

Compound 63:

### N-[benzylsulfonyl]-2-[4-[1-(1-(2-(1-piperidinyl)phenyl)pentyl)aminocarbonylmethyl]phenoxy]-2-methylpropanamide

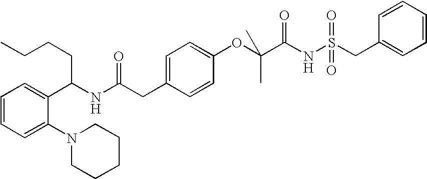

This compound was obtained by condensation between compound 10 and phenylmethanesulfonamide according to protocol L. It was purified by silica gel chromatography (dichloromethane/methanol 98/2).

Yield: 39%

Aspect: white powder

¹H NMR (300 MHz, DMSO d6, δ in ppm): 0.82 (t, 7.0 Hz, 3H); from 1.19 to 1.64 (m, 18H); 2.50 (m, 2H); 3.06 (m, 2H); 3.39 (m, 2H); 4.75 (m, 2H); 5.21 (m, 1H); 6.76 (d, 8.5 Hz, 2H); from 7.00 to 7.15 (m, 5H); 7.25 (d, 7.3 Hz, 1H); from 7.33 to 7.37 (m, 5H); 8.34 (d, 8.5 Hz, 1H); 11.84 (s(broad), 1H).

Compound 64:

### N-[phenylsulfonyl]-2-[4-[1-(3-methyl-1-(2-(1-piperidinyl)phenyl)butyl)aminocarbonylmethyl]phenoxy]-2-methylpropanamide

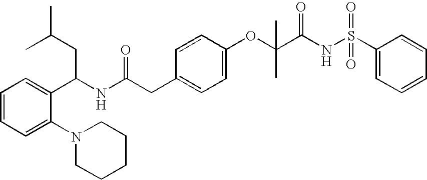

This compound was obtained by condensation between compound 1 and benzenesulfonamide according to protocol K. It was purified by silica gel chromatography (dichloromethane/methanol 99/1).

Yield: 22%

Aspect: white powder

¹H NMR (300 MHz, DMSO d6, δ in ppm): 0.88 (d, 6.1Hz, 6H); from 1.23 to 1.66 (m, 15H); 2.50 (m, 2H); 3.07 (m, 2H); 3.34 (m, 2H); 5.34 (m, 1H); 6.54 (d, 8.5 Hz, 2H); from 7.00 to 7.18 (m, 5H); 7.26 (d, 7.3 Hz, 1H); from 7.58 to 7.72 (m, 3H); 7.85 (d, 7.3 Hz, 2H); 8.35 (d, 8.5 Hz, 1H); 12.41 (s(broad), 1H).

Compound 65:

### 2-[4-[1-(1-(2-(1-piperidinyl)phenyl)butyl)aminocarbonylmethyl]phenoxy]-2-methylpropanoic acid

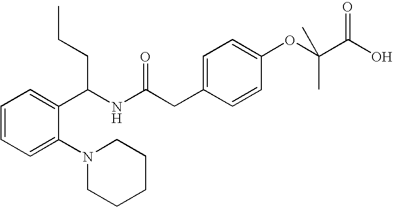

This compound was obtained by transformation of phenol derivative 4-8 according to protocol M. It was purified by silica gel chromatography (dichloromethane/methanol 9/1) and recrystallized in toluene.

Yield: 37%

Aspect: white powder

¹H NMR (300 MHz, CDCl₃, δ in ppm): 0.87 (t, 7.29 Hz, 3H); from 1.05 to 1.80 (m, 16H); 2.63 (m, 2H); 2.88 (m, 2H); 3.47 (s, 2H); 5.23 (m, 1H); 6.88 (d, 8.5 Hz, 2H); from 7.06 to 7.21 (m, 7H).

Compound 66:

### N-[methylsulfonyl]-2-[4-[1-(1-(2-(1-piperidinyl)phenyl)butyl)aminocarbonylmethyl]phenoxy]-2-methylpropanamide

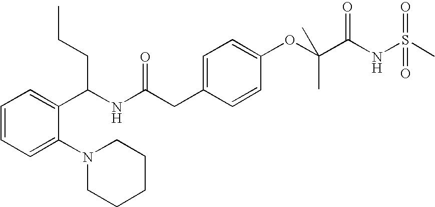

This compound was obtained by condensation between compound 65 and methanesulfonamide according to protocol L. It was purified by silica gel chromatography (dichloromethane/ethyl acetate 7/3).

Yield: 13%

Aspect: white powder

¹H NMR (300 MHz, CDCl₃, δ in ppm): 0.90 (t, 7.3 Hz, 3H); from 1.10 to 1.80 (m, 16H); 2.68 (m, 2H); 2.94 (m, 2H); 3.35 (s, 3H); 3.52 (s, 2H); 5.24 (m, 1H); 6.89 (d, 8.5 Hz, 2H); 7.10 (m, 3H); 7.23 (m, 4H).

Compound 67:

### N-[methylsulfonyl]-2-[4-[1-(1-(2-(methoxy)phenyl)pentyl)aminocarbonylmethyl]phenoxy]-2-methylpropanamide

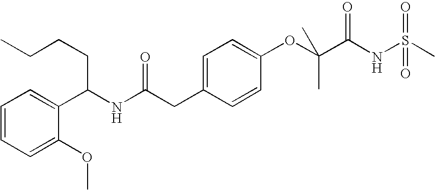

This compound was obtained by condensation between compound 54 and methanesulfonamide according to protocol L. It was purified by silica gel chromatography (dichloromethane/methanol 95/5).

Yield: 18%

Aspect: white powder

¹H NMR (300 MHz, CDCl₃, δ in ppm): 0.83 (t, 7.3 Hz, 3H); from 1.07 to 1.31 (m, 4H); 1.55 (s, 6H); 1.70 (m, 2H); 3.36 (s, 3H); 3.53 (m, 2H); 3.69 (s, 3H); 5.05 (m, 1H); 6.54 (m, 1H); from 6.84 to 6.94 (m, 4H); 7.11 (dd, 7.3 Hz, 1.4 Hz, 1H); 7.23 (m, 3H); 9.09 (s(broad), 1H).

Compound 68:

### 2-[4-[1-(1-(2-(isobutyloxy)phenyl)pentyl)aminocarbonylmethyl]phenoxy]-2-methylpropanoic acid

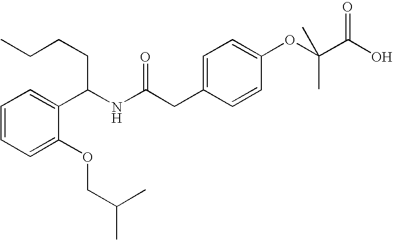

This product was synthesized in 2 steps from the phenol intermediate ethyl 2-[4-[1-(1-(2-(hydroxy)phenyl)pentyl)aminocarbonylmethyl]phenoxy]-2-methylpropanoate (intermediate for the synthesis of compound 44)

### Step 1: Ethyl 2-[4-[1-(1-(2-(isobutyloxy)phenyl)pentyl)aminocarbonylmethyl]phenoxy]-2-methylpropanoate

Ethyl 2-[4-[1-(1-(2-(hydroxy)phenyl)pentyl)aminocarbonylmethyl]phenoxy]-2-methylpropanoate (300 mg, 0.70 mmol) was solubilized in acetonitrile (5 ml). Potassium carbonate (290 mg, 2.1 mmol) and isobutyl bromide (156 μl, 1.4 mmol) were successively added and the mixture was stirred for 5 days at room temperature. The crude was filtered, evaporated, and purified by silica gel chromatography (dichloromethane/ethyl acetate 9/1).

Yield: 71%

Aspect: white solid

¹H NMR (300 MHz, CDCl₃, δ in ppm): 0.83 (t, 6.7 Hz, 3H); 0.99 (d, 6.7 Hz, 3H); 1.01 (d, 6.7 Hz, 3H); from 1.05 to 1.29 (m, 7H); from 1.58 to 1.94 (m, 9H); 3.49 (m, 2H); 3.67 (m, 2H); 4.24 (q, 7.3 Hz, 2H); 5.10 (m, 1H); 6.44 (d, 9.4 Hz, 1H); from 6.80 to 6.90 (m, 4H); from 7.05 to 7.25 (m, 4H).

### Step 2: 2-[4-[1-(1-(2-(isobutyloxy)phenyl)pentyl)aminocarbonylmethyl]phenoxy]-2-methylpropanoic acid.

This compound was obtained by saponification of the previously described intermediate according to protocol H. The expected product was purified by silica gel chromatography (dichloromethane/methanol 9/1).

Yield: 66%

Aspect: white powder

¹H NMR (300 MHz, DMSO d6, δ in ppm): 0.81 (t, 6.5 Hz, 3H); 0.99 (d, 6.7 Hz, 6H); from 1.23 to 1.64 (m, 12H); 2.01 (m, 1H); 3.38 (m, 2H); 3.72 (m, 2H); 5.14 (m, 1H); 6.74 (d, 8.5 Hz, 2H); from 6.83 to 6.90 (m, 2H); from 7.12 to 7.21 (m, 4H); 8.29 (d, 8.8 Hz, 1H); 12.96 (s(broad), 1H).

Compound 69:

### 2-[4-[1-(1-(2-(1-piperidinyl)phenyl)pentyl)aminocarbonylmethyl]phenoxy]-2-(ethoxycarbonyl)acetic acid

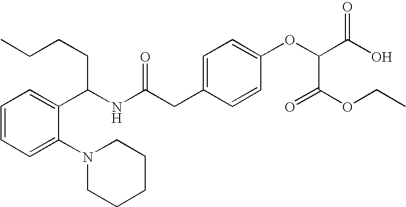

This compound was obtained in 3 steps from the phenol derivative 4-3.

### Step 1: Benzyl and ethyl bromomalonate

Benzyl and ethyl malonate (5.0 g, 22.5 mmol) and p-toluenesulfonic acid (6.4 g, 33.8 mmol) were solubilized in acetonitrile (50 ml) and N-bromosuccinimide (4.0 g, 22.5 mmol) was added portionwise. The mixture was refluxed for 50 minutes. It was then cooled down to room temperature and evaporated. The residue was taken with dichloromethane (75 ml) and washed with water (2×40 ml). The aqueous layer was re-extracted with dichloromethane (1×40 ml). Organic layers were combined and dried with magnesium sulfate (MgSO4), filtered and concentrated. The expected product was purified by silica gel chromatography (cyclohexane/ethyl acetate 95/5).

Yield: 44%

Aspect: colorless oil

¹H NMR (300 MHz, CDCl₃, δ in ppm): 1.26 (t, 7.0 Hz, 3H); 4.26 (q, 7.0 Hz, 2H); 4.89 (s, 1H); 5.28 (s, 2H); 7.39 (m, 5H).

### Step 2: Benzyl and ethyl 4-[1-(1-(2-(1-piperidinyl)phenyl)pentyl)aminocarbonylmethyl]phenoxy malonate

The phenol derivative 4-3 (1.06 g, 2.79 mmol) was solubilized in acetonitrile (35 ml) and cesium carbonate (1.82 g, 5.58 mol) was added. The mixture was stirred at room temperature for 15 min and benzyl ethyl bromomalonate (1.68 g, 5.58 mmol) was added. The mixture was stirred overnight at room temperature. Salts were filtered, washed with ethyl acetate, and the filtrate was concentrated. The expected product was purified by silica gel chromatography (cyclohexane/ethyl acetate 8/2).

Yield: 44%

Aspect: white powder

¹H NMR (300 MHz, CDCl₃, δ in ppm): 0.84 (t, 7.3 Hz, 3H); from 1.10 to 1.31 (m, 7H); from 1.50 to 1.80 (m, 8H); 2.65 (m, 2H); 2.92 (m, 2H); 3.49 (s, 2H); 4.27 (q, 7.3 Hz, 2H); from 5.22 to 5.29 (m, 4H); from 6.84 to 6.93 (m, 3H); 7.07 (m, 2H); from 7.17 to 7.23 (m, 4H); 7.36 (m, 5H).

### Step 3: 2-[4-[1-(1-(2-(1-piperidinyl)phenyl)pentyl)aminocarbonylmethyl]phenoxy]-2-(ethoxycarbonyl) acetic acid

This compound was obtained by hydrogenolysis of the previously described intermediate according to protocol E. The expected product was purified by silica gel chromatography (dichloromethane/methanol/acetic acid 9/1/0.1).

Yield: 80%

Aspect: white powder

¹H NMR (300 MHz, DMSO d6, δ in ppm): 0.83 (t, 6.7 Hz, 3H); from 1.13 to 1.27 (m, 7H); from 1.51 to 1.64 (m, 8H); 2.50 (m, 2H); 3.06 (m, 2H); 3.36 (m, 2H); from 3.99 to 4.13 (m, 2H); 4.75 (s, 1H); 5.20 (m, 1H); 6.74 (d, 8.5 Hz, 2H); from 7.00 to 7.17 (m, 5H); 7.24 (d, 7.6 Hz, 1H); 8.35 (d, 8.5 Hz, 1H).

Compound 70:

### 2-(4-((1H-tetrazol-5-yl)methoxy)phenyl)-N-[1-(1-(2-(1-piperidinyl)phenyl)pentyl)]acetamide

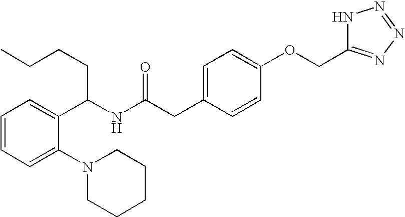

This compound was obtained in 2 steps from the phenol derivative 4-3.

### Step 1: 2-(4-((cyano)methoxy)phenyl)-N-[1-(1-(2-(1-piperidinyl)phenyl)pentyl)]acetamide

This intermediate was obtained upon substitution of 2-chloroacetonitrile by phenol 4-3 according to protocol D. It was purified by silica gel chromatography (cyclohexane/ethyl acetate 7/3).

Yield: 33%

Aspect: beige solid

¹H NMR (300 MHz, CDCl₃, δ in ppm): 0.85 (t, 7.3 Hz, 3H); from 1.13 to 1.35 (m, 4H); from 1.59 to 1.70 (m, 8H); 2.67 (m, 2H); 2.94 (m, 2H); 3.51 (s, 2H); 4.74 (s, 2H); 5.23 (m, 1H); 6.94 (d, 8.5 Hz, 2H); from 7.03 to 7.12 (m, 3H); from 7.22 to 7.24 (m, 4H).

### Step 2: 2-(4-((1H-tetrazol-5-yl)methoxy)phenyl)-N-[1-(1-(2-(1-piperidinyl)phenyl)pentyl)]acetamide

In a schlenk tube under anhydrous atmosphere, the previously described nitrile (240 mg, 0.57 mmol) was solubilized in toluene (5 ml). Trimethylsilyl azide (132 mg, 1.14 mmol) and bis(tributyltin) oxide (341 mg, 0.57 mmol) were added. The mixture was heated at 110° C. overnight. The crude was cooled and evaporated. The resulting residue was directly purified by silica gel chromatography (dichloromethane/methanol 9/1-7/3).

Yield: 90%

Aspect: white solid

¹H NMR (300 MHz, DMSO d6, δ in ppm): 0.83 (t, 7.0 Hz, 3H); from 1.10 to 1.28 (m, 4H); from 1.53 to 1.64 (m, 8H); 2.50 (m, 2H); 3.06 (m, 2H); 3.36 (m, 2H); 5.16 (s, 2H); 5.21 (m, 1H); 6.95 (d, 8.5 Hz, 2H); from 7.00 to 7.16 (m, 5H); 7.26 (d, 7.6 Hz, 1H); 8.33 (d, 8.2 Hz, 1H).

Compound 71:

### 2-[4-[1-(1-(2-(1-piperidinyl)phenyl)pentyl)aminocarbonylmethyl]phenoxy]acetic acid

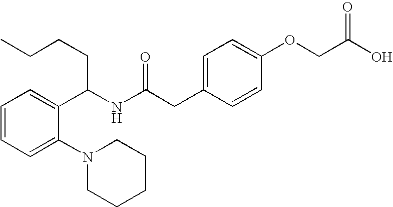

This product was synthesized in 2 steps:

### Step 1: Ethyl 2-[4-[1-(1-(2-(1-piperidinyl)phenyl)pentyl)aminocarbonylmethyl]phenoxy]acetate

This intermediate was obtained upon substitution of ethyl bromoacetate by phenol 4-3 according to protocol D. It was purified by silica gel chromatography (cyclohexane/ethyl acetate 8/2-6/4).

Yield: 80%

Aspect: white solid

¹H NMR (300 MHz, CDCl₃, δ in ppm): 0.84 (t, 7.0 Hz, 3H); from 1.05 to 1.34 (m, 7H); from 1.58 to 1.71 (m, 8H); 2.65 (m, 2H); 2.93 (m, 2H); 3.51 (s, 2H); 4.28 (q, 7.3 Hz, 2H); 4.62 (s, 2H); 5.22 (m, 1H); 6.88 (m, 3H); 7.08 (m, 2H); from 7.19 to 7.25 (m, 4H).

### Step 2: 2-[4-[1-(1-(2-(1-piperidinyl)phenyl)pentyl)aminocarbonylmethyl]phenoxy]acetic acid

This compound was obtained by saponification of the previously described intermediate according to protocol H. The expected product was purified by silica gel chromatography (dichloromethane/methanol 9/1).

Yield: 63%

Aspect: white powder

¹H NMR (300 MHz, DMSO d6, δ in ppm): 0.83 (t, 6.7 Hz, 3H); from 1.11 to 1.64 (m, 12H); 2.51 (m, 2H); 3.08 (m, 2H); 3.36 (m, 2H); 4.55 (s, 2H); 5.21 (m, 1H); 6.80 (d, 8.5 Hz, 2H); from 7.00 to 7.17 (m, 5H); 7.26 (d, 7.6 Hz, 1H); 8.34 (d, 8.5 Hz, 1H).

Compound 72:

### N-[methylsulfonyl]-2-[4-[1-(1-(2-(1-piperidinyl)phenyl)pentyl)aminocarbonylmethyl]phenoxy]acetamide

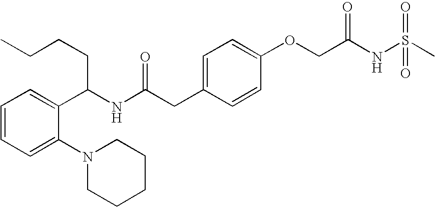

This compound was obtained by condensation between compound 71 and methanesulfonamide according to protocol L. It was purified by silica gel chromatography (dichloromethane/methanol 95/5-7/1).

Yield: 47%

Aspect: white powder

¹H NMR (300 MHz, DMSO d6, δ in ppm): 0.83 (t, 6.7 Hz, 3H); from 1.10 to 1.75 (m, 12H); 2.51 (m, 2H); from 3.00 to 3.10 (m, 5H); 3.36 (m, 2H); 4.51 (s, 2H); 5.21 (m, 1H); 6.79 (d, 8.8 Hz, 2H); from 7.00 to 7.15 (m, 5H); 7.26 (d, 7.6 Hz, 1H); 8.33 (d, 8.5 Hz, 1H).

Compound 73:

### 2-[2-methoxy-4-[1-(1-(2-(1-piperidinyl)phenyl)pentyl)aminocarbonylmethyl]phenoxy]-2-methylpropanoic acid

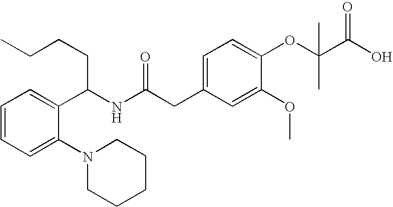

This product was synthesized in 2 steps:

### Step 1: Ethyl 2-[2-methoxy-4-[1-(1-(2-(1-piperidinyl)phenyl)pentyl)aminocarbonylmethyl]phenoxy]-2-methylpropanoate

This intermediate compound was obtained by condensation of amine 2-9 and acid 3-2 according to protocol F. It was purified by silica gel chromatography (dichloromethane/ethyl acetate 95/5).

Yield: 63%

Aspect: white powder

¹H NMR (300 MHz, CDCl₃, δ in ppm): 0.84 (t, 6.7 Hz, 3H); 1.28 (m, 7H); from 1.66 to 1.72 (m, 14H); 2.62 (m, 2H); 2.92 (m, 2H); 3.50 (s, 2H); 3.74 (s, 3H); 4.25 (q, 7.0 Hz, 2H); 5.24 (m, 1H); 6.70 (dd, 8.3 Hz, 1.8 Hz, 1H); from 6.78 to 6.85 (m, 3H); 7.04 (m, 2H); 7.20 (m, 2H).

### Step 2: 2-[2-methoxy-4-[1-(1-(2-(1-piperidinyl)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 95/5).

Yield: 59%

Aspect: white powder

¹H NMR (300 MHz, DMSO d6, δ in ppm): 0.82 (t, 7.3 Hz, 3H); from 1.15 to 1.70 (m, 18H); 2.51 (m, 2H); 3.06 (m, 2H); 3.34 (m, 2H); 3.69 (s, 3H); 5.21 (m, 1H); 6.71 (m, 2H); 6.89 (s, 1H); from 7.00 to 7.18 (m, 3H); 7.27 (d, 6.7 Hz, 1H); 8.35 (d, 8.5 Hz, 1H).

Compound 74:

### 2-[4-[1-(2-phenyl-1-(2-(1-piperidinyl)phenyl)ethyl)aminocarbonylmethyl]phenylthio]-2-methylpropanoic acid

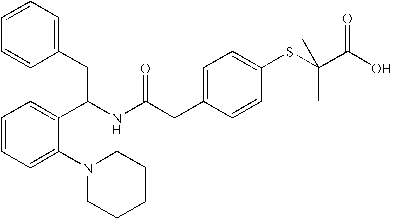

This product was synthesized in 2 steps from the intermediate 4-9:

### Step 1: Ethyl 2-[4-[1-(2-phenyl-1-(2-(1-piperidinyl)phenyl)ethyl)aminocarbonylmethyl]phenylthio]-2-methylpropanoate

Thiophenol 4-9 (200 mg, 0.46 mmol) was solubilized in N,N-dimethylformamide (8 ml) and the mixture was cooled to 0° C. with an ice bath. Sodium hydride (60% in oil, 22.3 mg, 0.56 mmol) was added and the mixture was stirred 20 minutes at room temperature. The mixture was cooled again to 0° C. and ethyl bromoisobutyrate (102 μl, 0.70 mmol) was added. The mixture was stirred overnight at room temperature. The same protocol was repeated twice (successive additions of NaH and ethyl bromoisobutyrate). After 48 hours at room temperature, the crude was hydrolyzed by water (15 ml) and extracted with ethyl acetate (2×10 ml). The organic layer was washed with NaClsat (3×8 ml), dried with magnesium sulfate (MgSO₄), filtered and concentrated. The resulting residue was purified by silica gel chromatography (dichloromethanelethyl acetate 98/2-95/5) to afford the expected product.

Yield: 25%

Aspect: white solid

¹H NMR (300 MHz, CDCl₃, δ in ppm): 1.23 (t, 7.0 Hz, 3H); from 1.45 to 1.72 (m, 12H); 2.62 (m, 2H); from 2.85 to 3.08 (m, 4H); 3.49 (s, 2H); 4.13 (q, 7.0 Hz, 2H); 5.58 (m, 1H); from 6.76 to 7.43 (m, 14H).

### Step 2: 2-[4-[1-(2-phenyl-1-(2-(1-piperidinyl)phenyl)ethyl)aminocarbonylmethyl]phenylthio]-2-methylpropanoic acid

This compound was obtained by saponification of the previously described intermediate according to protocol H. The expected product was purified by silica gel chromatography (dichloromethane/methanol 9/1).

Yield: 13%

Aspect: white powder

¹H NMR (300 MHz, CDCl₃, δ in ppm): from 1.51 to 1.73 (m, 12H); 2.60 (m, 2H); from 2.88 to 3.06 (m, 4H); 3.46 (s, 2H); 5.56 (m, 1H); from 6.94 to 7.25 (m, 12H); 7.44 (m, 2H).

Compound 75:

### 2-[4-[1-(1-(2-(diethylamino)phenyl)pentyl)aminocarbonylmethyl]phenoxy]-2-methylpropanoic acid

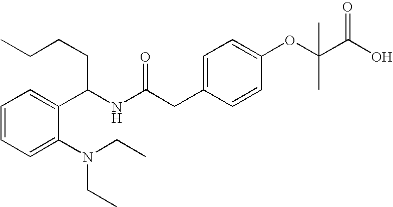

This compound was obtained by transformation of phenol derivative 4-10 according to protocol M. It was purified by silica gel chromatography (dichloromethane/methanol 9/1).

Yield: 75%

Aspect: white powder

¹H NMR (300 MHz, DMSO d6, δ in ppm): 0.81 (t, 6.7 Hz, 3H); 0.89 (t, 7.0 Hz, 6H); 1.24 (m, 4H); 1.45 (s, 6H); 1.53 (m, 2H); 2.92 (m, 4H); 3.35 (m, 2H); 5.30 (m, 1H); 6.73 (d, 8.5 Hz, 2H); from 7.02 to 7.19 (m, 5H); 7.31 (dd, 7.6 Hz, 1.4 Hz, 1H); 8.27 (d, 8.5 Hz,1H).

Compound 76:

### N-[methylsulfonyl]-2-[4-[1-(1-(2-(diethylamino)phenyl)pentyl)aminocarbonylmethyl]phenoxy]-2-methylpropanamide

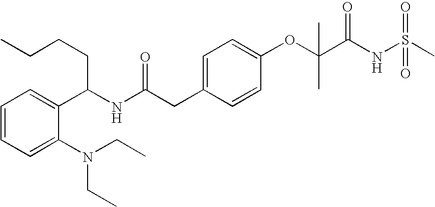

This compound was obtained by condensation between compound 75 and methanesulfonamide according to protocol K (replacing DCC by EDC). It was purified by silica gel chromatography (dichloromethane/methanol 98/2).

Yield: 28%

Aspect: white powder

¹H NMR (300 MHz, DMSO d6, δ in ppm): 0.81 (t, 6.7 Hz, 3H); 0.89 (t, 7.0 Hz, 6H); 1.24 (m, 4H); 1.44 (s, 6H); 1.53 (m, 2H); 2.92 (m, 4H); 3.23 (s, 3H); 3.37 (m, 2H); 5.31 (m, 1H); 6.79 (d, 8.5 Hz, 2H); from 7.03 to 7.19 (m, 5H); 7.31 (d, 7.6 Hz, 1H); 8.27 (d, 8.5 Hz, 1H); 11.97 (s(broad), 1H).

Compound 77:

### N-[methylsulfonyl]-2-[2-methoxy-4-[1-(1-(2-(1-piperidinyl)phenyl)pentyl)aminocarbonylmethyl]phenoxy]-2-methylpropanamide

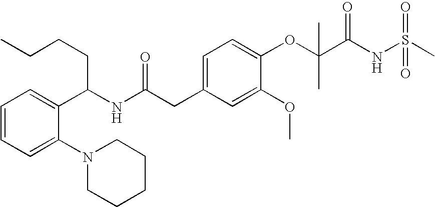

This compound was obtained by condensation between compound 73 and methanesulfonamide according to protocol K (replacing DCC by EDC). It was purified by silica gel chromatography (dichloromethane/methanol 98/2).

Yield: 43%

Aspect: white powder

¹H NMR (300 MHz, DMSO d6, δ in ppm): 0.83 (t, 7.0 Hz, 3H); 1.27 (m, 4H); 1.37 (s, 6H); 1.55 (m, 8H); 2.50 (m, 2H); 3.05 (m, 2H); 3.31 (s, 3H); 3.40 (s, 2H); 3.75 (s, 3H); 5.22 (m, 1H); 6.75 (dd, 8.2 Hz and 1.8 Hz, 1H); 6.88 (d, 8.2 Hz, 1H); from 6.96 to 7.18 (m, 4H); 7.27 (dd, 7.6 Hz and 1.5 Hz, 1H); 8.37 (d, 8.5 Hz, 1H); 11.49 (s(broad), 1H).

Compound 78:

### 2-[4-[1-(1-(2-(acetamido)phenyl)pentyl)aminocarbonylmethyl]phenoxy]-2-methylpropanoic acid

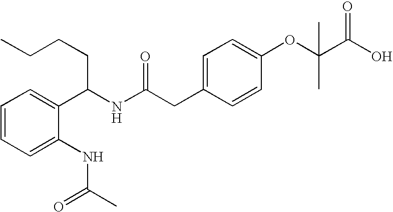

This product was synthesized in 4 steps:

### Step 1: Ethyl 2-[4-[1-(1-(2-(benzylamino)phenyl)pentyl)aminocarbonylmethyl]phenoxy]-2-methylpropanoate

This intermediate compound was obtained by condensation of amine 2-35 and acid 3-1 according to protocol G. It was purified by silica gel chromatography.

Yield: 90%

Aspect: white powder

¹H NMR (300 MHz, CDCl₃, δ in ppm): 0.89 (t, 7.0 Hz, 3H); from 1.23 to 1.42 (m, 7H); 1.61 to 1.95 (m, 8H); 3.48 (m, 2H); 4.25 (q, 7.0 Hz, 2H); 4.40 (m, 2H); 5.14 (m, 1H); 5.44 (d, 9.6 Hz, 1H); 5.65 (s(broad), 1H); from 6.56 to 6.67 (m, 2H); 6.82 (d, 8.5 Hz, 2H); 7.08 (m, 4H); from 7.22 to 7.35 (m, 5H).

### Step 2: Ethyl 2-[4-[1-(1-(2-(amino)phenyl)pentyl)aminocarbonylmethyl]phenoxy]-2-methylpropanoate

This compound was obtained by catalytic hydrogenation of the previously described intermediate according to protocol E. No further purification was performed.

Yield: quantitative

Aspect: pale rose solid

¹H NMR (300 MHz, CDCl₃, δ in ppm): 0.86 (t, 7.0 Hz, 3H); from 1.23 to 1.42 (m, 7H); 1.61 (s, 6H); from 1.70 to 1.85 (m, 2H); 3.47 (m, 2H); 4.24 (m, 4H); 5.05 (m, 1H); 5.62 (d, 9.4 Hz, 1H); from 6.64 to 6.71 (m, 2H); 6.81 (d, 8.5 Hz, 2H); from 7.00 to 7.12 (m, 4H).

### Step 3: Ethyl 2-[4-[1-(1-(2-( acetamido)phenyl)pentyl)aminocarbonylmethyl]phenoxy]-2-methylpropanoate

The previously described intermediate (800 mg, 1.88 mmol) was solubilized in dichloromethane (10 ml). Triethylamine (1.3 ml, 9.38 mmol) was added and the mixture was cooled to 0° C. Acetic anhydride (265 μl, 2.81 mmol) was added dropwise and the mixture was cooled to room temperature. After one night, solvant was evaporated and the residue treated by ethyl acetate (40 ml). The organic layer was washed with 1M HCl (3×10 ml) and NaClsat (2×10 ml). It was dried with magnesium sulfate, filtered and concentrated to afford the expected product.

Yield: 97%

Aspect: colorless oil

¹H NMR (300 MHz, CDCl₃, δ in ppm): 0.86 (t, 7.0 Hz, 3H); from 1.15 to 1.30 (m, 7H); 1.62 to 1.80 (m, 8H); 2.26 (s, 3H); 3.49 (m, 2H); 4.26 (q, 7.0 Hz, 2H); 5.00 (m, 1H); 5.58 (d, 7.9 Hz, 1H); 6.85 (d, 8.5 Hz, 2H); from 7.06 to 7.15 (m, 4H); 7.30 (m, 1H); 7.95 (d, 7.9 Hz, 1H); 9.81 (s,1H).

### Step 4: 2-[4-[1-(1-(2-(acetamido)phenyl)pentyl)aminocarbonylmethyl]phenoxy]-2-methylpropanoic acid

This compound was obtained by saponification of the previously described intermediate according to protocol H. The expected product was purified by silica gel chromatography (dichloromethane/methanol 9/1).

Yield: 39%

Aspect: white powder

¹H NMR (300 MHz, DMSO d6, δ in ppm): 0.82 (t, 6.7 Hz, 3H); 1.24 (m, 4H); 1.46 (s, 6H); 1.71 (m, 2H); 2.01 (s, 3H); 3.35 (m, 2H); 4.93 (m, 1H); 6.73 (d, 8.2 Hz, 2H); from 7.09 to 7.22 (m, 4H); 7.32 (dd, 7.3 Hz and 1.5 Hz, 1H); 7.52 (d, 7.6 Hz, 1H); 8.56 (d, 8.2 Hz, 1H); 9.69 (s, 1H); 13.19 (s(broad), 1H).

Compound 79:

### 2-[4-[1-(1-(2-(1-piperidinyl)phenyl)pentyl)carbonylaminomethyl]phenoxy]-2-methylpropanoic acid

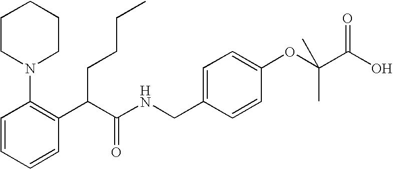

This compound was prepared in 2 steps:

### Step 1: ethyl 2-[4-[1-(1-(2-(1-piperidinyl)phenyl)pentyl)carbonylaminomethyl]phenoxy]-2-methylpropanoate

This intermediate was obtained by condensation of amine 2-21 and acid 3-6 according to protocol F. It was isolated by silica gel chromatography (cyclohexane/ethyl acetate 85/15).

Yield: 78%

Aspect: oil

¹H NMR (300 MHz, CDCl₃, δ in ppm): 0.91 (t, 7.3 Hz, 3H); from 1.24 to 1.59 (m, 19H); 1.80 (m, 1H); 2.29 (m, 1H); 2.44 (m, 2H); 2.88 (m, 2H); 4.00 (t, 7.6 Hz, 1H); 4.12 (dd, 14.6 Hz, 6.7 Hz, 1H); 4.23 (q, 7.0 Hz, 2H); 4.38 (dd, 14.6 Hz, 6.7 Hz, 1H); 6.68 (d, 8.5 Hz, 2H); 6.85 (d, 8.5 Hz, 2H); from 7.05 to 7.22 (m, 4H); 7.38 (dd, 7.6 Hz, 1.7 Hz, 1H).

### Step 2: 2-[4-[1-(1-(2-(1-piperidinyl)phenyl)pentyl)carbonylaminomethyl]phenoxy]-2-methylpropanoic acid

This compound was obtained by saponification of the previously described intermediate according to protocol H. The expected product was purified by silica gel chromatography (dichloromethane/methanol 9/1).

Yield: 80%

Aspect: white solid

¹H NMR (300 MHz, DMSO d6, δ in ppm): 0.83 (t, 7.3 Hz, 3H); from 1.13 to 1.62 (m, 17H); 1.94 (m, 1H); 2.68 (m, 4H); 4.03 (t, 7.6 Hz, 1H); 4.16 (d, 5.9 Hz, 2H); 6.71 (d, 8.8 Hz, 2H); 7.03 (m, 3H); from 7.10 to 7.20 (m, 2H); 7.36 (dd, 7.6 Hz, 1.5 Hz, 1H); 8.09 (t, 5.9 Hz, 1H).

Compound 80:

### N-[methylsulfonyl]-2-[4-[1-(1-(2-(1-piperidinyl)phenyl)pentyl)carbonylaminomethyl]phenoxy]-2-methylpropanamide

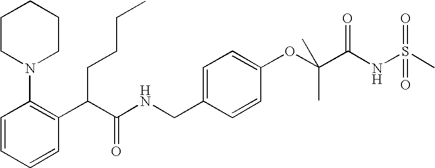

Compound 79 (414 mg, 0.89 mmol) was solubilized in dichloromethane (10 ml), cooled to 0° C., and thionyl chloride (71 μl, 0.98 mmol) was added. The mixture was warmed to room temperature and stirred for 1 hour. Triethylamine (371 μl, 2.66 mmol), DMAP (22 mg, 0.18 mmol), and methanesulfonamide (93 mg, 0.98 mmol) were successively added and the mixture was stirred at room temperature overnight. The crude was diluted with dichloromethane (30 ml) and washed with aqueous citric acid (200 g/l, 3×10 ml) and NaClsat (1×10 ml). The resulting organic layer was dried with magnesium sulfate (MgSO₄) and concentrated. The expected compound was isolated by silica gel chromatography (dichloromethane/methanol 99/1).

Yield: 18%

Aspect: white powder

¹H NMR (300 MHz, DMSO d6, δ in ppm): 0.83 (t, 7.3 Hz, 3H); from 1.10 to 1.35 (m, 4H); from 1.44 to 1.70 (m, 13H); 1.94 (m, 1H); 2.71 (m, 4H); 3.24 (s, 3H); 4.03 (t, 7.5 Hz, 1H); 4.18 (m, 2H); 6.78 (d, 8.5 Hz, 2H); from 7.02 to 7.19 (m, 5H); 7.36 (dd, 7.6 Hz, 1.5 Hz, 1H); 8.12 (t, 5.9 Hz, 1H); 11.97 (s, 1H).

Compound 81:

### 1-[4-[1-(1-(2-(1-piperidinyl)phenyl)pentyl)aminocarbonylmethyl]phenoxy]-1-cyclobutane carboxylic acid

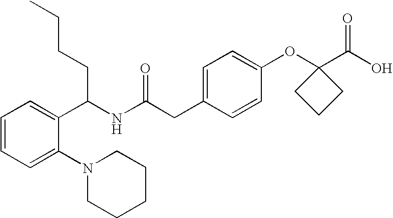

This product was prepared in 2 steps:

### Step 1: ethyl 1-[4-[1-(2-(1-piperidinyl)phenyl)pentyl)aminocarbonylmethyl]phenoxy]cyclobutanecarboxylate

This intermediate was obtained upon substitution of ethyl 1-bromocyclobutanecarboxylate by phenol 4-3 according to protocol D (using cesium carbonate instead of potassium carbonate). It was purified by silica gel chromatography (cyclohexane/ethyl acetate 8/2)

Yield: 59%

Aspect: white powder

¹H NMR (300 MHz, CDCl₃, δ in ppm): 0.84 (t, 7.1Hz, 3H); from 1.12 to 1.33 (m, 7H); from 1.51 to 1.70 (m, 8H); 2.01 (m, 2H); 2.45 (m, 2H); 2.64 (m, 2H); 2.75 (m, 2H); 2.92 (m, 2H); 3.48 (s, 2H); 4.20 (q, 7.0 Hz, 2H); 5.23 (m, 1H); 6.65 (d, 8.5 Hz, 2H); 6.79 (d, 9.0 Hz, 1H); from 7.05 to 7.26 (m, 6H).

### Step 2: 1-[4-[1-(1-(2-(1-piperidinyl)phenyl)pentyl)aminocarbonylmethyl]phenoxy]cyclobutanecarboxylic acid

This compound was obtained by saponification of the previously described intermediate according to protocol H. The expected product was purified by silicagel chromatography (dichloromethane/methanol 98/2).

Yield: 47%

Aspect: white powder

¹H NMR (300 MHz, DMSO d6, δ in ppm): 0.83 (t, 7.0 Hz, 3H); from 1.17 to 1.28 (m, 4H); from 1.51 to 1.64 (m, 8H); 1.89 (m, 2H); 2.28 (m, 2H); 2.50 (m, 2H); 2.63 (m, 2H); 3.06 (m, 2H); 3.34 (m, 2H); 5.20 (m, 1H); 6.55 (d, 8.8 Hz, 2H); from 7.01 to 7.18 (m, 5H); 7.25 (dd, 7.7 Hz et 1.5 Hz, 1H); 8.33 (d, 8.5 Hz, 1H); 13.06 (s(broad), 1H).

Compound 82:

### N-[methylsulfonyl]-1-[4-[1-(1-(2-(1-piperidinyl)phenyl)pentyl)aminocarbonylmethyl]phenoxy]cyclobutanecarboxamide

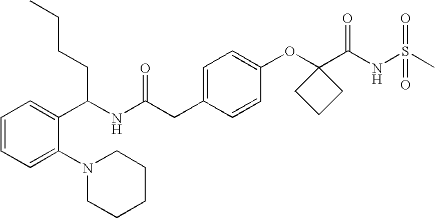

This compound was obtained by condensation between compound 81 and methanesulfonamide according to protocol L. It was purified by silica gel chromatography (dichloromethane/methanol 98/2).

Yield: 47%

Aspect: white powder

¹H NMR (300 MHz, DMSO d6, δ in ppm): 0.83 (t, 7.0 Hz, 3H); from 1.16 to 1.30 (m, 4H); from 1.51 to 1.91 (m, 10H); 2.26 (m, 2H); 2.50 (m, 2H); 2.66 (m, 2H); from 3.05 to 3.12 (m, 5H); 3.37 (m, 2H); 5.20 (m, 1H); 6.60 (d, 8.5 Hz, 2H); from 7.00 to 7.16 (m, 5H); 7.24 (d, 7.6 Hz,1H); 8.33 (d, 8.2 Hz, 1H); 11.95 (s, 1H).

### Exemple 6

### In vitro Evaluation of the PPAR Activating Properties of the Compounds According to the Invention

The PPAR activating properties of the inventive compounds were evaluated in vitro.

Principle

The PPAR activation was evaluated in vitro on a monkey kidney fibroblast line (COS-7) by measuring the transcriptional activity of chimeras constituted of the DNA binding domain of the Gal4 transcription factor of yeast and of the binding domain to the ligand of the different PPARs. The compounds were tested at doses between 0.01 and 100 μM on Gal4-PPARα, γ, δ chimeras. The induction factor (ratio between the luminescence induced by the compound and the luminescence induced by the control) was measured for each condition: the higher this factor, the more the compound has PPAR-activating properties.

Protocol

Cell Culture

The COS-7 cells from ATCC were cultivated in a DMEM medium supplemented with 10% (vol/vol) of foetal calf serum, 100 U/ml penicillin (Gibco, Paisley, UK) and 2 mM L-Glutamine (Gibco, Paisley, UK). The cells were incubated at 37° C. in a humid atmosphere containing 5% CO₂.

Description of the Plasmids Used in Transfection

The Gal4(RE)_TkpGL3, pGal4-hPPARα, pGal4-hPPARγ, pGal4-hPPARδ and pGal4-φ plasmids have been described in the literature (Raspe, Madsen et al. 1999). The pGal4-hPPARα, pGal4-hPPARγ and pGal4-hPPARδ constructs were obtained by cloning in the pGal4-φ vector of fragments of DNA amplified by PCR corresponding to the DEF domains of the human PPARα, PPARγ PPARδ nuclear receptors.

Transfection

COS-7 cells were plated in 96 wells plates (5×10⁴ cells/well) and transfected, in the presence of 10% foetal calf serum and 150 ng DNA per well, with a pGal4-PPAR/Gal4(RE)_TkpGL3 ratio of 1/10. The cells were then incubated for 24 hours with the compounds to be tested, without serum. At the end of the experiment, the cells were lysed and the luciferase activities were determined by means of Steady Glow Luciferase (Promega) according to the supplier's recommendations.

Results

Unexpectedly, the experimental data given below show that the inventive compounds bind to PPARs in vitro and induce activation of transcriptional activity.

### Example 6-1

### Transactivation of pGal4-hPPAR (α/γ/δ) at 10 μM

The inventive compounds were tested at 10 μM on the 3 PPAR isoforms. The results obtained are detailed in table (1). The transactivation measured is expressed as a response ratio versus an internal reference for each of the isoforms.

The PPAR activation was evaluated in vitro on a monkey kidney fibroblast line (COS-7), by measuring the transcriptional activity of chimeras made up of the DNA binding domain of the Gal4 transcription factor of yeast and of the binding domain to the ligand of the different PPARs.

The compounds were tested at 10 μM on the Gal4-PPARα, γ, δ chimeras. The induction factor, i.e. the ratio between the luminescence induced by the compound and the luminescence induced by the control, was measured for each condition. It was then standardised in comparison to an internal reference and the results were then expressed in percentages : the higher the activation percentage, the more the compound has PPAR-activating properties.

TABLE 1
In vitro evaluation of the PPAR activating properties
of the inventive compounds at 10 μM

| | % hGal4-PPAR transactivation | | |
|---|---|---|---|
| | (10 μM) | | |
| Compound | alpha | gamma | delta |
| 2 | NS | 27 | NS |
| 2A | NS | 59 | NS |
| 2B | NS | 19 | NS |
| 5 | 31 | 82 | NS |
| 7 | 40 | 34 | NS |
| 10 | 11 | 49 | NS |
| 11 | NS | 47 | NS |
| 15 | NS | 42 | NS |
| 19 | NS | 95 | NS |
| 21 | NS | 25 | 24 |
| 28 | 35 | 17 | NS |
| 32 | NS | 25 | NS |
| 33 | NS | 42 | 23 |
| 34 | NS | 30 | NS |
| 36 | 54 | 73 | NS |
| 38 | 13 | NS | 18 |
| 39 | 16 | 29 | 24 |
| 40 | 20 | 55 | NS |
| 42 | 32 | NS | NS |
| 43 | 27 | 18 | NS |
| 45 | 31 | 62 | NS |
| 46 | 58 | 12 | NS |
| 47 | 42 | NS | NS |
| 48 | NS | 17 | NS |
| 49 | NS | 87 | 57 |
| 50 | NS | NS | 11 |
| 52 | 42 | NS | NS |
| 53 | NS | 40 | NS |
| 54 | NS | 30 | NS |
| 55 | NS | 33 | NS |
| 57 | 29 | NS | NS |
| 59 | 22 | NS | NS |
| 60 | 46 | NS | NS |
| 61 | 20 | NS | NS |
| 63 | 26 | NS | NS |
| 64 | NS | 18 | NS |
| 65 | NS | 50 | NS |
| 66 | 40 | NS | NS |
| 67 | NS | NS | NS |
| 68 | NS | 37 | NS |
| 70 | NS | 13 | NS |
| 73 | NS | 42 | NS |

NS: Activation weaker than 10% vs internal standard

Analysis of this table shows that the inventive compounds are agonists of the PPAR nuclear receptors: the inventive compounds bind to and activate hPPARα, hPPARγ, and/or hPPARδ significantly. The transactivation levels obtained with inventive compounds are variable depending on the PPAR sub-type studied and differ from one compound to another.

One also observes, surprisingly, among the inventive compounds more or less selectivity with respect to PPAR isoforms at 10 μM:
Some inventive compounds are selective for a PPAR sub-type: this is the case, for example, with compound 19 on hPPARγ; the latter activates neither hPPARα nor hPPARδ. In the same way, compound 60 is hPPARα selective.
Some inventive compounds are simultaneously activators of two PPAR sub-types: for example, compound 36 activates hPPARα and hPPARγ, but not hPPARδ. In the same way, compound 49 activates hPPARγ and hPPARδ, but not hPPARα.
Some inventive compounds simultaneously activate all PPAR sub-types (pan agonist): for example, compound 39 is a ligand of hPPARα, hPPARγ and hPPARδ.

It should be noted that compounds 2A and 2B were evaluated at 10 μM and compared to compound 2. The results obtained show that PPAR activation is enantiomer-dependent: compounds 2A and 2B respectively induce a relative transactivation of 59 and 19%.

### Exemple 6-2

### Dose-Dependent Transactivation of pGal4-hPPAR

The inventive compounds were tested at doses between 0.01 and 100 μM on the 3 PPAR isoforms. The results obtained are detailed in FIGURES (1a), (1b) and (1c): evolution of the transactivation levels according to the dose for the three PPAR sub-types (α, γ, and δ respectively).

The inventors show a significant and dose-dependent increase of luciferase activity in the cells transfected with pGal4-hPPAR plasmids and treated with the compounds according to the invention.

Conclusion:

These results show that the inventive compounds significantly bind to and activate the hPPARα, hPPARγ, and/or hPPARδ receptors. The levels of transactivation obtained with the inventive compounds are variable depending on the structure of the compound tested and the PPAR sub-type.

### Exemple 7

### In Vitro Evaluation of the Affinity of the Inventive Compounds for the ATP-Dependent Potassium Channels

The subject matter of this study was to evaluate in vitro the interaction of the inventive compounds with the ATP-dependent potassium channels (K⁺_{ATP}), a known target of currently marketed insulinotropic medicines.

Principle

The results shown reflect the specific affinity of the inventive compounds on the ATP-dependent (K⁺_{ATP}) potassium channels. The specific binding measured corresponds to the difference between the total binding and the non-specific binding determined in the presence of an excess of non-marked reference ligand (glibenclamide 1 μM). The inventive compounds were tested at doses between 3 and 300 nM: the higher the percentage measured, the stronger the affinity of the compound for the ATP-dependent potassium channels.

Protocol

The binding test was carried out at CEREP (Celle L'Evescault, France (86)) according to a protocol inspired by Angel et al. (Angel and Bidet 1991): the potassium channels derive from a rat's cerebral cortex and the reference compound is [³H]glibenclamide at 0.1 nM. The protocol consists of incubation at 22° C. for one hour.

Results

Unexpectedly, the inventors measured a specific affinity between the inventive compounds and the ATP-dependants (K⁺_{ATP}) potassium channels.

The results presented on table (2) show the affinity of the inventive compounds at 300 nM: the radiomarked ligand was specifically displaced by the inventive compounds at significant levels (expressed in %).

TABLE (2)
In vitro evaluation of the affinity of inventive compounds
for the ATP-dependent potassium channels at 300 nM

| | K_{ATP} binding |
|---|---|
| Compound | (300 nM) |
| 3 | 75 |
| 6 | 47 |
| 7 | 71 |
| 8 | 48 |
| 16 | 76 |
| 18 | 69 |
| 20 | 85 |
| 21 | 68 |
| 22 | 88 |
| 23 | 97 |
| 24 | 93 |
| 25 | 91 |
| 26 | 92 |
| 27 | 95 |
| 28 | 30 |
| 29 | 15 |
| 32 | 93 |
| 33 | 51 |
| 34 | 81 |
| 35 | 22 |
| 36 | 45 |
| 37 | 76 |
| 41 | 12 |
| 44 | 48 |
| 45 | 66 |
| 51 | 32 |
| 52 | 83 |
| 53 | 25 |
| 54 | 79 |
| 55 | 27 |
| 56 | 49 |
| 57 | 18 |
| 58 | 94 |
| 59 | 73 |
| 60 | 49 |
| 61 | 23 |
| 62 | 46 |
| 63 | 67 |
| 64 | 57 |
| 65 | 90 |

The results presented in table (2) reflect the specific affinity of the inventive compounds on the ATP-dependent potassium channels (K⁺_{ATP}). The specific binding measured corresponds to the difference between the total binding and the non-specific binding determined in the presence of an excess of non-marked reference ligand (glibenclamide 1 μM). The inventive compounds were tested at doses between 3 and 100 nM: the higher the percentage measured, the stronger the affinity of the compound for the ATP-dependent potassium channels.

FIG. (2) shows the dose-dependent character of this specific affinity. This affinity was variable from one compound to another: for example, one can measure a IC₅₀ of 14 nM for compound 1 as opposed to 63 nM for compound 10.

It should be noted that compounds 1A and 1B were evaluated at 100 nM and compared to compound 1. The results obtained show an enantio-dependent affinity: compounds 1A and 1B respectively have an affinity of 92 and 49% at 100 nM.

### Exemple 8

### In Vitro Evaluation of the Insulinotropic Properties of the Inventive Compounds

The insulinotropic properties of the inventive compounds were evaluated in vitro.

Principle

Activation of the secretion of insulin was evaluated in vitro on a INS-1 pancreatic cell line by measuring the concentration of insulin secreted in the culture medium containing glucose at 2.8 mM concentration. The compounds were tested at doses between 0.01 and 10 μM. The results are shown by the induction factor for each condition, i.e. the ratio between the insulin concentration induced by the compound and the concentration induced by 2.8 mM glucose on its own: the higher this factor, the more the compound has insulinotropic properties.

Protocol

The INS-1 cells were cultivated for 72 hours in a RPMI 1640 medium supplemented with the following additives: glucose (5 mM); decomplemented FCS Biowest (10%); glutamine (1%); Penicillin-Streptomycin (1%); Sodium Pyruvate (1%); Hepes Buffer (10 mM); 2-mercaptoethanol (50 μM); aminoguanidine hydrochloride (1 mM); G418 (6 μL/10 mL medium). The medium was then changed by RPMI with 2.8 mM glucose (the same additives) for 24 hrs. After washing with Krebs buffer [NaCl (140 mM); KCl (3.6 mM); CaCl2 (1.5 mM); NaH2PO4 (0.5 mM); MgSO4 (0.5 mM); NaHCO3 (2 mM); Hepes (10 mM); BSA (0.1%); pH 7.4], a 30 min incubation at 37° C. was carried out in this buffer. The treatment using insulinotropic compounds was then carried out for 20 min at 37° C. Following this treatment, the concentration of insulin in the supernatant which comes from each well was measured using an ELISA kit (Insulin Elisa Kit—Crystal Chem., USA).

Results

Unexpectedly, the experimental data show that the inventive compounds stimulate the secretion of insulin in vitro.

The results shown in FIG. (3) provide evidence of an increase in the secretion of insulin in the INS-1 cells treated with the compounds according to the invention: the induction factors of the insulinotropic activity are significant, vary from one compound to the other, and are dose-dependent.

### Example 9

### Evaluation of the Insulinotropic Properties of the Inventive Compounds on Human Islets

The study presented in example 8 has demonstrated that the inventive compounds stimulate the secretion of insulin on INS-1 cells. The objective of the example which follows is to validate the insulinotropic properties of the compounds on isolated human pancreatic islets.

Principle

Isolated islets of Langerhans were put together with compounds according to the invention. They were incubated for one hour, and then the supernatant was taken off. The insulin contained in the supernatant (which corresponds to the insulin secreted by the islets over 1 hr) was titrated. It was compared to the basal secretion of insulin, i.e. to the quantity of insulin secreted by the islets over 1 hour in the absence of a compound. The higher the induced insulin/basal insulin ratio, the stronger the insulinotropic potential of the compound.

Protocol

This study was carried out in the cellular therapy of diabetes laboratory led by Professor Pattou (INSERM ERIT-M 0106, Faculty of medicine of Lille, France (59)). The protocols for the extraction of islets and culture are published (Riachy, Vandewalle et al. 2001). The islets were cultured in a CMRL1066 (Gibco BRL) medium containing 5.5 mmol/l glucose. They were put together with the compounds (+0.001% DMSO). The insulin secreted was measured after 1 hour incubation (titration of the supernatant after centrifugation and extraction as described in the reference) according to a radioimmunological titration method (BI-INSULIN IRMA kit, Sanofi Diagnostics Pasteur). Each condition was applied to 5 independent islets preparations, each containing 40 equivalent islets (40 IE). The results shown in FIG. (4) correspond to the average of the results obtained (±standard deviation).

Results

The inventors have shown that the inventive compounds have insulinotropic activity on isolated human pancreatic islets.

The results presented in FIG. (4) show that islets treated with the inventive compounds secrete more insulin than the control group of islets: islets treated with compound 1 at 1 μM secrete almost 4 times more insulin in 1 hour than non-treated islets. Moreover, this effect is dose-dependent, the same compound not having any significant insulinotropic activity at 0.1 μM.

### Example 10

### In Vivo Evaluation of the Insulinotropic Properties of the Compounds According to the Invention

The objective of this study was to evaluate in vivo the insulinostropic properties of the inventive compounds : was the efficiency of the compounds in vitro predictive of true pharmacological efficiency in vivo?

Principle:

In normal animals (as in man), the consequence of the administration of insulinotropic compounds is a significant increase in the plasmatic level of insulin. This induced hyperinsulinemia induces a reduction in glycemia. This example studies the evolution of glycemia and of insulinemia following oral administration of the inventive compounds in rats: the insulinotropic action of the compounds in vivo should induce a significant decrease of glycemia.

Protocol:

Male Sprague-Dawley rats (300-320 g) (CERJ—Le Genest St Isle-France) were used in order to carry out this experiment. The rats were fed with a standard diet of granular material; they had free access to food and drink and were kept in individual, ventilated cages in a 12 hr/12 hr light/dark rhythm. The animals were deprived of food for 16 hours prior to the experiment.

The compound was suspended in an aqueous carboxymethylcellulose solution (Sigma C4888) at 1% containing 0.1% Tween80 (Sigma P8074). The animals of the control group received the vehicle alone. The treated animals received a single administration of the compound (the suspension was administered by gavage at the rate of 10 ml/kg). Each group was made up of 11 animals. A 1^{st} blood sample was taken by puncturing the retro-orbital sinus of the animals under volatile isoflurane anaesthesia: this sample makes up the initial time of the experiment. The substance or the vehicle were immediately administered to the animals by gavage, and blood samples were taken over time.

The animals' glycemia was measured in real time using a glucometer (Glucotrend2-Roche Diagnostic-France). The insulinemias were measured using an ELISA kit (Insulin Elisa Kit—Crystal Chem. USA). The method used was of the ELISA type: plasmatic insulin was specifically bound to anti-insulin monoclonal antibodies immobilised on the microtitration plate; simultaneously, an anti-insulin polyclonal guinea pig antibody was added to the reaction. The revelation of the reaction was guaranteed by an antibody coupled to peroxidase. These antibodies bind specifically to the guinea pig's immunoglobulins. The production of oxidised chromogen causes an increase in absorbance in proportion to the quantity of insulin present in the sample.

Results:

The inventors have provided evidence showing that the inventive compounds are activators of the secretion of insulin. These molecules therefore are of major interest within the scope of type II diabetes and associated pathologies.

The results given in FIGS. (5a) and (5b) show that the inventive compounds have a hypoglycemic effect and that this property is directly linked to stimulation of the secretion of insulin in vivo: compound 1 administered at 50 mpk induced a significant plasmatic insulin peak 15 minutes following administration which caused a drastic decrease in glycemia (decrease of 48% between T=0 and T=120 min).

The results presented in FIG. (5c) show that the hypoglycemic effect established is dose-dependent: compound 52 administered at 500 mpk induced a significant hypoglycemia (decrease of 19% between T=0 and T=120 min) whereas the same compound at 50 mpk had no measurable effect.

Interestingly, a comparison of FIGS. (5a) and (5c) shows that the inventive compounds regulate glycemia with different kinetics and amplitudes: hypoglycemia induced by compound 1 was faster and more intense than that generated by compound 52.

### Example 11

### In Vivo Evaluation of the Regulation of the Expression of Genes by the Compounds According to the Invention

The objective of this study was to evaluate in vivo the increase in expression of PPARα target genes. Because these genes (the expression level of which is regulated by PPARα are directly involved in the metabolism of lipids and glucids, this type of increase would bear witness to a major therapeutic interest in the compounds according to the invention, notably within the scope of dyslipidemias.

Principle:

The PPARα nuclear receptor is essentially expressed in the liver, a tissue where the catabolism of fatty acids is important. This nuclear receptor modulates the expression of certain genes coding for proteins and enzymes which are heavily involved in the metabolism of lipids and glucids.

The capacity of the inventive compounds to induce transcriptional activity was evaluated in vivo in Hartley guinea pigs. The treatment of these animals by a PPARα agonist must induce an hepatic over-expression of the target genes directly under the control of the PPARα receptor. The genes which we studied in this experiment were ACO (acyl Co-enzymeA oxidase, a key enzyme in the mechanism of β-oxidation of fatty acids) and PDK-4 (pyruvate deshydrogenase kinase isoform 4, enzyme of glucidic metabolism). The higher the induction factor measured, the more the tested compound increases the hepatic expression of the gene being studied.

Protocol:

a—Treatment of the Animals

Male Dunkin Hartley guinea pigs (Harlan, Gannat, France) aged 5-6 weeks at the start of the experiment were put in groups of 6 animals selected such that the distribution of their body weight was uniform. The guinea pigs were given a standard diet (Harlan, Gannat, France). The animals were kept in a 12 hours/12 hours light/dark cycle at a constant temperature of 20±3° C. The animals had free access to water and food. The food consumed and the gain in weight were recorded. The compounds being tested were suspended in an aqueous carboxymethylcellulose solution (Sigma C4888) at 1% containing 0.1% Tween80 (Sigma P8074) and administered by intragastric gavage, once a day for 3 days at the dose of 200 mg/kg/day. At the end of the experiment, the animals were anaesthetised, and then euthanazed. The animal livers were taken, weighed and kept at −80° C. in order to carry out subsequent analysis.

b—Analysis of Gene Expression by Quanitative RT-PCR

Total RNA was extracted from fragments of liver using the NucleoSpin® 96 RNA kit (Macherey Nagel, Hoerdt, France) according to the manufacturer's instructions. Messenger RNAs were quantified by quantitative RT-PCR using the iQ SYBR Green Supermix kit (Biorad, Marnes-la-Coquette, France) on the following apparatus: MyiQ Single-Color Real-Time PCR Detection System (Biorad, Marnes-la-Coquette, France). The quantity of fluorescence emitted is directly in proportion to the quantity of complementary DNA present at the start of the reaction and amplified during the PCR. Pairs of specific primers of the genes ACO (5′-TTGGAAACCACTGCCACATA-3′(SEQ ID NO:1) and 5′-AGGACCAATGTCTCCCACAG-3′ (SEQ ID NO:2) ) and PDK4 (5′-AGAGCCTGATGGATTTGGTG-3′ (SEQ ID NO:3) and 5′-TTGATTGGTGACTGGGTCAA-3′ (SEQ ID NO:4) ) were used as probes. Pairs of specific primers of the gene 18S (5′-CGGACACGGACAGGATTGACAG-3′ (SEQ ID NO:5) and 5′-AATCTCGGGTGGCTGAACGC-3′ (SEQ ID NO:6) ) were used as control probes. The relative levels of expression were determined using the efficiency curves for each transcript. The relative quantity was then calculated and standardized in relation to the 18S signal. The induction factor, i.e. the ratio between the relative signal (induced by the compound according to the invention) and the average of the values relating to the control group, was then calculated for each sample. The higher this factor, the more the compound promotes hepatic gene expression. The final result is depicted as the average of the induction values in each experimental group.

Results:

The inventors provided evidence showing that the inventive compounds are regulators of the expression of genes in vivo.

The results presented in FIGS. (6a) and (6b) show that the inventive compounds induce a significant increase in the hepatic expression of the genes coding for ACO and PDK-4. These genes (the expression level of which is regulated by PPARα) coding for enzymes which are strongly involved in the metabolism of lipids and glucids, the inventive compounds show a major pharmacological interest within the scope of metabolic diseases such as dyslipidemias.

### Example 12

### In Vivo Evaluation of the Lipid-Lowering Properties of the Compounds According to the Invention

The objective of this study was to evaluate the lipid-lowering potential of the inventive compounds in vivo.

Principle:

The lipid-lowering effect of the inventive compounds was evaluated in vivo in Hartley guinea pigs. In guinea pigs, as in man, a diet enriched in cholesterol and fatty acids induces an increase in the plasmatic levels of lipids. Plasmatic lipid levels (cholesterol and triglycerides) after 14 days treatment were measured and compared to the levels observed for non-treated animals (controls).

Protocol:

a—Treatment of the Animals

Male Dunkin Hartley guinea pigs (Harlan, Gannat, France) aged 5-6 weeks at the start of the experiment were put in groups of 6 animals selected such that the distribution of their body weight and of their plasmatic lipid levels determined before the experiment were uniform. The guinea pigs were given a diet containing 0.2% cholesterol and 14% fats (Research Diets Inc.-USA). They were kept in a 12 hour/12 hour light/dark cycle at a constant temperature of 20±3° C. and had free access to water and food. The amount of food eaten and the increase in weight were recorded. The compounds tested were suspended in an aqueous carboxymethylcellulose solution (Sigma C4888) at 1% containing 0.1% Tween80 (Sigma P8074) and administered daily by intragastric gavage for 14 days at a dose of 200 mg/kg/day. At the end of the experiment the animals were anaesthetised after fasting for 16 hours and a blood sample was taken on anticoagulant (EDTA). The guinea pigs were then weighed and then euthanased. The plasma was prepared by centrifugation at 3000 revolutions/minute for 20 minutes, the samples were kept at +4° C. Liver samples were taken, frozen in liquid nitrogen, and kept at −80° C. for subsequent analysis.

b—Titration of Lipids

The plasmatic concentrations of the lipids (total cholesterol and triglycerides) were measured by enzymatic titrations (bioMérieux-Lyon-France) according to the supplier's recommendations.

Results:

The inventors provided evidence to show that the inventive compounds have lipid-lowering properties.

FIG. (7) depicts the efficacy of compound 52 administered at 200 mpk for 14 days in guinea pigs under high fat diet. Surprinsingly, plasmatic lipid levels are significantly lower for treated animal when compared to controls:
Triglycerides plasmatic levels are approximately 22% lower
Cholesterol plasmatic levels are approximately 45% lower

These results obtained in vivo bear witness to the therapeutic potential of the inventive compounds with regard to major pathologies such as dyslipidemias.

### BIBLIOGRAPHY

Angel, I. and S. Bidet (1991). “The binding site for [3H]glibenclamide in the rat cerebral cortex does not recognize K-channel agonists or antagonists other than sulphonylureas.” Fundam Clin Pharmacol 5(2): 107-15.

Brown, G. and A. Foubister (1984). “Receptor binding sites of hypoglycemic sulfonylureas and related [(acylamino)alkyl]benzoic acids.” J Med Chem 27(1): 79-81.

Chapman, M. (2003). “Fibrates in 2003: therapeutic action in atherogenic dyslipidaemia and future perspectives.” Atherosclerosis 171(1): 1-13.

Chinetti, G., J. Fruchart, et al. (2003). “Peroxisome proliferator-activated receptors and inflammation: from basic science to clinical applications.” Int J Obes Relat Metab Disord 27 Suppl 3: S41-5.

Denke, M. (2003). “Combination therapy.” J Manag Care Pharm 9(1 Suppl): 17-9.

Desvergne, B. and W. Wahli (1999). “Peroxisome Proliferator-Activated Receptors: Nuclear Control of Metabolism.” Endocr. Rev. 20(5): 649-688.

Dressel, U., T. L. Allen, et al. (2003). “The Peroxisome Proliferator-Activated Receptor {beta}/{delta} Agonist, GW501516, Regulates the Expression of Genes Involved in Lipid Catabolism and Energy Uncoupling in Skeletal Muscle Cells.” Mol. Endocrinol. 17(12): 2477-2493.

Etgen, G. and N. Mantlo (2003). “PPAR ligands for metabolic disorders.” Curr Top Med Chem 3(14): 1649-61.

Gin, H. and V. Rigalleau (2002). “Oral anti diabetic polychemotherapy in type 2 diabetes mellitus.” Diabetes Metab 28(5): 350-3.

Greene, T. W. and P. G. M. Wuts (1999). Protective Groups in Organic Synthesis. 3rd Ed.: John Wiley & Sons Inc.

Grell, W., R. Hurnaus, et al. (1993). “Phenylacetic acid benzylamides.” U.S. Pat. No. 5,216,167 et U.S. Pat. No. 5,312,924.

Grell, W., R. Hurnaus, et al. (1998). “Repaglinide and related hypoglycemic benzoic acid derivatives.” J Med Chem 41(26): 5219-46.

Guerre-Millo, M., P. Gervois, et al. (2000). “Peroxisome Proliferator-activated Receptor alpha Activators Improve Insulin Sensitivity and Reduce Adiposity.” J. Biol. Chem. 275(22): 16638-16642.

IAS (2003). “Harmonized Clinical Guidelines on Prevention of Atherosclerotic Vascular Disease.” International Atherosclerosis Society.

Laight, D. W. (2003). “Future anti-inflammatory metabolic and cardiovascular management of Type 2 diabetes mellitus.” Expert Opinion on Therapeutic Patents 13(11): 1683-1689.

McCormick, M. and L. Quinn (2002). “Treatment of type 2 diabetes mellitus: pharmacologic intervention.” J Cardiovasc Nurs 16(2): 55-67.

Pershadsingh, H. (2004). “Peroxisome proliferator-activated receptor-gamma: therapeutic target for diseases beyond diabetes: quo vadis?” Expert Opin Investig Drugs 13(3): 215-28.

Proks, P., F. Reimann, et al. (2002). “Sulfonylurea stimulation of insulin secretion.” Diabetes 51 Suppl 3: S368-76.

Ram, V. (2003). “Therapeutic significance of peroxisome proliferator-activated receptor modulators in diabetes.” Drugs Today (Barc) 39(8): 609-32.

Raspe, E., L. Madsen, et al. (1999). “Modulation of rat liver apolipoprotein gene expression and serum lipid levels by tetradecylthioacetic acid (TTA) via PPAR{alpha} activation.” J. Lipid Res. 40(11): 2099-2110.

Riachy, R., B. Vandewalle, et al. (2001). “Beneficial effect of 1,25 dihydroxyvitamin D3 on cytokine-treated human pancreatic islets.” J Endocrinol 169(1): 161-8.

Rufer, C. and W. Losert (1979). “Blood glucose lowering sulfonamides with asymmetric carbon atoms. 3. Related N-substituted carbamoylbenzoic acids.” J Med Chem 22(6): 750-2.

Spiegelman, B. (1998). “PPAR-gamma: adipogenic regulator and thiazolidinedione receptor.” Diabetes 47(4): 507-514.

Sugden, M. C. and M. J. Holness (2004). “Potential Role of Peroxisome Proliferator-Activated Receptor-{alpha} in the Modulation of Glucose-Stimulated Insulin Secretion.” Diabetes 53(90001): S71-81.

Verges, B. (2004). “Clinical interest of PPARs ligands.” Diabetes Metab 30(1): 7-12.

Willson, T. M., P. J. Brown, et al. (2000). “The PPARs: from orphan receptors to drug discovery.” J Med Chem 43(4): 527-50.

1. (canceled)

2. A substituted N-(benzyl)phenylacetamide derivative, wherein it is represented by formula (II) below: 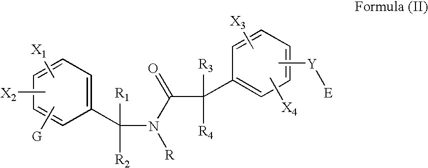
in which,
G represents:
A NRₐR_{b} radical, cyclic or not; or
A —NR′ₐCOR′_{b}; or
A —OR′ₐ, —SR′ₐ; or
A —SOR_{c} or —SO₂R_{c} radical;
Rₐ and R_{b}, identical or different, substituted or not, representing a hydrogen atom or an alkyl, alkenyl, aryl, aralkyl radical or a heterocycle;
Alternatively, Rₐ and R_{b} can form together a heterocycle with the nitrogen atom to which they are attached;
R′ₐand R′_{b}, identical or different, substituted or not, represent a hydrogen atom or an alkyl, alkenyl, aryl, aralkyl radical or a heterocycle;
Alternatively, R′ₐ and R′_{b} can form together a heterocycle with the nitrogen atom,
R_{c} representing an alkyl, alkenyl, aryl, aralkyl type radical or a heterocycle;
G possibly being able to form a heterocycle with X1;
R1 represents an alkyl, alkenyl, aryl, aralkyl type radical or a heterocycle;
R2, R3 and R4 independently represent a hydrogen atom, an alkyl, alkenyl, aryl, aralkyl type radical or a heterocycle;
R1 and R4 each, independently, being able to be linked to the molecular skeleton by a double linkage, R2 or R3 then being absent;
R represents a hydrogen atom or an alkyl, alkenyl, aryl, aralkyl type radical or a heterocycle;
Y represents an oxygen atom, a sulphur atom (possibly oxidised to sulphoxide or sulphone function), an amine group of the NR type (R being as defined above, in particular a hydrogen atom or an alkyl radical) or a selenium atom (possibly oxidised to selenoxide or selenone function);
E represents:
An alkyl or alkenyl chain, substituted by one or more W groups as defined below; or
A chain corresponding to the formula —(CH₂)ₘ—Y1-Z-Y2-(CH₂)ₙ—CR₅R₆—W;
in which,
R5 and R6 independently represent a hydrogen atom, a halogen atom or an alkyl, alkenyl, aryl, aralkyl, —OR′ₐ or —SR′ₐ radical;
alternatively R5 and R6 may form together a cycle,
m and n independently represent an integer between 0 and 10;
Y1 and Y2 independently represent a covalent linkage or a heteroatom chosen from nitrogen (of the NR type, R being as defined above, in particular a hydrogen atom or an alkyl radical), oxygen or sulphur;
Z represents a covalent linkage or an alkyl, alkenyl, aryl, aralkyl radical;
W represents:
a carboxyl radical or a derivative, preferably of the —COOH, —COOR′ₐ, —COSR′ₐ, —CONR′ₐR′_{b}, —CSNR′ₐR′_{b} type; or
an isosteric group of the carboxyl radical, preferably an acylsulphonamide (—CONHSO₂R_{c}) or tetrazolyl radical; or
sulphonic acid (—SO₃H) or a derivative of the —SO₃R′ₐ or —SO₂NR′ₐR′_{b} type;
R′ₐ, R′_{b} and R_{c} being as defined above;
X1, X2, X3 and X4 independently represent a hydrogen or halogen atom, an —NO₂ or —CN function, an alkyl, alkenyl, aryl, aralkyl, —OR′ₐ, —SR′ₐ, —NR′ₐR′_{b}, —SORC or —SO₂R_{c}, radical or a heterocycle;
R′ₐ, R′_{b} and R_{c} being as defined above;
X1 and X3 each being able to form a cycle (aromatic or not, heterocyclic or not) with X2 and X4 respectively;
the optical and geometric isomers, racemates, tautomers, salts, hydrates and mixtures thereof.

3. Derivative according to claim 2, wherein the G group is in ortho position (with respect to the —CR1R2- motif) of the phenyl radical to which it is attached.

4. Derivative according to claim 2, wherein the Y-E group is in para position (with respect to the —CR3R4- motif) of the phenyl radical to which it is attached.

5. Derivative according to claim 2, wherein the G group is in ortho position (with respect to the —CR1R2- motif) of the phenyl radical to which it is attached and the Y-E group is in para position (with respect to the —CR3R4- motif) of the phenyl radical to which it is attached.

6. Derivative according to claim 2, wherein the G group represents a cyclic —NRₐR_{b} radical, G beneficially forming a nitrogenous heterocycle of the cyclic alkyleneimino type, substituted or not, possibly containing several heteroatoms or an aromatic nitrogenous heterocycle substituted or not.

7. Derivative according to claim 2, wherein
G represents:
A —NRₐR_{b} radical, cyclic or not; or
A —OR′ₐ, —SR′ₐ radical;
Rₐ, R_{b} and R′ₐ and/or
R1 represents a radical of the alkyl, alkenyl, aryl or aralkyl type; and/or
R2, R3 and R4 independently represent a hydrogen atom, an alkyl, alkenyl, aryl or aralkyl type radical; and/or
R represents a hydrogen atom; and/or
Y represents an oxygen or sulphur atom; and/or
E represents a chain corresponding to the formula —(CH₂)ₘ—Y1-Z-Y2-(CH₂)ₙ—CR₅R₆—W in which,
R5 and R6 independently represent a hydrogen atom, a halogen atom or an alkyl, alkenyl, aryl, aralkyl, —OR′ₐ or —SR′ₐ radical;
alternatively R5 and R6 may form together a cycle,
m and n independently represent an integer between 0 and 10;
Y1 and Y2 independently represent a covalent linkage or a heteroatom chosen from nitrogen (of the NR type, R being as defined above, in particular a hydrogen atom or an alkyl radical), oxygen or sulphur;
Z represents a covalent linkage or an alkyl, alkenyl, aryl, aralkyl radical; and/or
W represents:
a carboxyl radical or a derivative, preferably of the —COOH, —COOR′ₐ, —COSR′ₐ, —CONR′ₐR′_{b}, —CSNR′ₐR′_{b} type, or
an isosteric group of the carboxyl radical, preferably an acylsulfonamide (—CONHSO₂R_{c}) or tetrazolyl radical;
R′ₐ and R′_{b} being as defined above; and/or
X1, X2, X3 and X4 independently represent a hydrogen or halogen atom, a —NO₂ or —CN function, an alkyl, alkenyl, aryl, aralkyl, —OR′ₐ, —SR′ₐ or a heterocycle;
R′ₐ, R′_{b} and R_{c} being as defined above.

8. Derivative according to claim 2, wherein the E group represents a chain corresponding to the formula —(CH₂)ₘ—Y1-Z-Y2-(CH₂)ₙ—CR₅R₆—W, in which at least one of the radicals R₅ and R₆ is different from the hydrogen atom.

9. Derivative according to claim 2, wherein the E group represents a chain corresponding to the formula —(CH₂)ₙ—CR₅R₆—W.

10. Derivative according to claim 2, wherein the E group represents a chain corresponding to the formula —(CH₂)ₙ—CR₅R₆—W with n=0.

11. Derivative according to claim 2, wherein W represents:
a carboxyl radical or a derivative, preferably of the —COOH or —COOR′ₐ, or
an acylsulfonamide (—CONHSO₂R_{c}) radical;
R′ₐ and R_{c}.

12. Derivative according to claim 2, wherein it is selected in the group consisting of:
2-[4-[1-(3-methyl-1-(2-(1-piperidinyl)phenyl)butyl)aminocarbonylmethyl]phenoxy]-2-methylpropanoic acid,
2-[4-[1-(1-(2-(1-piperidinyl)phenyl)ethyl)aminocarbonylmethyl]phenoxy]-2-methylpropanoic acid,
2-[4-[1-(1-phenyl-1-(2-(1-piperidinyl)phenyl)methyl)aminocarbonylmethyl]phenoxy]-2-methylpropanoic acid,
2-[2-methoxy-4-[1-(3-methyl-1-(2-(1-piperidinyl)phenyl)butyl)aminocarbonylmethyl]phenoxy]-2-methylpropanoic acid,
2-[4-[1-(1-(2-(1-piperidinyl)phenyl)heptyl)aminocarbonylmethyl]phenoxy]-2-methylpropanoic acid,
2-[4-[1-(1-cyclohexyl-1-(2-(1-piperidinyl)phenyl)methyl)aminocarbonylmethyl]phenoxy]-2-methylpropanoic acid,
2-[4-[1-(2-phenyl-1-(2-(1-piperidinyl)phenyl)ethyl)aminocarbonylmethyl]phenoxy]-2-methylpropanoic acid,
2-[4-[1-(3-methyl-1-(3-(1-piperidinyl)phenyl)butyl)aminocarbonylmethyl]phenoxy]-2-methylpropanoic acid, 2-[4-[1-(3-methyl-1-(4-(1-piperidinyl)phenyl)butyl)aminocarbonylmethyl]phenoxy]-2-methylpropanoic acid, 2-[4-[1-(1-(2-(1-piperidinyl)phenyl)pentyl)aminocarbonylmethyl]phenoxy]-2-methylpropanoic acid,
2-[2,6-dimethyl-4-[1-(3-methyl-1-(2-(1-piperidinyl)phenyl)butyl)aminocarbonylmethyl]phenoxy]-2-methylpropanoic acid,
2-[4-[1-(3-methyl-1-(2-(1-cyclohexylamino)phenyl)butyl)aminocarbonylmethyl]phenoxy]-2-methylpropanoic acid,
2-[4-[1-(1-(2-(1-pyrrolidinyl)phenyl)ethyl)aminocarbonylmethyl]phenoxy]-2-methylpropanoic acid,
2-[4-[1-(1-(2-(4-morpholinyl)phenyl)ethyl)aminocarbonylmethyl]phenoxy]-2-methylpropanoic acid,
2-[4-[1-(1-(2-(1-hexamethyleneimino)phenyl)ethyl)aminocarbonylmethyl]phenoxy]-2-methylpropanoic acid,
2-[4-[1-(3-methyl-1-(2-(1-pyrrolidinyl)phenyl)butyl)aminocarbonylmethyl]phenoxy]-2-methylpropanoic acid,
2-[4-[1-(3-methyl-1-(2-(4-morpholinyl)phenyl)butyl)aminocarbonylmethyl]phenoxy]-2-methylpropanoic acid,
2-[4-[1-(3-methyl-1-(2-(1-hexamethyleneimino)phenyl)butyl)aminocarbonylmethyl]phenoxy]-2-methylpropanoic acid,
2-[4-[1-(2-cyclohexyl-1-(2-(1-piperidinyl)phenyl)ethyl)aminocarbonylmethyl]phenoxy]-2-methylpropanoic acid,
2-[4-[1-(3-methyl-1-(2-(diethylamino)phenyl)butyl)aminocarbonylmethyl]phenoxy]-2-methylpropanoic acid,
2-[4-[1-(3-methyl-1-(2-(1-piperidinyl)-4-(trifluoromethyl)phenyl)butyl)aminocarbonylmethyl]phenoxy]-2-methylpropanoic acid,
2-[4-[1-(3-methyl-1-(2-(1-piperidinyl)-5-(chloro)phenyl)butyl)aminocarbonylmethyl]phenoxy]-2-methylpropanoic acid,
2-[4-[1-(3-methyl-1-(2-(1-piperidinyl)phenyl)butyl)aminocarbonylmethyl]phenoxy]acetic acid,
2-[4-[1-(3-methyl-1-(2-(1-piperidinyl)phenyl)butyl)aminocarbonylmethyl]phenoxy]propanoic acid,
2-[4-[1-(3-methyl-1-(2-(1-piperidinyl)phenyl)butyl)aminocarbonylmethyl]phenoxy]butanoic acid,
2-[4-[1-(3-methyl-1-(2-(1-piperidinyl)phenyl)butyl)aminocarbonylmethyl]phenoxy]-3-methylbutanoic acid,
2-[4-[1-(3-methyl-1-(2-(1-piperidinyl)phenyl)butyl)aminocarbonylmethyl]phenoxy]-2-phenylacetic acid,
5-[4-[1-(3-methyl-1-(2-(1-piperidinyl)phenyl)butyl)aminocarbonylmethyl]phenoxy]-2,2-dimethylpentanoic acid,
2-[3-[1-(3-methyl-1-(2-(1-piperidinyl)phenyl)butyl)aminocarbonylmethyl]phenoxy]-2-methylpropanoic acid,
2-[3-[1-(1-(2-(1-piperidinyl)phenyl)ethyl)aminocarbonylmethyl]phenoxy]-2-methylpropanoic acid,
2-[3-[1-(1-phenyl-1-(2-(1-piperidinyl)phenyl)methyl)aminocarbonylmethyl]phenoxy]-2-methylpropanoic acid,
2-[4-[1-(3-methyl-1-(2-(1-piperidinyl)phenyl)butyl)carbonylaminomethyl]phenoxy]-2-methylpropanoic acid,
2-[4-[1-(1-(2-(1-piperidinyl)-4-(bromo)phenyl)pentyl)aminocarbonylmethyl]phenoxy]-2-methylpropanoic acid,
2-[4-[1-methyl-1-[1-(3-methyl-1-(2-(1-piperidinyl)phenyl)butyl)aminocarbonyl]methyl]phenoxy]-2-methylpropanoic acid,
2-[4-[N-isobutyl-N-(1-(2-(1-piperidinyl)phenyl)pentyl)aminocarbonylmethyl]phenoxy]-2-methylpropanoic acid,
2-[4-[1-(1-(2-(phenylthio)phenyl)pentyl)aminocarbonylmethyl]phenoxy]-2-methylpropanoic acid,
2-[4-[1-(3-methyl-1-(2-(1-piperidinyl)-5-(bromo)phenyl)butyl)aminocarbonylmethyl]phenoxy]-2-methylpropanoic acid,
2-[4-[1-(1-(2-(1-piperidinyl)-4-(1H-indolyl)phenyl)pentyl)aminocarbonylmethyl]phenoxy]-2-methylpropanoic acid,
2-[4-[1-(3-methyl-1-(2-(1-piperidinyl)-4-(phenyl)phenyl)butyl)aminocarbonylmethyl]phenoxy]-2-methylpropanoic acid,
2-{4-{4-[4-[1-(3-methyl-1-(2-(1-piperidinyl)phenyl)butyl)aminocarbonylmethyl]phenoxy]butoxy}phenoxy}-2-methylpropanoic acid,
2-[4-[1-(1-(2-(phenylsulfonyl)phenyl)pentyl)aminocarbonylmethyl]phenoxy]-2-methylpropanoic acid,
3-[4-[1-(1-(2-(1-piperidinyl)phenyl)pentyl)aminocarbonylmethyl]phenoxy]-2,2-dimethylpropanoic acid,
2-[4-[1-(1-(2-((3S,5R)-3,5-dimethylpiperidine-1-yl)phenyl)pentyl)aminocarbonylmethyl]phenoxy]-2-methylpropanoic acid,
2-[4-[1-(1-(2-(hydroxy)phenyl)pentyl)aminocarbonylmethyl]phenoxy]-2-methylpropanoic acid,
2-[4-[1-(1-(3-fluoro-2-(1-piperidinyl)phenyl)pentyl)aminocarbonylmethyl]phenoxy]-2-methylpropanoic acid,
5-{N-ethyl-N-[4-[1-(3-methyl-1-(2-(1-piperidinyl)phenyl)butyl)aminocarbonylmethyl]phenyl]amino}-2,2-dimethylpentanoic acid,
5-[4-[1-(1-(2-(1-piperidinyl)phenyl)pentyl)aminocarbonylmethyl]phenoxy]-2,2-dichloropentanoic acid,
2-[4-[1-(2-(4-methoxyphenyl)-1-(2-(1-piperidinyl)phenyl)ethyl)aminocarbonylmethyl]phenoxy]-2-methylpropanoic acid,
2-[4-[1-(1-(2-(1-piperidinyl)-4-(1H-pyrrolyl)phenyl)pentyl)aminocarbonylmethyl]phenoxy]-2-methylpropanoic acid,
2-[4-[1-(1-(2-(1-piperidinyl)-4-(phenethyl)phenyl)pentyl)aminocarbonylmethyl]phenoxy]-2-methylpropanoic acid,
2-[4-[1-(3-phenyl-1-(2-(1-piperidinyl)phenyl)propyl)aminocarbonylmethyl]phenoxy]-2-methylpropanoic acid,
N-[methylsulfonyl]-2-[4-[1-(1-(2-(1-piperidinyl)phenyl)pentyl)aminocarbonylmethyl]phenoxy]-2-methylpropanamide,
5-[2-[1-(1-(2-(1-piperidinyl)phenyl)pentyl)aminocarbonylmethyl]phenoxy]-2,2-dimethylpentanoic acid,
2-[4-[1-(1-(2-(methoxy)phenyl)pentyl)aminocarbonylmethyl]phenoxy]-2-methylpropanoic acid,
2-[4-[1-(2-(3-methylphenyl)-1-(2-(1-piperidinyl)phenyl)ethyl)aminocarbonylmethyl]phenoxy]-2-methylpropanoic acid,
2-[2-chloro-4-[1-(3-methyl-1-(2-(1-piperidinyl)phenyl)butyl)aminocarbonylmethyl]phenoxy]-2-methylpropanoic acid,
6-[4-[1-(3-methyl-1-(2-(1-piperidinyl)phenyl)butyl)aminocarbonylmethyl]phenoxy]-2,2-dimethylhexanoic acid,
N-[methylsulfonyl]-2-[4-[1-(3-methyl-1-(2-(1-piperidinyl)phenyl)butyl)aminocarbonylmethyl]phenoxy]-2-methylpropanamide,
N-[trifluoromethylsulfonyl]-2-[4-[1-(3-methyl-1-(2-(1-piperidinyl)phenyl)butyl)aminocarbonylmethyl]phenoxy]-2-methylpropanamide,
N-[trifluoromethylsulfonyl]-2-[4-[1-(1-(2-(1-piperidinyl)phenyl)pentyl)aminocarbonylmethyl]phenoxy]-2-methylpropanamide,
N-[phenylsulfonyl]-2-[4-[1-(1-(2-(1-piperidinyl)phenyl)pentyl)aminocarbonylmethyl]phenoxy]-2-methylpropanamide,
N-[methylsulfonyl]-2-[4-[1-(2-phenyl-1-(2-(1-piperidinyl)phenyl)ethyl)aminocarbonylmethyl]phenoxy]-2-methylpropanamide,
N-[benzylsulfonyl]-2-[4-[1-(1-(2-(1-piperidinyl)phenyl)pentyl)aminocarbonylmethyl]phenoxy]-2-methylpropanamide,
N-[phenylsulfonyl]-2-[4-[1-(3-methyl-1-(2-(1-piperidinyl)phenyl)butyl)aminocarbonylmethyl]phenoxy]-2-methylpropanamide,
2-[4-[1-(1-(2-(1-piperidinyl)phenyl)butyl)aminocarbonylmethyl]phenoxy]-2-methylpropanoic acid,
N-[methylsulfonyl]-2-[4-[1-(1-(2-(1-piperidinyl)phenyl)butyl)aminocarbonylmethyl]phenoxy]-2-methylpropanamide,
N-[methylsulfonyl]-2-[4-[1-(1-(2-(methoxy)phenyl)pentyl)aminocarbonylmethyl]phenoxy]-2-methylpropanamide,
2-[4-[1-(1-(2-(isobutyloxy)phenyl)pentyl)aminocarbonylmethyl]phenoxy]-2-methylpropanoic acid,
2-[4-[1-(1-(2-(1-piperidinyl)phenyl)pentyl)aminocarbonylmethyl]phenoxy]-2-(ethoxycarbonyl)acetic acid,
2-(4-((1H-tetrazol-5-yl)methoxy)phenyl)-N-[1-(1-(2-(1-piperidinyl)phenyl)pentyl)]acetamide,
2-[4-[1-(1-(2-(1-piperidinyl)phenyl)pentyl)aminocarbonylmethyl]phenoxy]acetic acid,
N-[methylsulfonyl]-2-[4-[1-(1-(2-(1-piperidinyl)phenyl)pentyl)aminocarbonylmethyl]phenoxy]acetamide,
2-[2-methoxy-4-[1-(1-(2-(1-piperidinyl)phenyl)pentyl)aminocarbonylmethyl]phenoxy]-2-methylpropanoic acid,
2-[4-[1-(2-phenyl-1-(2-(1-piperidinyl)phenyl)ethyl)aminocarbonylmethyl]phenylthio]-2-methylpropanoic acid,
2-[4-[1-(1-(2-(diethylamino)phenyl)pentyl)aminocarbonylmethyl]phenoxy]-2-methylpropanoic acid,
N-[methylsulfonyl]-2-[4-[1-(1-(2-(diethylamino)phenyl)pentyl)aminocarbonylmethyl]phenoxy]-2-methylpropanamide,
N-[methylsulfonyl]-2-[2-methoxy-4-[1-(1-(2-(1-piperidinyl)phenyl)pentyl)aminocarbonylmethyl]phenoxy]-2-methylpropanamide,
2-[4-[1-(1-(2-(acetamido)phenyl)pentyl)aminocarbonylmethyl]phenoxy]-2-methylpropanoic acid,
2-[4-[1-(1-(2-(1-piperidinyl)phenyl)pentyl)carbonylaminomethyl]phenoxy]-2-methylpropanoic acid,
N-[methylsulfonyl]-2-[4-[1-(1-(2-(1-piperidinyl)phenyl)pentyl)carbonylaminomethyl]phenoxy]-2-methylpropanamide,
1-[4-[1-(1-(2-(1-piperidinyl)phenyl)pentyl)aminocarbonylmethyl]phenoxy]-1-cyclobutane carboxylic acid
N-[methylsulfonyl]-1-[4-[1-(1-(2-(1-piperidinyl)phenyl)pentyl)aminocarbonylmethyl]phenoxy]cyclobutanecarboxamide.

13. Method for preparing compounds represented by formula (I) as defined in claim 2, wherein it comprises at least the following step:
(i) A condensation step, preferably of a mono- or polysubstituted benzylamine type derivative with a mono- or polysubstituted phenylacetic acid (or derivative) type derivative; and possibly, before or after step (i),
(ii) one or more insertion and/or transformation steps of functional groups.

14. Derivative selected in the group consisting of:
4-[1-(3-methyl-1-(2-(1-piperidinyl)phenyl)butyl)aminocarbonylmethyl]phenol;
4-[1-methyl-1-[1-(3-methyl-1-(2-(1-piperidinyl)phenyl)butyl)aminocarbonyl]methyl]phenol;
4-[1-(1-(2-(1-piperidinyl)phenyl)pentyl)aminocarbonylmethyl]phenol;
4-[1-(1-(2-(1-piperidinyl)phenyl)heptyl)aminocarbonylmethyl]phenol;
N-ethyl-N-[4-[1-(3-methyl-1-(2-(1-piperidinyl)phenyl)butyl)aminocarbonylmethyl]phenyl]amine;
2-[1-(1-(2-(1-piperidinyl)phenyl)pentyl)aminocarbonylmethyl]phenol;
2-chloro-4-[1-(3-methyl-1-(2-(1-piperidinyl)phenyl)butyl)aminocarbonylmethyl]phenol;
4-[1-(1-(2-(1-piperidinyl)phenyl)butyl)aminocarbonylmethyl]phenol;
4-[1-(2-phenyl-1-(2-(1-piperidinyl)phenyl)ethyl)aminocarbonylmethyl]thiophenol;
4-[1-(1-(2-(diethylamino)phenyl)pentyl)aminocarbonylmethyl]phenol.

15. Derivative according to claim 2 as medicines.

16. Pharmaceutical composition comprising, in a pharmaceutically acceptable support, at least one compound represented by formula (I) such as defined in claim 2, possibly in association with one or more other therapeutic and/or cosmetic active ingredients.

17. Pharmaceutical composition comprising, in a pharmaceutically acceptable support, at least one compound represented by formula (I) such as defined in claim 2, in association with one or more compound selected in the group consisting of:
combination with other therapeutic and/or cosmetic agents such as
Antidiabetics
insulin
lipid-lowering and/or cholesterol-lowering molecules
anti-hypertension agents and hypotension agents
anti-platelet agents
anti-obesity agents
anti-inflammatories
anti-oxidant agents
agents used in the treatment of cardiac insufficiency
agents used for the treatment of coronary insufficiency
antineoplastics
anti-asthmatics
corticoids used in the treatment of pathologies of the skin
vasodilatators and/or anti-ischemic agents.

18. Pharmaceutical composition as defined in claim 16, for the treatment or prophylaxis of diabetes, dyslipidemias, insulin resistance, pathologies associated with Syndrome X, atherosclerosis, cardiovascular diseases, obesity, hypertension, inflammatory diseases.

19. Pharmaceutical composition as defined in claim 16, for preventing and/or treating cardiovascular risk factors linked to disorders of lipidic and/or glucidic metabolism.

20. Method of treatment and/or prophylaxis of pathologies linked to metabolism of the lipids and/or glucids comprising administering to a subject an effective quantity of at least one compound represented by formula (I) or an effective quantity of pharmaceutical composition such as defined in claim 16.


SEQUENCE LISTING (6 sequences)

SEQ ID NO: 1 - DNA, 20 bp, Artificial
  ACO specific primer
ttggaaaccactgccacata

SEQ ID NO: 2 - DNA, 20 bp, Artificial
  ACO specific primer
aggaccaatgtctcccacag

SEQ ID NO: 3 - DNA, 20 bp, Artificial
  PDK4 specific primer
agagcctgatggatttggtg

SEQ ID NO: 4 - DNA, 20 bp, Artificial
  PDK4 specific primer
ttgattggtgactgggtcaa

SEQ ID NO: 5 - DNA, 22 bp, Artificial
  18S specific primer
cggacacggacaggattgacag

SEQ ID NO: 6 - DNA, 20 bp, Artificial
  18S specific primer
aatctcgggtggctgaacgc